(19) United States
(12) Patent Application Publication
(10) Pub. No.: US 2025/0059175 A1
(43) Pub. Date: Feb. 20, 2025

(54) Mutant Pi3k-Alpha Inhibitors And Their Use As Pharmaceuticals

(71) Applicant: Prelude Therapeutics Incorporated, Wilmington, DE (US)

(72) Inventors: Andrew Buesking, Wilmington, DE (US); Ryan Holmes, Wilmington, DE (US)

(21) Appl. No.: 18/805,657

(22) Filed: Aug. 15, 2024

Related U.S. Application Data
(60) Provisional application No. 63/519,775, filed on Aug. 15, 2023.
(60) Provisional application No. 63/671,849, filed on Jul. 16, 2024.

(51) Int. Cl. C07D 413/14; A61K 31/4439; A61K 31/444; A61K 31/5355; A61K 31/5377; C07D 401/14; C07D 413/04; C07D 413/12

(52) U.S. Cl. CPC C07D 413/14; A61K 31/4439; A61K 31/444; A61K 31/5355; A61K 31/5377; C07D 401/14; C07D 413/04; C07D 413/12

(57) ABSTRACT

The disclosure is directed to compounds of Formula I

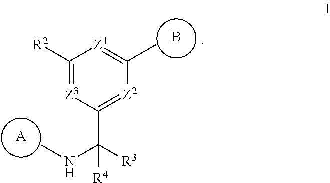

Pharmaceutical compositions comprising compounds of Formula I, as well as methods of their use and preparation, are also described.

### CROSS REFERENCE TO RELATED APPLICATIONS

This application is related to U.S. Patent Application No. 63/519,775, filed Aug. 15, 2023, and U.S. Patent Application No. 63/671,849, filed Jul. 16, 2024, the disclosures of each of which are incorporated herein in their entireties.

### TECHNICAL FIELD

The disclosure is directed to mutant PI3Kα inhibitors and methods of their use.

### BACKGROUND

Class I PI3Ks consist of a p85 regulatory subunit in complex with a p110 catalytic subunit (p110α, β, γ or δ) (1). p110α, coded by the PIK3CA gene shows a broad tissue distribution and the binding of a phosphorylated receptor tyrosine kinase (RTK) activates p110a through the release of a subset of inhibitory contacts with p85. P110α generate phosphatidylinositol3,4,5-trisphosphate (PtdIns(3, 4, 5)P3; also known as PIP3), which interact with 3-phosphoinositide-binding Pleckstrin homology (PH) domains found in diverse proteins, including protein kinases such as AKT resulting in its phosphorylation at Thr308 and Ser473 triggering a cascade of mitogenic signaling (2). This signaling results in a multitude of cellular effects including proliferation, survival, chemotaxis, cellular trafficking, motility, metabolism, inflammatory and allergic responses, transcription and translation (3).

PIK3CA hotspot mutations are one of the most frequent oncogenic mutations in cancer. Common hotspot mutations in PIK3CA helical (E542K, E545K) and kinase (H1047R) domains function by perturbing local interfaces between p85 and p110α and increasing dynamic events required for catalysis on membranes (1,4). Oncogenic mutations in the PIK3CA gene increase lipid kinase activity and transform cells and are the drivers of the pathology. These mutations are observed in a broad range of cancers including breast, colon, uterine, bladder, cervical, and lung cancer (5, 6, 7).

Given its key role in cancer PI3Ks have been the focus of extensive drug development. In 2017, the pan-class I PI3K inhibitor copanlisib (Aligopa/BAY 80-6946; Bayer) was approved for follicular lymphoma and in 2019, the PI3Kα inhibitor alpelisib (Piqray/NVP-BYL719; Novartis) was approved for the treatment of advanced breast cancer, in combination with the estrogen receptor (ER) downregulator fulvestrant (1,8). There are a number of PI3Kα selective inhibitors that are in late stage clinical trials (1).

Although these approvals and early clinical data have validated the pathway as a viable drug target, the development of PI3K inhibitors has proved challenging, with progress hampered by poor drug tolerance (9). The lack of clinical benefit and poor tolerability of pan-class I PI3K and dual PI3Kα/PI3Kδ, or even PI3Kα selective inhibitors have impacted the realization of full clinical utility of these compounds. The toxicity of PI3K inhibitors is dependent on their isoform selectivity profile. Inhibition of PI3Kα is associated with hyperglycemia and rash, whereas inhibition of PI3Kδ or PI3Kγ is associated with diarrhea, myelosuppression, and transaminitis (1). A recent study reported that while progression of disease is the largest contributor to alpelisib discontinuation, adverse events are the leading cause for early drug cessation (10). Shorter alpelisib exposure is associated with greater cancer progression. Therefore, selective inhibitors of PI3Kα mutants H1047R and/or E545K/E543K while sparing wild type PI3Kα, β, γ or δ could enhance the therapeutic margin and result in therapies that benefit cancer patients that carry these mutations.

Additional small molecule mutant PI3Kα selective inhibitors are needed.
(1) PI3K inhibitors are finally coming of age. Vanhaesebroeck B, Perry M W D, Brown J R, André F, Okkenhaug K. Nat Rev Drug Discov. 2021 October; 20(10):741-769. doi: 10.1038/s41573-021-00209-1. Epub 2021 Jun. 14. PMID: 34127844.
(2) AKT/PKB Signaling: Navigating the Network. Manning B D, Toker A. Cell. 2017 Apr. 20; 169(3):381-405. doi: 10.1016/j.cell.2017.04.001. PMID: 28431241.
(3) Fruman, D. A. et al. The PI3K pathway in human disease. Cell 170, 605-635 (2017). PMID: 28802037 PMCID: PMC5726441 DOI: 10.1016/j.cell.2017.07.029.
(4) PIK3C A mutations in advanced cancers: characteristics and outcomes. Janku F, Wheler J J, Naing A, Stepanek V M, Falchook G S, Fu S, Garrido-Laguna I, Tsimberidou A M, Piha-Paul S A, Moulder S L, Lee J J, Luthra R, Hong D S, Kurzrock R. Oncotarget. 2012 December; 3(12):1566-75. doi: 10.18632/oncotarget.716. PMID: 23248156.
(5) Frequency and spectrum of PIK3CA somatic mutations in breast cancer. Martínez-Sáez O, Chic N, Pascual T, Adamo B, Vidal M, González-Farré B, Sanfeliu E, Schettini F, Conte B, Brasó-Maristany F, Rodríguez A, Martínez D, Galván P, Rodríguez A B, Martinez A, Muñoz M, Prat A. Breast Cancer Res. 2020 May 13; 22(1):45. doi: 10.1186/s13058-020-01284-9. PMID: 32404150.
(6) Cancer-specific mutations in PIK3CA are oncogenic in vivo. Bader A G, Kang S, Vogt P K. Proc Natl Acad Sci USA. 2006 Jan. 31; 103(5):1475-9. doi: 10.1073/pnas.0510857103. Epub 2006 Jan. 23. PMID: 16432179.
(7) Oncogenic Signaling Pathways in The Cancer Genome Atlas. Sanchez-Vega F, Mina M, Armenia J, Chatila W K, Luna A, La K C, Dimitriadoy S, Liu D L, Kantheti H S, Saghafinia S, Chakravarty D, Daian F, Gao Q, Bailey M H, Liang W W, Foltz S M, Shmulevich I, Ding L, Heins Z, Ochoa A, Gross B, Gao J, Zhang H, Kundra R, Kandoth C, Bahceci I, Dervishi L, Dogrusoz U, Zhou W, Shen H, Laird P W, Way G P, Greene C S, Liang H, Xiao Y, Wang C, Iavarone A, Berger A H, Bivona T G, Lazar A J, Hammer G D, Giordano T, Kwong L N, McArthur G, Huang C, Tward A D, Frederick M J, McCormick F, Meyerson M; Cancer Genome Atlas Research Network, Van Allen E M, Cherniack A D, Ciriello G, Sander C, Schultz N. Cell. 2018 Apr. 5; 173(2):321-337.e10. doi: 10.1016/j.cell.2018.03.035. PMID: 29625050.
(8) Alpelisib for PIK3CA-Mutated, Hormone Receptor-Positive Advanced Breast Cancer. André F, Ciruelos E, Rubovszky G, Campone M, Loibl S, Rugo H S, Iwata H, Conte P, Mayer I A, Kaufman B, Yamashita T, Lu Y S, Inoue K, Takahashi M, Pipai Z, Longin A S, Mills D, Wilke C, Hirawat S, Juric D; SOLAR-1 Study Group. N Engl J Med. 2019 May 16; 380(20):1929-1940. doi: 10.1056/NEJMoa1813904. PMID: 31091374.
(9) A multidisciplinary approach to optimizing care of patients treated with alpelisib. Rugo H S, Lacouture M E, Goncalves M D, Masharani U, Aapro M S, O'Shaughnessy J A. Breast. 2022 February; 61:156-167. doi: 10.1016/j.breast.2021.12.016. Epub 2021 Dec. 27. PMID: 35016012.
(10) Factors leading to alpelisib discontinuation in patients with hormone receptor positive, human epidermal growth factor receptor-2 negative breast cancer. Cheung Y M, Cromwell G E, Tolaney S M, Min L, McDonnell M E. Breast Cancer Res Treat. 2022 April; 192(2):303-311. doi: 10.1007/s10549-021-06476-1. Epub 2022 Jan. 9. PMID: 35000092.

### SUMMARY OF THE INVENTION

The disclosure is directed to compounds of Formula I:

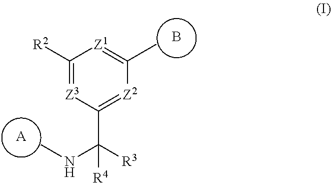

or a pharmaceutically acceptable salt, N-oxide, or stereoisomer thereof, wherein
Ring A is aryl or a 5-7 membered heteroaryl ring comprising 1-4 heteroatoms selected from N, O, and S, wherein in the aryl or 5-7 membered heteroaryl ring is optionally substituted with one or more groups independently selected from D, halogen, C₁-C₈ alkoxy, C₁-C₈ haloalkoxy, C₁-C₈ alkyl, haloalkyl, —OH, —CN, —NO2, —C2-C6 alkenyl, —C2-C6 alkynyl, aryl, heteroaryl, cycloalkyl, cycloalkenyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)2NR^{c}R^{d}, —S(O)(═NR^{b})R^{b}, —SF5, —P(O)R^{b}R^{b}, —P(O)R^{c}R^{d}, —P(O)(OR^{b})(OR^{b}), —B(OR^{c})(OR^{d}), —SiR^{b}₃, —S(O)2R^{b}, —C(O)NR^{b}OR^{b}, —S(O)₂OR^{b}, —OS(O)₂OR^{b}, and —OPO(OR^{b})(OR^{b}); wherein each C₁-C₈ alkyl or haloalkyl is optionally substituted by 1-6 groups selected from D, halogen, —OH, —CN, —OR^{a}, —SR^{a}, —NR^{a}R^{d}, or NR^{c}R^{d};
Ring B is 4-13 membered heterocycloalkyl, 4-10 membered heterocycloalkenyl, or 4-10 membered heteroaryl, wherein the 4-13 membered heterocycloalkyl, 4-10 membered heterocycloalkenyl, or 4-10 membered heteroaryl is optionally substituted with one or more groups independently selected from D, oxo, ═NR^{a}, ═N—OR^{a}, ═N—CN, ═S, halogen, C₁-C₈ alkoxide, C₁-C₁₀ alkyl, haloalkyl, —OH, —CN, —NO₂, —C₂-C₁₀ alkenyl, —C₂-C₁₀ alkynyl, aryl, heteroaryl, cycloalkyl, cycloalkenyl, heterocycloalkyl, heterocycloalkenyl, C₀-C₄alk-aryl, C₀-C₄alk-heteroaryl, —OR^{a}, —SR^{a}, —NR^{c}R^{d}, —NR^{a}R^{c}, —C(O)R^{b}, —OC(O)R^{b}, —C(O)OR^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —C(═NR^{b})NR^{c}R^{d}, —C(═NOR^{b})NR^{c}R^{d}, —C(═NCN)NR^{c}R^{d}, —P(OR^{c})₂, —P(O)R^{c}R^{b}, —P(O)R^{c}R^{d}, —P(O)OR^{c}OR^{b}, —S(O)R^{b}, —S(O)NR^{c}R^{d}, —S(O)2NR^{c}R^{d}, —S(O)(═NR^{b})R^{b}, —SF5, —P(O)R^{b}R^{b}, —P(O)(OR^{b})(OR^{b}), —B(OR^{c})(OR^{d}), —S(O)2R^{b}, —C(O)NR^{b}OR^{b}, —SiR^{b}₃, —S(O)₂OR^{b}, —OS(O)₂OR^{b}, —OPO(OR^{b})(OR^{b}) and -L-W; wherein each C₁-C₈ alkoxide, C₁-C₁₀ alkyl, haloalkyl, —C2-C10 alkenyl, —C2-C10 alkynyl, aryl, heteroaryl, cycloalkyl, cycloalkenyl, heterocycloalkyl, heterocycloalkenyl, C₀-C₄alk-aryl, or C₀-C₄alk-heteroaryl is optionally substituted by 1-6 R^{f} groups;
L is absent or is C₁-C₈ alkylene, —O—, —N(R^{a})—, —S— or 3-10 membered cycloalkylene;
W is a 5-10 membered heteroaryl ring comprising 1-4 heteroatoms selected from N, O, and S; a 5-12 membered heterocyclic group comprising 1-4 heteroatoms selected from N, O, and S; C₁-C₈ alkyl; haloalkyl; —C2-C6 alkenyl; —C2-C6 alkynyl; aryl; cycloalkyl; cycloalkenyl; heterocyclo-alkenyl; NR^{c}R^{d}; OR^{b}; or SR^{b}; each of which is optionally substituted by 1-6 R^{f} groups;
Z¹, Z², and Z³ are each independently CR² or N;
each R² is independently H, D, halogen, C₁-C₈ alkoxide, C₁-C₈ alkyl, haloalkyl, —OH, —CN, —NO2, —C2-C6 alkenyl, —C2-C6 alkynyl, aryl, heteroaryl, cycloalkyl, cycloalkenyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)2NR^{c}R^{d}, —S(O)(═NR^{b})R^{b}, —SF5, —P(O)R^{b}R^{b}, —P(O)R^{c}R^{d}, —P(O)(OR^{b})(OR^{b}), —B(OR^{c})(OR^{d}), SiR^{b}₃, —S(O)2R^{b}, —C(O)NR^{b}OR^{b}, —S(O)₂OR^{b}, —OS(O)₂OR^{b}, or —OPO(OR^{b})(OR^{c}); wherein said C₁-C₈ alkyl is optionally substituted by 1-6 groups selected from D, halogen, —OH, —CN, —OR^{a}, —SR^{a}, —NR^{a}R^{d}, or NR^{c}R^{d};
each R³ and R⁴ is independently H, D, C₁-C₈ alkyl, haloalkyl, or CN; wherein said C₁-C₈ alkyl is optionally substituted by 1-6 groups selected from D, halogen, —OH, —CN, —OR^{a}, —SR^{a}, —NR^{a}R^{d}, or —NR^{c}R^{d};
or R³ and R⁴, together with the atom to which they are both attached, are combined to form a C₃-C₇ cycloalkyl or C₄-C₈ heterocycloalkyl, wherein the C₃-C₇ cycloalkyl or C₄-C₈ heterocycloalkyl is optionally substituted by 1-6 groups selected from D, halogen, —OH, —CN, —OR^{a}, —SR^{a}, —NR^{a}R^{d}, or —NR^{c}R^{d};
each R^{a} is independently H, D, —C(O)R^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —C(═NR^{b})NR^{c}R^{d}, —C(═NOR^{b})NR^{c}R^{d}, —C(═NCN)NR^{c}R^{d}, —P(OR^{c})₂, —P(O)R^{c}R^{b}, —P(O)R^{c}R^{d}, —P(O)OR^{c}OR^{b}, —S(O)R^{b}, —S(O)NR^{c}R^{d}, —S(O)₂R^{b}, —S(O)₂NR^{c}R^{d}, —SiR^{b}₃, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl, wherein each —C₁-C₁₀alkyl, —C₂-C₁₀ alkenyl, —C₂-C₁₀ alkynyl, aryl, cycloalkyl, cycloalkenyl, heteroaryl, heterocycloalkyl, and heterocycloalkenyl of R^{a} is optionally substituted by 1-6 R^{f} groups;
each R^{b} is independently H, D, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl; wherein each —C₁-C₆ alkyl, —C₂-C₆ alkenyl, —C₂-C₆ alkynyl, aryl, cycloalkyl, cycloalkenyl, heteroaryl, heterocycloalkyl, and heterocycloalkenyl of R^{b} is optionally substituted by 1-6 R^{f} groups;
each R^{c} or R^{d} is independently H, D, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl; wherein each —C₁-C₁₀ alkyl, —C₂-C₆ alkenyl, —C₂-C₆ alkynyl, —OC₁-C₆alkyl, —O— cycloalkyl, aryl, heteroaryl, cycloalkyl, cycloalkenyl, heterocycloalkyl of R^{c} or R^{d} is optionally substituted by 1-6 R^{f} groups;
or R^{c} and R^{d}, together with the atom to which they are both attached, form a monocyclic or multicyclic heterocycloalkyl, or a monocyclic or multicyclic heterocycloalkenyl group, wherein the monocyclic or multicyclic heterocycloalkyl, or a monocyclic or multicyclic heterocycloalkenyl group is optionally substituted by 1-6 R^{f} groups;
each R^{f} is independently D, oxo, halogen, C₁-C₈ alkoxide, C₁-C₈ alkyl, haloalkyl, —OH, —CN, —NO2, —C2-C6 alkenyl, —C2-C6 alkynyl, aryl, heteroaryl, cycloalkyl, cycloalkenyl, heterocycloalkyl, heterocycloalkenyl, —OR^{g0}, —SR^{g0}, —NR^{g2}R^{g3}, —NR^{g0}R^{g2}, —C(O)R^{g1}, —OC(O)R^{g1}, —C(O)OR^{g1}, —C(O)NR^{g2}R^{g3}, —S(O)R^{g1}, —S(O)2NR^{g2}R^{g3}, —S(O)(═NR^{g1})R^{g1}, —SF5, —P(O)R^{g1}R^{g1}, —P(O)R^{g2}R^{g3}, —P(O)(OR^{g1})(OR^{g1}), SiR^{g1}₃, —B(OR^{g2})(OR^{g3}), —S(O)2R^{g1}, —C(O)NR^{g1}OR^{g1}, —S(O)₂OR^{g1}, —OS(O)₂OR^{g1}, or —OPO(OR^{g1})(OR^{g1}); wherein each C₁-C₈ alkoxide, C₁-C₈ alkyl, haloalkyl, —C2-C6 alkenyl, —C2-C6 alkynyl, aryl, heteroaryl, cycloalkyl, cycloalkenyl, heterocycloalkyl, or heterocycloalkenyl is optionally substituted by 1-6 R^{g} groups;
each R^{g0} is independently H, D, —C(O)R^{g1}, —C(O)OR^{g2}, —C(O)NR^{g2}R^{g3}, —C(═NR^{g1})NR^{g2}R^{g3}, —C(═NOR^{g1})NR^{g2}R^{g3}, —C(═NCN)NR^{g1}R^{g2}, —C(═NR^{g1})NR^{g2}R^{g3}, —C(═NOR^{g1})NR^{g2}R^{g3}, —C(═NCN)NR^{g2}R^{g3}, —P(OR^{g2})₂, —P(O)R^{g2}R^{g1}, —P(O)R^{g2}R^{g3}, —P(O)OR^{g2}OR^{g1}, —S(O)R^{g1}, —S(O)NR^{g2}R^{g3}, —S(O)₂R^{g1}, —S(O)₂NR^{g2}R^{g3}, —SiR^{g1}₃, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl, wherein each —C₁-C₁₀alkyl, —C₂-C₁₀ alkenyl, —C₂-C₁₀ alkynyl, aryl, cycloalkyl, cycloalkenyl, heteroaryl, heterocycloalkyl, and heterocycloalkenyl is optionally substituted by 1-6 R^{g} groups;
each R^{g1} is independently H, D, C₁-C₈ alkyl, haloalkyl, —C2-C6 alkenyl, —C2-C6 alkynyl, aryl, heteroaryl, cycloalkyl, cycloalkenyl, heterocycloalkyl, or heterocycloalkenyl, wherein each C₁-C₈ alkyl, haloalkyl, —C2-C6 alkenyl, —C2-C6 alkynyl, aryl, heteroaryl, cycloalkyl, cycloalkenyl, heterocycloalkyl, or heterocycloalkenyl is optionally substituted by 1-6 R^{g} groups;
each R^{g2} or R^{g3} is independently H, D, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl; wherein each —C₁-C₁₀ alkyl, —C₂-C₆ alkenyl, —C₂-C₆ alkynyl, —OC₁-C₆alkyl, —O— cycloalkyl, aryl, heteroaryl, cycloalkyl, cycloalkenyl, or heterocycloalkyl is optionally substituted by 1-6 R^{g} groups; or R^{g2} and R^{g3}, together with the atom to which they are both attached, form a monocyclic or multicyclic heterocycloalkyl, or a monocyclic or multicyclic heterocycloalkenyl group, wherein the monocyclic or multicyclic heterocycloalkyl, or monocyclic or multicyclic heterocycloalkenyl group is optionally substituted by 1-6 R^{g} groups;
each R^{g} is independently:
D; —CN; —NO2; oxo; halogen; —SF₅; —OR^{h}; —SR^{h}; —NR^{h}R^{h}; —SiR^{h}₃; —C(O)R^{h}; —OC(O)R^{h}; —C(O)OR^{h}; —OC(O)OR^{h}; —C(O)NR^{h}OR^{h}; —S(O)R^{h}; —S(O)₂R^{h}; —S(O)₂OR^{h}; —OS(O)₂OR^{h}; —S(O)(═NR^{h})R^{h}; —P(O)(OR^{h})(OR^{h}); —P(O)R^{h}R^{h}; OPO(OR^{h})(OR^{h}); —C(O)NR^{h}R^{h}; —OC(O)NR^{h}R^{h}; —S(O)₂NR^{h}R^{h}; —B(OR^{h})(OR^{h}); —OC(═NR^{h})NR^{h}R^{h}; —OC(═NOR^{h})NR^{h}R^{h}; —OC(═NCN)NR^{h}R^{h}; —OP(OR^{h})₂; —OP(O)R^{h}R^{h}; —OP(O)OR^{h}OR^{h}; —OS(O)R^{h}; —OS(O)NR^{h}R^{h}; —OS(O)₂R^{h}; —OS(O)₂NR^{h}R^{h}; —OSiR^{h}₃; —S—C(O)R^{h}; —S—C(O)OR^{h}; —S—C(O)NR^{h}R^{h}; —S—C(═NR^{h})NR^{h}R^{h}; —S—C(═NOR^{h})NR^{h}R^{h}; —C(═NCN)NR^{h}R^{h}; —S—P(OR^{h})₂; —S—P(O)R^{h}R^{h}; —S—P(O)OR^{h}OR^{h}; —S(O)R^{h}; —S(O)NR^{h}R^{h}; —S(O)₂R^{h}; —S(O)₂NR^{h}R^{h}; —SSiR^{h}₃; —NR^{h}C(O)R^{h}; —NR^{h}C(O)OR^{h}; —NR^{h}C(O)NR^{h}R^{h}; —NR^{h}C(═NR^{h})NR^{h}R^{h}; —NR^{h}C(═NOR^{h})NR^{h}R^{h}; —NR^{h}C(═NCN)NR^{h}R^{h}; —NR^{h}P(OR^{h})₂; —NR^{h}P(O)R^{h}R^{h}; —NR^{h}P(O)OR^{h}OR^{h}; —NR^{h}S(O)R^{h}; —NR^{h}S(O)NR^{h}R^{h}; —NR^{h}S(O)₂R^{h}; —NR^{h}S(O)₂NR^{h}R^{h}; —NR^{h}SiR^{h}₃; C₁-C₈ alkoxide; C₁-C₈ alkyl; haloalkyl; —C2-C6 alkenyl; —C2-C6 alkynyl; aryl; heteroaryl; cycloalkyl; cycloalkenyl; heterocycloalkyl; or heterocycloalkenyl; wherein the C₁-C₈ alkoxide, C₁-C₈ alkyl, haloalkyl, —C2-C6 alkenyl, —C2-C6 alkynyl, aryl, heteroaryl, cycloalkyl, cycloalkenyl, heterocycloalkyl, or heterocycloalkenyl is optionally substituted by 1-6 groups selected from D, halogen, —CN, —NO2, oxo, —OR^{h}; —SR^{h}; or —NR^{h}R^{h};
wherein each R^{h} is independently H, D, C₁-C₈alkyl, haloalkyl, —C2-C6 alkenyl, —C2-C6 alkynyl, aryl, heteroaryl, cycloalkyl, cycloalkenyl, heterocycloalkyl, or heterocycloalkenyl; or wherein two R^{h} attached to the same atom may form a monocyclic or multicyclic heterocycloalkyl, or a monocyclic or multicyclic heterocycloalkenyl group.

Stereoisomers of the compounds of Formula I, and the pharmaceutical salts and stereoisomers thereof, are also contemplated, described, and encompassed herein. Methods of using compounds of Formula I are described, as well as pharmaceutical compositions including the compounds of Formula I.

### DETAILED DESCRIPTION OF ILLUSTRATIVE EMBODIMENTS

The disclosure may be more fully appreciated by reference to the following description, including the following definitions and examples. Certain features of the disclosed compositions and methods which are described herein in the context of separate aspects, may also be provided in combination in a single aspect. Alternatively, various features of the disclosed compositions and methods that are, for brevity, described in the context of a single aspect, may also be provided separately or in any subcombination.

At various places in the present specification, substituents of compounds of the invention are disclosed in groups or in ranges. It is specifically intended that the invention include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. “C₀ alkyl” refers to a covalent bond.

It is further intended that the compounds of the invention are stable. As used herein “stable” refers to a compound that is sufficiently robust to survive isolation to a useful degree of purity from a reaction mixture, and preferably capable of formulation into an efficacious therapeutic agent.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable sub-combination.

The term “alkyl,” when used alone or as part of a substituent group, refers to a straight- or branched-chain hydrocarbon group having from 1 to 12 carbon atoms (“C₁-C₁₂”), preferably 1 to 6 carbons atoms (“C₁-C₆”), in the group. Examples of alkyl groups include methyl (Me, C₁alkyl), ethyl (Et, C₂alkyl), n-propyl (C₃alkyl), isopropyl (C₃alkyl), butyl (C₄alkyl), isobutyl (C₄alkyl), sec-butyl (C₄alkyl), tert-butyl (C₄alkyl), pentyl (C₅alkyl), isopentyl (C₅alkyl), tert-pentyl (C₅alkyl), hexyl (C₆alkyl), isohexyl (C₆alkyl), and the like. Alkyl groups of the disclosure are optionally substituted. Unless otherwise specified, in those embodiments wherein the alkyl group is substituted, the alkyl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the alkyl group is optionally substituted by 1-6 groups selected from D, halogen, —OH, —CN, —OR^{a}, —SR^{a}, —NR^{a}R^{d}, or NR^{c}R^{d}; or the alkyl group is optionally substituted by 1-6 R¹ groups.

The term “halo” or halogen refers to chloro, fluoro, bromo, or iodo.

The term “cycloalkyl” when used alone or as part of a substituent group refers to cyclic-containing, non-aromatic hydrocarbon groups having from 3 to 10 carbon atoms (“C₃-C₁₀”), preferably from 3 to 6 carbon atoms (“C₃-C₆”). Cycloalkyl groups of the disclosure include monocyclic groups, as well as multicyclic groups such as bicyclic and tricyclic groups. In those embodiments having at least one multicyclic cycloalkyl group, the cyclic groups can share one common atom (i.e., spirocyclic). In other embodiments having at least one multicyclic cycloalkyl group, the cyclic groups share two common atoms (e.g., fused or bridged). Examples of cycloalkyl groups include, for example, cyclopropyl (C₃), cyclobutyl (C₄), cyclopropylmethyl (C₄), cyclopentyl (C₅), cyclohexyl (C₆), 1-methylcyclopropyl (C₄), 2-methylcyclopentyl (C₄), adamantanyl (C₁₀), spiro[3.3]heptanyl, bicyclo[3.3.0]octanyl, and the like. Cycloalkyl groups of the disclosure are optionally substituted. Unless otherwise specified, in those embodiments wherein the cycloalkyl group is substituted, the cycloalkyl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the cycloalkyl group is optionally substituted by 1-6 groups selected from D, halogen, —OH, —CN, —OR^{a}, —SR^{a}, —NR^{a}R^{d}, or NR^{c}R^{d}; or the cycloalkyl group is optionally substituted by 1-6 R^{f} groups.

The term “cycloalkenyl” when used alone or as part of a substituent group refers to monocyclic or multicyclic, partially saturated ring structure having from 3 to 10 carbon atoms (“C₃-C₁₀”), preferably from 3 to 6 carbon atoms (“C₃-C₆”). Cycloalkenyl groups of the disclosure include monocyclic groups, as well as multicyclic groups such as bicyclic and tricyclic groups. In those embodiments having at least one multicyclic cycloalkenyl group, the cyclic groups can share one common atom (i.e., spirocyclic). In other embodiments having at least one multicyclic cycloalkenyl group, the cyclic groups share two common atoms (e.g., fused or bridged). The term —C₃-C₆ cycloalkenyl refers to a cycloalkenyl group having between three and six carbon atoms. The cycloalkenyl group may be attached at any carbon atom of the partially saturated ring such that the result is a stable structure. Cycloalkenyl groups include groups in which the partially saturated ring is fused to an aryl group. Examples of cycloalkenyl groups include, for example, cyclopropenyl (C₃), cyclobutenyl (C₄), cyclopropenylmethyl (C₄), cyclopentenyl (C₅), cyclohexenyl (C₆), 1-methylcyclopropenyl (C₄), 2-methylcyclopentenyl (C₄), adamantenyl (C₁₀), spiro[3.3]heptenyl, bicyclo[3.3.0]octenyl, indanyl, and the like. Cycloalkenyl groups of the disclosure are optionally substituted. Unless otherwise specified, in those embodiments wherein the cycloalkenyl group is substituted, the cycloalkenyl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the cycloalkenyl group is optionally substituted by 1-6 groups selected from D, halogen, —OH, —CN, —OR^{a}, —SR^{a}, —NR^{a}R^{d}, or NR^{c}R^{d}; or the cycloalkenyl group is optionally substituted by 1-6 R^{f} groups.

The term “heterocycloalkyl” when used alone or as part of a substituent group refers to any three to twelve membered monocyclic or multicyclic, saturated ring structure containing at least one heteroatom selected from the group consisting of O, N and S. Heterocycloalkyl groups of the disclosure include monocyclic groups, as well as multicyclic groups such as bicyclic and tricyclic groups. In those embodiments having at least one multicyclic heterocycloalkyl group, the cyclic groups can share one common atom (i.e., spirocyclic). In other embodiments having at least one multicyclic heterocycloalkyl group, the cyclic groups share two common atoms (e.g., fused or bridged). The term —C₃-C₆ heterocycloalkyl refers to a heterocycloalkyl group having between three and six carbon ring atoms. The heterocycloalkyl group may be attached at any heteroatom or carbon atom of the group such that the result is a stable structure. Examples of heterocycloalkyl groups include, but are not limited to, azepanyl, aziridinyl, azetidinyl, pyrrolidinyl, dioxolanyl, imidazolidinyl, pyrazolidinyl, piperazinyl, piperidinyl, dioxanyl, morpholinyl, dithianyl, thiomorpholinyl, oxazepanyl, oxiranyl, oxetanyl, quinuclidinyl, tetrahydrofuranyl, tetrahydro-pyranyl, piperazinyl, azepanyl, diazepanyl, oxepanyl, dioxepanyl, azocanyl diazocanyl, oxocanyl, dioxocanyl, azaspiro[2.2]pentanyl, oxaazaspiro[3.3]heptanyl, oxaspiro[3.3]heptanyl, dioxaspiro[3.3]heptanyl, 3-azabicyclo[3.1.0]hexanyl,

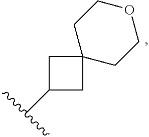 [SMILES: *C1CC2(CCOCC2)C1]

and the like. Heteroycloalkyl groups of the disclosure are optionally substituted. Unless otherwise specified, in those embodiments wherein the heterocycloalkyl group is substituted, the heterocycloalkyl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the heterocycloalkyl group is optionally substituted by 1-6 groups selected from D, halogen, —OH, —CN, —OR^{a}, —SR^{a}, —NR^{a}R^{d}, or NR^{c}R^{d}; or the heterocycloalkyl group is optionally substituted by 1-6 R^{f} groups.

The term “heterocycloalkenyl” when used alone or as part of a substituent group refers to any three to twelve membered monocyclic or multicyclic, partially saturated ring structure containing at least one heteroatom selected from the group consisting of O, N and S. Heterocycloalkenyl groups of the disclosure include monocyclic groups, as well as multicyclic groups such as bicyclic and tricyclic groups. In those embodiments having at least one multicyclic heterocycloalkyenyl group, the cyclic groups can share one common atom (i.e., spirocyclic). In other embodiments having at least one multicyclic heterocycloalkenyl group, the cyclic groups share two common atoms (e.g., fused or bridged). The term —C₃-C₆ heterocycloalkenyl refers to a heterocycloalkenyl group having between three and six carbon atoms. The heterocycloalkenyl group may be attached at any heteroatom or carbon atom of the partially saturated ring such that the result is a stable structure. Heterocycloalkenyl groups include groups in which the partially saturated ring is fused to an aryl group, such as, for example isoindoline,

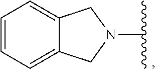 [SMILES: *N1Cc2ccccc2C1]

or in which the partially saturated ring is fused to a heteroaryl group, such as, for example, 6,7-dihydro-5H-pyrrolo[3,4-b]pyridine,

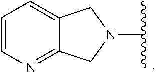 [SMILES: *N1Cc2cccnc2C1]

Heteroycloalkenyl groups of the disclosure are optionally substituted. Unless otherwise specified, in those embodiments wherein the heterocycloalkenyl group is substituted, the heterocycloalkenyl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the heterocycloalkenyl group is optionally substituted by 1-6 groups selected from D, halogen, —OH, —CN, —OR^{a}, —SR^{a}, —NR^{a}R^{d}, or NR^{c}R^{d}; or the heterocycloalkenyl group is optionally substituted by 1-6 R^{f} groups.

The term “heterocyclic group,” when used alone or as part of a substituent group, refers to a heterocycloalkyl group or a heterocycloalkenyl group.

The term “heteroaryl” when used alone or as part of a substituent group refers to a mono- or bicyclic-aromatic ring structure including carbon atoms as well as up to five heteroatoms selected from nitrogen, oxygen, and sulfur. Heteroaryl rings can include a total of 5, 6, 7, 8, 9, or 10 ring atoms. Examples of heteroaryl groups include but are not limited to, pyrrolyl, furyl, thiophenyl (thienyl), oxazolyl, imidazolyl, pyrazolyl, isoxazolyl, isothiazolyl, triazolyl, thiadiazolyl, pyrazolyl, pyridinyl, pyridazinyl, pyrimidinyl, pyrazinyl, pyranyl, furazanyl, indolizinyl, indolyl, and the like. Heteroaryl groups of the disclosure are optionally substituted. Unless otherwise specified, in those embodiments wherein the heteroaryl group is substituted, the heteroaryl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the heteroaryl group is optionally substituted by 1-6 groups selected from D, halogen, —OH, —CN, —OR^{a}, —SR^{a}, —NR^{a}R^{d}, or NR^{c}R^{d}; or the heteroaryl group is optionally substituted by 1-6 R^{f} groups.

The term “aryl” when used alone or as part of a substituent group refers to a mono- or bicyclic-aromatic carbon ring structure. Aryl rings can include a total of 5, 6, 7, 8, 9, or 10 ring atoms. Examples of aryl groups include but are not limited to, phenyl, napthyl, and the like. Aryl groups of the disclosure are optionally substituted. Unless otherwise specified, in those embodiments wherein the aryl group is substituted, the aryl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the aryl group is optionally substituted by 1-6 groups selected from D, halogen, —OH, —CN, —OR^{a}, —SR^{a}, —NR^{a}R^{d}, or NR^{c}R^{d}; or the aryl group is optionally substituted by 1-6 R^{f} groups.

When a range of carbon atoms is used herein, for example, C₁-C₆, all ranges, as well as individual numbers of carbon atoms are encompassed, for example, “C₁₋₃” includes C₁₋₃, C₁₋₂, C₂₋₃, C₁, C₂, and C₃. The term “C₁₋₆alk” refers to an aliphatic linker having 1, 2, 3, 4, 5, or 6 carbon atoms and includes, for example, —CH₂—, —CH(CH₃)—, —CH(CH₃)—CH₂—, and —C(CH₃)₂—. The term “—C₀alk-” refers to a bond.

The term “C₀-C₆alk” when used alone or as part of a substituent group refers to an aliphatic linker having 0, 1, 2, 3, 4, 5 or 6 carbon atoms. The term “—C₁alk-”, for example, refers to a —CH₂—. The term “—C₀alk-” refers to a bond.

Unless otherwise specified, in those embodiments wherein the —C₁-C₆alkyl, —C₁-C₁₀ alkyl, —C₁-C₈ alkoxide, —C₂-C₆alkenyl, —C₂-C₁₀alkenyl, —C₂-C₆alkynyl, —C₂-C₁₀alkynyl, aryl, heteroaryl, cycloalkyl, cycloalkenyl, heterocycloalkenyl, and heterocycloalkyl groups are substituted, they can be optionally substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the —C₁-C₆alkyl, —C₁-C₁₀ alkyl, —C₁-C₈ alkoxide, —C₂-C₆alkenyl, —C₂-C₁₀alkenyl, —C₂-C₆alkynyl, —C₂-C₁₀alkynyl, aryl, heteroaryl, cycloalkyl, cycloalkenyl, heterocycloalkenyl, and heterocycloalkyl groups are optionally substituted by 1-6 groups selected from D, halogen, —OH, —CN, —OR^{a}, —SR^{a}, —NR^{a}R^{d}, or NR^{c}R^{d}; or the —C₁-C₆alkyl, —C₁-C₁₀ alkyl, —C₁-C₈ alkoxide, —C₂-C₆alkenyl, —C₂-C₁₀alkenyl, —C₂-C₆alkynyl, —C₂-C₁₀ alkynyl, aryl, heteroaryl, cycloalkyl, cycloalkenyl, heterocycloalkenyl, and heterocycloalkyl groups are optionally substituted by 1-6 R^{f} groups.

As used herein, “alkoxy” refers to an —O-alkyl group. Example alkoxy groups include methoxy, ethoxy, propoxy (e.g., n-propoxy and isopropoxy), t-butoxy, and the like.

As used herein, “hydroxylalkyl” refers to an alkyl group substituted by OH.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Geometric isomers of olefins, C═N double bonds, and the like can also be present in the compounds described herein, and all such stable isomers are contemplated in the present invention. Geometric isomers of the compounds of the present invention are described and may be isolated as a mixture of isomers or as separated isomeric forms.

Compounds of the invention may also include tautomeric forms. All tautomeric forms are encompassed.

In some embodiments, the compounds of the present invention may exist as rotational isomers. In some embodiments, the compounds of the present invention exist as mixtures of rotational isomers in any proportion. In other embodiments, the compounds of the present invention exist as particular rotational isomers, substantially free of other rotational isomers.

Compounds of the invention can also include all isotopes of atoms occurring in the intermediates or final compounds. Isotopes include those atoms having the same atomic number but different mass numbers. For example, isotopes of hydrogen include tritium and deuterium.

In some embodiments, the compounds of the invention, and salts thereof, are substantially isolated. By “substantially isolated” is meant that the compound is at least partially or substantially separated from the environment in which it was formed or detected. Partial separation can include, for example, a composition enriched in the compound of the invention. Substantial separation can include compositions containing at least about 50%, at least about 60%, at least about 70%, at least about 80%, at least about 90%, at least about 95%, at least about 97%, or at least about 99% by weight of the compound of the invention, or salt thereof. Methods for isolating compounds and their salts are routine in the art.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 1 (1977) p. 1-19, each of which is incorporated herein by reference in its entirety.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

A “solvate” refers to a physical association of a compound of Formula I with one or more solvent molecules.

“Subject” includes humans. The terms “human,” “patient,” and “subject” are used interchangeably herein.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

“Compounds of the present disclosure,” and equivalent expressions, are meant to embrace compounds of Formula I as described herein, as well as its subgenera, which expression includes the stereoisomers (e.g., entantiomers, diastereomers) and constitutional isomers (e.g., tautomers) of compounds of Formula I as well as the pharmaceutically acceptable salts, where the context so permits. References to compounds of Formula I are meant to include subgenera thereof, such as, for example, compounds of Formula II, compounds of Formula III, and compounds of Formula IV.

As used herein, the term “isotopic variant” refers to a compound that contains proportions of isotopes at one or more of the atoms that constitute such compound that is greater than natural abundance. For example, an “isotopic variant” of a compound can be radiolabeled, that is, contain one or more radioactive isotopes, or can be labeled with non-radioactive isotopes such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers,” for example, diastereomers, enantiomers, and atropisomers. The compounds of this disclosure may possess one or more asymmetric centers; such compounds can therefore be produced as individual (R)- or (S)-stereoisomers at each asymmetric center, or as mixtures thereof. Unless indicated otherwise, the description or naming of a particular compound in the specification and claims is intended to include all stereoisomers and mixtures, racemic or otherwise, thereof. Where one chiral center exists in a structure, but no specific stereochemistry is shown for that center, both enantiomers, individually or as a mixture of enantiomers, are encompassed by that structure. Where more than one chiral center exists in a structure, but no specific stereochemistry is shown for the centers, all enantiomers and diastereomers, individually or as a mixture, are encompassed by that structure. The methods for the determination of stereochemistry and the separation of stereoisomers are well-known in the art.

The disclosure is directed to compounds of Formula I:

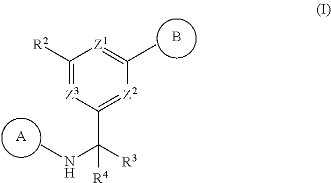

or a pharmaceutically acceptable salt, N-oxide, or stereoisomer thereof, wherein
Ring A is aryl or a 5-7 membered heteroaryl ring comprising 1-4 heteroatoms selected from N, O, and S, wherein in the aryl or 5-7 membered heteroaryl ring is optionally substituted with one or more groups independently selected from D, halogen, C₁-C₈ alkoxy, C₁-C₈ haloalkoxy, C₁-C₈ alkyl, haloalkyl, —OH, —CN, —NO2, —C2-C6 alkenyl, —C2-C6 alkynyl, aryl, heteroaryl, cycloalkyl, cycloalkenyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF5, —P(O)R^{b}R^{b}, —P(O)R^{c}R^{d}, —P(O)(OR^{b})(OR^{b}), —B(OR^{c})(OR^{d}), —SiR^{b}₃, —S(O)₂R^{b}, —C(O)NR^{b}OR^{b}, —S(O)₂OR^{b}, —OS(O)₂OR^{b}, and —OPO(OR^{b})(OR^{b}); wherein each C₁-C₈ alkyl or haloalkyl is optionally substituted by 1-6 groups selected from D, halogen, —OH, —CN, —OR^{a}, —SR^{a}, —NR^{a}R^{d}, or NR^{c}R^{d};
Ring B is 4-13 membered heterocycloalkyl, 4-10 membered heterocycloalkenyl, or 4-10 membered heteroaryl, wherein the 4-13 membered heterocycloalkyl, 4-10 membered heterocycloalkenyl, or 4-10 membered heteroaryl is optionally substituted with one or more groups independently selected from D, oxo, ═NR^{a}, ═N—OR^{a}, ═N—CN, ═S, halogen, C₁-C₈ alkoxide, C₁-C₁₀ alkyl, haloalkyl, —OH, —CN, —NO₂, —C₂-C₁₀ alkenyl, —C₂-C₁₀ alkynyl, aryl, heteroaryl, cycloalkyl, cycloalkenyl, heterocycloalkyl, heterocycloalkenyl, C₀-C₄alk-aryl, C₀-C₄alk-heteroaryl, —OR^{a}, —SR^{a}, —NR^{c}R^{d}, —NR^{a}R^{c}, —C(O)R^{b}, —OC(O)R^{b}, —C(O)OR^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —C(═NR^{b})NR^{c}R^{d}, —C(═NOR^{b})NR^{c}R^{d}, —C(═NCN)NR^{c}R^{d}, —P(OR^{c})₂, —P(O)R^{c}R^{b}, —P(O)R^{c}R^{d}, —P(O)OR^{c}OR^{b}, —S(O)R^{b}, —S(O)NR^{c}R^{d}, —S(O)₂NR^{c}R^{d}, —S(O)(═NR^{b})R^{b}, —SF5, —P(O)R^{b}R^{b}, —P(O)(OR^{b})(OR^{b}), —B(OR^{c})(OR^{d}), —S(O)₂R^{b}, —C(O)NR^{b}OR^{b}, —SiR^{b}₃, —S(O)₂OR^{b}, —OS(O)₂OR^{b}, —OPO(OR^{b})(OR^{b}) and -L-W; wherein each C₁-C₈ alkoxide, C₁-C₁₀ alkyl, haloalkyl, —C2-C10 alkenyl, —C2-C10 alkynyl, aryl, heteroaryl, cycloalkyl, cycloalkenyl, heterocycloalkyl, or heterocycloalkenyl is optionally substituted by 1-6 R^{f} groups;
L is absent or is C₁-C₈ alkylene, —O—, —N(R^{a})—, —S— or 3-10 membered cycloalkylene;
W is a 5-10 membered heteroaryl ring comprising 1-4 heteroatoms selected from N, O, and S; a 5-12 membered heterocyclic group comprising 1-4 heteroatoms selected from N, O, and S; C₁-C₈ alkyl; haloalkyl; —C2-C6 alkenyl; —C2-C6 alkynyl; aryl; cycloalkyl; cycloalkenyl; heterocyclo-alkenyl; NR^{c}R^{d}; OR^{b}; or SR^{b}; each of which is optionally substituted by 1-6 R^{f} groups;
Z¹, Z², and Z³ are each independently CR² or N;
each R² is independently H, D, halogen, C₁-C₈ alkoxide, C₁-C₈ alkyl, haloalkyl, —OH, —CN, —NO2, —C2-C6 alkenyl, —C2-C6 alkynyl, aryl, heteroaryl, cycloalkyl, cycloalkenyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF5, —P(O)R^{b}R^{b}, —P(O)R^{c}R^{d}, —P(O)(OR^{b})(OR^{c}), —B(OR^{c})(OR^{d}), SiR^{b}₃, —S(O)₂R^{b}, —C(O)NR^{b}OR^{b}, —S(O)₂OR^{b}, —OS(O)₂OR^{b}, or —OPO(OR^{b})(OR^{c}); wherein said C₁-C₈ alkyl is optionally substituted by 1-6 groups selected from D, halogen, —OH, —CN, —OR^{a}, —SR^{a}, —NR^{a}R^{d}, or NR^{c}R^{d};
each R³ and R⁴ is independently H, D, C₁-C₈ alkyl, haloalkyl, or CN; wherein said C₁-C₈ alkyl is optionally substituted by 1-6 groups selected from D, halogen, —OH, —CN, —OR^{a}, —SR^{a}, —NR^{a}R^{d}, or —NR^{c}R^{d};
or R³ and R⁴, together with the atom to which they are both attached, are combined to form a C₃-C₇ cycloalkyl or C₄-C₈ heterocycloalkyl, wherein the C₃-C₇ cycloalkyl or C₄-C₈ heterocycloalkyl is optionally substituted by 1-6 groups selected from D, halogen, —OH, —CN, —OR^{a}, —SR^{a}, —NR^{a}R^{d}, or —NR^{c}R^{d};
each R^{a} is independently H, D, —C(O)R^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —C(═NR^{b})NR^{c}R^{d}, —C(═NOR^{b})NR^{c}R^{d}, —C(═NCN)NR^{c}R^{d}, —P(OR^{c})₂, —P(O)R^{c}R^{b}, —P(O)R^{c}R^{d}, —P(O)OR^{c}OR^{b}, —S(O)R^{b}, —S(O)NR^{c}R^{d}, —S(O)₂R^{b}, —S(O)₂NR^{c}R^{d}, —SiR^{b}₃, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl, wherein each —C₁-C₁₀alkyl, —C₂-C₁₀ alkenyl, —C₂-C₁₀ alkynyl, aryl, cycloalkyl, cycloalkenyl, heteroaryl, heterocycloalkyl, and heterocycloalkenyl of R^{a} is optionally substituted by 1-6 R^{f} groups;
each R^{b} is independently H, D, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl; wherein each —C₁-C₆ alkyl, —C₂-C₆ alkenyl, —C₂-C₆ alkynyl, aryl, cycloalkyl, cycloalkenyl, heteroaryl, heterocycloalkyl, and heterocycloalkenyl of R^{b} is optionally substituted by 1-6 R^{f} groups;
each R^{c} or R^{d} is independently H, D, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl; wherein each —C₁-C₁₀ alkyl, —C₂-C₆ alkenyl, —C₂-C₆ alkynyl, —OC₁-C₆alkyl, —O— cycloalkyl, aryl, heteroaryl, cycloalkyl, cycloalkenyl, heterocycloalkyl of R^{c} or R^{d} is optionally substituted by 1-6 R^{f} groups;
or R^{c} and R^{d}, together with the atom to which they are both attached, form a monocyclic or multicyclic heterocycloalkyl, or a monocyclic or multicyclic heterocycloalkenyl group, wherein the monocyclic or multicyclic heterocycloalkyl, or a monocyclic or multicyclic heterocycloalkenyl group is optionally substituted by 1-6 R^{f} groups;
each R^{f} is independently D, oxo, halogen, C₁-C₈ alkoxide, C₁-C₈ alkyl, haloalkyl, —OH, —CN, —NO2, —C2-C6 alkenyl, —C2-C6 alkynyl, aryl, heteroaryl, cycloalkyl, cycloalkenyl, heterocycloalkyl, heterocycloalkenyl, —OR^{g0}, —SR^{g0}, —NR^{g2}R^{g3}, —NR^{g0}R^{g2}, —C(O)R^{g1}, —OC(O)R^{g1}, —C(O)OR^{g1}, —C(O)NR^{g2}R^{g3}, —S(O)R^{g1}, —S(O)₂NR^{g2}R^{g3}, —S(O)(═NR^{g1})R^{g1}, —SF5, —P(O)R^{g1}R^{g1}, —P(O)R^{g2}R^{g3}, —P(O)(OR^{g1})(OR^{g1}), SiR^{g1}₃, —B(OR^{g2})(OR^{g3}), —S(O)₂R^{g1}, —C(O)NR^{g1}OR^{g1}, —S(O)₂OR^{g1}, —OS(O)₂OR^{g1}, or —OPO(OR^{g1})(OR^{g1}); wherein each C₁-C₈ alkoxide, C₁-C₈ alkyl, haloalkyl, —C2-C6 alkenyl, —C2-C6 alkynyl, aryl, heteroaryl, cycloalkyl, cycloalkenyl, heterocycloalkyl, or heterocycloalkenyl is optionally substituted by 1-6 R^{g} groups;
each R^{g0} is independently H, D, —C(O)R^{g1}, —C(O)OR^{g2}, —C(O)NR^{g2}R^{g3}, —C(═NR^{g1})NR^{g2}R^{g3}, —C(═NOR^{g1})NR^{g2}R^{g3}, —C(═NCN)NR^{g1}R^{g2}, —C(═NR^{g1})NR^{g2}R^{g3}, —C(═NOR^{g1})NR^{g2}R^{g3}, —C(═NCN)NR^{g2}R^{g3}, —P(OR^{g2})₂, —P(O)R^{g2}R^{g1}, —P(O)R^{g2}R^{g3}, —P(O)OR^{g2}OR^{g1}, —S(O)R^{g1}, —S(O)NR^{g2}R^{g3}, —S(O)₂R^{g1}, —S(O)₂NR^{g2}R^{g3}, —SiR^{g1}₃, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl, wherein each —C₁-C₁₀alkyl, —C₂-C₁₀ alkenyl, —C₂-C₁₀ alkynyl, aryl, cycloalkyl, cycloalkenyl, heteroaryl, heterocycloalkyl, and heterocycloalkenyl is optionally substituted by 1-6 R^{g} groups;
each R^{g1} is independently H, D, C₁-C₈ alkyl, haloalkyl, —C2-C6 alkenyl, —C2-C6 alkynyl, aryl, heteroaryl, cycloalkyl, cycloalkenyl, heterocycloalkyl, or heterocycloalkenyl, wherein each C₁-C₈ alkyl, haloalkyl, —C2-C6 alkenyl, —C2-C6 alkynyl, aryl, heteroaryl, cycloalkyl, cycloalkenyl, heterocycloalkyl, or heterocycloalkenyl is optionally substituted by 1-6 R^{g} groups; each R^{g2} or R^{g3} is independently H, D, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl; wherein each —C₁-C₁₀ alkyl, —C₂-C₆ alkenyl, —C₂-C₆ alkynyl, —OC₁-C₆alkyl, —O— cycloalkyl, aryl, heteroaryl, cycloalkyl, cycloalkenyl, or heterocycloalkyl is optionally substituted by 1-6 R^{g} groups; or R^{g2} and R^{g3}, together with the atom to which they are both attached, form a monocyclic or multicyclic heterocycloalkyl, or a monocyclic or multicyclic heterocycloalkenyl group, wherein the monocyclic or multicyclic heterocycloalkyl, or monocyclic or multicyclic heterocycloalkenyl group is optionally substituted by 1-6 R^{g} groups;
each R^{g} is independently:
D; —CN; —NO2; oxo; halogen; —SF₅; —OR^{h}; —SR^{h}; —NR^{h}R^{h}; —SiR^{h}₃; —C(O)R^{h}; —OC(O)R^{h}; —C(O)OR^{h}; —OC(O)OR^{h}; —C(O)NR^{h}OR^{h}; —S(O)R^{h}; —S(O)₂R^{h}; —S(O)₂OR^{h}; —OS(O)₂OR^{h}; —S(O)(═NR^{h})R^{h}; —P(O)(OR^{h})(OR^{h}); —P(O)R^{h}R^{h}; OPO(OR^{h})(OR^{h}); —C(O)NR^{h}R^{h}; —OC(O)NR^{h}R^{h}; —S(O)₂NR^{h}R^{h}; —B(OR^{h})(OR^{h}); —OC(═NR^{h})NR^{h}R^{h}; —OC(═NOR^{h})NR^{h}R^{h}; —OC(═NCN)NR^{h}R^{h}; —OP(OR^{h})₂; —OP(O)R^{h}R^{h}; —OP(O)OR^{h}OR^{h}; —OS(O)R^{h}; —OS(O)NR^{h}R^{h}; —OS(O)₂R^{h}; —OS(O)₂NR^{h}R^{h}; —OSiR^{h}₃; —S—C(O)R^{h}; —S—C(O)OR^{h}; —S—C(O)NR^{h}R^{h}; —S—C(═NR^{h})NR^{h}R^{h}; —S—C(═NOR^{h})NR^{h}R^{h}; —C(═NCN)NR^{h}R^{h}; —S—P(OR^{h})₂; —S—P(O)R^{h}R^{h}; —S—P(O)OR^{h}OR^{h}; —S(O)R^{h}; —S(O)NR^{h}R^{h}; —S(O)₂R^{h}; —S(O)₂NR^{h}R^{h}; —SSiR^{h}₃; —NR^{h}C(O)R^{h}; —NR^{h}C(O)OR^{h}; —NR^{h}C(O)NR^{h}R^{h}; —NR^{h}C(═NR^{h})NR^{h}R^{h}; —NR^{h}C(═NOR^{h})NR^{h}R^{h}; —NR^{h}C(═NCN)NR^{h}R^{h}; —NR^{h}P(OR^{h})₂; —NR^{h}P(O)R^{h}R^{h}; —NR^{h}P(O)OR^{h}OR^{h}; —NR^{h}S(O)R^{h}; —NR^{h}S(O)NR^{h}R^{h}; —NR^{h}S(O)₂R^{h}; —NR^{h}S(O)₂NR^{h}R^{h}; —NR^{h}SiR^{h}₃; C₁-C₈ alkoxide; C₁-C₈ alkyl; haloalkyl; —C2-C6 alkenyl; —C2-C6 alkynyl; aryl; heteroaryl; cycloalkyl; cycloalkenyl; heterocycloalkyl; or heterocycloalkenyl; wherein the C₁-C₈ alkoxide, C₁-C₈ alkyl, haloalkyl, —C2-C6 alkenyl, —C2-C6 alkynyl, aryl, heteroaryl, cycloalkyl, cycloalkenyl, heterocycloalkyl, or heterocycloalkenyl is optionally substituted by 1-6 groups selected from D, halogen, —CN, —NO2, oxo, —OR^{h}; —SR^{h}; or —NR^{h}R^{h};
wherein each R^{h} is independently H, D, C₁-C₈alkyl, haloalkyl, —C2-C6 alkenyl, —C2-C6 alkynyl, aryl, heteroaryl, cycloalkyl, cycloalkenyl, heterocycloalkyl, or heterocycloalkenyl; or wherein two R^{h} attached to the same atom may form a monocyclic or multicyclic heterocycloalkyl, or a monocyclic or multicyclic heterocycloalkenyl group.

In some embodiments, Z¹, Z², and Z³ are each independently CR² or N. In some embodiments, each Z¹, Z² and Z³ is CR². In other embodiments, Z¹ and Z³ are CR². In other embodiments, at least one of Z¹, Z² and Z³ is N. In yet other embodiments, Z² is N. In yet other embodiments, Z² is CH.

In some embodiments, each R² is independently H, D, halogen, C₁-C₈ alkoxide, C₁-C₈ alkyl, haloalkyl, —OH, —CN, —NO2, —C2-C6 alkenyl, —C2-C6 alkynyl, aryl, heteroaryl, cycloalkyl, cycloalkenyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF5, —P(O)R^{b}R^{b}, —P(O)R^{c}R^{d}, —P(O)(OR^{b})(OR^{b}), —B(OR^{c})(OR^{d}), SiR^{b}₃, —S(O)₂R^{b}, —C(O)NR^{b}OR^{b}, —S(O)₂OR^{b}, —OS(O)₂OR^{b}, or —OPO(OR^{b})(OR^{b}); wherein said C₁-C₈ alkyl is optionally substituted by 1-6 groups selected from D, halogen, —OH, —CN, —OR^{a}, —SR^{a}, —NR^{a}R^{d}, or NR^{c}R^{d}.

In some embodiments, at least one R² is H. In some embodiments, at least one R² is D. In some embodiments, at least one R² is halogen. In some embodiments, at least one R² is C₁-C₈ alkoxide. In some embodiments, at least one R² is C₁-C₈ alkyl. In some embodiments, the C₁-C₈ alkyl is optionally substituted by 1-6 groups selected from D, halogen, —OH, —CN, —OR^{a}, —SR^{a}, —NR^{a}R^{d}, or NR^{c}R^{d}. In other embodiments, at least one R² is haloalkyl. In other embodiments, at least one R² is —OH. In other embodiments, at least one R² is —CN. In other embodiments, at least one R² is —NO2. In other embodiments, at least one R² is —C2-C6 alkenyl. In other embodiments, at least one R² is —C2-C6 alkynyl. In other embodiments, at least one R² is aryl. In other embodiments, at least one R² is hetereoaryl. In other embodiments, at least one R² is cycloalkyl. In other embodiments, at least one R² is cycloalkenyl. In other embodiments, at least one R² is heterocycloalkyl. In some embodiments, at least one R² is heterocyclo-alkenyl. In other embodiments, at least one R² is —OR^{a}. In other embodiments, at least one R² is —SR^{a}. In other embodiments, at least one R² is —NR^{c}R^{d}. In other embodiments, at least one R² is —NR^{a}R^{c}. In other embodiments, at least one R² is —C(O)R^{b}. In other embodiments, at least one R² is —OC(O)R^{b}. In other embodiments, at least one R² is —C(O)OR^{b}. In other embodiments, at least one R² is —C(O)NR^{c}R^{d}. In other embodiments, at least one R² is —S(O)R^{b}. In other embodiments, at least one R² is —S(O)₂NR^{c}R^{d}. In other embodiments, at least one R² is —S(O)(═NR^{b})R^{b}. In other embodiments, at least one R² is —SF5. In other embodiments, at least one R² is —P(O)R^{b}R^{b}. In other embodiments, at least one R² is —P(O)R^{c}R^{d}. In other embodiments, at least one R² is —P(O)(OR^{b})(OR^{b}). In other embodiments, at least one R² is —B(OR^{c})(OR^{d}). In other embodiments, at least one R² is —S(O)₂R^{b}. In other embodiments, at least one R² is —C(O)NR^{b}OR^{b}. In other embodiments, at least one R² is —S(O)₂OR^{b}. In other embodiments, at least one R² is —OS(O)₂OR^{b}. In other embodiments, at least one R² is —OPO(OR^{b})(OR^{b}).

In some embodiments, at least one R² is H, C₁-C₈ alkyl, CD₃, CF₃, halogen, CN or CHF₂. In some embodiments, at least one R² is H. In some embodiments, at least one R² is C₁-C₈alkyl. In other embodiments, at least one R² is methyl. In other embodiments, at least one R² is CD₃. In other embodiments, at least one R² is CF₃. In yet other embodiments, at least one R² is Br. In yet other embodiments, at least one R² is F. In yet other embodiments, at least one R² is CN. In yet other embodiments, at least one R² is CHF₂.

In some embodiments, R³ is H, D, C₁-C₈ alkyl, haloalkyl, or CN; wherein said C₁-C₈ alkyl is optionally substituted by 1-6 groups selected from D, halogen, —OH, —CN, —OR^{a}, —SR^{a}, —NR^{a}R^{d}, or NR^{c}R^{d}.

In some embodiments, R³ is H. In some embodiments, R³ is D. In some embodiments, R³ is C₁-C₈ alkyl. In some embodiments, the C₁-C₈ alkyl is optionally substituted by 1-6 groups selected from D, halogen, —OH, —CN, —OR^{a}, —SR^{a}, —NR^{a}R^{d}, or NR^{c}R^{d}. In some embodiments, R³ is haloalkyl. In some embodiments, R³ is CN.

In other embodiments, R³ is H or C₁-C₈ alkyl. In other embodiments, R³ is H. In other embodiments, R³ is C₁-C₈alkyl. In other embodiments, R³ is methyl.

In some embodiments, R⁴ is H, D, C₁-C₈ alkyl, haloalkyl, or CN; wherein said C₁-C₈ alkyl is optionally substituted by 1-6 groups selected from D, halogen, —OH, —CN, —OR^{a}, —SR^{a}, —NR^{a}R^{d}, or NR^{c}R^{d}.

In some embodiments, R⁴ is H. In some embodiments, R⁴ is D. In some embodiments, R⁴ is C₁-C₈ alkyl. In some embodiments, the C₁-C₈ alkyl is optionally substituted by 1-6 groups selected from D, halogen, —OH, —CN, —OR^{a}, —SR^{a}, —NR^{a}R^{d}, or NR^{c}R^{d}. In some embodiments, R⁴ is haloalkyl. In some embodiments, R⁴ is CN.

In other embodiments, R⁴ is H or C₁-C₈ alkyl. In other embodiments, R⁴ is H. In other embodiments, R⁴ is C₁-C₈alkyl. In other embodiments, R⁴ is methyl.

In some embodiments, R³ and R⁴ together with the atom to which they are attached are combined to form a C₃-C₇ cycloalkyl or C₄-C₈ heterocycloalkyl wherein the C₃-C₇ cycloalkyl or C₄-C₈ heterocycloalkyl is optionally substituted by 1-6 groups selected from D, halogen, —OH, —CN, —OR^{a}, —SR^{a}, —NR^{a}R^{d}, or —NR^{c}R^{d}. In some embodiments, R³ and R⁴ together with the atom to which they are attached are combined to form a C₃-C₇ cycloalkyl. In some embodiments, R³ and R⁴ together with the atom to which they are attached are combined to form a C₄-C₈ heterocycloalkyl.

In some embodiments, each R^{a} is independently H, D, —C(O)R^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —C(═NR^{b})NR^{c}R^{d}, —C(═NOR^{b})NR^{c}R^{d}, —C(═NCN)NR^{c}R^{d}, —P(OR^{c})₂, —P(O)R^{c}R^{b}, —P(O)R^{c}R^{d}, —P(O)OR^{c}OR^{b}, —S(O)R^{b}, —S(O)NR^{c}R^{d}, —S(O)₂R^{b}, —S(O)₂NR^{c}R^{d}, —SiR^{b}₃, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl, wherein each —C₁-C₁₀alkyl, —C₂-C₁₀ alkenyl, —C₂-C₁₀ alkynyl, aryl, cycloalkyl, cycloalkenyl, heteroaryl, heterocycloalkyl, and heterocycloalkenyl of R^{a} is optionally substituted by 1-6 R^{f} groups.

In some embodiments, R^{a} is H. In some embodiments, R^{a} is D. In some embodiments, R^{a} is —CN. In some embodiments, R^{a} is —CN. In some embodiments, R^{a} is —C(O)R^{b}. In some embodiments, R^{a} is —C(O)OR^{c}. In some embodiments, R^{a} is —C(O)NR^{c}R^{d}. In some embodiments, R^{a} is —C(═NR^{b})NR^{b}R^{c}. In some embodiments, R^{a} is C(═NOR^{b})NR^{b}R^{c}. In some embodiments, R^{a} is —C(═NCN)NR^{b}R^{c}.

In other embodiments, R^{a} is —P(OR^{c})₂, —P(O)R^{c}R^{b}, —P(O)R^{c}R^{d}, —P(O)OR^{c}OR^{b}, —S(O)R^{b}, —S(O)NR^{c}R^{d}, —S(O)₂R^{b}, —S(O)₂NR^{c}R^{d}, SiR^{b}₃, and the like. In yet other embodiments, R^{a} is —C₁-C₁₀alkyl, —C₂-C₁₀ alkenyl, —C₂-C₁₀ alkynyl, aryl, cycloalkyl, cycloalkenyl, heteroaryl, heterocycloalkyl, heterocycloalkenyl, and the like. In some embodiments, each —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl of R^{a} is optionally substituted by 1-6 R^{f} groups.

In some embodiments, each R^{b} is independently each R^{b} is independently H, D, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl; wherein each —C₁-C₆ alkyl, —C₂-C₆ alkenyl, —C₂-C₆ alkynyl, aryl, cycloalkyl, cycloalkenyl, heteroaryl, heterocycloalkyl, and heterocycloalkenyl of R^{b} is optionally substituted by 1-6 R^{f} groups.

In some embodiments, R^{b} is H. In some embodiments, R^{b} is D. In some embodiments, R^{b} is —C₁-C₆ alkyl. In some embodiments, R^{b} is —C₂-C₆ alkenyl. In some embodiments, R^{b} is —C₂-C₆ alkynyl. In other embodiments, R^{b} is aryl. In other embodiments, R^{b} is cycloalkyl. In other embodiments, R^{b} is cycloalkenyl. In other embodiments, R^{b} is heteroaryl. In other embodiments, R^{b} is heterocycloalkyl. In other embodiments, R^{b} is heterocycloalkenyl. In some embodiments, each —C₁-C₆ alkyl, —C₂-C₆ alkenyl, —C₂-C₆ alkynyl, aryl, cycloalkyl, cycloalkenyl, heteroaryl, heterocycloalkyl, and heterocycloalkenyl of R^{b} is optionally substituted by 1-6 R^{f} groups.

In some embodiments, each R^{c} or R^{d} is independently H, D, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl, wherein each —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl of R^{c} or R^{d} is optionally substituted by 1-6 R^{f} groups.

In some embodiments, R^{c} or R^{d} is H. In some embodiments, R^{c} or R^{d} is D. In some embodiments, R^{c} or R^{d} is —C₁-C₁₀ alkyl. In some embodiments, R^{c} or R^{d} is —C₂-C₆ alkenyl. In some embodiments, R^{c} or R^{d} is —C₂-C₆ alkynyl. In other embodiments, R^{c} or R^{d} is —OC₁-C₆alkyl. In other embodiments, R^{c} or R^{d} is —O-cycloalkyl. In other embodiments, R^{c} or R^{d} is aryl. In other embodiments, R^{c} or R^{d} is cycloalkyl. In other embodiments, R^{c} or R^{d} is cycloalkenyl. In other embodiments, R^{c} or R^{d} is heteroaryl. In other embodiments, R^{c} or R^{d} is heterocycloalkyl. In other embodiments, R^{c} or R^{d} is heterocycloalkenyl. In other embodiments of Formula I, R^{c} or R^{d} is —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, or heterocycloalkyl optionally substituted by 1-6 R^{f} groups.

In yet other embodiments, R^{c} and R^{d} in Formula I, together with the atom to which they are both attached, form a monocyclic or multicyclic heterocycloalkyl, or a monocyclic or multicyclic heterocyclo-alkenyl group, wherein the monocyclic or multicyclic heterocycloalkyl, or a monocyclic or multicyclic heterocycloalkenyl group is optionally substituted by 1-6 R^{f} groups. In yet other embodiments, R^{c} and R^{d} form a monocyclic heterocycloalkyl. In yet other embodiments, R^{c} and R^{d} form a multicyclic heterocycloalkyl. In yet other embodiments, R^{c} and R^{d} form a monocyclic heterocycloalkenyl group. In yet other embodiments, R^{c} and R^{d} form a multicyclic heterocycloalkenyl group. In some embodiments, the monocyclic or multicyclic heterocycloalkyl, or a monocyclic or multicyclic heterocycloalkenyl group is optionally substituted by 1-6 R^{f} groups.

In some embodiments, each R^{f} is independently D, oxo, halogen, C₁-C₈ alkoxide, C₁-C₈ alkyl, haloalkyl, —OH, —CN, —NO2, —C2-C6 alkenyl, —C2-C6 alkynyl, aryl, heteroaryl, cycloalkyl, cycloalkenyl, heterocycloalkyl, heterocycloalkenyl, —OR^{g0}, —SR^{g0}, —NR^{g2}R^{g3}, —NR^{g1}R^{g2}, —C(O)R^{g1}, —OC(O)R^{g1}, —C(O)OR^{g1}, —C(O)NR^{g2}R^{g3}, —S(O)R^{g1}, —S(O)₂NR^{g2}R^{g3}, —S(O)(═NR^{g1})R^{g1}, —SF5, —P(O)R^{g1}R^{g1}, —P(O)R^{g2}R^{g3}, —P(O)(OR^{g1})(OR^{g1}), SiR^{g1}₃, —B(OR^{g2})(OR^{g3}), —S(O)₂R^{g1}, —C(O)NR^{g1}OR^{g1}, —S(O)₂OR^{g1}, —OS(O)₂OR^{g1}, or —OPO(OR^{g1})(OR^{g1}); wherein each C₁-C₈ alkoxide, C₁-C₈ alkyl, haloalkyl, —C2-C6 alkenyl, —C2-C6 alkynyl, aryl, heteroaryl, cycloalkyl, cycloalkenyl, heterocycloalkyl, or heterocycloalkenyl is optionally substituted by 1-6 R^{g} groups.

In some embodiments, R^{f} is D. In some embodiments, R^{f} is oxo. In some embodiments, R^{f} is halogen. In some embodiments, R^{f} is C₁-C₈ alkoxide. In some embodiments, R^{f} is C₁-C₈ alkyl. In some embodiments, the C₁-C₈ alkyl is optionally substituted by 1-6 R^{g} groups. In some embodiments, R^{f} is haloalkyl. In some embodiments, R^{f} is —OH. In some embodiments, R^{f} is —CN. In some embodiments, R^{f} is —NO2. In some embodiments, R^{f} is —C2-C6 alkenyl. In some embodiments, R^{f} is —C2-C6 alkynyl. In some embodiments, R^{f} is aryl. In some embodiments, R^{f} is heteroaryl. In some embodiments, R^{f} is cycloalkyl. In other embodiments, R^{f} is cycloalkenyl. In other embodiments, R^{f} is heterocycloalkyl. In other embodiments, R^{f} is heterocycloalkenyl. In other embodiments, R^{f} is —OR^{g1}. In other embodiments, R^{f} is —SR^{g1}. In other embodiments, R^{f} is —NR^{g2}R^{g3}. In other embodiments, R^{f} is —NR^{g1}R^{g2}. In other embodiments, R^{f} is —C(O)R^{g1}. In other embodiments, R^{f} is —OC(O)R^{g1}. In other embodiments, R^{f} is —C(O)OR^{g1}. In other embodiments, R^{f} is —C(O)NR^{g2}R^{g3}. In yet other embodiments, R^{f} is —S(O)R^{g1}. In yet other embodiments, R^{f} is —S(O)₂NR^{g2}R^{g3}. In yet other embodiments, R^{f} is —S(O)(═NR^{g1})R^{g1}. In yet other embodiments, R^{f} is —SF5. In yet other embodiments, R^{f} is —P(O)R^{g1}R^{g1}. In yet other embodiments, R^{f} is —P(O)R^{g2}R^{g3}. In yet other embodiments, R^{f} is —P(O)(OR^{g1})(OR^{g1}). In yet other embodiments, R^{f} is —B(OR^{g2})(OR^{g3}). In yet other embodiments, R^{f} is —S(O)₂R^{g1}. In yet other embodiments, R^{f} is —C(O)NR^{g1}OR^{g1}. In yet other embodiments, R^{f} is —S(O)₂OR^{g1}. In yet other embodiments, R^{f} is —OS(O)₂OR^{g1}. In yet other embodiments, R^{f} is —OPO(OR^{g1})(OR^{g1}).

In some embodiments, each C₁-C₈ alkoxide, C₁-C₈ alkyl, haloalkyl, —C2-C6 alkenyl, —C2-C6 alkynyl, aryl, heteroaryl, cycloalkyl, cycloalkenyl, heterocycloalkyl, or heterocycloalkenyl of R^{f} is substituted by 1-6 R^{g} groups.

In some embodiments, each R^{g0} is independently H, D, —C(O)R^{g1}, —C(O)OR^{g2}, —C(O)NR^{g2}R^{g3}, —C(═NR^{g1})NR^{g2}R^{g3}, —C(═NOR^{g1})NR^{g2}R^{g3}, —C(═NCN)NR^{g1}R^{g2}, —C(═NR^{g1})NR^{g2}R^{g3}, —C(═NOR^{g1})NR^{g2}R^{g3}, —C(═NCN)NR^{g2}R^{g3}, —P(OR^{g2})₂, —P(O)R^{g2}R^{g1}, —P(O)R^{g2}R^{g3}, —P(O)OR^{g2}OR^{g1}, —S(O)R^{g1}, —S(O)NR^{g2}R^{g3}, —S(O)₂R^{g1}, —S(O)₂NR^{g2}R^{g3}, —SiR^{g1}₃, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl, wherein each —C₁-C₁₀alkyl, —C₂-C₁₀ alkenyl, —C₂-C₁₀ alkynyl, aryl, cycloalkyl, cycloalkenyl, heteroaryl, heterocycloalkyl, and heterocycloalkenyl is optionally substituted by 1-6 R^{g} groups.

In some embodiments, R^{g0} is H. In some embodiments, R^{g0} is D. In some embodiments, R^{g0} is —C(O)R^{g1}. In some embodiments, R^{g0} is —C(O)OR^{g2}. In some embodiments, R^{g0} is —C(O)NR^{g2}R^{g3}. In some embodiments, R^{g0} is —C(═NR^{g1})NR^{g2}R^{g3}. In some embodiments, R^{g0} is —C(═NOR^{g1})NR^{g2}R^{g3}. In some embodiments, R^{g0} is —C(═NCN)NR^{g1}R^{g2}. In some embodiments, R^{g0} is —C(═NR^{g1})NR^{g2}R^{g3}. In some embodiments, R^{g0} is —C(═NOR^{g1})NR^{g2}R^{g3}. In some embodiments, R^{g0} is —C(═NCN)NR^{g2}R^{g3}. In some embodiments, R^{g0} is —P(OR^{g2})₂. In some embodiments, R^{g0} is —P(O)R^{g2}R^{g1}. In some embodiments, R^{g0} is —P(O)R^{g2}R^{g3}. In some embodiments, R^{g0} is —P(O)OR^{g2}OR^{g1}. In some embodiments, R^{g0} is —S(O)R^{g1}. In some embodiments, R^{g0} is —S(O)NR^{g2}R^{g3}. In some embodiments, R^{g0} is —S(O)₂R^{g1}. In some embodiments, R^{g0} is —S(O)₂NR^{g2}R^{g3}. In some embodiments, R^{g0} is —SiR^{g1}. In some embodiments, R^{g0} is —C₁-C₁₀alkyl. In some embodiments, R^{g0} is —C₂-C₁₀ alkenyl. In some embodiments, R^{g0} is —C₂-C₁₀ alkynyl. In some embodiments, R^{g0} is aryl. In some embodiments, R^{g0} is cycloalkyl. In some embodiments, R^{g0} is cycloalkenyl. In some embodiments, R^{g0} is heteroaryl. In some embodiments, R^{g0} is heterocycloalkyl. In some embodiments, R^{g0} is or heterocycloalkenyl. In some embodiments, each —C₁-C₁₀alkyl, —C₂-C₁₀ alkenyl, —C₂-C₁₀ alkynyl, aryl, cycloalkyl, cycloalkenyl, heteroaryl, heterocycloalkyl, and heterocycloalkenyl is optionally substituted by 1-6 R^{g} groups.

In some embodiments, each R^{g1} is independently H, D, C₁-C₈ alkyl, haloalkyl, —C2-C6 alkenyl, —C2-C6 alkynyl, aryl, heteroaryl, cycloalkyl, cycloalkenyl, heterocycloalkyl, or heterocycloalkenyl, wherein each C₁-C₈ alkyl, haloalkyl, —C2-C6 alkenyl, —C2-C6 alkynyl, aryl, heteroaryl, cycloalkyl, cycloalkenyl, heterocycloalkyl, or heterocycloalkenyl is optionally substituted by 1-6 R^{g} groups.

In some embodiments, R^{g1} is H. In some embodiments, R^{g1} is D. In some embodiments, R^{g1} is C₁-C₈ alkyl. In some embodiments, R^{g1} is haloalkyl. In some embodiments, R^{g1} is —C2-C6 alkenyl. In some embodiments, R^{g1} is —C2-C6 alkynyl. In some embodiments, R^{g1} is aryl. In some embodiments, R^{g1} is heteroaryl. In some embodiments, R^{g1} is cycloalkyl. In some embodiments, R^{g1} is cycloalkenyl. In some embodiments, R^{g1} is heterocycloalkyl. In some embodiments, R^{g1} is heterocycloalkenyl. In some embodiments, the C₁-C₈ alkyl, haloalkyl, —C2-C6 alkenyl, —C2-C6 alkynyl, aryl, heteroaryl, cycloalkyl, cycloalkenyl, heterocycloalkyl, or heterocycloalkenyl is optionally substituted by 1-6 R^{g} groups.

In some embodiments, each R^{g2} is independently H, D, —C₁-C₁₀ alkyl, —C₂-C₆ alkenyl, —C₂-C₆alkynyl, —OC₁-C₆alkyl, —O-cycloalkyl, aryl, heteroaryl, cycloalkyl, cycloalkenyl, heterocycloalkyl, or heterocycloalkenyl; wherein each —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, or heterocycloalkyl is optionally substituted by 1-6 R^{g} groups.

In some embodiments, R^{g2} is H. In some embodiments, R^{g2} is D. In some embodiments, R^{g2} is —C₁-C₁₀ alkyl. In some embodiments, R^{g2} is —C₂-C₆ alkenyl. In some embodiments, R^{g2} is —C₂-C₆alkynyl. In some embodiments, R^{g2} is —OC₁-C₆alkyl. In some embodiments, R^{g2} is —O-cycloalkyl. In some embodiments, R^{g2} is aryl. In some embodiments, R^{g2} is heteroaryl. In some embodiments, R^{g2} is cycloalkyl. In some embodiments, R^{g2} is cycloalkenyl. In some embodiments, R^{g2} is heterocycloalkyl. In some embodiments, R^{g2} is heterocycloalkenyl. In some embodiments, the —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, or heterocycloalkyl is optionally substituted by 1-6 R^{g} groups.

In some embodiments, each R^{g3} is independently H, D, —C₁-C₁₀ alkyl, —C₂-C₆ alkenyl, —C₂-C₆alkynyl, —OC₁-C₆alkyl, —O-cycloalkyl, aryl, heteroaryl, cycloalkyl, cycloalkenyl, heterocycloalkyl, or heterocycloalkenyl; wherein each —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, or heterocycloalkyl is optionally substituted by 1-6 R^{g} groups.

In some embodiments, R^{g3} is H. In some embodiments, R^{g3} is D. In some embodiments, R^{g3} is —C₁-C₁₀ alkyl. In some embodiments, R^{g3} is —C₂-C₆ alkenyl. In some embodiments, R^{g3} is —C₂-C₆alkynyl. In some embodiments, R^{g3} is —OC₁-C₆alkyl. In some embodiments, R^{g3} is —O-cycloalkyl. In some embodiments, R^{g3} is aryl. In some embodiments, R^{g3} is heteroaryl. In some embodiments, R^{g3} is cycloalkyl. In some embodiments, R^{g3} is cycloalkenyl. In some embodiments, R^{g3} is heterocycloalkyl. In some embodiments, R^{g3} is heterocycloalkenyl. In some embodiments, the —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, or heterocycloalkyl is optionally substituted by 1-6 R^{g} groups.

In some embodiments, R^{g2} and R^{g3}, together with the atom to which they are both attached, form a monocyclic or multicyclic heterocycloalkyl, or a monocyclic or multicyclic heterocycloalkenyl group, wherein the monocyclic or multicyclic heterocycloalkyl, or monocyclic or multicyclic heterocycloalkenyl group is optionally substituted by 1-6 R^{g} groups.

In some embodiments, R^{g2} and R^{g3}, together with the atom to which they are both attached, form a monocyclic heterocycloalkyl. In some embodiments, R^{g2} and R^{g3}, together with the atom to which they are both attached, form a multicyclic heterocycloalkyl. In some embodiments, R^{g2} and R^{g3}, together with the atom to which they are both attached, form a monocyclic heterocycloalkenyl group. In some embodiments, R^{g2} and R^{g3}, together with the atom to which they are both attached, form a multicyclic heterocycloalkenyl group. In some embodiments, the monocyclic or multicyclic heterocycloalkyl, or monocyclic or multicyclic heterocycloalkenyl group is optionally substituted by 1-6 R^{g} groups.

In some embodiments, each R^{g} is independently D; —CN; —NO2; oxo; halogen; —SF₅; —OR^{h}; —SR^{h}; —NR^{h}R^{h}; —SiR^{h}₃; —C(O)R^{h}; —OC(O)R^{h}; —C(O)OR^{h}; —OC(O)OR^{h}; —C(O)NR^{h}OR^{h}; —S(O)R^{h}; —S(O)₂R^{h}; —S(O)₂OR^{h}; —OS(O)₂OR^{h}; —S(O)(═NR^{h})R^{h}; —P(O)(OR^{h})(OR^{h}); —P(O)R^{h}R^{h}; —OPO(OR^{h})(OR^{h}); —C(O)NR^{h}R^{h}; —OC(O)NR^{h}R^{h}; —S(O)₂NR^{h}R^{h}; —B(OR^{h})(OR^{h}); —OC(═NR^{h})NR^{h}R^{h}. —OC(═NOR^{h})NR^{h}R^{h}; —OC(═NCN)NR^{h}R^{h}; —OP(OR^{h})₂; —OP(O)R^{h}R^{h}; —OP(O)OR^{h}OR^{h}; —OS(O)R^{h}; —OS(O)NR^{h}R^{h}; —OS(O)₂R^{h}; —OS(O)₂NR^{h}R^{h}; —OSiR^{h}₃; —S—C(O)R^{h}; —S—C(O)OR^{h}; —S—C(O)NR^{h}R^{h}; —S—C(═NR^{h})NR^{h}R^{h}; —S—C(═NOR^{h})NR^{h}R^{h}; —S—C(═NCN)NR^{h}R^{h}; —S—P(OR^{h})₂; —S—P(O)R^{h}R^{h}; —S—P(O)OR^{h}OR^{h}; —S(O)R^{h}; —S(O)NR^{h}R^{h}; —S(O)₂R^{h}; —S(O)₂NR^{h}R^{h}; —SSiR^{h}₃; —NR^{h}C(O)R^{h}; —NR^{h}C(O)OR^{h}; —NR^{h}C(O)NR^{h}R^{h}; —NR^{h}C(═NR^{h})NR^{h}R^{h}; —NR^{h}C(═NOR^{h})NR^{h}R^{h}; —NR^{h}C(═NCN)NR^{h}R^{h}; —NR^{h}P(OR^{h})₂; —NR^{h}P(O)R^{h}R^{h}; —NR^{h}P(O)OR^{h}OR^{h}; —NR^{h}S(O)R^{h}; —NR^{h}S(O)NR^{h}R^{h}; —NR^{h}S(O)₂R^{h}; —NR^{h}S(O)₂NR^{h}R^{h}; —NR^{h}SiR^{h}₃; C₁-C₈ alkoxide; C₁-C₈ alkyl; haloalkyl; —C2-C6 alkenyl; —C2-C6 alkynyl; aryl; heteroaryl; cycloalkyl; cycloalkenyl; heterocycloalkyl; or heterocycloalkenyl; wherein the C₁-C₈ alkoxide, C₁-C₈ alkyl, haloalkyl, —C2-C6 alkenyl, —C2-C6 alkynyl, aryl, heteroaryl, cycloalkyl, cycloalkenyl, heterocycloalkyl, or heterocycloalkenyl is optionally substituted by 1-6 groups selected from D, halogen, —CN, —NO2, oxo, —OR^{h}; —SR^{h}; or —NR^{h}R^{h}.

In some embodiments, R^{g} is D. In some embodiments, R^{g} is —CN. In some embodiments, R^{g} is —NO2. In some embodiments, R^{g} is oxo. In some embodiments, R^{g} is halogen. In some embodiments, R^{g} is —SF₅. In some embodiments, R^{g} is —OR^{h}. In some embodiments, R^{g} is —SR^{h}. In some embodiments, R^{g} is —NR^{h}R^{h}. In some embodiments, R^{g} is —SiR^{h}₃. In some embodiments, R^{g} is —C(O)R^{h}. In some embodiments, R^{g} is —OC(O)R^{h}. In some embodiments, R^{g} is —C(O)OR^{h}. In some embodiments, R^{g} is —OC(O)OR^{h}. In some embodiments, R^{g} is —C(O)NR^{h}OR^{h}. In some embodiments, R^{g} is —S(O)R^{h}. In some embodiments, R^{g} is —S(O)₂R^{h}. In some embodiments, R^{g} is —S(O)₂OR^{h}. In some embodiments, R^{g} is —OS(O)₂OR^{h}. In some embodiments, R^{g} is —S(O)(═NR^{h})R^{h}. In some embodiments, R^{g} is —P(O)(OR^{h})(OR^{h}). In some embodiments, R^{g} is —P(O)R^{h}R^{h}. In some embodiments, R^{g} is —OPO(OR^{h})(OR^{h}). In some embodiments, R^{g} is —C(O)NR^{h}R^{h}. In some embodiments, R^{g} is —OC(O)NR^{h}R^{h}. In some embodiments, R^{g} is —S(O)₂NR^{h}R^{h}. In some embodiments, R^{g} is —B(OR^{h})(OR^{h}). In some embodiments, R^{g} is —OC(═NR^{h})NR^{h}R^{h}. In some embodiments, R^{g} is —OC(═NOR^{h})NR^{h}R^{h}. In some embodiments, R^{g} is —OC(═NCN)NR^{h}R^{h}. In some embodiments, R^{g} is —OP(OR^{h})₂. In some embodiments, R^{g} is —OP(O)R^{h}R^{h}. In some embodiments, R^{g} is —OP(O)OR^{h}OR^{h}. In some embodiments, R^{g} is —OS(O)R^{h}. In some embodiments, R^{g} is —OS(O)NR^{h}R^{h}. In some embodiments, R^{g} is —OS(O)₂R^{h}. In some embodiments, R^{g} is —OS(O)₂NR^{h}R^{h}. In some embodiments, R^{g} is —OSiR^{h}₃. In some embodiments, R^{g} is —S—C(O)R^{h}. In some embodiments, R^{g} is —S—C(O)OR^{h}. In some embodiments, R^{g} is —S—C(O)NR^{h}R^{h}. In some embodiments, R^{g} is —S—C(═NR^{h})NR^{h}R^{h}. In some embodiments, R^{g} is —S—C(═NOR^{h})NR^{h}R^{h}. In some embodiments, R^{g} is —S—C(═NCN)NR^{h}R^{h}. In some embodiments, R^{g} is —S—P(OR^{h})₂. In some embodiments, R^{g} is —S—P(O)R^{h}R^{h}. In some embodiments, R^{g} is —S—P(O)OR^{h}OR^{h}. In some embodiments, R^{g} is —S(O)R^{h}. In some embodiments, R^{g} is —S(O)NR^{h}R^{h}. In some embodiments, R^{g} is —S(O)₂R^{h}. In some embodiments, R^{g} is —S(O)₂NR^{h}R^{h}. In some embodiments, R^{g} is —SSiR^{h}₃. In some embodiments, R^{g} is —NR^{h}C(O)R^{h}. In some embodiments, R^{g} is —NR^{h}C(O)OR^{h}. In some embodiments, R^{g} is —NR^{h}C(O)NR^{h}R^{h}. In some embodiments, R^{g} is —NR^{h}C(═NR^{h})NR^{h}R^{h}. In some embodiments, R^{g} is —NR^{h}C(═NOR^{h})NR^{h}R^{h}. In some embodiments, R^{g} is —NR^{h}C(═NCN)NR^{h}R^{h}. In some embodiments, R^{g} is —NR^{h}P(OR^{h})₂. In some embodiments, R^{g} is —NR^{h}P(O)R^{h}R^{h}. In some embodiments, R^{g} is —NR^{h}P(O)OR^{h}OR^{h}. In some embodiments, R^{g} is —NR^{h}S(O)R^{h}. In some embodiments, R^{g} is —NR^{h}S(O)NR^{h}R^{h}. In some embodiments, R^{g} is —NR^{h}S(O)₂R^{h}. In some embodiments, R^{g} is —NR^{h}S(O)₂NR^{h}R^{h}. In some embodiments, R^{g} is —NR^{h}SiR^{h}₃. In some embodiments, R^{g} is C₁-C₈ alkoxide. In some embodiments, R^{g} is C₁-C₈ alkyl. In some embodiments, R^{g} is haloalkyl. In some embodiments, R^{g} is —C2-C6 alkenyl. In some embodiments, R^{g} is —C2-C6 alkynyl. In some embodiments, R^{g} is aryl. In some embodiments, R^{g} is heteroaryl. In some embodiments, R^{g} is cycloalkyl. In some embodiments, R^{g} is cycloalkenyl. In some embodiments, R^{g} is heterocycloalkyl. In some embodiments, R^{g} is heterocycloalkenyl. In some embodiments, the C₁-C₈ alkoxide, C₁-C₈ alkyl, haloalkyl, —C2-C6 alkenyl, —C2-C6 alkynyl, aryl, heteroaryl, cycloalkyl, cycloalkenyl, heterocycloalkyl, or heterocycloalkenyl is optionally substituted by 1-6 groups selected from D, halogen, —CN, —NO2, oxo, —OR^{h}, —SR^{h}, or —NR^{h}R^{h}.

In some embodiments, each R^{h} is independently H, D, C₁-C₈alkyl, haloalkyl, —C2-C6 alkenyl, —C2-C6 alkynyl, aryl, heteroaryl, cycloalkyl, cycloalkenyl, heterocycloalkyl, or heterocycloalkenyl; or wherein two R^{h} attached to the same atom may form a monocyclic or multicyclic heterocycloalkyl, or a monocyclic or multicyclic heterocycloalkenyl group.

In some embodiments, R^{h} is H. In some embodiments, R^{h} is D. In some embodiments, R^{h} is C₁-C₈alkyl. In some embodiments, R^{h} is haloalkyl. In some embodiments, R^{h} is —C2-C6 alkenyl. In some embodiments, R^{h} is —C2-C6 alkynyl. In some embodiments, R^{h} is aryl. In some embodiments, R^{h} is heteroaryl. In some embodiments, R^{h} is cycloalkyl. In some embodiments, R^{h} is cycloalkenyl. In some embodiments, R^{h} is heterocycloalkyl. In some embodiments, R^{h} is heterocycloalkenyl. In some embodiments, two R^{h} attached to the same atom form a monocyclic heterocycloalkyl. In some embodiments, two R^{h} attached to the same atom form a multicyclic heterocycloalkyl. In some embodiments, two R^{h} attached to the same atom form a monocyclic heterocycloalkenyl group. In some embodiments, two R^{h} attached to the same atom form a multicyclic heterocycloalkenyl group.

In some embodiments, Ring A is aryl or a 5-7 membered heteroaryl ring comprising 1-4 heteroatoms selected from N, O, and S, wherein in the aryl or 5-7 membered heteroaryl ring is optionally substituted with one or more groups independently selected from D, halogen, C₁-C₈ alkoxy, C₁-C₈ haloalkoxy, C₁-C₈ alkyl, haloalkyl, —OH, —CN, —NO2, —C2-C6 alkenyl, —C2-C6 alkynyl, aryl, heteroaryl, cycloalkyl, cycloalkenyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF5, —P(O)R^{b}R^{b}, —P(O)R^{c}R^{d}, —P(O)(OR^{b})(OR^{b}), —B(OR^{c})(OR^{d}), —SiR^{b}₃, —S(O)₂R^{b}, —C(O)NR^{b}OR^{b}, —S(O)₂OR^{b}, —OS(O)₂OR^{b}, and —OPO(OR^{b})(OR^{b}); wherein each C₁-C₈ alkyl or haloalkyl is optionally substituted by 1-6 groups selected from D, halogen, —OH, —CN, —OR^{a}, —SR^{a}, —NR^{a}R^{d}, or NR^{c}R^{d}.

In some embodiments, Ring A is aryl optionally substituted with one or more groups independently selected from D, halogen, C₁-C₈ alkoxy, C₁-C₈ haloalkoxy, C₁-C₈ alkyl, haloalkyl, —OH, —CN, —NO2, —C2-C6 alkenyl, —C2-C6 alkynyl, aryl, heteroaryl, cycloalkyl, cycloalkenyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF5, —P(O)R^{b}R^{b}, —P(O)R^{c}R^{d}, —P(O)(OR^{b})(OR^{b}), —B(OR^{c})(OR^{d}), —SiR^{b}₃, —S(O)₂R^{b}, —C(O)NR^{b}OR^{b}, —S(O)₂OR^{b}, —OS(O)₂OR^{b}, and —OPO(OR^{b})(OR^{b}); wherein each C₁-C₈ alkyl or haloalkyl is optionally substituted by 1-6 groups selected from D, halogen, —OH, —CN, —OR^{a}, —SR^{a}, —NR^{a}R^{d}, or NR^{c}R^{d}. In other embodiments, Ring A in Formula (I) is a 5-7 membered heteroaryl ring comprising 1-4 heteroatoms selected from N, O, and S and optionally substituted with one or more groups independently selected from D, halogen, C₁-C₈ alkoxy, C₁-C₈ haloalkoxy, C₁-C₈ alkyl, haloalkyl, —OH, —CN, —NO2, —C2-C6 alkenyl, —C2-C6 alkynyl, aryl, heteroaryl, cycloalkyl, cycloalkenyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF5, —P(O)R^{b}R^{b}, —P(O)R^{c}R^{d}, —P(O)(OR^{b})(OR^{b}), —B(OR^{c})(OR^{d}), —SiR^{b}₃, —S(O)₂R^{b}, —C(O)NR^{b}OR^{b}, —S(O)₂OR^{b}, —OS(O)₂OR^{b}, and —OPO(OR^{b})(OR^{b}); wherein each C₁-C₈ alkyl or haloalkyl is optionally substituted by 1-6 groups selected from D, halogen, —OH, —CN, —OR^{a}, —SR^{a}, —NR^{a}R^{d}, or NR^{c}R^{d}.

In some embodiments, Ring A is

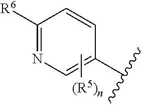

wherein n is 1, 2, or 3; each R⁵ is independently H, D, halogen, C₁-C₈ alkoxide, C₁-C₈ alkyl, haloalkyl, —OH, —CN, —NO2, —C2-C6 alkenyl, —C2-C6 alkynyl, aryl, heteroaryl, cycloalkyl, cycloalkenyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF5, —P(O)R^{b}R^{b}, —P(O)R^{c}R^{d}, —P(O)(OR^{b})(OR^{b}), SiR^{b}₃, —B(OR^{c})(OR^{d}), —S(O)₂R^{b}, —C(O)NR^{b}OR^{b}, —S(O)₂OR^{b}, —OS(O)₂OR^{b}, or —OPO(OR^{b})(OR^{b}); wherein said C₁-C₈ alkyl or haloalkyl is optionally substituted by 1-6 groups selected from D, halogen, —OH, —CN, —OR^{a}, —SR^{a}, —NR^{a}R^{d}, or NR^{c}R^{d}; and R⁶ is —F, —Cl, —Br, —I, C₁-C₄-alkyl, C₁-C₄-alkoxy, C₁-C₄-haloalkoxy, or —CN.

In other embodiments, Ring A is

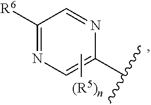

wherein n is 1, 2, or 3; each R⁵ is independently H, D, halogen, C₁-C₈ alkoxide, C₁-C₈ alkyl, haloalkyl, —OH, —CN, —NO2, —C2-C6 alkenyl, —C2-C6 alkynyl, aryl, heteroaryl, cycloalkyl, cycloalkenyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF5, —P(O)R^{b}R^{b}, —P(O)R^{c}R^{d}, —P(O)(OR^{b})(OR^{b}), SiR^{b}₃, —B(OR^{c})(OR^{d}), —S(O)₂R^{b}, —C(O)NR^{b}OR^{b}, —S(O)₂OR^{b}, —OS(O)₂OR^{b}, or —OPO(OR^{b})(OR^{b}); wherein said C₁-C₈ alkyl or haloalkyl is optionally substituted by 1-6 groups selected from D, halogen, —OH, —CN, —OR^{a}, —SR^{a}, —NR^{a}R^{d}, or NR^{c}R^{d}; and R⁶ is —F, —Cl, —Br, —I, C₁-C₄-alkyl, C₁-C₄-alkoxy, C₁-C₄-haloalkoxy, or —CN.

In some embodiments, Ring A is

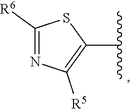

wherein n is 1, 2, or 3; each R⁵ is independently H, D, halogen, C₁-C₈ alkoxide, C₁-C₈ alkyl, haloalkyl, —OH, —CN, —NO2, —C2-C6 alkenyl, —C2-C6 alkynyl, aryl, heteroaryl, cycloalkyl, cycloalkenyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF5, —P(O)R^{b}R^{b}, —P(O)R^{c}R^{d}, —P(O)(OR^{b})(OR^{b}), SiR^{b}₃, —B(OR^{c})(OR^{d}), —S(O)₂R^{b}, —C(O)NR^{b}OR^{b}, —S(O)₂OR^{b}, —OS(O)₂OR^{b}, or —OPO(OR^{b})(OR^{b}); wherein said C₁-C₈ alkyl or haloalkyl is optionally substituted by 1-6 groups selected from D, halogen, —OH, —CN, —OR^{a}, —SR^{a}, —NR^{a}R^{d}, or NR^{c}R^{d}; and R⁶ is —F, —Cl, —Br, —I, C₁-C₄-alkyl, C₁-C₄-alkoxy, C₁-C₄-haloalkoxy, or —CN.

In some embodiments, R⁶ is —F, —Cl, —Br, —I, —C₁-C₄-alkyl, —C₁-C₄-alkoxy, C₁-C₄-haloalkoxy, or —CN. In some embodiments, R⁶ is —F. In some embodiments, R⁶ is —Cl. In other embodiments, R⁶ is —Br. In other embodiments, R⁶ is —I. In other embodiments, R⁶ is —CN. In yet other embodiments, R⁶ is —C₁-C₄-alkyl. In yet other embodiments, R⁶ is —C₁-C₄-alkoxy. In yet other embodiments, R⁶ is C₁-C₄-haloalkoxy.

In some embodiments, R⁶ is —CH₃, —OCH₃, or —OCF₂H. In some embodiments, R⁶ is —CH₃. In some embodiments, R⁶ is OCH₃. In some embodiments, R⁶ is —OCF₂H.

In some embodiments, n is 1, 2, or 3. In some embodiments, n is 1. In other embodiments, n is 2. In yet other embodiments, n is 3.

In some embodiments, each R⁵ is independently H, D, halogen, C₁-C₈ alkoxide, C₁-C₈ alkyl, haloalkyl, —OH, —CN, —NO2, —C2-C6 alkenyl, —C2-C6 alkynyl, aryl, heteroaryl, cycloalkyl, cycloalkenyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF5, —P(O)R^{b}R^{b}, —P(O)R^{c}R^{d}, —P(O)(OR^{b})(OR^{b}), SiR^{b}₃, —B(OR^{c})(OR^{d}), —S(O)₂R^{b}, —C(O)NR^{b}OR^{b}, —S(O)₂OR^{b}, —OS(O)₂OR^{b}, or —OPO(OR^{b})(OR^{b}); wherein said C₁-C₈ alkyl or haloalkyl is optionally substituted by 1-6 groups selected from D, halogen, —OH, —CN, —OR^{a}, —SR^{a}, —NR^{a}R^{d}, or NR^{c}R^{d}.

In some embodiments, at least one R⁵ is H. In some embodiments, at least one R⁵ is D. In some embodiments, at least one R⁵ is halogen. In some embodiments, at least one R⁵ is C₁-C₈ alkoxide. In some embodiments, at least one R⁵ is C₁-C₈ alkyl. In some embodiments, the C₁-C₈ alkyl is optionally substituted by 1-6 groups selected from D, halogen, —OH, —CN, —OR^{a}, —SR^{a}, —NR^{a}R^{d}, or NR^{c}R^{d}.

In other embodiments, at least one R⁵ is haloalkyl. In other embodiments, at least one R⁵ is —OH. In other embodiments, at least one R⁵ is —CN. In other embodiments, at least one R⁵ is —NO2. In other embodiments, at least one R⁵ is —C2-C6 alkenyl. In other embodiments, at least one R⁵ is —C2-C6 alkynyl. In other embodiments, at least one R⁵ is aryl. In other embodiments, at least one R⁵ is hetereoaryl. In other embodiments, at least one R⁵ is cycloalkyl. In other embodiments, at least one R⁵ is cycloalkenyl. In other embodiments, at least one R⁵ is heterocycloalkyl. In some embodiments, at least one R⁵ is heterocycloalkenyl. In other embodiments, at least one R⁵ is —OR^{a}. In other embodiments, at least one R⁵ is —SR^{a}. In other embodiments, at least one R⁵ is —NR^{c}R^{d}. In other embodiments, at least one R⁵ is —NR^{a}R^{c}. In other embodiments, at least one R⁵ is —C(O)R^{b}. In other embodiments, at least one R⁵ is —OC(O)R^{b}. In other embodiments, at least one R⁵ is —C(O)OR^{b}. In other embodiments, at least one R⁵ is —C(O)NR^{c}R^{d}. In other embodiments, at least one R⁵ is —S(O)R^{b}. In other embodiments, at least one R⁵ is —S(O)₂NR^{c}R^{d}. In other embodiments, at least one R⁵ is —S(O)(═NR^{b})R^{b}. In other embodiments, at least one R⁵ is —SF5. In other embodiments, at least one R⁵ is —P(O)R^{b}R^{b}. In other embodiments, at least one R⁵ is —P(O)R^{c}R^{d}. In other embodiments, at least one R⁵ is —P(O)(OR^{b})(OR^{b}). In other embodiments, at least one R⁵ is —B(OR^{c})(OR^{d}). In other embodiments, at least one R⁵ is —S(O)₂R^{b}. In other embodiments, at least one R⁵ is —C(O)NR^{b}OR^{b}. In other embodiments, at least one R⁵ is —S(O)₂OR^{b}. In other embodiments, at least one R⁵ is —OS(O)₂OR^{b}. In other embodiments, at least one R⁵ is —OPO(OR^{b})(OR^{b}).

In some embodiments, at least one R⁵ is —CO₂H, —CONH₂, —COOCH₃, —C(O)H or —CN. In some embodiments, at least one R⁵ is —CO₂H. In other embodiments, at least one R⁵ is —CONH₂. In other embodiments, at least one R⁵ is —COOCH₃. In yet other embodiments, at least one R⁵ is —C(O)H. In yet other embodiments, at least one R⁵ or —CN.

In some embodiments, at least one R⁵ is a carboxylic acid group or isostere thereof. In some embodiments, at least one R⁵ is a carboxylic acid group. In some embodiments, at least one R⁵ is —CO₂H. In other embodiments, at least one R⁵ is an isostere of a carboxylic acid group.

In some embodiments, Ring B is 4-13 membered heterocycloalkyl, 4-10 membered heterocycloalkenyl, or 4-10 membered heteroaryl, wherein the 4-13 membered heterocycloalkyl, 4-10 membered heterocycloalkenyl, or 4-10 membered heteroaryl is optionally substituted with one or more groups independently selected from D, oxo, ═NR^{a}, ═N—OR^{a}, ═N—CN, ═S, halogen, C₁-C₈ alkoxide, C₁-C₁₀ alkyl, haloalkyl, —OH, —CN, —NO₂, —C₂-C₁₀ alkenyl, —C₂-C₁₀ alkynyl, aryl, heteroaryl, cycloalkyl, cycloalkenyl, heterocycloalkyl, heterocycloalkenyl, C₀-C₄alk-aryl, C₀-C₄alk-heteroaryl, —OR^{a}, —SR^{a}, —NR^{c}R^{d}, —NR^{a}R^{c}, —C(O)R^{b}, —OC(O)R^{b}, —C(O)OR^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —C(═NR^{b})NR^{c}R^{d}, —C(═NOR^{b})NR^{c}R^{d}, —C(═NCN)NR^{c}R^{d}, —P(OR^{c})₂, —P(O)R^{c}R^{b}, —P(O)R^{c}R^{d}, —P(O)OR^{c}OR^{b}, —S(O)R^{b}, —S(O)NR^{c}R^{d}, —S(O)₂NR^{c}R^{d}, —S(O)(═NR^{b})R^{b}, —SF5, —P(O)R^{b}R^{b}, —P(O)(OR^{b})(OR^{b}), —B(OR^{c})(OR^{d}), —S(O)₂R^{b}, —C(O)NR^{b}OR^{b}, —SiR^{b}₃, —S(O)₂OR^{b}, —OS(O)₂OR^{b}, —OPO(OR^{b})(OR^{b}) and -L-W; wherein each C₁-C₈ alkoxide, C₁-C₁₀ alkyl, haloalkyl, —C2-C10 alkenyl, —C2-C10 alkynyl, aryl, heteroaryl, cycloalkyl, cycloalkenyl, heterocycloalkyl, or heterocycloalkenyl is optionally substituted by 1-6 R¹ groups.

In some embodiments, Ring B in is a 4-13 membered heterocycloalkyl optionally substituted with one or more groups independently selected from D, oxo, ═NR^{a}, ═N—OR^{a}, ═N—CN, ═S, halogen, C₁-C₈ alkoxide, C₁-C₁₀ alkyl, haloalkyl, —OH, —CN, —NO₂, —C₂-C₁₀ alkenyl, —C₂-C₁₀ alkynyl, aryl, heteroaryl, cycloalkyl, cycloalkenyl, heterocycloalkyl, heterocycloalkenyl, C₀-C₄alk-aryl, C₀-C₄alk-heteroaryl, —OR^{a}, —SR^{a}, —NR^{c}R^{d}, —NR^{a}R^{c}, —C(O)R^{b}, —OC(O)R^{b}, —C(O)OR^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —C(═NR^{b})NR^{c}R^{d}, —C(═NOR^{b})NR^{c}R^{d}, —C(═NCN)NR^{c}R^{d}, —P(OR^{c})₂, —P(O)R^{c}R^{b}, —P(O)R^{c}R^{d}, —P(O)OR^{c}OR^{b}, —S(O)R^{b}, —S(O)NR^{c}R^{d}, —S(O)₂NR^{c}R^{d}, —S(O)(═NR^{b})R^{b}, —SF5, —P(O)R^{b}R^{b}, —P(O)(OR^{b})(OR^{b}), —B(OR^{c})(OR^{d}), —S(O)₂R^{b}, —C(O)NR^{b}OR^{b}, —SiR^{b}₃, —S(O)₂OR^{b}, —OS(O)₂OR^{b}, —OPO(OR^{b})(OR^{b}) and -L-W; wherein each C₁-C₈ alkoxide, C₁-C₁₀ alkyl, haloalkyl, —C2-C10 alkenyl, —C2-C10 alkynyl, aryl, heteroaryl, cycloalkyl, cycloalkenyl, heterocycloalkyl, or heterocycloalkenyl is optionally substituted by 1-6 R^{f} groups.

In other embodiments, Ring B is a 4-10 membered heterocycloalkenyl optionally substituted with one or more groups independently selected from D, oxo, ═NR^{a}, ═N—OR^{a}, ═N—CN, ═S, halogen, C₁-C₈ alkoxide, C₁-C₁₀ alkyl, haloalkyl, —OH, —CN, —NO₂, —C₂-C₁₀ alkenyl, —C₂-C₁₀ alkynyl, aryl, heteroaryl, cycloalkyl, cycloalkenyl, heterocycloalkyl, heterocycloalkenyl, C₀-C₄alk-aryl, C₀-C₄alk-heteroaryl, —OR^{a}, —SR^{a}, —NR^{c}R^{d}, —NR^{a}R^{c}, —C(O)R^{b}, —OC(O)R^{b}, —C(O)OR^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —C(═NR^{b})NR^{c}R^{d}, —C(═NOR^{b})NR^{c}R^{d}, —C(═NCN)NR^{c}R^{d}, —P(OR^{c})₂, —P(O)R^{c}R^{b}, —P(O)R^{c}R^{d}, —P(O)OR^{c}OR^{b}, —S(O)R^{b}, —S(O)NR^{c}R^{d}, —S(O)₂NR^{c}R^{d}, —S(O)(═NR^{b})R^{b}, —SF5, —P(O)R^{b}R^{b}, —P(O)(OR^{b})(OR^{b}), —B(OR^{c})(OR^{d}), —S(O)₂R^{b}, —C(O)NR^{b}OR^{b}, —SiR^{b}₃, —S(O)₂OR^{b}, —OS(O)₂OR^{b}, —OPO(OR^{b})(OR^{b}) and -L-W; wherein each C₁-C₈ alkoxide, C₁-C₁₀ alkyl, haloalkyl, —C2-C10 alkenyl, —C2-C10 alkynyl, aryl, heteroaryl, cycloalkyl, cycloalkenyl, heterocycloalkyl, or heterocycloalkenyl is optionally substituted by 1-6 R^{f} groups.

In other embodiments, Ring B is a 4-10 membered heteroaryl optionally substituted with one or more groups independently selected from D, oxo, ═NR^{a}, ═N—OR^{a}, ═N—CN, ═S, halogen, C₁-C₈ alkoxide, C₁-C₁₀ alkyl, haloalkyl, —OH, —CN, —NO₂, —C₂-C₁₀ alkenyl, —C₂-C₁₀ alkynyl, aryl, heteroaryl, cycloalkyl, cycloalkenyl, heterocycloalkyl, heterocycloalkenyl, C₀-C₄alk-aryl, C₀-C₄alk-heteroaryl, —OR^{a}, —SR^{a}, —NR^{c}R^{d}, —NR^{a}R^{c}, —C(O)R^{b}, —OC(O)R^{b}, —C(O)OR^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —C(═NR^{b})NR^{c}R^{d}, —C(═NOR^{b})NR^{c}R^{d}, —C(═NCN)NR^{c}R^{d}, —P(OR^{c})₂, —P(O)R^{c}R^{b}, —P(O)R^{c}R^{d}, —P(O)OR^{c}OR^{b}, —S(O)R^{b}, —S(O)NR^{c}R^{d}, —S(O)₂NR^{c}R^{d}, —S(O)(═NR^{b})R^{b}, —SF5, —P(O)R^{b}R^{b}, —P(O)(OR^{b})(OR^{b}), —B(OR^{c})(OR^{d}), —S(O)₂R^{b}, —C(O)NR^{b}OR^{b}, —SiR^{b}₃, —S(O)₂OR^{b}, —OS(O)₂OR^{b}, —OPO(OR^{b})(OR^{b}) and -L-W; wherein each C₁-C₈ alkoxide, C₁-C₁₀ alkyl, haloalkyl, —C2-C10 alkenyl, —C2-C10 alkynyl, aryl, heteroaryl, cycloalkyl, cycloalkenyl, heterocycloalkyl, or heterocycloalkenyl is optionally substituted by 1-6 R^{f} groups.

In some embodiments, Ring B is an oxazolidinone.

In some embodiments, the compound of formula (I) is a compound of formula (II):

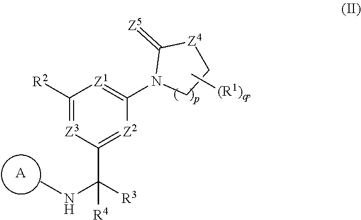

or a pharmaceutically acceptable salt, N-oxide, or stereoisomer thereof; wherein:
Z⁴ is O, NR^{a}, C(R¹)₂, or S;
Z⁵ is O, NR^{a}, N—OR^{a}, N—CN, or S;
p is 1, 2 or 3; and
q is 0, 1, 2, 3, 4, 5, 6, 7, or 8; and
each R¹ is independently H, D, oxo, ═NR^{a}, ═N—OR^{a}, ═N—CN, ═S, halogen, C₁-C₈ alkoxide, C₁-C₁₀ alkyl, haloalkyl, —OH, —CN, —NO₂, —C₂-C₁₀ alkenyl, —C₂-C₁₀ alkynyl, aryl, heteroaryl, cycloalkyl, cycloalkenyl, heterocycloalkyl, heterocycloalkenyl, C₀-C₄alk-aryl, C₀-C₄alk-heteroaryl, —OR^{a}, —SR^{a}, —NR^{c}R^{d}, —NR^{a}R^{c}, —C(O)R^{b}, —OC(O)R^{b}, —C(O)OR^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —C(═NR^{b})NR^{c}R^{d}, —C(═NOR^{b})NR^{c}R^{d}, —C(═NCN)NR^{c}R^{d}, —P(OR^{c})₂, —P(O)R^{c}R^{b}, —P(O)R^{c}R^{d}, —P(O)OR^{c}OR^{b}, —S(O)R^{b}, —S(O)NR^{c}R^{d}, —S(O)₂NR^{c}R^{d}, —S(O)(═NR^{b})R^{b}, —SF5, —P(O)R^{b}R^{b}, —P(O)(OR^{b})(OR^{b}), SiR^{b}₃, —B(OR^{c})(OR^{d}), —S(O)₂R^{b}, —C(O)NR^{b}OR^{b}, —SiR^{b}₃, —S(O)₂OR^{b}, —OS(O)₂OR^{b}, —OPO(OR^{b})(OR^{b}) or -L-W; wherein each C₁-C₈ alkoxide, C₁-C₁₀ alkyl, haloalkyl, —C2-C10 alkenyl, —C2-C10 alkynyl, aryl, heteroaryl, cycloalkyl, cycloalkenyl, heterocycloalkyl, heterocycloalkenyl, C₀-C₄alk-aryl, or C₀-C₄alk-heteroaryl is optionally substituted by 1-6 R^{f} groups.

In some embodiments, the compounds of formula (II) are compounds of Formula (III):

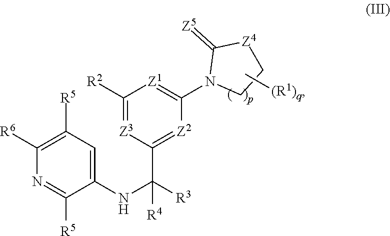

or pharmaceutically acceptable salts, N-oxides, or stereoisomers thereof.

In some embodiments, R¹ in Formula II or Formula III is H. In some embodiments, R¹ in Formula II or Formula III is D. In some embodiments, R¹ in Formula II or Formula III is oxo. In some embodiments, R¹ in Formula II or Formula III is ═NR^{a}. In some embodiments, R¹ in Formula II or Formula III is ═N—OR^{a}. In some embodiments, R¹ in Formula II or Formula III is ═N—CN. In some embodiments, R¹ in Formula II or Formula III is ═S. In some embodiments, R¹ in Formula II or Formula III is halogen. In some embodiments, R¹ in Formula II or Formula III is C₁-C₈ alkoxide. In some embodiments, R¹ in Formula II or Formula III is C₁-C₈ alkyl. In some embodiments, R¹ in Formula II or Formula III is haloalkyl. In some embodiments, R¹ in Formula II or Formula III is —OH. In some embodiments, R¹ in Formula II or Formula III is —CN. In some embodiments, R¹ in Formula II or Formula III is —NO2. In some embodiments, R¹ in Formula II or Formula III is —C2-C6 alkenyl. In some embodiments, R¹ in Formula II or Formula III is —C2-C6 alkynyl. In some embodiments, R¹ in Formula II or Formula III is aryl. In some embodiments, R¹ in Formula II or Formula III is heteroaryl. In some embodiments, R¹ in Formula II or Formula III is cycloalkyl. In other embodiments, R¹ in Formula II or Formula III is cycloalkenyl. In other embodiments, R¹ in Formula II or Formula III is heterocycloalkyl. In other embodiments, R¹ in Formula II or Formula III is heterocycloalkenyl. In other embodiments, R¹ in Formula II or Formula III is —OR^{a}. In other embodiments, R¹ in Formula II or Formula III is —SR^{a}. In other embodiments, R¹ in Formula II or Formula III is —NR^{c}R^{d}. In other embodiments, R¹ in Formula II or Formula III is —NR^{a}R^{c}. In other embodiments, R¹ in Formula II or Formula III is —C(O)R^{b}. In other embodiments, R¹ in Formula II or Formula III is —OC(O)R^{b}. In other embodiments, R¹ in Formula II or Formula III is —C(O)OR^{b}. In other embodiments, R¹ in Formula II or Formula III is —C(O)NR^{c}R^{d}. In yet other embodiments, R¹ in Formula II or Formula III is —S(O)R^{b}. In yet other embodiments, R¹ in Formula II or Formula III is —S(O)₂NR^{c}R^{d}. In yet other embodiments, R¹ in Formula II or Formula III is —S(O)(═NR^{b})R^{b}. In yet other embodiments, R¹ in Formula II or Formula III is —SF5. In yet other embodiments, R¹ in Formula II or Formula III is —P(O)R^{b}R^{b}. In yet other embodiments, R¹ in Formula II or Formula III is —P(O)R^{c}R^{d}. In yet other embodiments, R¹ in Formula II or Formula III is —P(O)R^{b}R^{c}. In yet other embodiments, R¹ in Formula II or Formula III is —P(O)(OR^{b})(OR^{b}). In yet other embodiments, R¹ in Formula II or Formula III is —B(OR^{c})(OR^{d}). In yet other embodiments, R¹ in Formula II or Formula III is —S(O)₂R^{b}. In yet other embodiments, R¹ in Formula II or Formula III is —C(O)NR^{b}OR^{b}. In yet other embodiments, R¹ in Formula II or Formula III is —S(O)₂OR^{b}. In yet other embodiments, R¹ in Formula II or Formula III is —OS(O)₂OR^{b}. In yet other embodiments, R¹ in Formula II or Formula III is —OPO(OR^{b})(OR^{b}).

In some embodiments, R¹ in Formula II or Formula III is -L-W.

In some embodiments, L in Formula I, II, or III is absent or is C₁-C₈ alkylene, O, NR^{a}, S or 3-10 membered cycloalkyl. In some embodiments, L in Formula I, II, or III is absent. In some embodiments, L in Formula I, II, or III is C₁-C₈ alkylene. In other embodiments, L in Formula I, II, or III is O. In other embodiments, L in Formula I, II, or III is NR^{a}. In yet other embodiments, L in Formula I, II, or III is S. In yet other embodiments, L in Formula I, II, or III is 3-10 membered cycloalkyl. In some embodiments, L in Formula I, II, or III is a methylene group.

In some embodiments, W in Formula I, II, or III is a 5-10 membered heteroaryl ring comprising 1-4 heteroatoms selected from N, O, and S; a 5-12 membered heterocyclic group comprising 1-4 heteroatoms selected from N, O, and S; C₁-C₈ alkyl; haloalkyl; —C2-C6 alkenyl; —C₂-C₆ alkynyl; aryl; heteroaryl; cycloalkyl; cycloalkenyl; heterocycloalkenyl; NR^{c}R^{d}; OR^{b}; or SR^{b}; each of which is optionally substituted by 1-6 R^{f} groups.

In some embodiments, W in Formula I, II, or III is aryl optionally substituted by 1-6 R^{f} groups. In other embodiments, W in Formula I, II, or III is phenyl optionally substituted by 1-5 R^{f} groups. In other embodiments, W in Formula I, II, or III is phenyl, 4-fluorophenyl or 2,4-difluorophenyl. In yet other embodiments, W in Formula I, II, or III is phenyl. In yet other embodiments, W in Formula I, II, or III is 4-fluorophenyl. In yet other embodiments, W in Formula I, II, or III is 2,4-difluorophenyl.

In some embodiments, W in Formula I, II, or III is a 5-12 membered heterocyclic group comprising 1-4 heteroatoms selected from N, O, and S. In some embodiments, the 5-12 membered heterocyclic group is an isoindoline group or a piperidine group. In some embodiments, the 5-12 membered heterocyclic group is an isoindoline group. In some embodiments, the 5-12 membered heterocyclic group is a piperidine group. In some embodiments, the 5-12 membered heterocyclic group is a 3-azabicyclo[3.1.0]hexane, pyrrolidine, azetidine, piperazine, 6,7-dihydro-5H-pyrrolo[3,4-b]pyridine or 1,4,5,6-tetrahydropyrrolo[3,4-c]pyrazole group. In some embodiments, the 5-12 membered heterocyclic group is a 3-azabicyclo[3.1.0]hexane. In some embodiments, the 5-12 membered heterocyclic group is a pyrrolidine group. In some embodiments, the 5-12 membered heterocyclic group is an azetidine group. In some embodiments, the 5-12 membered heterocyclic group is a piperazine group. In some embodiments, the 5-12 membered heterocyclic group is 6,7-dihydro-5H-pyrrolo[3,4-b]pyridine group. In some embodiments, the 5-12 membered heterocyclic group is a 1,4,5,6-tetrahydropyrrolo[3,4-c]pyrazole group. In some embodiments, W in Formula (I) is NR^{c}R^{d}. In some embodiments, W in Formula I, II, or III is substituted by 1-6 R^{f} groups.

In other embodiments, W in Formula I, II, or III is a 5-10 membered heteroaryl ring comprising 1-4 heteroatoms selected from N, O, and S. In other embodiments, W in Formula I, II, or III is C₁-C₈ alkyl. In other embodiments, W in Formula I, II, or III is haloalkyl. In other embodiments, W in Formula I, II, or III is —C2-C6 alkenyl. In other embodiments, W in Formula I, II, or III is —C2-C6 alkynyl. In other embodiments, W in Formula I, II, or III is heteroaryl. In other embodiments, W in Formula I, II, or III is cycloalkyl. In other embodiments, W in Formula I, II, or III is cycloalkenyl. In other embodiments, W in Formula I, II, or III is heterocycloalkenyl. In other embodiments, W in Formula I, II, or III is NR^{c}R^{d}. In other embodiments, W in Formula I, II, or III is NR^{c}R^{d}. In other embodiments, W in Formula I, II, or III is OR^{b}. In other embodiments, W in Formula I, II, or III is SR^{b}. In some embodiments, W in Formula I, II, or III is substituted by 1 or 2 R^{f} groups.

In some embodiments, Z⁴ in Formula (II) is O, NR^{a}, C(R¹)₂, or S. In some embodiments, Z⁴ in Formula (II) is O. In other embodiments, Z⁴ in Formula (II) is NR^{a}. In other embodiments, Z⁴ in Formula (II) is C(R¹)₂. In other embodiments, Z⁴ in Formula (II) is S.

In some embodiments, Z⁵ in Formula (II) is O, NR^{a}, N—OR^{a}, or S. In some embodiments, Z⁵ in Formula (II) is O. In other embodiments, Z⁵ in Formula (II) is NR^{a}. In other embodiments, Z⁵ in Formula (II) is N—OR^{a}. In other embodiments, Z⁵ in Formula (II) is S.

In some embodiments, p in Formula (II) is 1, 2, or 3. In some embodiments, p in Formula (II) is 1. In other embodiments, p in Formula (II) is 2. In yet other embodiments, p in Formula (II) is 3.

In some embodiments, q in Formula (II) is 0, 1, 2, 3, 4, 5, 6, 7, or 8. In some embodiments, q in Formula (II) is 1. In other embodiments, q in Formula (II) is 2. In yet other embodiments, q in Formula (II) is 3. In some embodiments, q in Formula (II) is 4. In other embodiments, q in Formula (II) is 5. In yet other embodiments, q in Formula (II) is 6. In some embodiments, q in Formula (II) is 7. In other embodiments, q in Formula (II) is 8. In yet other embodiments, q in Formula (II) is 0.

In some embodiments, Z⁴ in Formula (III) is O, NR^{a}, C(R¹)₂, or S. In some embodiments, Z⁴ in Formula (III) is O. In other embodiments, Z⁴ in Formula (III) is NR^{a}. In other embodiments, Z⁴ in Formula (III) is C(R¹)₂. In other embodiments, Z⁴ in Formula (III) is S.

In some embodiments, Z⁵ in Formula (III) is O, NR^{a}, or S. In some embodiments, Z⁵ in Formula (III) is O. In other embodiments, Z⁵ in Formula (III) is NR^{a}. In other embodiments, Z⁵ in Formula (III) is N—OR^{a}. In other embodiments, Z⁵ in Formula (III) is S.

In some embodiments, p in Formula (III) is 1, 2, or 3. In some embodiments, p in Formula (III) is 1. In other embodiments, p in Formula (III) is 2. In yet other embodiments, p in Formula (III) is 3.

In some embodiments, q in Formula (III) is 0, 1, 2, 3, 4, 5, 6, 7, or 8. In some embodiments, q in Formula (III) is 1. In other embodiments, q in Formula (III) is 2. In yet other embodiments, q in Formula (III) is 3. In some embodiments, q in Formula (III) is 4. In other embodiments, q in Formula (III) is 5. In yet other embodiments, q in Formula (III) is 6. In some embodiments, q in Formula (III) is 7. In other embodiments, q in Formula (III) is 8. In yet other embodiments, q in Formula (III) is 0.

In some embodiments, R⁶ in Formula (II) or Formula (III) is —F or —Cl.

In some embodiments, at least one R⁵ in Formula (II) or Formula (III) is —CO₂H.

In some embodiments, R³ in Formula (II) or Formula (III) is methyl or CD₃.

In some embodiments, at least one R⁴ in Formula (II) or Formula (III) is H.

In some embodiments, Z⁴ in Formula (II) or Formula (III) is O.

In some embodiments, Z⁵ in Formula (II) or Formula (III) is O.

In some embodiments, at least one Z⁴ and Z⁵ in Formula (II) or Formula (III) are O.

In some embodiments, each Z⁴ and Z⁵ in Formula (II) or Formula (III) are O.

In some embodiments, Z⁴ in Formula (II) or Formula (III) is NR^{a}.

In some embodiments, at least one Z⁴ in Formula (II) or Formula (III) is NR^{a} and Z⁵ in Formula (II) or Formula (III) is O.

In some embodiments, q in Formula (II) or Formula (III) is 1.

In some embodiments, the compounds of formula (III) are compounds of Formula (IV) or Formula (V)

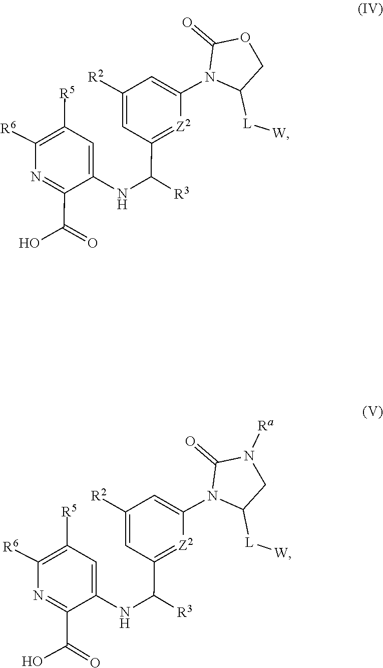

or a pharmaceutically acceptable salt, N-oxide, or stereoisomer thereof.

In some embodiments, the compounds of formula (III) are compounds of Formula (IV):

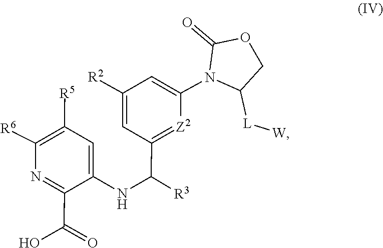

or pharmaceutically acceptable salts, N-oxides, or stereoisomers thereof.

In some embodiments, L in Formula (IV) is absent or is C₁-C₈ alkylene, O, NR^{a}, S or 3-10 membered cycloalkyl. In some embodiments, L in Formula (IV) is absent. In some embodiments, L in Formula (IV) is C₁-C₈ alkylene. In other embodiments, L in Formula (IV) is O. In other embodiments, L in Formula (IV) is NR^{a}. In yet other embodiments, L in Formula (IV) is S. In yet other embodiments, L in Formula (IV) is 3-10 membered cycloalkyl. In some embodiments, L in Formula (IV) is a methylene group.

In some embodiments, W in Formula (IV) is aryl optionally substituted by 1-6 R^{f} groups. In other embodiments, W in Formula (IV) is phenyl optionally substituted by 1-5 R^{f} groups. In other embodiments, W in Formula (IV) is phenyl.

In other embodiments, W in Formula (IV) is phenyl, 4-fluorophenyl or 2,4-difluorophenyl. In yet other embodiments, W in Formula (IV) is phenyl. In yet other embodiments, W in Formula (IV) is 4-fluorophenyl. In yet other embodiments, W in Formula (IV) is 2,4-difluorophenyl.

In some embodiments, R⁶ in Formula (IV) is —F.

In some embodiments, R⁶ in Formula (IV) is —Cl.

In some embodiments, R³ in Formula (IV) is methyl or CD₃. In some embodiments, R³ in Formula (IV) is methyl. In other embodiments, R³ in Formula (IV) is CD₃.

In some embodiments, the compounds of formula (III) are compounds of Formula (V):

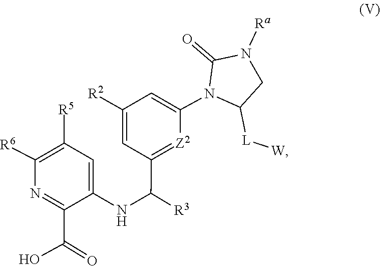

or pharmaceutically acceptable salts, N-oxides, or stereoisomers thereof.

In some embodiments, R^{a} in Formula (V) is H, C₁-C₆ alkyl, or 3-6 membered cycloalkyl. In some embodiments, R^{a} in Formula (V) is H. In some embodiments, R^{a} in Formula (V) is 3-6 membered cycloalkyl. In other embodiments, R^{a} in Formula (V) is C₁-C₆ alkyl. In other embodiments, R^{a} in Formula (V) is methyl.

In some embodiments, L in Formula (V) is absent or is C₁-C₈ alkylene, O, NR^{a}, S or 3-10 membered cycloalkyl. In some embodiments, L in Formula (V) is absent. In some embodiments, L in Formula (V) is C₁-C₈ alkylene. In other embodiments, L in Formula (V) is O. In other embodiments, L in Formula (V) is NR^{a}. In yet other embodiments, L in Formula (V) is S. In yet other embodiments, L in Formula (V) is 3-10 membered cycloalkyl. In some embodiments, L in Formula (V) is a methylene group.

In some embodiments, W in Formula (V) is aryl optionally substituted by 1-6 R^{f} groups. In other embodiments, W in Formula (V) is phenyl optionally substituted by 1-5 R^{f} groups. In other embodiments, W in Formula (V) is phenyl.

In other embodiments, W in Formula (V) is phenyl, 4-fluorophenyl or 2,4-difluorophenyl. In yet other embodiments, W in Formula (V) is phenyl. In yet other embodiments, W in Formula (V) is 4-fluorophenyl. In yet other embodiments, W in Formula (V) is 2,4-difluorophenyl.

In some embodiments, R⁶ in Formula (V) is —F.

In some embodiments, R⁶ in Formula (V) is —Cl.

In some embodiments, R³ in Formula (V) is methyl or CD₃. In some embodiments, R³ in Formula (IV) is methyl. In other embodiments, R³ in Formula (V) is CD₃.

In some embodiments, the compounds of Formula (I) are the pharmaceutically acceptable salts. In some embodiments, the compounds of Formula (I) are solvates. In some embodiments, the compounds of Formula (I) are N-oxides. In some embodiments, the compounds of Formula (I) are stereoisomers.

In some embodiments, the compounds of Formula (I) are:
3-((1-(3-((S)-4-Benzyl-2-oxooxazolidin-3-yl)-5-methylphenyl)ethyl)amino)-6-chloro-picolinic acid;
3-(1-(3-((S)-4-Benzyl-2-oxooxazolidin-3-yl)-5-methylphenyl)ethyl)amino)-6-chloro-picolinic acid;
3-((1-(3-((S)-4-Benzyl-2-oxooxazolidin-3-yl)-5-methylphenyl)ethyl)amino)-6-fluoro-picolinic acid;
3-((1-(3-((S)-4-benzyl-2-oxooxazolidin-3-yl)-5-fluorophenyl)ethyl)amino)-6-chloro-picolinic acid;
3-((1-(3-((S)-4-Benzyl-2-oxooxazolidin-3-yl)-5-fluorophenyl)ethyl)amino)-6-fluoro-picolinic acid;
3-((1-(3-((S)-4-Benzyl-2-oxooxazolidin-3-yl)-5-chlorophenyl)ethyl)amino)-6-chloro-picolinic acid;
3-((1-(3-((S)-4-Benzyl-2-oxooxazolidin-3-yl)-5-chlorophenyl)ethyl)amino)-6-fluoro-picolinic acid;
3-((1-(3-((S)-4-Benzyl-2-oxooxazolidin-3-yl)-5-(trifluoromethyl)phenyl)ethyl)amino)-6-fluoropicolinic acid;
3-(((R)-1-(3-((S)-4-(4-Bromobenzyl)-2-oxooxazolidin-3-yl)-5-methylphenyl)ethyl)amino)-6-chloropicolinic acid;
3-(((R)-1-(3-((S)-4-Benzyl-2-oxooxazolidin-3-yl)-5-fluorophenyl)ethyl)amino)-6-chloro-picolinic acid;
3-[1-[6-[(4S)-4-Benzyl-2-oxo-1,3-oxazolidin-3-yl]-4-methyl-2-pyridinyl]ethylamino]-6-fluoropyridine-2-carboxylic acid;
3-(((R)-1-(6-((S)-4-Benzyl-2-oxooxazolidin-3-yl)-4-methylpyridin-2-yl)ethyl)amino)-6-chloropicolinic acid;
3-((1-(6-((S)-4-Benzyl-2-oxooxazolidin-3-yl)-4-methylpyridin-2-yl)ethyl)amino)picolinic acid;
3-((1-(6-((S)-4-Benzyl-2-oxooxazolidin-3-yl)-4-methylpyridin-2-yl)ethyl)amino)-6-methylpicolinic acid;
3-((1-(6-((S)-4-Benzyl-2-oxooxazolidin-3-yl)-4-methylpyridin-2-yl)ethyl)amino)-6-(trifluoromethyl)picolinic acid;
3-((1-(6-((S)-4-Benzyl-2-oxooxazolidin-3-yl)-4-methylpyridin-2-yl)ethyl)amino)-6-methoxypicolinic acid;
2-((1-(6-((S)-4-Benzyl-2-oxooxazolidin-3-yl)-4-methylpyridin-2-yl)ethyl)amino)benzoic acid;
3-((1-(6-((S)-4-Benzyl-2-oxooxazolidin-3-yl)-4-methylpyridin-2-yl)ethyl)amino)-6-bromopicolinic acid;
3-((1-(6-((S)-4-Benzyl-2-oxooxazolidin-3-yl)-4-methylpyridin-2-yl)ethyl)amino)-6-cyanopicolinic acid;
   or a pharmaceutically acceptable salt, N-oxide, or stereoisomer thereof.

In some embodiments, the compounds of Formula (I) are:
6-Chloro-3-(((R)-1-(6-((S)-4-(2-fluoro-3-methylbenzyl)-2-oxooxazolidin-3-yl)-4-methyl-pyridin-2-yl)ethyl)amino)picolinic acid;
6-Chloro-3-(((R)-1-(6-((S)-4-((3-fluoropyridin-4-yl)methyl)-2-oxooxazolidin-3-yl)-4-methylpyridin-2-yl)ethyl)amino)picolinic acid;
3-(((R)-1-(6-((S)-4-(2-Bromobenzyl)-2-oxooxazolidin-3-yl)-4-methylpyridin-2-yl)ethyl) amino)-6-chloropicolinic acid;
6-Chloro-3-(((R)-1-(4-methyl-6-((S)-2-oxo-4-(4-(trifluoromethyl)benzyl)oxazolidin-3-yl)pyridin-2-yl)ethyl)amino)picolinic acid;
6-Chloro-3-(((R)-1-(6-((S)-4-(4-fluorobenzyl)-2-oxooxazolidin-3-yl)-4-methylpyridin-2-yl)ethyl)amino)picolinic acid;
3-(((R)-1-(6-((S)-4-(4-(tert-Butoxycarbonyl)benzyl)-2-oxooxazolidin-3-yl)-4-methylpyridin-2-yl)ethyl)amino)-6-chloropicolinic acid;
6-Chloro-3-(((R)-1-(6-((S)-4-(2,4-difluorobenzyl)-2-oxooxazolidin-3-yl)-4-methylpyridin-2-yl)ethyl)amino)picolinic acid;
6-Chloro-3-(((R)-1-(6-((S)-4-(2-fluoro-4-methoxybenzyl)-2-oxooxazolidin-3-yl)-4-methyl-pyridin-2-yl)ethyl)amino)picolinic acid;
6-Chloro-3-(((R)-1-(4-methyl-6-((S)-2-oxo-4-(pyridin-4-ylmethyl)oxazolidin-3-yl)pyridin-2-yl)ethyl)amino)picolinic acid;
6-Chloro-3-(((R)-1-(6-((S)-4-((5-fluoro-2-methoxypyridin-4-yl)methyl)-2-oxooxazolidin-3-yl)-4-methylpyridin-2-yl)ethyl)amino)picolinic acid;
6-Chloro-3-(((R)-1-(6-((S)-4-((5-fluoro-2-methylpyridin-4-yl)methyl)-2-oxooxazolidin-3-yl)-4-methylpyridin-2-yl)ethyl)amino)picolinic acid;
6-Chloro-3-(((R)-1-(4-methyl-6-((S)-4-(naphthalen-1-ylmethyl)-2-oxooxazolidin-3-yl)pyridin-2-yl)ethyl)amino)picolinic acid;
6-Chloro-3-(((R)-1-(4-methyl-6-((S)-4-((1-methyl-1H-pyrazol-4-yl)methyl)-2-oxooxazolidin-3-yl)pyridin-2-yl)ethyl)amino)picolinic acid;
6-Chloro-3-(((R)-1-(4-methyl-6-((S)-4-((1-methyl-1H-pyrazol-3-yl)methyl)-2-oxooxazolidin-3-yl)pyridin-2-yl)ethyl)amino)picolinic acid;
6-Chloro-3-(((R)-1-(4-methyl-6-((S)-4-((1-methyl-1H-pyrazol-5-yl)methyl)-2-oxooxazolidin-3-yl)pyridin-2-yl)ethyl)amino)picolinic acid;
6-Chloro-3-(((R)-1-(6-((S)-4-((1,3-dimethyl-1H-pyrazol-4-yl)methyl)-2-oxooxazolidin-3-yl)-4-methylpyridin-2-yl)ethyl)amino)picolinic acid;
6-Chloro-3-(((R)-1-(6-((S)-4-((3-fluoro-1-methyl-1H-pyrazol-4-yl)methyl)-2-oxooxazolidin-3-yl)-4-methylpyridin-2-yl)ethyl)amino)picolinic acid;
6-Chloro-3-(((R)-1-(4-methyl-6-((S)-4-((1-methyl-1H-indol-3-yl)methyl)-2-oxooxazolidin-3-yl)pyridin-2-yl)ethyl)amino)picolinic acid;
6-Chloro-3-(((R)-1-(4-methyl-6-((S)-4-((1-methyl-1H-indazol-3-yl)methyl)-2-oxooxazolidin-3-yl)pyridin-2-yl)ethyl)amino)picolinic acid;
6-Chloro-3-(((R)-1-(4-methyl-6-((S)-4-((1-methyl-1H-pyrrolo[2,3-b]pyridin-3-yl)methyl)-2-oxooxazolidin-3-yl)pyridin-2-yl)ethyl)amino)picolinic acid;
6-Chloro-3-(((R)-1-(4-methyl-6-((S)-2-oxo-4-(pyrazolo[1,5-a]pyridin-5-ylmethyl) oxazolidin-3-yl)pyridin-2-yl)ethyl)amino)picolinic acid;
6-Chloro-3-(((R)-1-(6-((S)-4-(imidazo[1,5-a]pyridin-6-ylmethyl)-2-oxooxazolidin-3-yl)-4-methylpyridin-2-yl)ethyl)amino)picolinic acid;
6-Chloro-3-(((R)-1-(4-methyl-6-((S)-4-((1-methyl-1H-indazol-5-yl)methyl)-2-oxooxazolidin-3-yl)pyridin-2-yl)ethyl)amino)picolinic acid;
6-Chloro-3-(((R)-1-(4-methyl-6-((S)-4-((2-methyl-2H-indazol-5-yl)methyl)-2-oxooxazolidin-3-yl)pyridin-2-yl)ethyl)amino)picolinic acid;
6-Chloro-3-(((R)-1-(6-((S)-4-(imidazo[1,2-a]pyridin-8-ylmethyl)-2-oxooxazolidin-3-yl)-4-methylpyridin-2-yl)ethyl)amino)picolinic acid;
6-Chloro-3-(((R)-1-(4-methyl-6-((S)-2-oxo-4-(pyrazolo[1,5-a]pyridin-4-ylmethyl) oxazolidin-3-yl)pyridin-2-yl)ethyl)amino)picolinic acid;
6-Chloro-3-(((R)-1-(4-methyl-6-((S)-2-oxo-4-(pyrazolo[1,5-a]pyridin-3-ylmethyl) oxazolidin-3-yl)pyridin-2-yl)ethyl)amino)picolinic acid;
6-Chloro-3-(((R)-1-(6-((S)-4-(cyclobutylmethyl)-2-oxooxazolidin-3-yl)-4-methylpyridin-2-yl)ethyl)amino)picolinic acid;
6-Chloro-3-(((R)-1-(6-((S)-4-(3-cyanobenzyl)-2-oxooxazolidin-3-yl)-4-methylpyridin-2-yl)ethyl)amino)picolinic acid;
6-Chloro-3-(((R)-1-(6-((S)-4-(4-cyano-2-fluorobenzyl)-2-oxooxazolidin-3-yl)-4-methyl-pyridin-2-yl)ethyl)amino)picolinic acid;
3-(((R)-1-(6-((S)-4-((1H-Indol-3-yl)methyl)-2-oxooxazolidin-3-yl)-4-methylpyridin-2-yl)ethyl)amino)-6-chloropicolinic acid;
6-Chloro-3-(((R)-1-(6-((S)-4-(4-(dimethylcarbamoyl)benzyl)-2-oxooxazolidin-3-yl)-4-methylpyridin-2-yl)ethyl)amino)picolinic acid;
6-Chloro-3-(((R)-1-(6-((S)-4-(4-(methoxy(methyl)carbamoyl)benzyl)-2-oxooxazolidin-3-yl)-4-methylpyridin-2-yl)ethyl)amino)picolinic acid;
6-Chloro-3-(((R)-1-(4-methyl-6-((S)-2-oxo-4-phenethyloxazolidin-3-yl)pyridin-2-yl)ethyl) amino)picolinic acid;
3-(((R)-1-(6-((S)-4-Benzyl-4-methyl-2-oxooxazolidin-3-yl)-4-methylpyridin-2-yl)ethyl) amino)-6-chloropicolinic acid;
3-(((1R)-1-(6-(2-benzyl-5-oxopyrrolidin-1-yl)-4-methylpyridin-2-yl)ethyl)amino)-6-chloropicolinic acid;
3-(((R)-1-(6-((S)-4-Benzyl-2-oxo-1,3-oxazinan-3-yl)-4-methylpyridin-2-yl)ethyl)amino)-6-chloropicolinic acid;
3-(((R)-1-(6-((S)-3-Benzyl-5-oxomorpholino)-4-methylpyridin-2-yl)ethyl)amino)-6-chloropicolinic acid;
6-Chloro-3-(((R)-1-(6-((S)-5-(4-fluorobenzyl)-3-methyl-2-oxoimidazolidin-1-yl)-4-methylpyridin-2-yl)ethyl)amino)picolinic acid;
6-Chloro-3-(((R)-1-(6-((S)-5-(2,4-difluorobenzyl)-3-methyl-2-oxoimidazolidin-1-yl)-4-methylpyridin-2-yl)ethyl)amino)picolinic acid;
3-(((R)-1-(6-((S)-5-Benzyl-3-methyl-2-oxoimidazolidin-1-yl)-4-methylpyridin-2-yl)ethyl) amino)-6-chloropicolinic acid;
6-Chloro-3-(((R)-1-(3-fluoro-5-((S)-4-(2-fluorobenzyl)-2-oxooxazolidin-3-yl)phenyl)ethyl) amino)picolinic acid;
6-Chloro-3-(((R)-1-(3-fluoro-5-((S)-4-(3-methylbenzyl)-2-oxooxazolidin-3-yl)phenyl)ethyl) amino)picolinic acid;
   or a pharmaceutically acceptable salt, N-oxide, or stereoisomer thereof.

It will be apparent that the compounds of Formula I, including all subgenera described herein, may have multiple stereogenic centers. As a result, there exist multiple stereoisomers (enantiomers and diastereomers) of the compounds of Formula I (and subgenera described herein). The present disclosure contemplates and encompasses each stereoisomer of any compound of Formula I (and subgenera described herein), as well as mixtures of said stereoisomers.

Pharmaceutically acceptable salts, N-oxides, solvates, or stereoisomers of the compounds of Formula I (including all subgenera described herein) are also within the scope of the disclosure. Thus, in some embodiments, the compounds of the disclosure are pharmaceutically acceptable salts of the compounds of Formula I (including all subgenera described herein). In other embodiments, the compounds of the disclosure are not salts. In some embodiments, the compounds of the disclosure are N-oxides of the compounds of Formula I (including all subgenera described herein). In other embodiments, the compounds of the disclosure are not N-oxides of the compounds of Formula I (including all subgenera described herein). In some embodiments, the compounds of the disclosure are solvates of the compounds of Formula I (including all subgenera described herein). In other embodiments, the compounds of the disclosure are not solvates of the compounds of Formula I (including all subgenera described herein).

Isotopic variants of the compounds of Formula I (including all subgenera described herein) are also contemplated by the present disclosure.

In some embodiments, the compounds of the disclosure include a carboxylic acid moiety. In some aspects, the present disclosure also encompasses carboxylic acid prodrugs of these embodiments. Carboxylic acid prodrugs include, but are not limited to, C₁-C₆ alkyl esters (e.g., methyl, ethyl, isopropyl, butyl, and isoamyl), 2-aminoethyl esters (e.g., 2-morpholinoethyl), C₆-C₁₀ aryl esters (e.g., phenyl, indanyl, and guaiacol), (acyloxy)alkyl esters, [(alkoxycarbonyl)oxy]alkyl esters, and (5-methyl-2-oxo-1,3-dioxolen-4-yl)methyl esters. See, e.g., Maag, H. (2007). Prodrugs of Carboxylic Acids. In: Stella, V. J., Borchardt, R. T., Hageman, M. J., Oliyai, R., Maag, H., Tilley, J. W. (eds) Prodrugs. Biotechnology: Pharmaceutical Aspects, vol V. Springer, New York, NY. https://doi.org/10.1007/978-0-387-49785-3_20.

### Pharmaceutical Compositions and Methods of Administration

In some embodiments, the disclosure is directed to pharmaceutical compositions comprising compounds of Formula I, or a pharmaceutically acceptable salt, N-oxide, or stereoisomer thereof.

The subject pharmaceutical compositions are typically formulated to provide a therapeutically effective amount of a compound of the present disclosure as the active ingredient, or a pharmaceutically acceptable salt, ester, prodrug, solvate, hydrate or derivative thereof. Where desired, the pharmaceutical compositions contain pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

The subject pharmaceutical compositions can be administered alone or in combination with one or more other agents, which are also typically administered in the form of pharmaceutical compositions. Where desired, the one or more compounds of the invention and other agent(s) may be mixed into a preparation or both components may be formulated into separate preparations to use them in combination separately or at the same time.

In some embodiments, the concentration of one or more compounds provided in the pharmaceutical compositions of the present invention is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v or v/v.

In some embodiments, the concentration of one or more compounds of the invention is greater than 90%, 80%, 70%, 60%, 50%, 40%, 30%, 20%, 19.75%, 19.50%, 19.25%, 19%, 18.75%, 18.50%, 18.25% 18%, 17.75%, 17.50%, 17.25% 17%, 16.75%, 16.50%, 16.25%, 16%, 15.75%, 15.50%, 15.25% 15%, 14.75%, 14.50%, 14.25% 14%, 13.75%, 13.50%, 13.25%, 13%, 12.75%, 12.50%, 12.25%, 12%, 11.75%, 11.50%, 11.25% 11%, 10.75%, 10.50%, 10.25% 10%, 9.75%, 9.50%, 9.25%, 9%, 8.75%, 8.50%, 8.25% 8%, 7.75%, 7.50%, 7.25%, 7%, 6.75%, 6.50%, 6.25%, 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 1.25%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v, or v/v.

In some embodiments, the concentration of one or more compounds of the invention is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of one or more compounds of the invention is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount of one or more compounds of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g (or a number in the range defined by and including any two numbers above).

In some embodiments, the amount of one or more compounds of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g (or a number in the range defined by and including any two numbers above).

In some embodiments, the amount of one or more compounds of the invention is in the range of 0.0001-10 g, 0.0005-9 g, 0.001-8 g, 0.005-7 g, 0.01-6 g, 0.05-5 g, 0.1-4 g, 0.5-4 g, or 1-3 g.

The compounds according to the invention are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. An exemplary dosage is 10 to 30 mg per day. The exact dosage will depend upon the route of administration, the form in which the compound is administered, the subject to be treated, the body weight of the subject to be treated, and the preference and experience of the attending physician.

A pharmaceutical composition of the invention typically contains an active ingredient (i.e., a compound of the disclosure) of the present invention or a pharmaceutically acceptable salt and/or coordination complex thereof, and one or more pharmaceutically acceptable excipients, carriers, including but not limited to inert solid diluents and fillers, diluents, sterile aqueous solution and various organic solvents, permeation enhancers, solubilizers and adjuvants.

Described below are non-limiting exemplary pharmaceutical compositions and methods for preparing the same.

### Pharmaceutical Compositions for Oral Administration.

In some embodiments, the invention provides a pharmaceutical composition for oral administration containing a compound of the invention, and a pharmaceutical excipient suitable for oral administration.

In some embodiments, the invention provides a solid pharmaceutical composition for oral administration containing: (i) an effective amount of a compound of the invention; optionally (ii) an effective amount of a second agent; and (iii) a pharmaceutical excipient suitable for oral administration. In some embodiments, the composition further contains: (iv) an effective amount of a third agent.

In some embodiments, the pharmaceutical composition may be a liquid pharmaceutical composition suitable for oral consumption. Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This invention further encompasses anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions.

Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP—phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10-oleate, Tween 40, Tween 60, sucrose monostearate, sucrose mono laurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the compound of the present invention and to minimize precipitation of the compound of the present invention. This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxy-ethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a subject using conventional techniques, such as distillation or evaporation. Thus, if present, the solubilizer can be in a weight ratio of 10%, 25% o, 50%), 100% o, or up to about 200%> by weight, based on the combined weight of the drug, and other excipients. If desired, very small amounts of solubilizer may also be used, such as 5%>, 2%>, 1%) or even less. Typically, the solubilizer may be present in an amount of about 1%> to about 100%, more typically about 5%> to about 25%> by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)-aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

### Pharmaceutical Compositions for Injection.

In some embodiments, the invention provides a pharmaceutical composition for injection containing a compound of the present invention and a pharmaceutical excipient suitable for injection. Components and amounts of agents in the compositions are as described herein.

The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Pharmaceutical Compositions for Topical (e.g., Transdermal) Delivery.

In some embodiments, the invention provides a pharmaceutical composition for transdermal delivery containing a compound of the present invention and a pharmaceutical excipient suitable for transdermal delivery.

Compositions of the present invention can be formulated into preparations in solid, semisolid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation.

Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present invention employs transdermal delivery devices (“patches”). Such transdermal patches may be used to provide continuous or discontinuous infusion of a compound of the present invention in controlled amounts, either with or without another agent.

The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

### Pharmaceutical Compositions for Inhalation.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

### Other Pharmaceutical Compositions.

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

Administration of the compounds or pharmaceutical composition of the present invention can be affected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. Compounds can also be administered intraadiposally or intrathecally.

The amount of the compound administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to 7 g/day, preferably about 0.05 to about 2.5 g/day. In some instances, dosage levels below the lower limit of the aforesaid range may be more than adequate, while in other cases still larger doses may be employed without causing any harmful side effect, e.g., by dividing such larger doses into several small doses for administration throughout the day.

In some embodiments, a compound of the invention is administered in a single dose.

Typically, such administration will be by injection, e.g., intravenous injection, in order to introduce the agent quickly. However, other routes may be used as appropriate. A single dose of a compound of the invention may also be used for treatment of an acute condition.

In some embodiments, a compound of the invention is administered in multiple doses. Dosing may be about once, twice, three times, four times, five times, six times, or more than six times per day. Dosing may be about once a month, once every two weeks, once a week, or once every other day. In another embodiment a compound of the invention and another agent are administered together about once per day to about 6 times per day. In another embodiment the administration of a compound of the invention and an agent continues for less than about 7 days. In yet another embodiment the administration continues for more than about 6, 10, 14, 28 days, two months, six months, or one year. In some cases, continuous dosing is achieved and maintained as long as necessary.

Administration of the compounds of the invention may continue as long as necessary. In some embodiments, a compound of the invention is administered for more than 1, 2, 3, 4, 5, 6, 7, 14, or 28 days. In some embodiments, a compound of the invention is administered for less than 28, 14, 7, 6, 5, 4, 3, 2, or 1 day. In some embodiments, a compound of the invention is administered chronically on an ongoing basis, e.g., for the treatment of chronic effects.

An effective amount of a compound of the invention may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

The compositions of the invention may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the invention may slow or inhibit the migration and proliferation of smooth muscle cells in the arterial wall which contribute to restenosis. A compound of the invention may be administered, for example, by local delivery from the struts of a stent, from a stent graft, from grafts, or from the cover or sheath of a stent. In some embodiments, a compound of the invention is admixed with a matrix. Such a matrix may be a polymeric matrix and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly (ether-ester) copolymers (e.g. PEO-PLLA); polydimethylsiloxane, poly(ethylene-vinylacetate), acrylate-based polymers or copolymers (e.g. polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. Compounds of the invention may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the invention in a suitable solvent, followed by evaporation of the solvent. Excess drug on the surface of the stent may be removed via an additional brief solvent wash. In yet other embodiments, compounds of the invention may be covalently linked to a stent or graft. A covalent linker may be used which degrades in vivo, leading to the release of the compound of the invention. Any bio-labile linkage may be used for such a purpose, such as ester, amide or anhydride linkages. Compounds of the invention may additionally be administered intravascularly from a balloon used during angioplasty. Extravascular administration of the compounds via the pericard or via advential application of formulations of the invention may also be performed to decrease restenosis.

A variety of stent devices which may be used as described are disclosed, for example, in the following references, all of which are hereby incorporated by reference: U.S. Pat. Nos. 5,451,233; 5,040,548; 5,061,273; 5,496,346; 5,292,331; 5,674,278; 3,657,744; 4,739,762; 5,195,984; 5,292,331; 5,674,278; 5,879,382; 6,344,053.

The compounds of the invention may be administered in dosages. It is known in the art that due to intersubject variability in compound pharmacokinetics, individualization of dosing regimen is necessary for optimal therapy. Dosing for a compound of the invention may be found by routine experimentation in light of the instant disclosure.

When a compound of the invention is administered in a composition that comprises one or more agents, and the agent has a shorter half-life than the compound of the invention unit dose forms of the agent and the compound of the invention may be adjusted accordingly.

The subject pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compound in sterile aqueous solutions, for example, aqueous propylene glycol or dextrose solutions. Such dosage forms can be suitably buffered, if desired.

### Methods of Use

The method typically comprises administering to a subject a therapeutically effective amount of a compound of the invention. The therapeutically effective amount of the subject combination of compounds may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of proliferation or downregulation of activity of a target protein. The specific dose will vary depending on the particular compounds chosen, the dosing regimen to be followed, whether it is administered in combination with other compounds, timing of administration, the tissue to which it is administered, and the physical delivery system in which it is carried.

As used herein, the term “IC₅₀” refers to the half maximal inhibitory concentration of an inhibitor in inhibiting biological or biochemical function. This quantitative measure indicates how much of a particular inhibitor is needed to inhibit a given biological process (or component of a process, i.e., an enzyme, cell, cell receptor or microorganism) by half. In other words, it is the half maximal (50%) inhibitory concentration (IC) of a substance (50% IC, or IC₅₀). EC₅₀ refers to the plasma concentration required for obtaining 50% of a maximum effect in vivo.

In some aspects, the present disclosure provides a method of modulating PI3K (e.g., PI3Kα) activity (e.g., in vitro or in vivo), comprising contacting a cell with a therapeutically effective amount of a compound as described herein or a pharmaceutically acceptable salt, N-oxide, or stereoisomer thereof.

In some aspects, the present disclosure provides a method of treating or preventing a disease or disorder disclosed herein in a subject in need thereof, comprising administering to the subject a therapeutically effective amount of a compound as described herein or a pharmaceutically acceptable salt, N-oxide, or stereoisomer thereof, or a pharmaceutical composition of the present disclosure.

In some aspects, the present disclosure provides a method of treating a disease or disorder disclosed herein in a subject in need thereof, comprising administering to the subject a therapeutically effective amount of a compound as described herein or a pharmaceutically acceptable salt, N-oxide, or stereoisomer thereof, or a pharmaceutical composition of the present disclosure.

In some embodiments, the disease or disorder is associated with an implicated PI3K activity. In some embodiments, the disease or disorder is a disease or disorder in which PI3K activity is implicated.

In some embodiments, the disease or disorder is a cancer.

In some embodiments, the cancer is selected from acute lymphoblastic leukemia (ALL), acute myeloid leukemia (AML), adrenocortical carcinoma, aids-related cancers, aids-related lymphoma, anal cancer, astrocytoma, basal cell carcinoma, bile duct cancer, bladder cancer, bone cancer, osteosarcoma, malignant fibrous histiocytoma, brain tumors, breast cancer, bronchial tumors, Burkitt lymphoma, carcinoid tumor, cancer of unknown primary, cardiac (heart) tumors, atypical teratoid/rhabdoid tumor, primary CNS lymphoma, cervical cancer, cholangiocarcinoma, chordoma, chronic lymphocytic leukemia (CLL), chronic myelogenous leukemia (CML), colorectal cancer, craniopharyngioma, cutaneous t-cell lymphoma, mycosis fungoides, Sezary syndrome, ductal carcinoma in situ (DCIS), embryonal tumors, medulloblastoma, endometrial cancer, ependymoma, esophageal cancer, esthesioneuroblastoma, Ewing sarcoma, extracranial germ cell tumor, extragonadal germ cell tumor, fallopian tube cancer, gallbladder cancer, gastric cancer, gastrointestinal carcinoid tumor, malignant gastrointestinal stromal tumors (GIST), germ cell tumors, gestational trophoblastic disease, hairy cell leukemia, head and neck cancer, hepatocellular cancer, Langerhans cell histiocytosis, Hodgkin lymphoma, islet cell tumors, pancreatic neuroendocrine tumors, Kaposi sarcoma, kidney cancer, laryngeal cancer, leukemia, liver cancer, lung cancer, lymphoma, male breast cancer, intraocular melanoma, Merkel cell carcinoma, malignant mesothelioma, metastatic cancer, metastatic squamous neck cancer, midline tract carcinoma with nut gene changes, mouth cancer, multiple endocrine neoplasia syndromes, multiple myeloma/plasma cell neoplasms, myelodysplastic syndromes, myelodysplastic neoplasms, myeloproliferative neoplasms, chronic myeloproliferative neoplasm, nasal cavity and paranasal sinus cancer, nasopharyngeal cancer, neuroblastoma, non-Hodgkin lymphoma, non-small cell lung cancer, oral cancer, lip and oral cavity cancer, oropharyngeal cancer, malignant fibrous histiocytoma of bone, ovarian cancer, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm, multiple myeloma, pleuropulmonary blastoma, primary central nervous system (CNS) lymphoma, primary peritoneal cancer, prostate cancer, rectal cancer, recurrent cancer, renal cell (kidney) cancer, retinoblastoma, rhabdomyosarcoma, salivary gland cancer, sarcoma, childhood vascular tumors, skin cancer, small cell lung cancer, small intestine cancer, soft tissue sarcoma, squamous cell carcinoma of the skin, testicular cancer, oropharyngeal cancer, hypopharyngeal cancer, thymoma, thymic carcinoma, thyroid cancer, tracheobronchial tumors, transitional cell cancer of the renal pelvis and ureter, urethral cancer, uterine sarcoma, vaginal cancer, vascular tumors, vulvar cancer, and Wilms tumor.

In some embodiments, the cancer is Endometrial cancer, Breast cancer, oesophageal squamous-cell cancer, Cervical squamous-cell carcinoma, Cervical adenocarcinoma, Colorectal adenocarcinoma, Bladder Urothelial Carcinoma, Glioblastoma, Ovarian cancer, Non-small-cell Lung cancer, Esophagogastric cancer, Nerve-sheath tumor, Head and neck squamous-cell carcinoma, Melanoma, Esophagogastric adenocarcinoma, Soft-tissue sarcoma, Prostate cancer, Fibrolamellar carcinoma, Hepatocellular carcinoma, Diffuse glioma, Colorectal cancer, Pancreatic cancer, Cholangiocarcinoma, B-cell lymphoma, Mesothelioma, Adrenocortical carcinoma, Renal non-clear-cell carcinoma, Renal clear-cell carcinoma, Germ-cell carcinoma, Thymic tumor, Pheochromocytoma, Miscellaneous neuroepithelial tumor, thyroid cancer, leukemia, or encapsulated glioma.

In some embodiments, the cancer is endometrial cancer, gastric cancer, leukemia, lymphoma, sarcoma, colorectal cancer, lung cancer, ovarian cancer, skin cancer, head and neck cancer, breast cancer, brain cancer, cervical cancer, bladder cancer, esophageal cancer, pancreatic cancer, bone cancer, hepatobiliary cancer, medulloblastoma, kidney cancer or prostate cancer.

In some embodiments, the cancer is a breast cancer, a prostate cancer, or a brain cancer.

In some embodiments, the cancer is a breast cancer. In some embodiments, the cancer is a prostate cancer. In some embodiments, the cancer is a brain cancer.

In some embodiments, the breast cancer is metastatic breast cancer. In some embodiments, the breast cancer is ductal carcinoma in situ (DCIS). In some embodiments, the breast cancer is invasive ductal carcinoma. In some embodiments, the breast cancer is triple negative breast cancer. In some embodiments, the breast cancer is medullary carcinoma. In some embodiments, the breast cancer is tubular carcinoma. In some embodiments, the breast cancer is mucinous carcinoma. In some embodiments, the breast cancer is Paget disease of the breast or nipple. In some embodiments, the breast cancer is inflammatory breast cancer (IBC).

In some embodiments, the prostate cancer is an adenocarcinoma. In some embodiments, the prostate cancer is a small cell carcinoma. In some embodiments, the prostate cancer is a neuroendocrine tumor. In some embodiments, the prostate cancer is a transitional cell carcinoma. In some embodiments, the prostate cancer is a sarcoma.

In some embodiments, the brain cancer is an acoustic neuroma. In some embodiments, the brain cancer is an astrocytoma. In some embodiments, the brain cancer is a brain metastasis. In some embodiments, the brain cancer is choroid plexus carcinoma. In some embodiments, the brain cancer is craniopharyngioma. In some embodiments, the brain cancer is an embryonal tumor. In some embodiments, the brain cancer is an ependymoma. In some embodiments, the brain cancer is a glioblastoma. In some embodiments, the brain cancer is a glioma. In some embodiments, the brain cancer is a medulloblastoma. In some embodiments, the brain cancer is a meningioma. In some embodiments, the brain cancer is an oligodendroglioma. In some embodiments, the brain cancer is a pediatric brain tumor. In some embodiments, the brain cancer is a pineoblastoma. In some embodiments, the brain cancer is a pituitary tumor.

In some embodiments, the disease or disorder associated with PI3K includes, but is not limited to, CLOVES syndrome (congenial lipomatous overgrowth, vascular malformations, epidermal naevi, scoliosis/skeletal and spinal syndrome), PIK3CA-related overgrowth syndrome (PROS), breast cancer, brain cancer, prostate cancer, endometrial cancer, gastric cancer, leukemia, lymphoma, sarcoma, colorectal cancer, lung cancer, ovarian cancer, skin cancer, or head and neck cancer.

In some embodiments, the diseases or disorder associated with PI3K is CLOVES syndrome (congenital lipomatous overgrowth, vascular malformations, epidermal naevi, scoliosis/skeletal and spinal syndrome).

In some embodiments, the disease or disorder associated with PI3K is PIK3CA-related overgrowth syndrome (PROS).

In some embodiments, the disease or disorder associated with PI3K is breast cancer, brain cancer, prostate cancer, endometrial cancer, gastric cancer, leukemia, lymphoma, sarcoma, colorectal cancer, lung cancer, ovarian cancer, skin cancer, or head and neck cancer.

In some embodiments, the disease or disorder associated with PI3K is breast cancer, brain cancer, prostate cancer, endometrial cancer, gastric cancer, colorectal cancer, lung cancer, ovarian cancer, skin cancer, or head and neck cancer.

In some embodiments, the disease or disorder associated with PI3K is leukemia, lymphoma, or sarcoma.

In some embodiments, the cancer is endometrial cancer, head and neck cancer, or a sarcoma.

In some embodiments, the cancer is endometrial cancer. In some embodiments the cancer is head and neck cancer. In some embodiments, the cancer is a sarcoma.

In some embodiments, the sarcoma is soft tissue sarcoma, osteosarcoma, chondrosarcoma, Ewing sarcoma, hemangioendothelioma, angiosarcoma, fibrosarcoma, myofibrosarcoma, chordoma, adamantinoma, liposarcoma, leiomyosarcoma, malignant peripheral nerve sheath tumor, rhabdomyosarcoma, synovial sarcoma, or malignant solitary fibrous tumor.

In some embodiments, the sarcoma is soft tissue sarcoma. In some embodiments the soft tissue sarcoma is liposarcoma, atypical lipomatous tumor, dermatofibrosarcoma protuberans, malignant solitary fibrous tumor, inflammatory myofibroblastic tumor, low-grade myofibroblastic sarcoma, fibrosarcoma, myxofibrosarcoma, low-grade fibromyxoid sarcoma, giant cell tumor of soft tissues, leiomyosarcoma, malignant glomus tumor, rhabdomyosarcoma, hemangioendothelioma, angiosarcoma of soft tissue, extraskeletal osteosarcoma, gastrointestinal stromal tumor, malignant gastrointestinal stromal tumor (GIST), malignant peripheral nerve sheath tumor, malignant Triton tumor, malignant granular cell tumor, malignant ossifying fibromyxoid tumor, stromal sarcoma, myoepithelial carcinoma, malignant phosphaturic mesenchymal tumor, synovial sarcoma, epithelioid sarcoma, alveolar soft part sarcoma, clear cell sarcoma of soft tissue, extraskeletal myxoid chondrosarcoma, extraskeletal Ewing sarcoma, desmoplastic small round cell tumor, extrarenal rhabdoid tumor, perivascular epithelioid cell tumor, intimal sarcoma, undifferentiated spindle cell sarcoma, undifferentiated pleomorphic sarcoma, undifferentiated round cell sarcoma, undifferentiated epithelioid sarcoma, or undifferentiated sarcoma, not otherwise specified.

In some aspects, the present disclosure provides a method of treating or preventing a cancer in a subject in need thereof, comprising administering to the subject a therapeutically effective amount of a compound as described herein or a pharmaceutically acceptable salt, N-oxide, or stereoisomer thereof, or a pharmaceutical composition of the present disclosure.

In some aspects, the present disclosure provides a method of treating a cancer in a subject in need thereof, comprising administering to the subject a therapeutically effective amount of a compound as described herein or a pharmaceutically acceptable salt, N-oxide, or stereoisomer thereof, or a pharmaceutical composition of the present disclosure.

In some aspects, the present disclosure provides a method of treating or preventing a breast cancer in a subject in need thereof, comprising administering to the subject a therapeutically effective amount of a compound as described herein or a pharmaceutically acceptable salt, N-oxide, or stereoisomer thereof, or a pharmaceutical composition of the present disclosure.

In some aspects, the present disclosure provides a method of treating a breast cancer in a subject in need thereof, comprising administering to the subject a therapeutically effective amount of a compound as described herein or a pharmaceutically acceptable salt, N-oxide, or stereoisomer thereof, or a pharmaceutical composition of the present disclosure.

In some aspects, the present disclosure provides a method of treating or preventing a prostate cancer in a subject in need thereof, comprising administering to the subject a therapeutically effective amount of a compound as described herein or a pharmaceutically acceptable salt, N-oxide, or stereoisomer thereof, or a pharmaceutical composition of the present disclosure.

In some aspects, the present disclosure provides a method of treating a prostate cancer in a subject in need thereof, comprising administering to the subject a therapeutically effective amount of a compound as described herein or a pharmaceutically acceptable salt, N-oxide, or stereoisomer thereof, or a pharmaceutical composition of the present disclosure.

In some aspects, the present disclosure provides a method of treating or preventing a brain cancer in a subject in need thereof, comprising administering to the subject a therapeutically effective amount of a compound as described herein or a pharmaceutically acceptable salt, N-oxide, or stereoisomer thereof, or a pharmaceutical composition of the present disclosure.

In some aspects, the present disclosure provides a method of treating a brain cancer in a subject in need thereof, comprising administering to the subject a therapeutically effective amount of a compound as described herein or a pharmaceutically acceptable salt, N-oxide, or stereoisomer thereof, or a pharmaceutical composition of the present disclosure.

In some aspects, the present disclosure provides a compound as described herein or a pharmaceutically acceptable salt, N-oxide, or stereoisomer thereof for use in modulating PI3K (e.g., PI3Kα) activity (e.g., in vitro or in vivo).

In some aspects, the present disclosure provides a compound as described herein or a pharmaceutically acceptable salt, N-oxide, or stereoisomer thereof for use in treating or preventing a disease or disorder disclosed herein.

In some aspects, the present disclosure provides a compound as described herein or a pharmaceutically acceptable salt, N-oxide, or stereoisomer thereof for use in treating a disease or disorder disclosed herein.

In some aspects, the present disclosure provides a compound as described herein or a pharmaceutically acceptable salt, N-oxide, or stereoisomer thereof for use in treating or preventing a cancer in a subject in need thereof.

In some aspects, the present disclosure provides a compound as described herein or a pharmaceutically acceptable salt, N-oxide, or stereoisomer thereof for use in treating a cancer in a subject in need thereof.

In some aspects, the present disclosure provides a compound as described herein or a pharmaceutically acceptable salt, N-oxide, or stereoisomer thereof for use in treating or preventing a breast cancer in a subject in need thereof.

In some aspects, the present disclosure provides a compound as described herein or a pharmaceutically acceptable salt, N-oxide, or stereoisomer thereof for use in treating a breast cancer in a subject in need thereof.

In some aspects, the present disclosure provides a compound as described herein or a pharmaceutically acceptable salt, N-oxide, or stereoisomer thereof for use in treating or preventing a prostate cancer in a subject in need thereof.

In some aspects, the present disclosure provides a compound as described herein or a pharmaceutically acceptable salt, N-oxide, or stereoisomer thereof for use in treating a prostate cancer in a subject in need thereof.

In some aspects, the present disclosure provides a compound as described herein or a pharmaceutically acceptable salt, N-oxide, or stereoisomer thereof for use in treating or preventing a brain cancer in a subject in need thereof.

In some aspects, the present disclosure provides a compound as described herein or a pharmaceutically acceptable salt, N-oxide, or stereoisomer thereof for use in treating a brain cancer in a subject in need thereof.

In some aspects, the present disclosure provides use of a compound as described herein or a pharmaceutically acceptable salt, N-oxide, or stereoisomer thereof in the manufacture of a medicament for modulating PI3K (e.g., PI3Kα) activity (e.g., in vitro or in vivo).

In some aspects, the present disclosure provides use of a compound as described herein or a pharmaceutically acceptable salt, N-oxide, or stereoisomer thereof in the manufacture of a medicament for treating or preventing a disease or disorder disclosed herein.

In some aspects, the present disclosure provides use of a compound as described herein or a pharmaceutically acceptable salt, N-oxide, or stereoisomer thereof in the manufacture of a medicament for treating a disease or disorder disclosed herein.

In some aspects, the present disclosure provides use of a compound as described herein or a pharmaceutically acceptable salt, N-oxide, or stereoisomer thereof in the manufacture of a medicament for treating or preventing a cancer in a subject in need thereof.

In some aspects, the present disclosure provides use of a compound as described herein or a pharmaceutically acceptable salt, N-oxide, or stereoisomer thereof in the manufacture of a medicament for treating a cancer in a subject in need thereof.

In some aspects, the present disclosure provides use of a compound as described herein or a pharmaceutically acceptable salt, N-oxide, or stereoisomer thereof in the manufacture of a medicament for treating or preventing a breast cancer in a subject in need thereof.

In some aspects, the present disclosure provides use of a compound as described herein or a pharmaceutically acceptable salt, N-oxide, or stereoisomer thereof in the manufacture of a medicament for treating a breast cancer in a subject in need thereof.

In some aspects, the present disclosure provides use of a compound as described herein or a pharmaceutically acceptable salt, N-oxide, or stereoisomer thereof in the manufacture of a medicament for treating or preventing a prostate cancer in a subject in need thereof.

In some aspects, the present disclosure provides use of a compound as described herein or a pharmaceutically acceptable salt, N-oxide, or stereoisomer thereof in the manufacture of a medicament for treating a prostate cancer in a subject in need thereof.

In some aspects, the present disclosure provides use of a compound as described herein or a pharmaceutically acceptable salt, N-oxide, or stereoisomer thereof in the manufacture of a medicament for treating or preventing a brain cancer in a subject in need thereof.

In some aspects, the present disclosure provides use of a compound as described herein or a pharmaceutically acceptable salt, N-oxide, or stereoisomer thereof in the manufacture of a medicament for treating a brain cancer in a subject in need thereof.

The present disclosure provides compounds that function as modulators of PI3K activity. The present disclosure therefore provides a method of modulating PI3K activity in vitro or in vivo, said method comprising contacting a cell with a therapeutically effective amount of a compound, or a pharmaceutically acceptable salt, N-oxide, or stereoisomer thereof, as defined herein.

In some embodiments, PI3K is modulation is inhibition of PI3K.

In some embodiments, the PI3K inhibitor is a compound as described herein or a pharmaceutically acceptable salt, N-oxide, or stereoisomer thereof. In some embodiments, the PI3K inhibitor is a PI3Kα inhibitor. In some embodiments, the PI3K inhibitor is a PI3Kα H1047R mutant inhibitor. In some embodiments, the PI3K inhibitor is alpha/beta non-selective. In some embodiments, the PI3K inhibitor is alpha selective. In some embodiments, the PI3K inhibitor is beta selective.

Effectiveness of compounds of the disclosure can be determined by industry-accepted assays/disease models according to standard practices of elucidating the same as described in the art and are found in the current general knowledge.

The present disclosure also provides a method of treating a disease or disorder in which PI3K activity is implicated in a patient in need of such treatment, said method comprising administering to said patient a therapeutically effective amount of a compound, or a pharmaceutically acceptable salt, N-oxide, or stereoisomer thereof, or a pharmaceutical composition as defined herein.

The disclosure provides a method of modulating the activity of the PI3Kα allosteric active site, wherein the modulation is induced through peripheral site targeting. In some embodiments, the peripheral site is targeted with an agent selected from a small molecule, a peptide, a peptidomimetic, a protein, a protein mimetic, a nucleic acid, an antibody, an antibody-drug conjugate, a nucleoprotein complex, an immunotherapy, or a combination thereof.

The disclosure is also directed to the following Aspects:

Aspect 1. A compound of Formula (I)

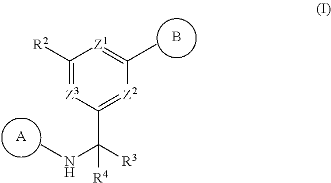

or a pharmaceutically acceptable salt, N-oxide, or stereoisomer thereof, wherein:
Ring A is aryl or a 5-7 membered heteroaryl ring comprising 1-4 heteroatoms selected from N, O, and S, wherein in the aryl or 5-7 membered heteroaryl ring is optionally substituted with one or more groups independently selected from D, halogen, C₁-C₈ alkoxy, C₁-C₈ haloalkoxy, C₁-C₈ alkyl, haloalkyl, —OH, —CN, —NO2, —C2-C6 alkenyl, —C2-C6 alkynyl, aryl, heteroaryl, cycloalkyl, cycloalkenyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF5, —P(O)R^{b}R^{b}, —P(O)R^{c}R^{d}, —P(O)(OR^{b})(OR^{b}), —B(OR^{c})(OR^{d}), —SiR^{b}₃, —S(O)₂R^{b}, —C(O)NR^{b}OR^{b}, —S(O)₂OR^{b}, —OS(O)₂OR^{b}, and —OPO(OR^{b})(OR^{b}); wherein each C₁-C₈ alkyl or haloalkyl is optionally substituted by 1-6 groups selected from D, halogen, —OH, —CN, —OR^{a}, —SR^{a}, —NR^{a}R^{d}, or NR^{c}R^{d};
Ring B is 4-13 membered heterocycloalkyl, 4-10 membered heterocycloalkenyl, or 4-10 membered heteroaryl, wherein the 4-13 membered heterocycloalkyl, 4-10 membered heterocycloalkenyl, or 4-10 membered heteroaryl is optionally substituted with one or more groups independently selected from D, oxo, ═NR^{a}, ═N—OR^{a}, ═N—CN, ═S, halogen, C₁-C₈ alkoxy, C₁-C₁₀ alkyl, haloalkyl, —OH, —CN, —NO₂, —C₂-C₁₀ alkenyl, —C₂-C₁₀ alkynyl, aryl, heteroaryl, cycloalkyl, cycloalkenyl, heterocycloalkyl, heterocycloalkenyl, C₀-C₄alk-aryl, C₀-C₄alk-heteroaryl, —OR^{a}, —SR^{a}, —NR^{c}R^{d}, —NR^{a}R^{c}, —C(O)R^{b}, —OC(O)R^{b}, —C(O)OR^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —C(═NR^{b})NR^{c}R^{d}, —C(═NOR^{b})NR^{c}R^{d}, —C(═NCN)NR^{c}R^{d}, —P(OR^{c})₂, —P(O)R^{c}R^{b}, —P(O)R^{c}R^{d}, —P(O)OR^{c}OR^{b}, —S(O)R^{b}, —S(O)NR^{c}R^{d}, —S(O)₂NR^{c}R^{d}, —S(O)(═NR^{b})R^{b}, —SF5, —P(O)R^{b}R^{b}, —P(O)(OR^{b})(OR^{b}), —B(OR^{c})(OR^{d}), —S(O)₂R^{b}, —C(O)NR^{b}OR^{b}, —SiR^{b}₃, —S(O)₂OR^{b}, —OS(O)₂OR^{b}, —OPO(OR^{b})(OR^{b}) and -L-W; wherein each C₁-C₈ alkoxy, C₁-C₁₀ alkyl, haloalkyl, —C2-C10 alkenyl, —C2-C10 alkynyl, aryl, heteroaryl, cycloalkyl, cycloalkenyl, heterocycloalkyl, heterocycloalkenyl, C₀-C₄alk-aryl, or C₀-C₄alk-heteroaryl is optionally substituted by 1-6 R¹ groups;
L is absent or is C₁-C₈ alkylene, —O—, —N(R^{a})—, —S— or 3-10 membered cycloalkylene;
W is a 5-10 membered heteroaryl ring comprising 1-4 heteroatoms selected from N, O, and S, a 5-12 membered heterocyclic group comprising 1-4 heteroatoms selected from N, O, and S, C₁-C₈ alkyl, haloalkyl, —C2-C6 alkenyl, —C2-C6 alkynyl, aryl, cycloalkyl, cycloalkenyl, heterocyclo-alkenyl, NR^{c}R^{d}, OR^{b}, or SR^{b}; wherein each group is optionally substituted by 1-6 R^{f} groups;
Z¹, Z², and Z³ are each independently CR² or N;
each R² is independently H, D, halogen, C₁-C₈ alkoxy, C₁-C₈ alkyl, haloalkyl, —OH, —CN, —NO2, —C2-C6 alkenyl, —C2-C6 alkynyl, aryl, heteroaryl, cycloalkyl, cycloalkenyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF5, —P(O)R^{b}R^{b}, —P(O)R^{c}R^{d}, —P(O)(OR^{b})(OR^{c}), —B(OR^{c})(OR^{d}), SiR^{b}₃, —S(O)₂R^{b}, —C(O)NR^{b}OR^{b}, —S(O)₂OR^{b}, —OS(O)₂OR^{b}, or —OPO(OR^{b})(OR^{c}); wherein said C₁-C₈ alkyl is optionally substituted by 1-6 groups selected from D, halogen, —OH, —CN, —OR^{a}, —SR^{a}, —NR^{a}R^{d}, or NR^{c}R^{d};
each R³ and R⁴ is independently H, D, C₁-C₈ alkyl, haloalkyl, or CN; wherein said C₁-C₈ alkyl is optionally substituted by 1-6 groups selected from D, halogen, —OH, —CN, —OR^{a}, —SR^{a}, —NR^{a}R^{d}, or —NR^{c}R^{d};
or R³ and R⁴, together with the atom to which they are both attached, are combined to form a C₃-C₇ cycloalkyl or C₄-C₈ heterocycloalkyl, wherein the C₃-C₇ cycloalkyl or C₄-C₈ heterocycloalkyl is optionally substituted by 1-6 groups selected from D, halogen, —OH, —CN, —OR^{a}, —SR^{a}, —NR^{a}R^{d}, or —NR^{c}R^{d};
each R^{a} is independently H, D, —C(O)R^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —C(═NR^{b})NR^{c}R^{d}, —C(═NOR^{b})NR^{c}R^{d}, —C(═NCN)NR^{c}R^{d}, —P(OR^{c})₂, —P(O)R^{c}R^{b}, —P(O)R^{c}R^{d}, —P(O)OR^{c}OR^{b}, —S(O)R^{b}, —S(O)NR^{c}R^{d}, —S(O)₂R^{b}, —S(O)₂NR^{c}R^{d}, —SiR^{b}₃, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl, wherein each —C₁-C₁₀alkyl, —C₂-C₁₀ alkenyl, —C₂-C₁₀ alkynyl, aryl, cycloalkyl, cycloalkenyl, heteroaryl, heterocycloalkyl, and heterocycloalkenyl of R^{a} is optionally substituted by 1-6 R^{f} groups;
each R^{b} is independently H, D, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl; wherein each —C₁-C₆ alkyl, —C₂-C₆ alkenyl, —C₂-C₆ alkynyl, aryl, cycloalkyl, cycloalkenyl, heteroaryl, heterocycloalkyl, and heterocycloalkenyl of R^{b} is optionally substituted by 1-6 R^{f} groups;
each R^{c} or R^{d} is independently H, D, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl; wherein each —C₁-C₁₀ alkyl, —C₂-C₆ alkenyl, —C₂-C₆ alkynyl, —OC₁-C₆alkyl, —O— cycloalkyl, aryl, heteroaryl, cycloalkyl, cycloalkenyl, heterocycloalkyl of R^{c} or R^{d} is optionally substituted by 1-6 R^{f} groups;
or R^{c} and R^{d}, together with the atom to which they are both attached, form a monocyclic or multicyclic heterocycloalkyl, or a monocyclic or multicyclic heterocycloalkenyl group, wherein the monocyclic or multicyclic heterocycloalkyl, or a monocyclic or multicyclic heterocycloalkenyl group is optionally substituted by 1-6 R¹ groups;
each R^{f} is independently D, oxo, halogen, C₁-C₈ alkoxy, C₁-C₈ alkyl, haloalkyl, —OH, —CN, —NO2, —C2-C6 alkenyl, —C2-C6 alkynyl, aryl, heteroaryl, cycloalkyl, cycloalkenyl, heterocycloalkyl, heterocycloalkenyl, —OR^{g0}, —SR^{g0}, —NR^{g2}R^{g3}, —NR^{g0}R^{g2}, —C(O)R^{g1}, —OC(O)R^{g1}, —C(O)OR^{g1}, —C(O)NR^{g2}R^{g3}, —S(O)R^{g1}, —S(O)₂NR^{g2}R^{g3}, —S(O)(═NR^{g1})R^{g1}, —SF5, —P(O)R^{g1}R^{g1}, —P(O)R^{g2}R^{g3}, —P(O)(OR^{g1})(OR^{g1}), SiR^{g1}₃, —B(OR^{g2})(OR^{g3}), —S(O)₂R^{g1}, —C(O)NR^{g1}OR^{g1}, —S(O)₂OR^{g1}, —OS(O)₂OR^{g1}, or —OPO(OR^{g1})(OR^{g1}); wherein each C₁-C₈ alkoxy, C₁-C₈ alkyl, haloalkyl, —C2-C6 alkenyl, —C2-C6 alkynyl, aryl, heteroaryl, cycloalkyl, cycloalkenyl, heterocycloalkyl, or heterocycloalkenyl is optionally substituted by 1-6 R^{g} groups;
each R^{g0} is independently H, D, —C(O)R^{g1}, —C(O)OR^{g2}, —C(O)NR^{g2}R^{g3}, —C(═NR^{g1})NR^{g1}R^{g2}, —C(═NOR^{g1})NR^{g1}R^{g2}, —C(═NCN)NR^{g1}R^{g2}, —C(═NR^{g1})NR^{g2}R^{g3}, —C(═NOR^{g1})NR^{g2}R^{g3}, —C(═NCN)NR^{g2}R^{g3}, —P(OR^{g2})₂, —P(O)R^{g2}R^{g1}, —P(O)R^{g2}R^{g3}, —P(O)OR^{g2}OR^{g1}, —S(O)R^{g1}, —S(O)NR^{g2}R^{g3}, —S(O)₂R^{g1}, —S(O)₂NR^{g2}R^{g3}, —SiR^{g1}₃, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl, wherein each —C₁-C₁₀alkyl, —C₂-C₁₀ alkenyl, —C₂-C₁₀ alkynyl, aryl, cycloalkyl, cycloalkenyl, heteroaryl, heterocycloalkyl, and heterocycloalkenyl is optionally substituted by 1-6 R^{g} groups;
each R^{g1} is independently H, D, C₁-C₈ alkyl, haloalkyl, —C2-C6 alkenyl, —C2-C6 alkynyl, aryl, heteroaryl, cycloalkyl, cycloalkenyl, heterocycloalkyl, or heterocycloalkenyl, wherein each C₁-C₈ alkyl, haloalkyl, —C2-C6 alkenyl, —C2-C6 alkynyl, aryl, heteroaryl, cycloalkyl, cycloalkenyl, heterocycloalkyl, or heterocycloalkenyl is optionally substituted by 1-6 R^{g} groups;
each R^{g2} or R^{g3} is independently H, D, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl; wherein each —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, or heterocycloalkyl is optionally substituted by 1-6 R^{g} groups; or R^{g2} and R^{g3}, together with the atom to which they are both attached, form a monocyclic or multicyclic heterocycloalkyl, or a monocyclic or multicyclic heterocycloalkenyl group, wherein the monocyclic or multicyclic heterocycloalkyl, or monocyclic or multicyclic heterocycloalkenyl group is optionally substituted by 1-6 R^{g} groups;
each R^{g} is independently:
D, —CN, —NO2, oxo, halogen, —SF₅, —OR^{h}, —SR^{h}, —NR^{h}R^{h}, —SiR^{h}₃, —C(O)R^{h}, —OC(O)R^{h}, —C(O)OR^{h}, —OC(O)OR^{h}, —C(O)NR^{h}OR^{h}, —S(O)R^{h}, —S(O)₂R^{h}, —S(O)₂OR^{h}, —OS(O)₂OR^{h}, —S(O)(═NR^{h})R^{h}, —P(O)(OR^{h})(OR^{h}), —P(O)R^{h}R^{h}, —OPO(OR^{h})(OR^{h}), —C(O)NR^{h}R^{h}, —OC(O)NR^{h}R^{h}, —S(O)₂NR^{h}R^{h}, —B(OR^{h})(OR^{h}), —OC(═NR^{h})NR^{h}R^{h}, —OC(═NOR^{h})NR^{h}R^{h}, —OC(═NCN)NR^{h}R^{h}, —OP(OR^{h})₂, —OP(O)R^{h}R^{h}, —OP(O)OR^{h}OR^{h}, —OS(O)R^{h}, —OS(O)NR^{h}R^{h}, —OS(O)₂R^{h}, —OS(O)₂NR^{h}R^{h}, —OSiR^{h}₃, —S—C(O)R^{h}, —S—C(O)OR^{h}, —S—C(O)NR^{h}R^{h}, —S(O)R^{h}, —S(O)NR^{h}R^{h}, —S(O)₂R^{h}, —S(O)₂NR^{h}R^{h}, —SSiR^{h}₃, —NR^{h}C(O)R^{h}, —NR^{h}C(O)OR^{h}, —NR^{h}C(O)NR^{h}R^{h}, —NR^{h}C(═NR^{h})NR^{h}R^{h}, —NR^{h}C(═NOR^{h})NR^{h}R^{h}, —NR^{h}C(═NCN)NR^{h}R^{h}, —NR^{h}P(OR^{h})₂, —NR^{h}P(O)R^{h}R^{h}, —NR^{h}P(O)OR^{h}OR^{h}, —NR^{h}S(O)R^{h}, —NR^{h}S(O)NR^{h}R^{h}, —NR^{h}S(O)₂R^{h}, —NR^{h}S(O)₂NR^{h}R^{h}, —NR^{h}SiR^{h}₃, C₁-C₈ alkoxy, C₁-C₈ alkyl, haloalkyl, —C2-C6 alkenyl, —C2-C6 alkynyl, aryl, heteroaryl, cycloalkyl, cycloalkenyl, heterocycloalkyl, or heterocycloalkenyl, wherein the C₁-C₈ alkoxy, C₁-C₈ alkyl, haloalkyl, —C2-C6 alkenyl, —C2-C6 alkynyl, aryl, heteroaryl, cycloalkyl, cycloalkenyl, heterocycloalkyl, or heterocycloalkenyl is optionally substituted by 1-6 groups selected from D, halogen, —CN, —NO2, oxo, —OR^{h}, —SR^{h}, or —NR^{h}R^{h};
   wherein each R^{h} is independently H, D, C₁-C₈alkyl, haloalkyl, —C2-C6 alkenyl, —C2-C6 alkynyl, aryl, heteroaryl, cycloalkyl, cycloalkenyl, heterocycloalkyl, or heterocycloalkenyl; or wherein two R^{h} attached to the same atom may form a monocyclic or multicyclic heterocycloalkyl, or a monocyclic or multicyclic heterocycloalkenyl group.

Aspect 2. The compound of aspect 1, that is a compound of Formula (II)

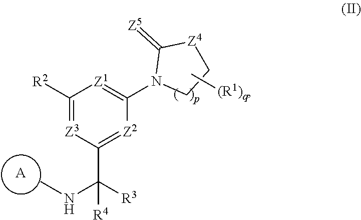

or a pharmaceutically acceptable salt, N-oxide, or stereoisomer thereof; wherein
Z⁴ is O, NR^{a}, C(R¹)₂, or S;
Z⁵ is O, NR^{a}, N—OR^{a}, N—CN, or S;
p is 1, 2 or 3; and
q is 0, 1, 2, 3, 4, 5, 6, 7, or 8;
each R¹ is independently selected from H, D, oxo, ═NR^{a}, ═N—OR^{a}, ═N—CN, ═S, halogen, C₁-C₈ alkoxy, C₁-C₁₀ alkyl, haloalkyl, —OH, —CN, —NO₂, —C₂-C₁₀ alkenyl, —C₂-C₁₀ alkynyl, aryl, heteroaryl, cycloalkyl, cycloalkenyl, heterocycloalkyl, heterocycloalkenyl, C₀-C₄alk-aryl, C₀-C₄alk-heteroaryl, —OR^{a}, —SR^{a}, —NR^{c}R^{d}, —NR^{a}R^{c}, —C(O)R^{b}, —OC(O)R^{b}, —C(O)OR^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —C(═NR^{b})NR^{c}R^{d}, —C(═NOR^{b})NR^{c}R^{d}, —C(═NCN)NR^{c}R^{d}, —P(OR^{c})₂, —P(O)R^{c}R^{b}, —P(O)R^{c}R^{d}, —P(O)OR^{c}OR^{b}, —S(O)R^{b}, —S(O)NR^{c}R^{d}, —S(O)₂NR^{c}R^{d}, —S(O)(═NR^{b})R^{b}, —SF5, —P(O)R^{b}R^{b}, —P(O)(OR^{b})(OR^{b}), —B(OR^{c})(OR^{d}), —S(O)₂R^{b}, —C(O)NR^{b}OR^{b}, —SiR^{b}₃, —S(O)₂OR^{b}, —OS(O)₂OR^{b}, —OPO(OR^{b})(OR^{b}) and -L-W; wherein each C₁-C₈ alkoxy, C₁-C₁₀ alkyl, haloalkyl, —C2-C10 alkenyl, —C2-C10 alkynyl, aryl, heteroaryl, cycloalkyl, cycloalkenyl, heterocycloalkyl, heterocycloalkenyl, C₀-C₄alk-aryl, or C₀-C₄alk-heteroaryl is optionally substituted by 1-6 R^{f} groups.

Aspect 3. The compound of aspect 2, wherein p is 1.

Aspect 4. The compound of aspect 2, wherein p is 2.

Aspect 5. The compound of any one of the preceding aspects, wherein Ring A is:

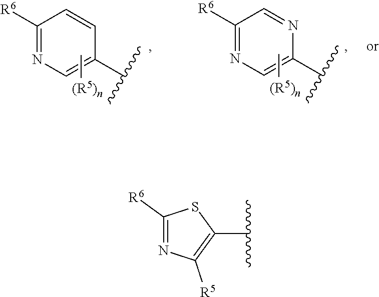

wherein
n is 1, 2, or 3;
each R⁵ is independently selected from H, D, halogen, C₁-C₈ alkoxy, C₁-C₈ haloalkoxy, C₁-C₈ alkyl, haloalkyl, —OH, —CN, —NO2, —C2-C6 alkenyl, —C2-C6 alkynyl, aryl, heteroaryl, cycloalkyl, cycloalkenyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF5, —P(O)R^{b}R^{b}, —P(O)R^{c}R^{d}, —P(O)(OR^{b})(OR^{b}), SiR^{b}₃, —B(OR^{c})(OR^{d}), —S(O)₂R^{b}, —C(O)NR^{b}OR^{b}, —S(O)₂OR^{b}, —OS(O)₂OR^{b}, and —OPO(OR^{b})(OR^{b}); wherein said C₁-C₈ alkyl or haloalkyl is optionally substituted by 1-6 groups selected from D, halogen, —OH, —CN, —OR^{a}, —SR^{a}, —NR^{a}R^{d}, or NR^{c}R^{d}; and
R⁶ is —F, —Cl, —Br, —I, C₁-C₄-alkyl, C₁-C₄-alkoxy, C₁-C₄-haloalkoxy, or —CN.

Aspect 6. The compound of aspect 5, wherein n is 1.

Aspect 7. The compound of aspect 5, wherein n is 2.

Aspect 8. The compound of aspect 5, wherein n is 3.

Aspect 9. The compound of aspect 5, that is a compound of Formula (III)

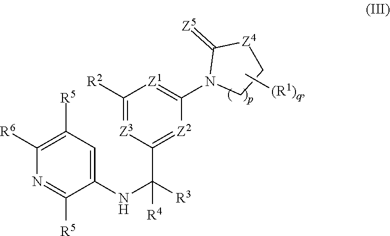

or a pharmaceutically acceptable salt, N-oxide, or stereoisomer thereof.

Aspect 10. The compound of aspect 9, that is a compound of Formula (IV)

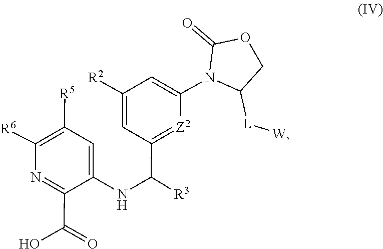

or a pharmaceutically acceptable salt, N-oxide, or stereoisomer thereof.

Aspect 11. The compound of any one of aspects 5-9, wherein at least one R⁵ is —CO₂H, —CONH₂, —COOCH₃, —C(O)H, or —CN.

Aspect 12. The compound of aspect 11, wherein at least one R⁵ is —CO₂H.

Aspect 13. The compound of any one of the preceding aspects, wherein at least one R² is H.

Aspect 14. The compound of any one of aspects 1-13 wherein at least one R² is C₁-C₈ alkyl, CD₃, CF₃, halogen, CN or CHF₂.

Aspect 15. The compound of aspect 14, wherein at least one R² is methyl or CD₃.

Aspect 16. The compound of aspect 14, wherein at least one R² is F.

Aspect 17. The compound of aspect 14, wherein at least one R² is Cl.

Aspect 18. The compound of aspect 14, wherein at least one R² is CHF₂.

Aspect 19. The compound of aspect 14, wherein at least one R² is CF₃.

Aspect 20. The compound of any one of the preceding aspects, wherein R³ is H or C₁-C₈ alkyl.

Aspect 21. The compound of any one of the preceding aspects, wherein R³ is methyl or CD₃.

Aspect 22. The compound of any one of the preceding aspects, wherein R⁴ is H or C₁-C₈ alkyl.

Aspect 23. The compound of any one of the preceding aspects, wherein R⁴ is H.

Aspect 24. The compound of any one of the preceding aspects, wherein each Z¹, Z² and Z³ is CR².

Aspect 25. The compound of any one of aspects 1-24, wherein Z¹ and Z³ are CR².

Aspect 26. The compound of any one of aspects 1-23, wherein at least one of Z¹, Z² and Z³ is N.

Aspect 27. The compound any one of aspects 1-23 or 25-26, wherein Z² is N.

Aspect 28. The compound of any one of aspects 1-26 wherein Z² is CH.

Aspect 29. The compound of any one of aspects 5-28, wherein R⁶ is —F.

Aspect 30. The compound of any one of aspects 5-28, wherein R⁶ is or —Cl.

Aspect 31. The compound of any one of aspects 5-28, wherein R⁶ is —CH₃.

Aspect 32. The compound of any one of aspects 5-28, wherein R⁶ is —OCH₃.

Aspect 33. The compound of any one of aspects 5-28, wherein R⁶ is —OCF₂H.

Aspect 34. The compound of any one of aspects 2-33, wherein Z⁴ is O.

Aspect 35. The compound of any one of aspects 2-34, wherein Z⁵ is O.

Aspect 36. The compound of any one of aspects 2-35, wherein Z⁴ and Z⁵ are O.

Aspect 37. The compound of any one of aspects 2-36, wherein q is 1.

Aspect 38. The compound of aspect 1, wherein Ring B is substituted with at least one -L-W.

Aspect 39. The compound of any one of aspects 2-37, wherein at least one R¹ is -L-W.

Aspect 40. The compound of any one of the preceding aspects, wherein W is aryl optionally substituted by 1-6 R^{f} groups.

Aspect 41. The compound of aspect 40, wherein W is phenyl optionally substituted by 1-5 R^{f} groups.

Aspect 42. The compound of aspect 41, wherein W is phenyl.

Aspect 43. The compound of any one of aspects 1-39, wherein W is a 5-12 membered heterocyclic group comprising 1-4 heteroatoms selected from N, O, and S, wherein the 5-12 membered heterocyclic group is optionally substituted by 1-6 R^{f} groups.

Aspect 44. The compound of any one of aspects 1-39, wherein W is a 5-10 membered heteroaryl ring comprising 1-4 heteroatoms selected from N, O, and S, wherein the 5-10 membered heteroaryl ring is optionally substituted by 1-6 R^{f} groups.

Aspect 45. The compound of any one of the preceding aspects, wherein L is absent or is C₁-C₈ alkylene.

Aspect 46. The compound of aspect 45, wherein L is a methylene group.

Aspect 47. The compound of aspect 1, that is
3-((1-(3-((S)-4-Benzyl-2-oxooxazolidin-3-yl)-5-methylphenyl)ethyl)amino)-6-chloro-picolinic acid;
3-(1-(3-((S)-4-Benzyl-2-oxooxazolidin-3-yl)-5-methylphenyl)ethyl)amino)-6-chloro-picolinic acid;
3-((1-(3-((S)-4-Benzyl-2-oxooxazolidin-3-yl)-5-methylphenyl)ethyl)amino)-6-fluoro-picolinic acid;
3-((1-(3-((S)-4-benzyl-2-oxooxazolidin-3-yl)-5-fluorophenyl)ethyl)amino)-6-chloro-picolinic acid;
3-((1-(3-((S)-4-Benzyl-2-oxooxazolidin-3-yl)-5-fluorophenyl)ethyl)amino)-6-fluoro-picolinic acid;
3-((1-(3-((S)-4-Benzyl-2-oxooxazolidin-3-yl)-5-chlorophenyl)ethyl)amino)-6-chloro-picolinic acid;
3-((1-(3-((S)-4-Benzyl-2-oxooxazolidin-3-yl)-5-chlorophenyl)ethyl)amino)-6-fluoro-picolinic acid;
3-((1-(3-((S)-4-Benzyl-2-oxooxazolidin-3-yl)-5-(trifluoromethyl)phenyl)ethyl)amino)-6-fluoropicolinic acid;
3-(((R)-1-(3-((S)-4-(4-Bromobenzyl)-2-oxooxazolidin-3-yl)-5-methylphenyl)ethyl) amino)-6-chloropicolinic acid;
3-(((R)-1-(3-((S)-4-Benzyl-2-oxooxazolidin-3-yl)-5-fluorophenyl)ethyl)amino)-6-chloro-picolinic acid;
3-[1-[6-[(4S)-4-Benzyl-2-oxo-1,3-oxazolidin-3-yl]-4-methyl-2-pyridinyl]ethylamino]-6-fluoropyridine-2-carboxylic acid;
3-(((R)-1-(6-((S)-4-Benzyl-2-oxooxazolidin-3-yl)-4-methylpyridin-2-yl)ethyl)amino)-6-chloropicolinic acid;
3-((1-(6-((S)-4-Benzyl-2-oxooxazolidin-3-yl)-4-methylpyridin-2-yl)ethyl)amino)picolinic acid;
3-((1-(6-((S)-4-Benzyl-2-oxooxazolidin-3-yl)-4-methylpyridin-2-yl)ethyl)amino)-6-methylpicolinic acid;
3-((1-(6-((S)-4-Benzyl-2-oxooxazolidin-3-yl)-4-methylpyridin-2-yl)ethyl)amino)-6-(trifluoromethyl)picolinic acid;
3-((1-(6-((S)-4-Benzyl-2-oxooxazolidin-3-yl)-4-methylpyridin-2-yl)ethyl)amino)-6-methoxypicolinic acid;
2-((1-(6-((S)-4-Benzyl-2-oxooxazolidin-3-yl)-4-methylpyridin-2-yl)ethyl)amino)benzoic acid;
3-((1-(6-((S)-4-Benzyl-2-oxooxazolidin-3-yl)-4-methylpyridin-2-yl)ethyl)amino)-6-bromopicolinic acid;
3-((1-(6-((S)-4-Benzyl-2-oxooxazolidin-3-yl)-4-methylpyridin-2-yl)ethyl)amino)-6-cyanopicolinic acid;
   or a pharmaceutically acceptable salt, N-oxide, or stereoisomer thereof.

Aspect 48. The compound of any one of the preceding aspects, in the form of a pharmaceutically acceptable salt.

Aspect 49. A pharmaceutical composition comprising a compound according to any one of the preceding aspects, or a pharmaceutically acceptable salt thereof, and a pharmaceutically acceptable excipient.

Aspect 50. A method of treating a disease or disorder associated with modulation of phosphoinositide 3-kinase (PI3K), comprising administering to a patient in need thereof a therapeutically effective amount of a compound of any one of aspects 1-48 or a pharmaceutical composition of aspect 49.

Aspect 51. The method of aspect 50, wherein the PI3K is PI3Kα.

Aspect 52. The method of aspect 50 or aspect 51, wherein the PI3K associated with the disease or disorder has a H1047R mutation.

Aspect 53. The method of any one of aspects 50-52, wherein the disease or disorder is a cancer.

Aspect 54. The method of aspect 53, wherein the cancer is endometrial cancer, gastric cancer, leukemia, lymphoma, sarcoma, colorectal cancer, lung cancer, ovarian cancer, skin cancer, head and neck cancer, breast cancer, brain cancer, cervical cancer, bladder cancer, esophageal cancer, pancreatic cancer, bone cancer, hepatobiliary cancer, medulloblastoma, kidney cancer or prostate cancer.

Aspect 55. The method of any one of aspects 50-52, wherein the disease or disorder is CLOVES syndrome (congenital lipomatous overgrowth, vascular malformations, epidermal naevi, scoliosis/skeletal and spinal syndrome), or PIK3CA-related overgrowth syndrome (PROS).

Aspect 56. A method of inhibiting phosphoinositide 3-kinase (PI3K), comprising administering to a patient in need thereof a therapeutically effective amount of a compound of any one of aspects 1-48 or a pharmaceutical composition of aspect 49.

Aspect 57. A method of treating cancer or a disorder, the method comprising administering to a patient in need thereof a therapeutically effective amount of a compound of any one of aspects 1-48 or a pharmaceutical composition of aspect 49.

Aspect 58. The method of aspect 57, wherein the cancer is endometrial cancer, gastric cancer, leukemia, lymphoma, sarcoma, colorectal cancer, lung cancer, ovarian cancer, skin cancer, head and neck cancer, breast cancer, brain cancer, or prostate cancer.

Aspect 59. The method of aspect 58, wherein the cancer is breast cancer.

Aspect 60. The method of aspect 57, wherein the disorder is CLOVES syndrome (congenital lipomatous overgrowth, vascular malformations, epidermal naevi, scoliosis/skeletal and spinal syndrome) or PIK3CA-related overgrowth syndrome (PROS).

Aspect 61. The method of aspect 60, wherein the disorder is CLOVES syndrome (congenital lipomatous overgrowth, vascular malformations, epidermal naevi, scoliosis/skeletal and spinal syndrome).

Aspect 62. The method of aspect 60, wherein the disorder is PIK3CA-related overgrowth syndrome (PROS).

Aspect 63. A method of degrading a phosphoinositide 3-kinase (PI3K) protein comprising contacting the PI3K protein with a compound of any one of aspects 1-48 or a pharmaceutical composition of aspect 49.

Aspect 64. The method of aspect 63, wherein the PI3K is PI3Kα.

### Synthesis

Compounds of the invention, including salts thereof, can be prepared using known organic synthesis techniques and can be synthesized according to any of numerous possible synthetic routes.

The reactions for preparing compounds of the invention can be carried out in suitable solvents which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd. Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatography (HPLC) or thin layer chromatography.

The expressions, “ambient temperature,” “room temperature,” and “r.t.” as used herein, are understood in the art, and refer generally to a temperature, e.g., a reaction temperature, that is about the temperature of the room in which the reaction is carried out, for example, a temperature from about 20° C. to about 30° C.

Compounds of the invention can be prepared using numerous preparatory reactions known in the literature. The Schemes below provide general guidance in connection with preparing the compounds of the invention. One skilled in the art would understand that the preparations shown in the Schemes can be modified or optimized using general knowledge of organic chemistry to prepare various compounds of the invention. Example synthetic methods for preparing compounds of the invention are provided in the Schemes below.

The following Examples are provided to illustrate some of the concepts described within this disclosure. While the Examples are considered to provide an embodiment, it should not be considered to limit the more general embodiments described herein.

### EXAMPLES

### General Synthetic Procedures

Compounds of Formula (I) can be prepared from optionally protected 1-1 as shown in Scheme I. Compounds 1-1 where Lg is halogen (e.g., Cl, Br, or I) or pseudohalogen (e.g., OTf, OTs, or OMs) can be transformed into compounds 1-2 under standard Stille conditions (e.g., palladium(II) catalyst, such as bis(triphenylphosphine)palladium(II) dichloride and an appropriate stannane, such as tributyl(1-ethoxyvinyl)tin), or Heck conditions (e.g., palladium catalyst, such as palladium(II) acetate; a ligand, such as 1,3-bis(diphenylphosphino)propane; and an appropriate olefin, such as butyl vinyl ether). Compounds 1-2 can be converted to compounds 1-4 under either reductive conditions (e.g., in the presence of a suitable reducing agent, such as sodium borohydride) or nucleophilic addition conditions with nucleophiles 1-3 where M¹ is a metal (e.g., Li, MgCl, MgBr, ZnCl, or ZnBr). Alcohols 1-4 can be transformed to compounds 1-5 where Lg¹ is halogen (e.g., Cl, Br, or I) under standard deoxygenative halogenation conditions (e.g., thionyl chloride, phosphorous tribromide, or triphenylphosphine and iodine) or a pseudohalogen (e.g., OTf, OTs, or OMs) under standard sulfonylation conditions (e.g., in the presence of a sulfonylating agent, such as methanesulfonyl chloride, p-toluenesulfonyl chloride, or trifluoromethanesulfonic anhydride, and a base, such as triethylamine). Reaction of compounds 1-5 with optionally protected amines 1-6, optionally in the presence of a base (e.g., triethylamine), can provide compounds of Formula (I). In some instances, subsequent optional global or selective deprotection can also afford compounds of Formula (I).

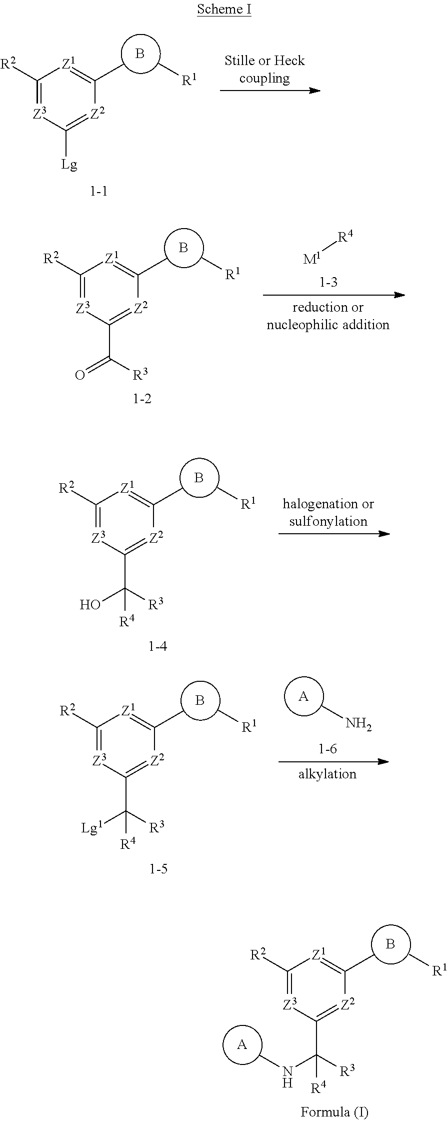

Compounds of Formula (I) can be prepared from as shown in Scheme II. Annulation of amino acids 2-1 can afford bicycles 2-2. Reaction of compounds 2-2 with alcohols 2-3, which can be prepared as described in Scheme I, under standard Mitsunobu conditions, such as in the presence of an azodicarboxylate (e.g., diisopropyl azodicarboxylate) and a phosphine (e.g., triphenylphosphine) can afford compounds 2-4. Hydrolysis under standard conditions, such as in the presence of a base (e.g., K₂CO₃ or NaOH) or an acid (e.g., HCl) or upon workup, can afford compounds 2-5.

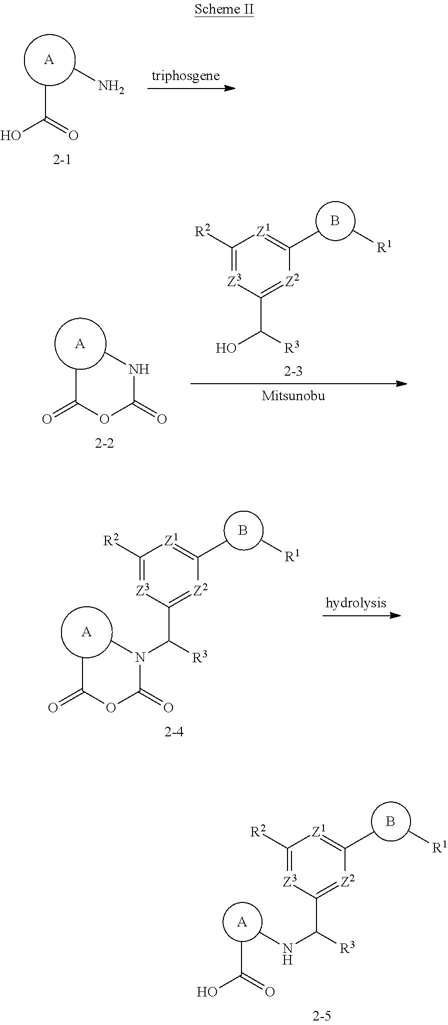

Intermediates for the synthesis of compounds of Formula (I) can be prepared from arenes 3-1 as shown in Scheme III. Arenes 3-1 where Lg² is halogen (e.g., Cl, Br, or I) or pseudohalogen (e.g., OTf, OTs, or OMs) can be coupled with compounds 3-2 under standard Buchwald-Hartwig coupling conditions, such as in the presence of a catalyst (e.g., XPhos Pd G3), and a base (e.g., Cs₂CO₃ or K₃PO₄) or standard Ullman coupling conditions, such as in the presence of a copper catalyst (e.g., CuI), a ligand (e.g., N1,N2-dimethylethane-1,2-diamine or N,N-dimethylglycine), and a base (e.g., K₂CO₃) to provide intermediates 3-3. Alternatively, arenes 3-1 can be coupled to compounds 3-4 where M² is a boronic acid, boronate ester, potassium trifluoroborate, or an appropriately substituted metal, such as Sn(Bu)₃, Sn(Me)₃, or ZnCl, under standard Suzuki conditions (e.g., in the presence of a palladium catalyst, such as [1,1′-bis(diphenyl-phosphino)ferrocene]dichloropalladium(II) and a base, such as K₃PO₄) or standard Stille conditions (e.g., in the presence of a palladium(0) catalyst, such as tetrakis(triphenyl-phosphine)palladium(0)) or standard Negishi conditions (e.g., in the presence of a palladium catalyst, such as tetrakis (triphenylphosphine)palladium(0) or [1,1′-bis(diphenylphosphino) ferrocene]dichloropalladium(II)), to give compounds 1-1.

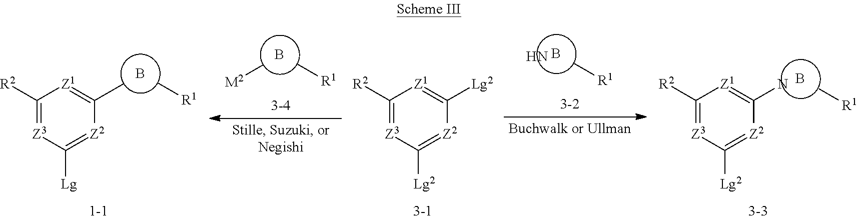

Compounds of Formula (I) can be prepared from arenes 3-1 as shown in Scheme IV. Arenes 3-1 can be transformed into compounds 4-1 under standard Stille conditions (e.g., palladium(II) catalyst, such as bis(triphenylphosphine)palladium(II) dichloride and an appropriate stannane, such as tributyl(1-ethoxyvinyl)tin), or Heck conditions (e.g., palladium catalyst, such as palladium(II) acetate; a ligand, such as 1,3-bis(diphenylphosphino)-propane; and an appropriate olefin, such as butyl vinyl ether). Condensation of carbonyls 4-1 with sulfinamides 4-2 that are optionally stereoenriched and where Rai is C₁-C₆ alkyl group in the presence of Lewis acid (e.g., Ti(OEt)₄) and consequent reduction with a reducing agent (e.g., NaBH₄ or L-selectride) can provide compounds 4-3 optionally in a diastereoselective fashion. Compounds 4-3 can be converted to amines 4-4 under standard deprotection conditions (e.g., in the presence of an acid, such as HCl in dioxane, and an appropriate solvent, such as methanol). Reaction of amines 4-4 with compounds 4-5 where X¹ is a halogen (e.g., Cl or F) and Z⁴ is N or CR⁵ and R^{b1} is a H or C₁-C₆ alkyl group under standard nucleophilic aromatic substitution conditions, such as in the presence of a suitable base (e.g., N,N-diisoproylethylamine) can afford compounds 4-6. Arenes 4-6 can be coupled with oxazolidinones 4-7 under standard Buchwald-Hartwig coupling conditions, such as in the presence of a catalyst (e.g., XPhos Pd G3), and a base (e.g., Cs₂CO₃ or K₃PO₄) or standard Ullman coupling conditions, such as in the presence of a copper catalyst (e.g., CuI), a ligand (e.g., N1,N2-dimethylethane-1,2-diamine or N,N-dimethylglycine), and a base (e.g., K₂CO₃) to provide intermediates 4-8. Esters 4-8 can be optionally deprotected under standard conditions, such as in the presence of an acid (e.g., TFA) or base (e.g., LiOH), to afford compounds 4-9.

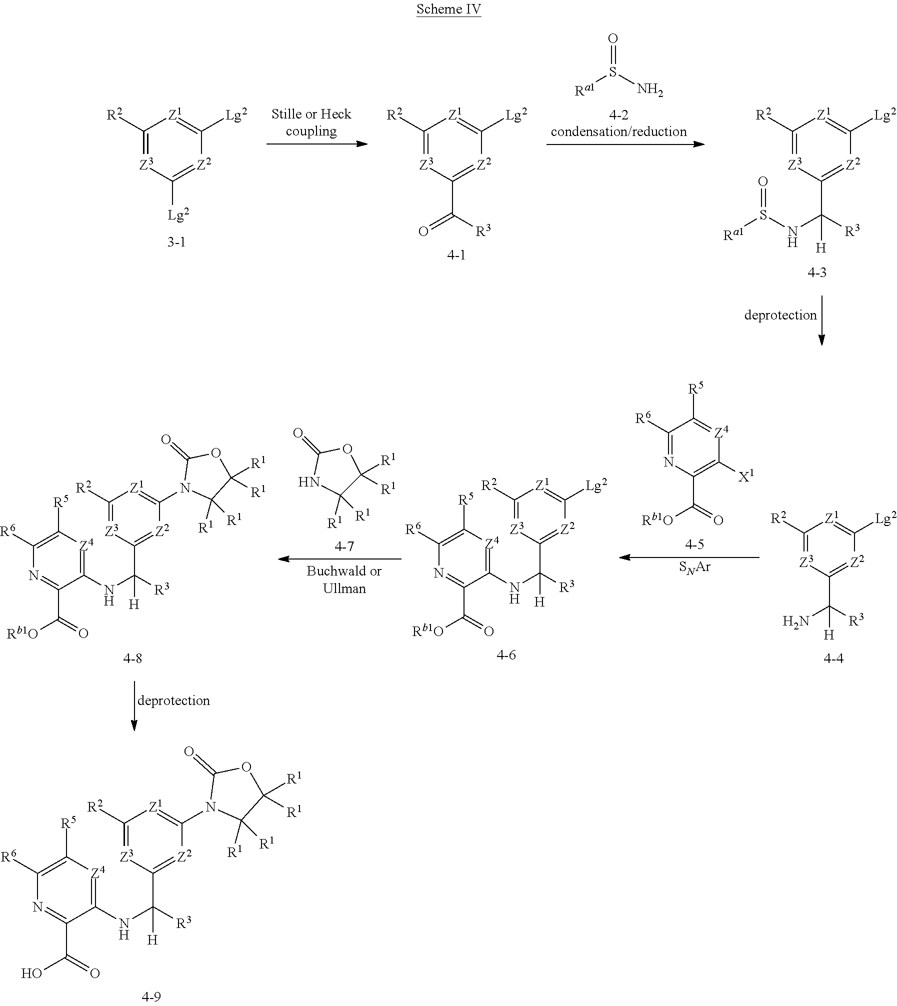

Intermediates for the synthesis of compounds of Formula (I) can be prepared from protected amino acids 5-1 as shown in Scheme V. Amino acid derivatives 5-1 where R^{c1} is part of suitable carbamate protecting group (e.g., tert-butyl, benzyl, or 9-methyl-9H-fluorene) can be deprotected under standard conditions, such as in the presence of an acid (e.g., TFA) or base (e.g., LiOH), to afford compounds 5-2. Compounds 5-2 can be coupled with an amine 5-3 under standard amide bond formation conditions such as in the presence of an amide coupling reagent (e.g., N,N′-carbonyldiimidazole or HATU and a base (e.g. diisopropylethylamine) to provide compounds 5-4. Reduction and subsequent intramolecular cyclization of compounds 5-4 with a suitable hydride source, such as in the presence of an aluminum reducing agent (e.g., sodium bis(2-methoxyethoxy)aluminum hydride) can afford imidazolidinones 5-5.

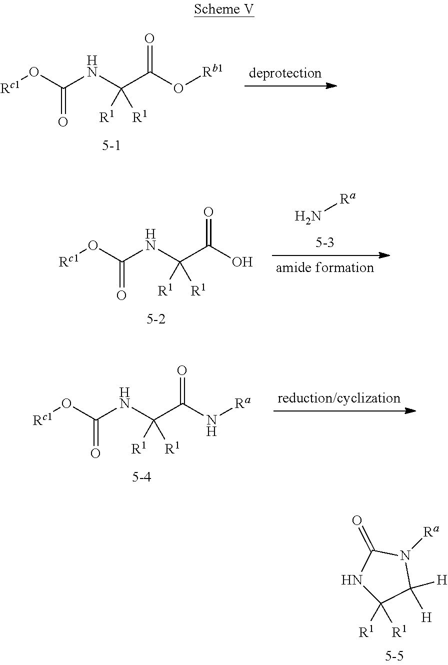

Compounds of Formula (I) can be prepared from arenes 4-6 that can be coupled with imidazolidinones 6-1 under standard Buchwald-Hartwig coupling conditions, such as in the presence of a catalyst (e.g., XPhos Pd G3), and a base (e.g., Cs₂CO₃ or K₃PO₄) or standard Ullman coupling conditions, such as in the presence of a copper catalyst (e.g., CuI), a ligand (e.g., N1,N2-dimethylethane-1,2-diamine or N,N-dimethylglycine), and a base (e.g., K₂CO₃) to provide intermediates 6-2. Esters 6-2 can be optionally deprotected under standard conditions, such as in the presence of an acid (e.g., TFA) or base (e.g., LiOH), to afford compounds 6-3.

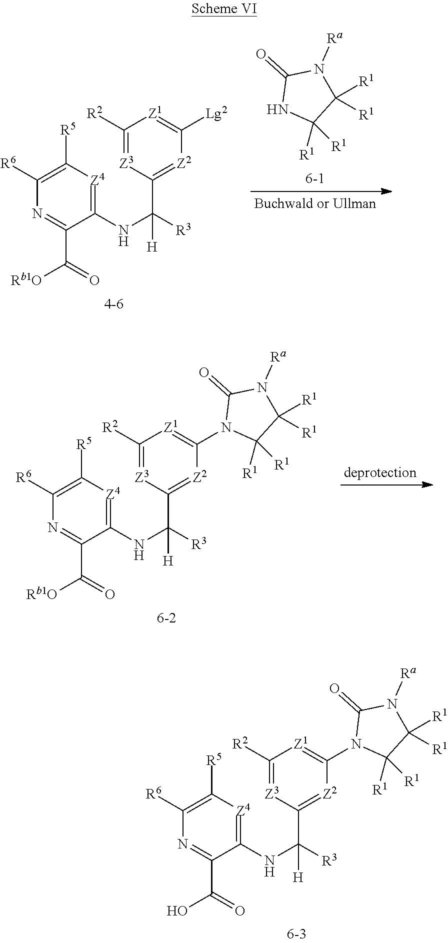

### Example 1. 3-((1-(3-((S)-4-Benzyl-2-oxooxazolidin-3-yl)-5-methylphenyl)ethyl)amino)-6-chloropicolinic acid

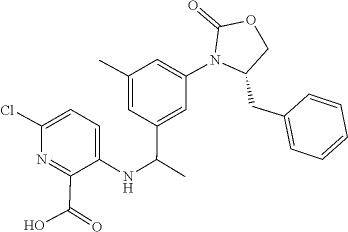 [SMILES: Cc1cc(C(C)Nc2ccc(Cl)nc2C(=O)O)cc(N2C(=O)OC[C@@H]2Cc2ccccc2)c1]

### Step 1. tert-Butyl 3-amino-6-chloropicolinate

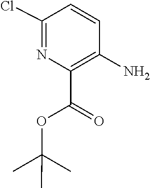

To a solution of 3-amino-6-chloropyridine-2-carboxylic acid (2.00 g, 11.6 mmol) in toluene (20 mL) was added tert-butyl N,N′-diisopropylcarbamimidate (9.29 g, 46.4 mmol) dropwise. The reaction mixture was stirred at 65° C. for 15 min. The mixture was cooled to room temperature, diluted with water (20 mL), and extracted with EtOAc (20 mL×3). The combined organic layers were dried over Na₂SO₄, filtered, and concentrated. The residue was purified by silica gel chromatography eluting with EtOAc/heptane (0-20%) to afford the title compound (1.70 g, 7.43 mmol, 64.1% yield) as a yellow solid. LC-MS calc. for C₁₀H₁₄ClN₂O₂[M+H]⁺ m/z=229.1; Found 229.4.

### Step 2. (S)-3-(3-Acetyl-5-methylphenyl)-4-benzyloxaolidin-2-one

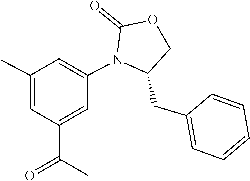 [SMILES: CC(=O)c1cc(C)cc(N2C(=O)OC[C@@H]2Cc2ccccc2)c1]

To a solution of 1-(3-bromo-5-methylphenyl)ethanone (300 mg, 1.40 mmol) in 1,4-dioxane (6.0 mL) was added (S)-4-benzyl-2-oxazolidinone (500 mg, 2.80 mmol), copper(I) iodide (54 mg, 0.28 mmol), N1,N2-dimethylethane-1,2-diamine (0.034 mL, 0.310 mmol) and potassium carbonate (390 mg, 2.80 mmol). The resulting mixture was sparged with nitrogen (3×) and was stirred at 100° C. overnight. The reaction mixture was cooled to room temperature and diluted with with sat. NH₄Cl solution (aq.) (20 mL) and EtOAc (20 mL). The layers were separated, and the aqueous layer was extracted with EtOAc (20 mL×2). The combined organic layers were washed with brine, dried over sodium sulfate, filtered, and concentrated. The crude product was purified by silica gel chromatography (0-50% EtOAc/heptane) to afford the title compound (440 mg, >99%) as a beige solid. LCMS calc. for C₁₉H₂₀NO₃ [M+H]⁺ m/z=310.1; found 310.4.

### Step 3. (4S)-4-Benyl-3-(3-(1-hydroxyethyl)-5-methylphenyl)oxaolidin-2-one

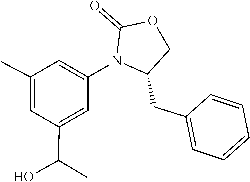 [SMILES: Cc1cc(C(C)O)cc(N2C(=O)OC[C@@H]2Cc2ccccc2)c1]

To a solution of (S)-3-(3-acetyl-5-methylphenyl)-4-benzyl-1,3-oxazolidin-2-one (436 mg, 1.41 mmol) in DCM (3 mL) and methanol (3 mL) was added sodium borohydride (179 mg, 4.72 mmol). The resulting mixture was stirred for 2 h. The reaction was quenched by adding water (30 mL), and the resulting mixture diluted with EtOAc (30 mL). The layers were separated, and the aqueous layer was extracted with EtOAc (30 mL×2). The combined organic layers were washed with brine, dried over sodium sulfate, filtered, and concentrated. The crude product was purified by silica gel chromatography (20-60% EtOAc/heptane) to afford the title compound (211 mg, 0.678 mmol, 48.1% yield) as a colorless oil. LCMS calc. for C₁₉H₂₂NO₃ [M+H]⁺ m/z=312.2; found 312.4.

### Step 4: (4S)-4-Benyl-3-(3-(1-bromoethyl)-5-methylphenyl)oxaolidin-2-one

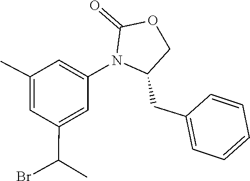 [SMILES: Cc1cc(C(C)Br)cc(N2C(=O)OC[C@@H]2Cc2ccccc2)c1]

To a solution of (4S)-4-benzyl-3-[3-(1-hydroxyethyl)-5-methylphenyl]-1,3-oxazolidin-2-one (160 mg, 0.514 mmol) in DCM (3 mL) was added phosphorus tribromide (0.146 mL, 1.54 mmol). The resulting mixture was stirred at room temperature for 2 hours. The reaction was diluted with sat. NaHCO₃ (aq.) (10 mL) solution followed by 10 mL of EtOAc. The layers were separated, and the aqueous layer was extracted with EtOAc (10 mL×2). The combined organic layers were washed with brine, dried over sodium sulfate, filtered, and concentrated to afford the title compound (192 mg, 0.513 mmol, 99.8% yield) as a colorless oil. The crude product was directly used in the next step without further purification. HPLC on a C18 column (5-95% MeCN/0.1% TFA (aq.)): t_{R}: 6.67 min.

### Step 5. tert-Butyl 3-((1-(3-((S)-4-benzyl-2-oxooxaolidin-3-yl)-5-methylphenyl)ethyl)amino)-6-chloropicolinate

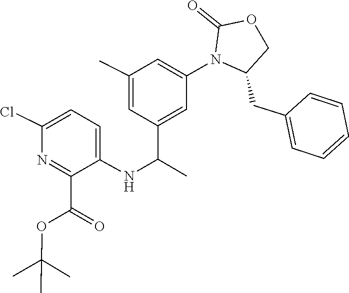 [SMILES: Cc1cc(C(C)Nc2ccc(Cl)nc2C(=O)OC(C)(C)C)cc(N2C(=O)OC[C@@H]2Cc2ccccc2)c1]

To a solution of (4S)-4-benzyl-3-[3-(1-bromoethyl)-5-methylphenyl]-1,3-oxazolidin-2-one (95.0 mg, 0.254 mmol) in acetonitrile (2 mL) was added potassium carbonate (70.2 mg, 0.508 mmol) followed by tert-butyl 3-amino-6-chloropyridine-2-carboxylate (116 mg, 0.508 mmol, Example 1, Step 1). The resulting mixture was stirred at 60° C. for 3 d. The mixture was diluted with water (10 mL) and EtOAc (10 mL). The layers were separated, and the aqueous layer was extracted with EtOAc (10 mL×2). The combined organic layers were washed with brine, dried over sodium sulfate, filtered, and concentrated. The crude product was further purified by silica gel chromatography (0-60% EtOAc/heptane) to afford the title compound (29.0 mg, 0.0556 mmol, 21.9% yield) as a beige solid. LCMS calc. for C₂₉H₃₃ClN₃O₄[M+H]⁺ m/z=522.2, 524.2; found 522.4, 524.4.

### Step 6. 3-((1-(3-((S)-4-Benyl-2-oxooxazolidin-3-yl)-5-methylphenyl)ethyl)amino)-6-chloropicolinic acid

To a solution of tert-butyl 3-[1-[3-[(4S)-4-benzyl-2-oxo-1,3-oxazolidin-3-yl]-5-methylphenyl]ethylamino]-6-chloropyridine-2-carboxylate (29 mg, 0.0556 mmol) in DCM (1 mL) was added trifluoroacetic acid (0.250 mL, 3.27 mmol). The resulting mixture was stirred for 3 h. The reaction mixture was concentrated. The crude product was purified by prep-HPLC (20-100% MeCN/0.1% TFA (aq.)) to afford the title compound as a TFA salt (8.2 mg, 0.018 mmol, 32% yield) as a light yellow solid. ¹H NMR (300 MHz, DMSO-d₆) δ 12.96 (br s, 1H), 8.33-8.30 (m, 1H), 7.44-7.17 (m, 6H), 7.15-6.92 (m, 4H), 4.92-4.70 (m, 2H), 4.36-4.31 (m, 1H), 4.17-4.10 (m, 1H), 2.95-2.58 (m, 2H), 2.32 (s, 3H), 1.55-1.51 (m, 3H). LCMS calc. for C₂₅H₂₅ClN₃O₄[M+H]⁺ m/z=466.2, 468.2; found 466.4, 468.4.

### Example 2. 3-((1-(3-((S)-4-Benzyl-2-oxooxazolidin-3-yl)-5-methylphenyl)ethyl)amino)-6-chloropicolinic acid (Isomer 2)

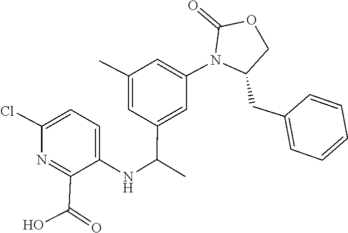 [SMILES: Cc1cc(C(C)Nc2ccc(Cl)nc2C(=O)O)cc(N2C(=O)OC[C@@H]2Cc2ccccc2)c1]

### Step 1. Methyl 3-((1-(3-((S)-4-benyl-2-oxooxazolidin-3-yl)-5-methylphenyl)ethyl)amino)-6-chloropicolinate

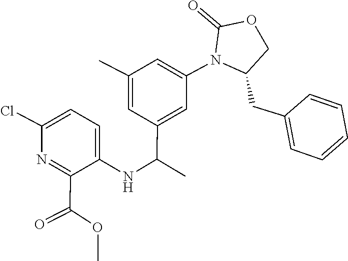 [SMILES: COC(=O)c1nc(Cl)ccc1NC(C)c1cc(C)cc(N2C(=O)OC[C@@H]2Cc2ccccc2)c1]

The title compound was synthesized by procedures analogous to those outlined in Example 1, Steps 2-5, utilizing methyl 3-amino-6-chloropicolinate instead of tert-butyl 3-amino-6-chloropicolinate in Step 5. Purification by prep HPLC on a C18 column (20-100% MeCN/0.1% TFA (aq.)) afforded the title compound as a TFA salt. LC-MS calc. for C₂₆H₂₇ClN₃O₄[M+H]⁺: m/z=480.2; Found=480.5.

### Step 2. 3-((1-(3-((S)-4-Benzyl-2-oxooxaolidin-3-yl)-5-methylphenyl)ethyl)amino)-6-chloropicolinic acid (Isomer 2)

The isomers of methyl 3-((1-(3-((S)-4-benzyl-2-oxooxazolidin-3-yl)-5-methylphenyl)ethyl)amino)-6-chloropicolinate, TFA salt (20.0 mg, 0.034 mmol) were separated using chiral prep-HPLC on a Lux Cellulose-4 column (30 mL/min, 70:15:15 hexane/MeOH/IPA) (Isomer 1 t_{R}: 5.68 min; Isomer 2 t_{R}: 6.63 min) For isomer 2, the fractions were combined and concentrated. The resulting residue was dissolved in 1,4-dioxane (0.40 mL) and LiOH (aq.) (0.20 mL, 0.20 mmol, 1M). The mixture was stirred for 2 h. The reaction mixture was diluted with MeCN, filtered, and purified by prep HPLC on a CSH—C18 column (51-71% MeCN/0.1% TFA (aq.)) to afford the title compound as a TFA salt (3.8 mg, 6.5 μmol, 19% yield). ¹H NMR (400 MHz, DMSO-d₆) δ 8.29 (d, J=6.2 Hz, 1H), 7.42-7.32 (m, 2H), 7.30 (s, 1H), 7.25-7.15 (m, 3H), 7.14-7.05 (m, 3H), 7.02 (s, 1H), 4.91-4.80 (m, 1H), 4.79-4.67 (m, 1H), 4.36 (t, J=8.6 Hz, 1H), 4.14 (dd, J=9.0, 4.3 Hz, 1H), 2.95-2.84 (m, 1H), 2.84-2.73 (m, 1H), 2.32 (s, 3H), 1.53 (d, J=6.7 Hz, 3H). LC-MS calc. for C₂₅H₂₅ClN₃O₄[M+H]⁺: m/z=466.2; Found: 466.1.

### Example 3. 3-((1-(3-((S)-4-Benzyl-2-oxooxazolidin-3-yl)-5-methylphenyl)ethyl)amino)-6-fluoropicolinic acid

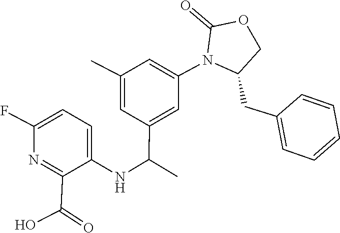 [SMILES: Cc1cc(C(C)Nc2ccc(F)nc2C(=O)O)cc(N2C(=O)OC[C@@H]2Cc2ccccc2)c1]

### Step 1. Ethyl 3-amino-6-fluoropicolinate

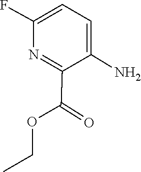

Triethylamine (8.2 mL, 58 mmol) was added to solution of dichloro 1,1′-bisdiphenyl-phosphino)ferrocene palladium (II) dichloromethane (3.2 g, 3.9 mmol) and 6-fluoro-2-iodopyridin-3-amine (4.6 g, 19 mmol) in EtOH (240 mL), and the reaction mixture was stirred under an atmosphere of CO for 48 h. The atmosphere of CO was removed, and the mixture was filtered and concentrated. The crude residue was purified by silica gel chromatography (0-10% EtOAc/DCM). The resulting solid was further purified by trituration with MTBE, and the filtrate was concentrated to afford the title compound as a tan solid (900 mg, 4.9 mmol, 25% yield). LC-MS calc. for C₈H₁₀FN₂O₂[M+H]⁺: m/z=185.1; Found: 185.0.

### Step 2. Ethyl 3-((1-(3-((S)-4-benyl-2-oxooxazolidin-3-yl)-5-methylphenyl)ethyl)amino)-6-fluoropicolinate

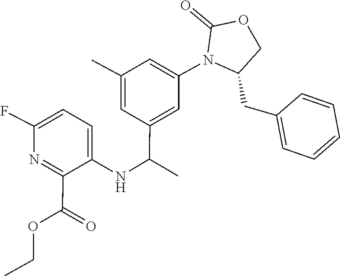 [SMILES: CCOC(=O)c1nc(F)ccc1NC(C)c1cc(C)cc(N2C(=O)OC[C@@H]2Cc2ccccc2)c1]

The title compound was synthesized by procedures analogous to those outlines in Example 1, Steps 2-5, utilizing ethyl 3-amino-6-fluoropicolinate instead of tert-butyl 3-amino-6-chloropicolinate in Step 5. LC-MS calc. for C₂₇H₂₉FN₃O₄[M+H]⁺: m/z=478.2; Found: 478.5.

### Step 3. 3-((1-(3-((S)-4-Benzyl-2-oxooxaolidin-3-yl)-5-methylphenyl)ethyl)amino)-6-fluoropicolinic acid

To a solution of ethyl 3-[1-[3-[(4S)-4-benzyl-2-oxo-1,3-oxazolidin-3-yl]-5-methylphenyl]ethylamino]-6-fluoropyridine-2-carboxylate (80.0 mg, 0.167 mmol) in THF (2 mL)/water (2 mL) was added lithium hydroxide monohydrate (70.0 mg, 1.67 mmol). The resulting mixture was stirred at 40° C. for 2 h. The solvent was removed under reduced pressure, and the crude product was directly purified by prep-HPLC (20-100% MeCN/0.1% TFA (aq.)) to afford the title compound as the TFA salt (3.3 mg, 0.0073 mmol, 4.4% yield), a white solid. ¹H NMR (300 MHz, DMSO-d₆) δ 13.00 (br, 1H), 8.25-8.22 (m, 1H), 7.42-7.38 (m, 1H), 7.35-6.95 (m, 9H), 4.97-4.68 (m, 2H), 4.39-4.32 (m, 1H), 4.19-4.14 (m, 1H), 2.97-2.69 (m, 2H), 2.34 (s, 3H), 1.58-1.53 (m, 3H). LCMS calc. for C₂₅H₂₅FN₃O₄[M+H]⁺ m/z=450.2; found 450.5.

### Example 4. 3-((1-(3-((S)-4-Benzyl-2-oxooxazolidin-3-yl)-5-fluorophenyl)ethyl)amino)-6-chloropicolinic acid

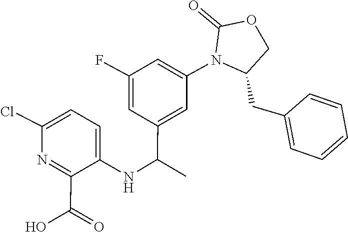 [SMILES: CC(Nc1ccc(Cl)nc1C(=O)O)c1cc(F)cc(N2C(=O)OC[C@@H]2Cc2ccccc2)c1]

### Step 1. (4S)-4-Benzyl-3-(3-fluoro-5-(1-hydroxyethyl)phenyl)oxaolidin-2-one

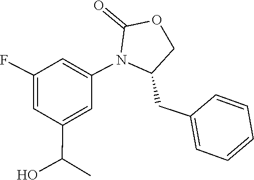 [SMILES: CC(O)c1cc(F)cc(N2C(=O)OC[C@@H]2Cc2ccccc2)c1]

The title compound was synthesized by procedures analogous to those outlines in Example 1, Steps 2-4, utilizing 1-(3-bromo-5-fluorophenyl)ethan-1-one instead of 1-(3-bromo-5-methylphenyl)ethan-1-one in Step 2. LC-MS calc. for C18H₁₉FNO₃ [M+H]⁺: m/z=316.1; Found: 316.3.

### Step 2. 3-((1-(3-((S)-4-benyl-2-oxooxaolidin-3-yl)-5-fluorophenyl)ethyl)amino)-6-chloropicolinic acid

Methanesulfonyl chloride (37 μL, 0.48 mmol) was added to a solution of (4S)-4-benzyl-3-(3-fluoro-5-(1-hydroxyethyl)phenyl)oxazolidin-2-one (100 mg, 0.32 mmol) and triethylamine (0.13 mmol, 0.95 mmol) in DCM (3.2 mL), and the reaction mixture was stirred for 1.5 h. Then methyl 3-amino-6-chloropyridine-2-carboxylate (71 mg, 0.38 mmol) was added, and the mixture was stirred for 18 h. The reaction mixture was concentrated, and the residue resuspended in 1,4-dioxane (2.0 mL) and 1 N LiOH (aq.) (1.6 mL, 1.6 mmol). After stirring for 2 h, the resulting suspension was diluted with water (5.0 mL) and DCM (5.0 mL). The two phases were separated, and the aqueous layer was extracted with DCM (5.0 mL×2). The combined organic layers were dried over sodium sulfate, filtered, and concentrated. The crude residue was purified by prep HPLC (15-35% MeCN/0.15% NH₄OH (aq.)) to afford the title compound (3.6 mg, 7.2 μmol, 2.3% yield) as an ammonium salt. LCMS calc. for C₂₄H₂₂ClFN₃O₄[M+H]⁺: m/z=470.1; Found: 470.0.

### Examples 5-8

Examples 5-8 listed in Tables 1 and 2 were synthesized according to procedures analogous to Example 1 and Example 4. All examples in these tables were prepared as the ammonium unless otherwise noted.

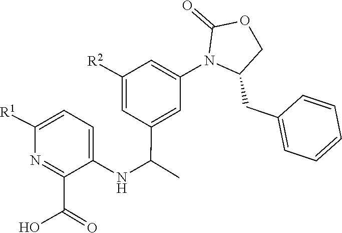

TABLE 1
Examples 5-8

| Example | R¹ | R² | LCMS [M + H]⁺ |
|---|---|---|---|
| 5 | F | F | 454.1 |
| 6 | Cl | Cl | 486.0 |
| 7 | F | Cl | 470.1 |
| 8 | F | CF₃ | 504.1 |

TABLE 2
Examples 5-8

| Example | Compound Name |
|---|---|
| 5 | 3-((1-(3-((S)-4-Benzyl-2-oxooxazolidin- |
| | 3-yl)-5-fluorophenyl)ethyl) amino)-6- |
| | fluoropicolinic acid |
| 6 | 3-((1-(3-((S)-4-Benzyl-2-oxooxazolidin- |
| | 3-yl)-5-chlorophenyl)ethyl) amino)-6- |
| | chloropicolinic acid |
| 7 | 3-((1-(3-((S)-4-Benzyl-2-oxooxazolidin- |
| | 3-yl)-5-chlorophenyl)ethyl) amino)-6- |
| | fluoropicolinic acid |
| 8 | 3-((1-(3-((S)-4-Benzyl-2-oxooxazolidin- |
| | 3-yl)-5-(trifluoromethyl)phenyl) |
| | ethyl)amino)-6-fluoropicolinic acid, TFA salt |

### Example 9. 3-(((R)-1-(3-((S)-4-(4-Bromobenzyl)-2-oxooxazolidin-3-yl)-5-methylphenyl)ethyl) amino)-6-chloropicolinic acid

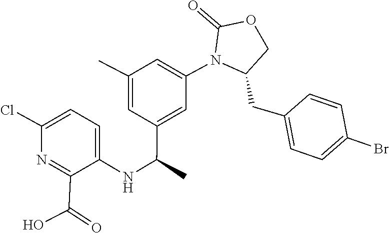 [SMILES: Cc1cc([C@@H](C)Nc2ccc(Cl)nc2C(=O)O)cc(N2C(=O)OC[C@@H]2Cc2ccc(Br)cc2)c1]

### Step 1: (S)-2-Amino-3-(4-bromophenyl)propan-1-ol

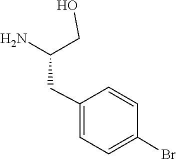 [SMILES: N[C@H](CO)Cc1ccc(Br)cc1]

To a solution of (S)-2-amino-3-(4-bromophenyl)propanoic acid (1.00 g, 4.10 mmol) in THF (20 mL) was added borane dimethyl sulfide complex (1.24 g, 16.4 mmol) dropwise at 0° C. The reaction mixture was stirred overnight at room temperature. The reaction mixture was cooled to 0° C., and the reaction was quenched with MeOH (2.0 mL). The mixture was warmed to room temperature and stirred for 30 min. The reaction mixture was concentrated to afford the title compound (1.10 g), which was used for the next reaction without further purification. LC-MS calc. for C₉H₁₃BrNO [M+H]+: m/z=230.0, 232.0; Found: 230.3, 232.3.

### Step 2: (4S)-4-[(4-Bromophenyl)methyl]-1,3-oxaolidin-2-one

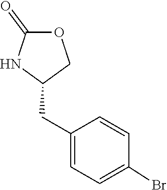 [SMILES: O=C1N[C@@H](Cc2ccc(Br)cc2)CO1]

To a solution of (S)-2-amino-3-(4-bromophenyl)propan-1-ol (1020 mg, 4.43 mmol) in DCM (20 mL) was added triethylamine (1.85 mL, 13.3 mmol) and then portion wise triphosgene (526 mg, 1.77 mmol) at 0° C. The resulting mixture was stirred for 30 min at room temperature. The reaction was quenched with sat. NH₄Cl (aq.) (5.0 mL) solution, and the reaction mixture was stirred for 30 min. The resulting layers were separated, and the aqueous layer was extracted by EtOAc (2×20 mL). The combined organic layers were washed with sat. NaHCO₃ (aq.) solution, dried over MgSO₄, and concentrated. The crude residue was purified by silica gel chromatography (0-100% EtOAc/heptane) to yield the title compound (397 mg, 1.55 mmol, 34.0% yield) as white solid. LC-MS calc. for C₁₀H₁₁BrNO₂ [M+H]+: m/z=256.0, 258.0; Found: 256.3, 258.3.

### Step 3: (S)—N-[(1R)-1-(3-Bromo-5-methylphenyl)ethyl]-2-methylpropane-2-sulfinamide

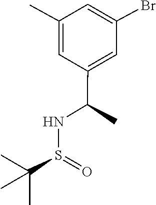 [SMILES: Cc1cc(Br)cc([C@@H](C)N[S@@](=O)C(C)(C)C)c1]

To a solution of 1-(3-bromo-5-methylphenyl)ethanone (3.00 g, 14.1 mmol) in THF (45.0 mL) were added (S)-(−)-tert-butanesulfinamide (1.79 g, 14.8 mmol) and titanium(IV) ethoxide (6.42 g, 28.2 mmol), and the mixture was stirred at 65° C. for 2 h. The reaction mixture was cooled to −40° C., and L-selectride (5.35 g, 28.2 mmol) was added at −40° C. The mixture was allowed to warm to room temperature and was stirred for 4 h. The reaction was quenched with sat. NH₄Cl (aq.) (45 mL), and the mixture extracted with EtOAc (3×50 mL). The combined organic layer was washed with brine, dried over MgSO₄, filtered, and concentrated. The crude product was purified by silica gel chromatography (0-50% EtOAc/heptane) to yield the title compound (953 mg, 1.14 mmol, 8.12% yield) as yellow oil. LCMS calc. for C₁₃H₂₁BrNOS [M+H]⁺ m/z=318.0, 320.0; found 318.2, 320.2.

### Step 4: (1R)-1-(3-Bromo-5-methylphenyl)ethanamine, HCl salt

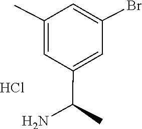

Methanol (0.290 mL, 7.12 mmol) was added to a solution of (S)—N-[(1R)-1-(3-bromo-5-methylphenyl)ethyl]-2-methylpropane-2-sulfinamide (1.51 g, 4.74 mmol) in 4 N HCl in dioxane (11.8 ml, 47.4 mmol). The mixture was stirred for 16 h. To the mixture was added diethyl ether. The resulting solid was collected by filtration, washed with diethyl ether, and dried to afford the title compound as an HCl salt (910 mg, 3.63 mmol, 76.6% yield) as a white solid. LCMS calc. for C₉H₁₃BrN [M+H]⁺ m/z=214.0, 216.0; found 214.0, 216.0.

### Step 5: Methyl 3-[[(1R)-1-(3-bromo-5-methylphenyl)ethyl]amino]-6-chloropyridine-2-carboxylate

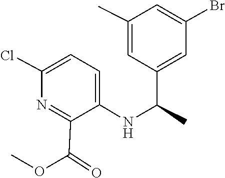 [SMILES: COC(=O)c1nc(Cl)ccc1N[C@H](C)c1cc(C)cc(Br)c1]

To a solution of (1R)-1-(3-bromo-5-methylphenyl)ethanamine, HCl salt (1.44 g, 5.72 mmol) and methyl 6-chloro-3-fluoropyridine-2-carboxylate (1.53 g, 8.09 mmol) in DMSO (20 mL) was added N,N-diisopropylethylamine (3.52 mL, 20.2 mmol). The reaction mixture was stirred at 100° C. overnight. The mixture was cooled to room temperature and then diluted with water (30 mL) and EtOAc (30 mL). The two phases were separated, and the aqueous layer was extracted with EtOAc (30 ml×2). The combined organic layers were washed with brine, dried over Na₂SO₄, filtered, and concentrated. The crude mixture was purified by prep-HPLC (20-100% MeCN/0.1% TFA (aq.)) to afford the title compound as a TFA salt (1.24 g, 3.23 mmol, 56.4% yield), a light yellow solid. LCMS calc. for C₁₆H₁₇BrClN₂O₂[M+H]⁺ m/z=383.0, 385.0; found 383.3, 385.3.

### Step 6. Methyl 3-(((R)-1-(3-((S)-4-(4-bromobenzyl)-2-oxooxaolidin-3-yl)-5-methyl-phenyl)ethyl)amino)-6-chloropicolinate

To a solution of methyl 3-[[(1R)-1-(3-bromo-5-methylphenyl)ethyl]amino]-6-chloro-pyridine-2-carboxylate (37.0 mg, 0.0964 mmol) in 1,4-dioxane (0.9 mL) was added (4S)-4-[(4-bromophenyl)methyl]-1,3-oxazolidin-2-one (25.9 mg, 0.101 mmol), copper(I) iodide (18.4 mg, 0.0964 mmol), N1,N2-dimethylethane-1,2-diamine (11.4 μL, 0.106 mmol), and potassium carbonate (53.3 mg, 0.386 mmol). The resulting mixture was sparged with nitrogen (3×) and stirred at 100° C. for 18 h. The mixture was cooled to room temperature and diluted with sat. NH₄Cl (aq.) (5 mL) and EtOAc (5 mL). The layers were separated, and the aqueous layer was extracted with EtOAc (5 mL×2). The combined organic layers were washed with brine, dried over sodium sulfate, filtered, and concentrated to afford the title compound (25.0 mg, 0.0447 mmol, 46.4% yield) as a brown solid.

LCMS calc. for C₂₆H₂₆BrClN₃O₄[M+H]⁺ m/z=558.1, 560.1; found 558.4, 560.3.

### Step 7. 3-(((R)-1-(3-((S)-4-(4-Bromobenzyl)-2-oxooxaolidin-3-yl)-5-methylphenyl)ethyl) amino)-6-chloropicolinic acid

The title compound was synthesized by procedures analogous to those outlines in Example 3, Step 3. LC-MS calc. for C₂₅H₂₄BrClN₃O₄[M+H]⁺: m/z=544.1, 546.1; Found: 544.2, 546.2.

### Example 10. 3-(((R)-1-(3-((S)-4-Benzyl-2-oxooxazolidin-3-yl)-5-fluorophenyl)ethyl)amino)-6-chloropicolinic acid

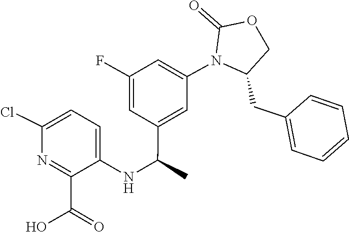 [SMILES: C[C@@H](Nc1ccc(Cl)nc1C(=O)O)c1cc(F)cc(N2C(=O)OC[C@@H]2Cc2ccccc2)c1]

The title compound was synthesized by procedures analogous to those outlines in Example 9, Steps 3-7, utilizing 1-(3-bromo-5-fluorophenyl)ethan-1-one instead of 1-(3-bromo-5-methylphenyl)ethan-1-one in Step 4. ¹H NMR (400 MHz, DMSO-d₆) δ 8.30 (s, 1H), 7.61-7.31 (m, 3H), 7.22 (s, 3H), 7.15-7.00 (m, 4H), 4.96-4.86 (m, 1H), 4.85-4.75 (m, 1H), 4.38 (t, J=8.4 Hz, 1H), 4.18 (d, J=8.9 Hz, 1H), 2.93 (d, J=13.8 Hz, 1H), 2.85 (dd, J=13.9, 6.9 Hz, 1H), 1.54 (d, J=6.5 Hz, 3H). LC-MS calc. for C₂₄H₂₂ClFN₃O₄[M+H]⁺: m/z=470.1, 472.1; Found: 469.9, 472.0.

### Example 11. 3-[1-[6-[(4S)-4-Benzyl-2-oxo-1,3-oxazolidin-3-yl]-4-methyl-2-pyridinyl]ethylamino]-6-fluoropyridine-2-carboxylic acid (Isomer 2)

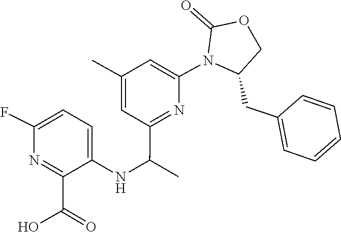 [SMILES: Cc1cc(C(C)Nc2ccc(F)nc2C(=O)O)nc(N2C(=O)OC[C@@H]2Cc2ccccc2)c1]

### Step 1. (4S)-4-Benzyl-3-(6-bromo-4-methylpyridin-2-yl)-1,3-oxaolidin-2-one

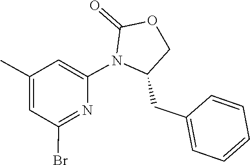 [SMILES: Cc1cc(Br)nc(N2C(=O)OC[C@@H]2Cc2ccccc2)c1]

To a solution of 2,6-dibromo-4-methylpyridine (1.00 g, 3.99 mmol) and (S)-4-benzyl-2-oxazolidinone (777 mg, 4.38 mmol) in 1,4-dioxane (15 mL) was added copper(I) iodide (152 mg, 0.797 mmol), N1,N2-dimethylethane-1,2-diamine (0.944 mL, 8.77 mmol) and potassium carbonate (1.10 g, 7.97 mmol). The reaction mixture was bubbled with N₂ for 1 min and stirred at 100° C. overnight. The mixture was diluted with sat. NH₄Cl (aq.) (10 mL) and extracted with EtOAc (10 mL×3). The combined organic layers were washed with brine, dried over Na₂SO₄, filtered, and concentrated. The residue was purified by silica gel chromatography (0-40% EtOAc/heptane) to yield the title compound (797 mg, 2.29 mmol, 57.6% yield). LCMS calc. for C₁₆H₁₆BrN₂O₂ [M+H]⁺: m/z=347.0, 349.0; found 347.3, 349.3.

### Step 2. (4S)-3-(6-Acetyl-4-methylpyridin-2-yl)-4-benyl-1,3-oxaolidin-2-one

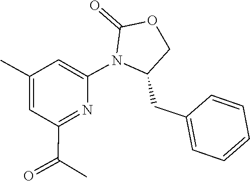 [SMILES: CC(=O)c1cc(C)cc(N2C(=O)OC[C@@H]2Cc2ccccc2)n1]

To a solution of (4S)-4-benzyl-3-(6-bromo-4-methylpyridin-2-yl)-1,3-oxazolidin-2-one (867 mg, 2.50 mmol) and tributyl(1-ethoxyvinyl)tin (1.27 mL, 3.75 mmol) in 1,4-dioxane (15 mL) was added bis(triphenylphosphine)palladium(II) dichloride (175 mg, 0.250 mmol). The reaction mixture was bubbled with N₂ for 1 min and stirred at 100° C. overnight. The mixture was cooled to room temperature, diluted with sat. KF (aq.) solution, and stirred for 1 h. The solid was removed by filtration. The filtrate was diluted with DCM (10 mL) and washed with water (10 mL) and brine (10 mL), dried over Na₂SO₄, filtered, and concentrated. The residue was dissolved in THF (5 mL), and 1N HCl (5.0 mL, 5.0 mmol) was added. The resulting mixture was further stirred for 15 min. The reaction was diluted with water (5 mL) and extracted with DCM (5 mL×3). The combined organic layers were dried over Na₂SO₄, filtered, and concentrated. The residue was purified by silica gel chromatography (0-50% EtOAc/heptane) to yield the title compound (625 mg, 2.01 mmol, 80.6% yield). LCMS calc. for C18H₁₉N₂O₃ [M+H]⁺: m/z=311.1; found 311.4.

### Step 3. (4S)-4-Benyl-3-[6-(1-hydroxyethyl)-4-methylpyridin-2-yl]-1,3-oxaolidin-2-one

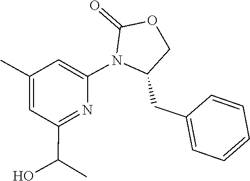 [SMILES: Cc1cc(C(C)O)nc(N2C(=O)OC[C@@H]2Cc2ccccc2)c1]

The title compound was synthesized by procedures analogous to those outlines in Example 1, Step 3. LCMS calc. for C18H₂₁N₂O₃ [M+H]⁺: m/z=313.2; found 313.4.

### Step 4. 1-[6-[(4S)-4-Benyl-2-oxo-1,3-oxazolidin-3-yl]-4-methylpyridin-2-yl]ethyl methanesulfonate

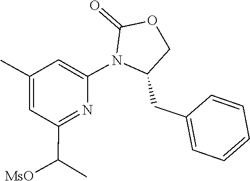 [SMILES: Cc1cc(C(C)OS(C)(=O)=O)nc(N2C(=O)OC[C@@H]2Cc2ccccc2)c1]

To a solution of (4S)-4-benzyl-3-[6-(1-hydroxyethyl)-4-methylpyridin-2-yl]-1,3-oxazolidin-2-one (100 mg, 0.320 mmol) and triethylamine (0.134 mL, 0.960 mmol) in DCM (2 mL) was added methanesulfonyl chloride (0.0372 mL, 0.480 mmol) at 0° C. The reaction mixture was stirred at room temperature for 30 min. The mixture was diluted with sat. NaHCO₃ (aq.) (1 mL), and the mixture extracted with DCM (1 mL×3). The combined organic layers were dried over Na₂SO₄, filtered, and concentrated to afford the title compound (120 mg, 0.307 mmol, 96.0% yield). LCMS calc. for C₁₉H₂₃N₂O₅S [M+H]⁺: m/z=391.1; found 391.5.

### Step 5. Ethyl 3-[1-[6-[(4S)-4-benzyl-2-oxo-1,3-oxaolidin-3-yl]-4-methyl-2-pyridinyl]ethylamino]-6-fluoropyridine-2-carboxylate (Isomer 2)

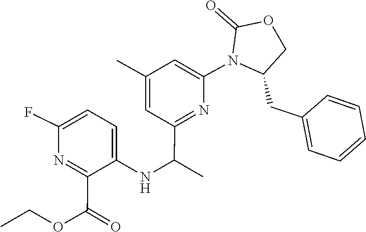 [SMILES: CCOC(=O)c1nc(F)ccc1NC(C)c1cc(C)cc(N2C(=O)OC[C@@H]2Cc2ccccc2)n1]

To a solution of 1-[6-[(4S)-4-benzyl-2-oxo-1,3-oxazolidin-3-yl]-4-methylpyridin-2-yl]ethyl methanesulfonate (120 mg, 0.307 mmol) and ethyl 3-amino-6-fluoropyridine-2-carboxylate (67.9 mg, 0.369 mmol, Example 3, Step 1) in acetonitrile (2 mL) was added potassium carbonate (85.0 mg, 0.615 mmol) and sodium iodide (92.1 mg, 0.615 mmol). The reaction mixture was stirred at 60° C. for 48 h. The reaction mixture was diluted with DCM (5 mL), washed with water (5 mL) and brine (5 mL), dried over Na₂SO₄, filtered, and concentrated. The residue was purified by prep-HPLC on a C18 column (20-70% MeCN/0.1% TFA (aq.)) to afford two isomers of the title compound as the TFA salts: isomer 1 (21.0 mg, 0.0439 mmol, 14.3% yield, t_{R}: 6.67 min) and isomer 2 (15.0 mg, 0.0313 mmol, 10.2% yield, t_{R}: 6.78 min). Isomer 2: LCMS calc. for C₂₆H₂₈FN₄O₄ [M+H]⁺: m/z=479.2; found 479.4.

### Step 6. 3-[1-[6-[(4S)-4-Benyl-2-oxo-1,3-oxaolidin-3-yl]-4-methyl-2-pyridinyl]ethylamino]-6-fluoropyridine-2-carboxylic acid (isomer 2)

The title compound was synthesized by procedures analogous to those outlines in Example 3, Step 3. ¹H NMR (400 MHz, DMSO-d₆) δ 8.58 (bs, 1H), 7.83 (s, 1H), 7.53 (dd, J=9.4, 7.0 Hz, 1H), 7.35-7.19 (m, 6H), 7.07 (s, 1H), 5.15-5.05 (m, 1H), 4.96-4.85 (m, 1H), 4.39 (t, J=8.5 Hz, 1H), 4.24 (dd, J=8.6, 3.1 Hz, 1H), 3.27 (d, J=13.7 Hz, 1H), 3.02 (dd, J=13.2, 8.3 Hz, 1H), 2.35 (s, 3H), 1.51 (d, J=6.5 Hz, 3H). LCMS calc. for C₂₄H₂₄FN₄O₄[M+H]⁺: m/z=451.2; found 451.4.

### Example 12. 3-(((R)-1-(6-((S)-4-Benzyl-2-oxooxazolidin-3-yl)-4-methylpyridin-2-yl)ethyl) amino)-6-chloropicolinic acid (Isomer 1)

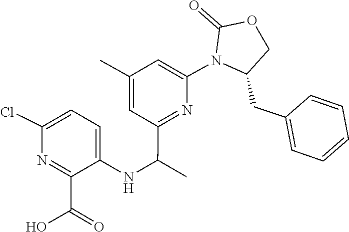 [SMILES: Cc1cc(C(C)Nc2ccc(Cl)nc2C(=O)O)nc(N2C(=O)OC[C@@H]2Cc2ccccc2)c1]

### Step 1. 6-Chloro-2H-pyrido[3,2-d][1,3]oxaine-2,4(1H)-dione

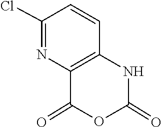 [SMILES: O=c1[nH]c2ccc(Cl)nc2c(=O)o1]

A solution of triphosgene (430 mg, 1.5 mmol) in THF (7.3 mL) was added to a solution of 3-amino-6-chloropicolinic acid (100 mg, 0.58 mmol) in THF (3.9 mL) at 70° C. The solution was stirred at 70° C. for 4 h and then cooled to room temperature. The solid was collected by filtration, washed with MTBE, and dried under vacuum to afford the title compound (74 mg, 0.37 mmol, 64% yield). ¹H NMR (400 MHz, DMSO-d₆) δ 11.94 (s, 1H), 7.81 (d, J=8.7 Hz, 1H), 7.60 (d, J=8.7 Hz, 1H).

### Step 2. 1-[1-[6-[(4S)-4-Benyl-2-oxo-1,3-oxaolidin-3-yl]-4-methyl-2-pyridinyl]ethyl]-6-chloropyrido[3,2-d][1,3]oxaine-2,4-dione

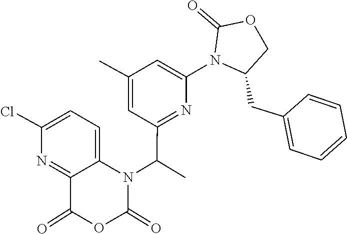 [SMILES: Cc1cc(C(C)n2c(=O)oc(=O)c3nc(Cl)ccc32)nc(N2C(=O)OC[C@@H]2Cc2ccccc2)c1]

To a solution of 6-chloro-1H-pyrido[3,2-d][1,3]oxazine-2,4-dione (191 mg, 0.96 mmol) and triphenylphosphine (252 mg, 0.960 mmol) in THF (2 mL) was added diisopropyl azodicarboxylate (0.19 mL, 0.96 mmol) dropwise. The reaction mixture was stirred for 10 min. Then (4S)-4-benzyl-3-[6-(1-hydroxyethyl)-4-methyl-2-pyridinyl]-1,3-oxazolidin-2-one (150 mg, 0.48 mmol, Example 11, Step 3) was added. The resulting mixture was stirred overnight. The reaction mixture was diluted with DCM (5 mL), washed with water (2 mL) and brine (2 mL), dried over Na₂SO₄, filtered, and concentrated. The residue was purified by prep-HPLC on a C18 column (20-60% MeCN/0.1% TFA (aq.)) to yield two isomers of the title compound as the TFA salts: isomer 1 (17.1 mg, 0.0347 mmol, 7.20% yield, t_{R}: 6.06 min) and isomer 2 (35.2 mg, 0.0714 mmol, 14.9% yield, t_{R}: 6.10 min). Isomer 1: LCMS calc. for C₂₅H₂₂ClN₄O₅[M+H]⁺: m/z=493.1, 495.1; found 493.4, 495.4.

### Step 3. 3-[1-[6-[(4S)-4-Benzyl-2-oxo-1,3-oxaolidin-3-yl]-4-methyl-2-pyridinyl]ethylamino]-6-chloropyridine-2-carboxylic acid (isomer 1)

To a solution of 1-[1-[6-[(4S)-4-benzyl-2-oxo-1,3-oxazolidin-3-yl]-4-methyl-2-pyridinyl]ethyl]-6-chloropyrido[3,2-d][1,3]oxazine-2,4-dione (17.1 mg, 0.0347 mmol, isomer 1) in THF (1 mL) was added 1N NaOH (aq.) (0.5 mL, 0.5 mmol). The reaction mixture was stirred for 15 min. The reaction mixture was diluted with MeOH and purified by prep-HPLC on a C18 column (20-75% MeCN/0.1% TFA (aq.)) to the title compound as a TFA salt (9.80 mg, 0.0209 mmol, 60.6% yield). ¹H NMR (300 MHz, DMSO-d₆) δ 13.02 (s, 1H), 8.74 (d, J=7.6 Hz, 1H), 7.86 (s, 1H), 7.47-7.37 (m, 2H), 7.36-7.22 (m, 5H), 7.09 (s, 1H), 5.13 (t, J=3.0 Hz, 1H), 5.00-4.87 (m, 1H), 4.41 (t, J=8.4 Hz, 1H), 4.26 (dd, J=8.7, 3.1 Hz, 1H), 3.28 (dd, J=13.2, 2.8 Hz, 1H), 3.04 (dd, J=13.2, 8.3 Hz, 1H), 2.37 (s, 3H), 1.52 (d, J=6.5 Hz, 3H). LCMS calc. for C₂₄H₂₄ClN₄O₄[M+H]⁺: m/z=467.1, 469.1; found 467.4, 469.4.

### Examples 13-16

Examples 13-16 listed in Tables 3 and 4 were synthesized according to procedures analogous to Example 11. All examples in these tables were purified from mixtures of diastereomers by prep-HPLC on a C18 column. All examples were prepared as the TFA salt unless otherwise noted.

TABLE 3
Examples 13-16

| Ex- | | | | dr |
|---|---|---|---|---|
| am- | | LCMS | | (isomer 1: |
| ple | R | [M + H]⁺ | Isolation Conditions | isomer 2) |
| 13 | H | 433.1 | 30-50% MeCN/0.1% | 1.2:1 |
| | | | TFA (aq.) isomer 1 | |
| | | | t_{R}: 4.42 min; isomer 2 t_{R}: 4.79 min. | |
| 14 | CH₃ | 447.1 | 30-50% MeCN/0.1% | 1.6:1 |
| | | | TFA (aq.) isomer 1 | |
| | | | t_{R}: 4.59 min; isomer 2 t_{R}: 4.99 min. | |
| 15 | CF₃ | 501.0 | 58-78% MeCN/0.1% | 1.8:1 |
| | | | TFA (aq.) isomer 1 | |
| | | | t_{R}: 4.72 min; isomer 2 t_{R}: 5.15 min. | |
| 16 | OCH₃ | 463.1 | 56-76% MeCN/0.1% | 1.7:1 |
| | | | TFA (aq.) isomer 1 | |
| | | | t_{R}: 4.61 min; isomer 2 tt_{R}: 5.03 min. | |

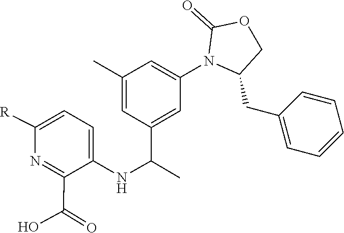

TABLE 4
Examples 13-16

| Example | Compound Name | NMR |
|---|---|---|
| 13 | 3-((1-(6-((S)-4-Benzyl- | ¹H NMR (400 MHz, |
| | 2-oxooxazolidin-3- | DMSO-d₆) δ 8.91 |
| | yl)-4-methylpyridin-2- | (bs, 1H), 7.91-7.79 |
| | yl)ethyl)amino) | (m, 2H), 7.55-7.41 |
| | picolinic acid (isomer 2) | (m, 2H), 7.35-7.18 |
| | | (m, 5H), 7.09 |
| | | (s, 1H), 5.18-5.08 |
| | | (m, 1H), 4.97-4.86 |
| | | (m, 1H), 4.40 (t, J = |
| | | 8.5 Hz, 1H), 4.26- |
| | | 4.19 (m, 1H), 3.27 (d, |
| | | J = 13.3 Hz, 1H), |
| | | 3.04 (dd, J = 13.2, 8.2 |
| | | Hz, 1H), 2.36 (s, |
| | | 3H), 1.53 (d, |
| | | J = 6.5 Hz, 3H). |
| 14 | 3-((1-(6-((S)-4-Benzyl- | — |
| | 2-oxooxazolidin-3- | |
| | yl)-4-methylpyridin-2- | |
| | yl)ethyl)amino)-6- | |
| | methylpicolinic acid (isomer 2) | |
| 15 | 3-((1-(6-((S)-4-Benzyl- | — |
| | 2-oxooxazolidin-3- | |
| | yl)-4-methylpyridin- | |
| | 2-yl)ethyl)amino)-6- | |
| | (trifluoromethyl)picolinic | |
| | acid (isomer 2) | |
| 16 | 3-((1-(6-((S)-4-Benzyl- | — |
| | 2-oxooxazolidin-3- | |
| | yl)-4-methylpyridin- | |
| | 2-yl)ethyl)amino)-6- | |
| | methoxypicolinic acid (isomer 2) | |

### Example 17. 2-((1-(6-((S)-4-Benzyl-2-oxooxazolidin-3-yl)-4-methylpyridin-2-yl)ethyl) amino)benzoic acid

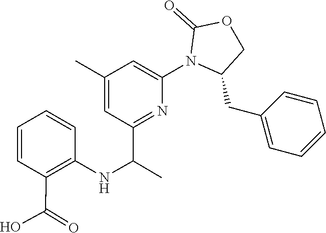 [SMILES: Cc1cc(C(C)Nc2ccccc2C(=O)O)nc(N2C(=O)OC[C@@H]2Cc2ccccc2)c1]

The title compound was synthesized by procedures analogous to those outlines in Example 11, Steps 1-5, utilizing 2-aminobenzoic acid instead of ethyl 3-amino-6-fluoropicolinate. LC-MS calc. for C₂₅H₂₆N₃O₄ [M+H]⁺: m/z=432.2; Found: 432.1.

### Example 18. 3-((1-(6-((S)-4-Benzyl-2-oxooxazolidin-3-yl)-4-methylpyridin-2-yl)ethyl)amino)-6-bromopicolinic acid (Isomer 2)

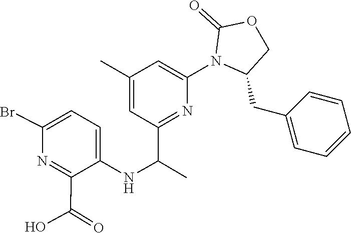 [SMILES: Cc1cc(C(C)Nc2ccc(Br)nc2C(=O)O)nc(N2C(=O)OC[C@@H]2Cc2ccccc2)c1]

### Step 1. Ethyl 3-((1-(6-((S)-4-benzyl-2-oxooxazolidin-3-yl)-4-methylpyridin-2-yl)ethyl) amino)-6-bromopicolinate

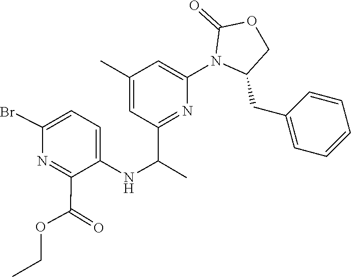 [SMILES: CCOC(=O)c1nc(Br)ccc1NC(C)c1cc(C)cc(N2C(=O)OC[C@@H]2Cc2ccccc2)n1]

The title compound was synthesized by procedures analogous to those outlined in Example 11, Steps 1-5, utilizing ethyl 3-amino-6-bromopicolinate instead of ethyl 3-amino-6-fluoropicolinate. Separation by prep-HPLC on a C18 column (68-88% MeCN/0.1% TFA (aq.)) afforded two isomers of the title compound as the TFA salts: isomer 1 (t_{R}: 4.43 min) and isomer 2 (13.0 mg, 19.9 μmol, 5.39% yield, t_{R}: 4.82 min). Isomer 2: LC-MS calc. for C₂₆H₂₈BrN₄O₄ [M+H]⁺: m/z=539.1; 541.0; Found: 539.0; 541.0.

### Step 2. 3-((1-(6-((S)-4-Benzyl-2-oxooxaolidin-3-yl)-4-methylpyridin-2-yl)ethyl)amino)-6-bromopicolinic acid (Isomer 2)

The title compound was synthesized by procedures analogous to those outlined in Example 3, Step 3. LC-MS calc. for C₂₄H₂₄BrN₄O₄[M+H]⁺: m/z=511.1; 513.0; Found: 511.0; 513.0.

### Example 19. 3-((1-(6-((S)-4-Benzyl-2-oxooxazolidin-3-yl)-4-methylpyridin-2-yl)ethyl)amino)-6-cyanopicolinic acid

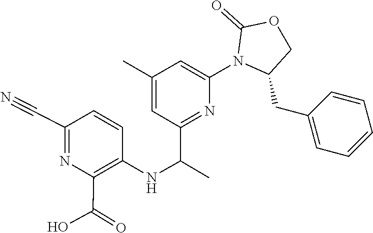 [SMILES: Cc1cc(C(C)Nc2ccc(C#N)nc2C(=O)O)nc(N2C(=O)OC[C@@H]2Cc2ccccc2)c1]

Zinc powder (0.27 mg, 4.2 μmol), zinc cyanide (3.1 mg, 3.3 μmol), Pd₂(dba)₃ (1.5 mg, 1.7 μmol), 1,1′-ferrocenediyl-bis(diphenylphosphine) (0.56 mg, 1.0 μmol), and ethyl 3-((1-(6-((S)-4-benzyl-2-oxooxazolidin-3-yl)-4-methylpyridin-2-yl)ethyl)amino)-6-bromopicolinate (isomer 2) (9.0 mg, 17 μmol, Example 18, Step 1) were suspended in NMP (0.56 mL) and the mixture was heated to 80° C. for 18 h. The mixture was cooled to room temperature, and 1 N LiOH (aq.) (0.17 mL, 0.17 mmol) was added. After stirring for 30 min, the resulting suspension was diluted with water (5.0 mL) and DCM (5.0 mL). The two phases were separated, and the aqueous layer was extracted with DCM (5.0 mL×2). The combined organic layers were dried over sodium sulfate, filtered, and concentrated. The crude residue was purified by prep HPLC on a C18 column (51-71% MeCN/0.1% TFA (aq.)) to afford the title compound as a TFA salt (2.2 mg, 3.5 μmol, 21% yield), a brown solid. LC-MS calc. for C₂₅H₂₄N₅O₄ [M+H]⁺: m/z=458.2; Found: 458.1.

### Example 20. 6-Chloro-3-(((R)-1-(6-((S)-4-(2-fluoro-3-methylbenzyl)-2-oxooxazolidin-3-yl)-4-methylpyridin-2-yl)ethyl)amino)picolinic acid

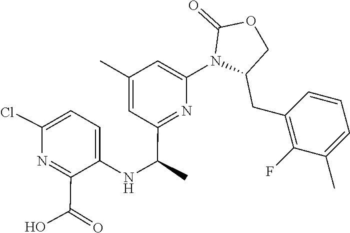 [SMILES: Cc1cc([C@@H](C)Nc2ccc(Cl)nc2C(=O)O)nc(N2C(=O)OC[C@@H]2Cc2cccc(C)c2F)c1]

### Step 1: (S)-2-((tert-Butoxycarbonyl)amino)-3-(2-fluoro-3-methylphenyl)propanoic acid

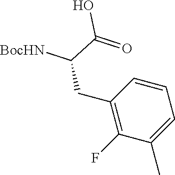 [SMILES: Cc1cccc(C[C@H](NC(=O)OC(C)(C)C)C(=O)O)c1F]

To a solution of (S)-2-amino-3-(2-fluoro-3-methylphenyl)propanoic acid (200 mg, 1.01 mmol) in THF (4.0 mL) and water (4.0 mL) was added K₂CO₃ (280 mg, 2.03 mmol) and di-tert-butyl dicarbonate (442 mg, 2.03 mmol). The reaction mixture was stirred at 60° C. for 12 h. The reaction mixture was diluted with 1N HCl (aq.) until pH ˜7 and the resulting mixture was extracted with ethyl acetate (15 mL×2). The combined organic layers were washed with brine and concentrated. The crude residue was purified by silica gel chromatography (5-50% EtOAc/hexane) to the title compound (259 mg, 0.871 mmol, 85.9% yield) as a yellow oil. LCMS calc. for C₁₅H₂₁FNO₄ [M+H]⁺: m/z=298.1; Found: 298.2.

### Step 2: tert-Butyl (S)-(1-(2-fluoro-3-methylphenyl)-3-hydroxypropan-2-yl)carbamate

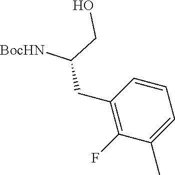 [SMILES: Cc1cccc(C[C@@H](CO)NC(=O)OC(C)(C)C)c1F]

To a solution of (S)-2-((tert-butoxycarbonyl)amino)-3-(2-fluoro-3-methylphenyl)-propanoic acid (250 mg, 0.839 mmol) in THF (5.0 mL) was added LiAlH₄ (95.9 mg, 2.52 mmol) at 0° C. The reaction mixture was warmed to rt and stirred for 2 h. The mixture was cooled to 0° C. and diluted with sat. potassium sodium tartrate solution (aq.) (10 mL). The mixture was stirred at rt for 20 min then extracted with EtOAc (10 mL×3). The combined organic layers were dried over sodium sulfate, filtered, and concentrated. The crude residue was purified by silica gel column chromatography (30-50% EtOAc/hexane) to afford the title compound (110 mg, 0.388 mmol, 46.2% yield) as a yellow oil. LCMS calc. for C₁₅H₂₃FNO₃ [M+H]⁺: m/z=284.2; Found: 284.2.

### Step 3: (S)-4-(2-Fluoro-3-methylbenyl)oxaolidin-2-one

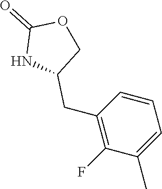

To a solution of tert-butyl (S)-(1-(2-fluoro-3-methylphenyl)-3-hydroxypropan-2-yl)carbamate (150 mg, 0.530 mmol) in THF (2.0 mL) was added NaH (106 mg, 2.65 mmol) in portions at 0° C. The reaction mixture was stirred at rt for 2 h before being poured into sat. NH₄Cl solution (aq.) (10 mL) and extracted with EtOAc (10 mL×2). The combined organic layers were washed with brine (20 mL), dried over sodium sulfate, filtered and concentrated. The crude residue was purified by silica gel prep-TLC (50% EtOAc/hexane) to afford the title compound (100 mg, 0.478 mmol, 90.3% yield) as a yellow oil. LCMS calc. for C₁₁H₁₃FNO₂ [M+H]⁺: m/z=210.1; Found: 210.1.

### Step 4: 1-(6-Bromo-4-methylpyridin-2-yl)ethan-1-one

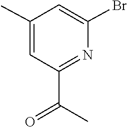

To a solution of 2,6-dibromo-4-picoline (10.0 g, 39.8 mmol) in 1,4-dioxane (100 mL) was added tributyl(1-ethoxyvinyl)stannane (13.5 mL, 39.8 mmol) and Pd(PPh₃)₂Cl₂ (2.80 g, 3.99 mmol). The mixture was purged with N₂ three times then heated to 110° C. for 16 h. The reaction mixture was cooled to rt and diluted with 2M HCl (aq.) (80 mL) and stirred for 1 h. The mixture was diluted with sat. KF solution (aq.) (100 mL) and extracted with EtOAc (100 mL×3). The combined organic layers were dried over sodium sulfate, filtered, and concentrated. The crude residue was purified by silica gel chromatography (5-10% EtOAc/petroleum ether) to afford the title compound (4.80 g, 22.4 mmol, 56.3% yield) as a yellow oil. ¹H NMR (400 MHz, CDCl₃) δ 7.79 (d, J=0.4 Hz, 1H), 7.51-7.45 (m, 1H), 2.68 (s, 3H), 2.39 (s, 3H). LCMS calc. for C₈H₉BrNO [M+H]⁺: m/z=214.0, 216.0; Found: 214.1, 216.1.

### Step 5: (S,E)-N-(1-(6-Bromo-4-methylpyridin-2-yl)ethylidene)-2-methylpropane-2-sulfinamide

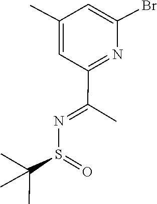 [SMILES: C/C(=N\[S@@](=O)C(C)(C)C)c1cc(C)cc(Br)n1]

To a solution of 1-(6-bromo-4-methylpyridin-2-yl)ethan-1-one (5.38 g, 25.1 mmol) in THF (40 mL) was added (S)-2-methylpropane-2-sulfinamide (3.65 g, 30.1 mmol) and tetraethoxytitanium (11.5 g, 50.2 mmol). The mixture was purged with N₂ three times then heated to 65° C. for 16 h. The reaction mixture was diluted with water (100 mL) and the resulting mixture was extracted with EtOAc (100 mL×2). The combined organic layers were washed with brine and concentrated. The crude residue was purified by silica gel chromatography (0-10% EtOAc/hexane) to afford the title compound (7.20 g, 22.7 mmol, 90.3% yield) as a yellow oil. LCMS calc. for C₁₂H₁₈BrN₂OS [M+H]⁺: m/z=317.0, 319.0; Found: 317.1, 319.1.

### Step 6: (S)—N—((R)-1-(6-Bromo-4-methylpyridin-2-yl)ethyl)-2-methylpropane-2-sulfinamide

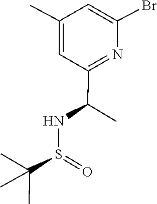 [SMILES: Cc1cc(Br)nc([C@@H](C)N[S@@](=O)C(C)(C)C)c1]

To a solution of (S,E)-N-(1-(6-bromo-4-methylpyridin-2-yl)ethylidene)-2-methylpropane-2-sulfinamide (5.70 g, 18.0 mmol) in THF (100 mL) was added a solution of L-Selectride (35.93 mL, 35.93 mmol, 1M in THF) dropwise at −40° C. The reaction mixture was warmed to rt and stirred for 1 h. The reaction was diluted with water (100 mL) and extracted with EtOAc (100 mL×2). The combined organic layers were washed with brine and concentrated. The crude residue was purified by silica gel chromatography (30-50% EtOAc/hexane) to afford the title compound (4.80 g, 15.0 mmol, 83.7% yield) as a yellow oil. LCMS calc. for C₁₂H₂₀BrN₂OS [M+H]⁺: m/z=319.0, 321.0; Found: 319.1, 321.1.

### Step 7: (R)-1-(6-Bromo-4-methylpyridin-2-yl)ethan-1-amine hydrochloride

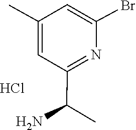

To a solution of (S)—N—((R)-1-(6-bromo-4-methylpyridin-2-yl)ethyl)-2-methylpropane-2-sulfinamide (4.80 g, 15.0 mmol) in 4M HCl in dioxane (35.0 mL) was added MeOH (3.0 ml) and the mixture was stirred at rt for 16 h. The reaction mixture was concentrated to −10 mL, diluted with diethyl ether (50 mL), and stirred for 30 min. The solid was collected by filtration and dried under vacuum to afford the title compound (3.20 g, 12.7 mmol, 72.3% yield) as a light-yellow solid. LCMS calc. for C₈H₁₂BrN₂ [M+H]⁺: m/z=215.0, 217.0; Found: 215.1, 217.1.

### Step 8: Methyl (R)-3-((1-(6-bromo-4-methylpyridin-2-yl)ethyl)amino)-6-chloropicolinate

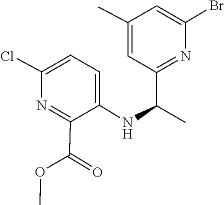 [SMILES: COC(=O)c1nc(Cl)ccc1N[C@H](C)c1cc(C)cc(Br)n1]

To a solution of (R)-1-(6-bromo-4-methylpyridin-2-yl)ethan-1-amine, hydrochloride (4.20 g, 16.7 mmol) in DMSO (42 mL) was added DIPEA (15.1 g, 116 mmol) and methyl 6-chloro-3-fluoropicolinate (3.17 g, 16.7 mmol). The reaction mixture was heated to 100° C. for 16 h before being cooled to rt, diluted with water (50 ml), and extracted with EtOAc (50 mL×3). The combined organic layers were washed with brine (50 ml×3), dried over sodium sulfate, filtered, and concentrated. The crude residue was purified by silica gel chromatography (0-20% EtOAc/hexane) to afford the title compound (4.60 g, 12.0 mmol, 71.6% yield) as a yellow oil. ¹H NMR (400 MHz, CDCl₃) δ 8.23 (d, J=5.6 Hz, 1H), 7.21 (s, 1H), 7.15 (d, J=8.8 Hz, 1H), 7.03 (s, 1H), 6.85 (d, J=8.8 Hz, 1H), 4.59 (p, J=6.4 Hz, 1H), 3.99 (s, 3H), 2.26 (s, 3H), 1.62 (d, J=6.8 Hz, 3H). LCMS calc. for C₁₅H₁₆BrClN₃O₂[M+H]⁺: m/z=384.0, 386.0; Found: 384.2, 386.2.

### Step 9: Methyl 6-chloro-3-(((R)-1-(6-((S)-4-(2-fluoro-3-methylbenzyl)-2-oxooxazolidin-3-yl)-4-methylpyridin-2-yl)ethyl)amino)picolinate

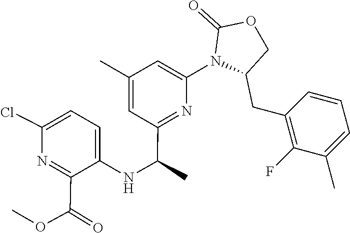 [SMILES: COC(=O)c1nc(Cl)ccc1N[C@H](C)c1cc(C)cc(N2C(=O)OC[C@@H]2Cc2cccc(C)c2F)n1]

To a solution of (S)-4-(2-fluoro-3-methylbenzyl)oxazolidin-2-one (100 mg, 0.478 mmol) in toluene (2.0 mL) was added methyl (R)-3-((1-(6-bromo-4-methylpyridin-2-yl)ethyl)amino)-6-chloropicolinate (184 mg, 0.479 mmol), CuI (91.0 mg, 0.476 mmol), DMEDA (63.2 mg, 0.717 mmol) and K₂CO₃ (132 mg, 0.957 mmol). The mixture was purged with N₂ three times and heated to 120° C. for 3 h. The reaction mixture was cooled to rt, diluted with water (15 mL), and extracted with EtOAc (15 mL×3). The combined organic layers were washed with brine (20 mL), dried over sodium sulfate, filtered and concentrated. The crude residue was purified by silica gel prep-TLC (66% EtOAc/hexane) to afford the title compound (80.0 mg, 0.156 mmol, 32.6% yield) as a yellow solid. LCMS calc. for C₂₆H₂₇ClFN₄O₄[M+H]⁺: m/z=513.2; Found: 513.2.

### Step 10: 6-Chloro-3-(((R)-1-(6-((S)-4-(2-fluoro-3-methylbenyl)-2-oxooxazolidin-3-yl)-4-methylpyridin-2-yl)ethyl)amino)picolinic acid

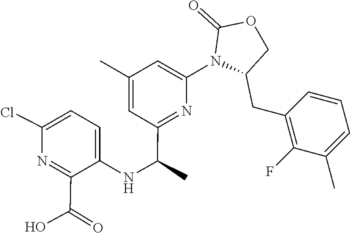 [SMILES: Cc1cc([C@@H](C)Nc2ccc(Cl)nc2C(=O)O)nc(N2C(=O)OC[C@@H]2Cc2cccc(C)c2F)c1]

To a solution of methyl 6-chloro-3-(((R)-1-(6-((S)-4-(2-fluoro-3-methylbenzyl)-2-oxooxazolidin-3-yl)-4-methylpyridin-2-yl)ethyl)amino)picolinate (80.0 mg, 0.156 mmol) in THF (1.0 mL) was added a solution of LiOH monohydrate (0.780 mL, 0.780 mmol, 1M in water). The reaction mixture was heated to 50° C. for 2 h, then diluted with 6M HCl (aq.) to pH ˜3 and concentrated. The residue was dissolved in DMF (2.0 ml), filtered, and the filtrate was purified by prep-HPLC on a C18 column (40-100% MeCN/0.1% TFA (aq.)) to afford the TFA salt of the title compound (41.0 mg, 0.082 mmol, 52.7% yield) as a white solid. ¹H NMR (400 MHz, CDCl₃) δ 10.83-10.47 (bs, 1H), 8.59 (d, J=6.4 Hz, 1H), 7.97 (s, 1H), 7.22 (d, J=8.8 Hz, 1H), 7.11-7.06 (m, 2H), 7.03-6.93 (m, 2H), 6.85 (s, 1H), 5.31 (dd, J=17.6, 9.6 Hz, 1H), 4.68-4.55 (m, 1H), 4.36 (t, J=8.4 Hz, 1H), 4.27 (dd, J=9.2, 3.2 Hz, 1H), 3.47 (dd, J=13.6, 3.2 Hz, 1H), 2.99 (dd, J=13.6, 8.8 Hz, 1H), 2.37 (s, 3H), 2.27 (d, J=2.0 Hz, 3H), 1.62 (d, J=6.8 Hz, 3H). LCMS calc. for C₂₅H₂₅ClFN₄O₄[M+H]⁺: m/z=499.2; Found: 499.4.

### Example 21. 6-Chloro-3-(((R)-1-(6-((S)-4-((3-fluoropyridin-4-yl)methyl)-2-oxooxazolidin-3-yl)-4-methylpyridin-2-yl)ethyl)amino)picolinic acid

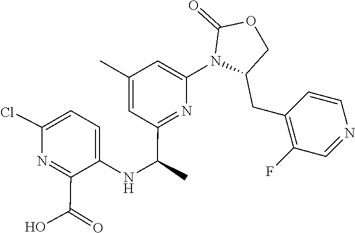 [SMILES: Cc1cc([C@@H](C)Nc2ccc(Cl)nc2C(=O)O)nc(N2C(=O)OC[C@@H]2Cc2ccncc2F)c1]

### Step 1: Methyl (S)-2-((tert-butoxycarbonyl)amino)-3-(3-fluoropyridin-4-yl)propanoate

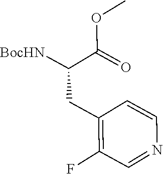 [SMILES: COC(=O)[C@H](Cc1ccncc1F)NC(=O)OC(C)(C)C]

A 100 mL RBF was charged with Zn⁰ (874 mg, 13.5 mmol) and dried at 150° C. under vacuum for 5 min. The flask was cooled rt, then DMF (20 mL) was added and mixture was purged with nitrogen three times. To the mixture was added I₂ (54.0 mg, 0.213 mmol), then the reaction mixture was stirred at rt for 30 seconds until the brown color disappeared. Methyl (R)-2-((tert-butoxycarbonyl)amino)-3-iodopropanoate (2.21 g, 6.73 mmol) was added in four portions and the mixture was purged with nitrogen four times. The reaction mixture was stirred at 25° C. for 1 h before being filtered and transferred to a RBF containing 3-fluoro-4-iodopyridine (1.00 g, 4.48 mmol), PdCl₂ (80.0 mg, 0.452 mmol), and XPhos (428 mg, 0.899 mmol). The resulting mixture was purged with nitrogen four times and stirred at 25° C. for 18 h before being diluted with water (80 ml) and extracted with EtOAc (60 ml×3). The combined organic layers were washed with brine (60 ml×3), dried over sodium sulfate, filtered, and concentrated. The crude residue was purified by silica gel column chromatography (0-30% EtOAc/10% DCM in hexane) to afford the title compound (1.10 g, 3.69 mmol, 82.2% yield) as a light-yellow solid. ¹HNMR (400 MHz, DMSO-d₆) δ 8.46 (d, J=1.2 Hz, 1H), 8.32 (d, J=4.0 Hz, 1H), 7.39-7.33 (m, 2H), 4.31-4.25 (m, 1H), 3.61 (s, 3H), 3.13 (dd, J=16.0, 4.0 Hz, 1H), 2.91 (dd, J=12.0, 8.0 Hz, 1H), 1.28 (s, 9H). LCMS calc. for C₁₄H₂₀FN₂O₄[M+H]⁺: m/z=299.1; Found: 299.1.

### Step 2: tert-Butyl (S)-(1-(3-fluoropyridin-4-yl)-3-hydroxypropan-2-yl)carbamate

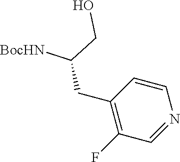 [SMILES: CC(C)(C)OC(=O)N[C@H](CO)Cc1ccncc1F]

To a solution of methyl (S)-2-((tert-butoxycarbonyl)amino)-3-(3-fluoropyridin-4-yl)propanoate (400 mg, 1.41 mmol) in THF (10 mL) was added LiAlH₄ (267 mg, 7.04 mmol) at 0° C. The reaction mixture was stirred at rt for 2 h before being diluted with sat. potassium sodium tartrate solution (aq.) (15 mL) and extracted with EtOAc (15 mL×3). The combined organic layers were washed with brine, dried over Na₂SO₄, and concentrated. The crude residue was purified by silica gel prep-TLC (50% EtOAc/hexane) to afford the title compound (330 mg, 1.22 mmol, 86.7% yield). LCMS calc. for C₁₃H₂₀FN₂O₃[M+H]⁺: m/z=271.1; Found: 271.3.

### Step 3: (S)-4-((3-Fluoropyridin-4-yl)methyl)oxazolidin-2-one

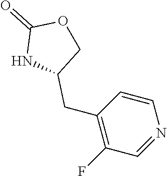 [SMILES: O=C1N[C@@H](Cc2ccncc2F)CO1]

To a solution of tert-butyl (S)-(1-(3-fluoropyridin-4-yl)-3-hydroxypropan-2-yl)carbamate (330 mg, 1.22 mmol) in THF (5.0 mL) was added NaH (244 mg, 6.10 mmol, 60 wt % in mineral oil) at 0° C. The reaction mixture was stirred at rt for 2 h before being diluted with sat. NH₄Cl solution (aq.) (15 mL) and extracted with EtOAc (15.0 mL×3). The combined organic layers were washed with brine, dried over Na₂SO₄, filtered, and concentrated. The crude residue was purified by silica gel prep-TLC (50% EtOAc/hexane) to afford the title compound (90.0 mg, 0.458 mmol, 37.5% yield). LCMS calc. for C₉H₁₀FN₂O₂[M+H]⁺: m/z=197.1; Found: 197.1.

### Step 4: Methyl 6-chloro-3-(((R)-1-(6-((S)-4-((3-fluoropyridin-4-yl)methyl)-2-oxooxaolidin-3-yl)-4-methylpyridin-2-yl)ethyl)amino)picolinate

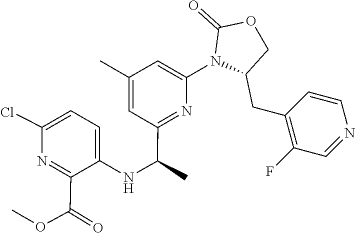 [SMILES: COC(=O)c1nc(Cl)ccc1N[C@H](C)c1cc(C)cc(N2C(=O)OC[C@@H]2Cc2ccncc2F)n1]

To a solution of methyl (R)-3-((1-(6-bromo-4-methylpyridin-2-yl)ethyl)amino)-6-chloropicolinate (150 mg, 0.390 mmol, Example 20, Step 8) in toluene (2.0 mL) was added K₂CO₃ (107 mg, 0.780 mmol), CuI (74.1 mg, 0.390 mmol), (S)-4-((3-fluoropyridin-4-yl)methyl)oxazolidin-2-one (65.0 mg, 0.330 mmol), and DMEDA (51.4 mg, 0.580 mmol). The reaction mixture was heated to 120° C. for 2 h then filtered and concentrated. The crude residue was purified by silica gel prep-TLC (50% EtOAc/hexane) to afford the title compound (90.0 mg, 0.180 mmol, 46.1% yield). LCMS calc. for C₂₄H₂₄ClFN₅O₄[M+H]⁺: m/z=500.1; Found: 500.1.

### Step 5: 6-Chloro-3-(((R)-1-(6-((S)-4-((3-fluoropyridin-4-yl)methyl)-2-oxooxaolidin-3-yl)-4-methylpyridin-2-yl)ethyl)amino)picolinic acid

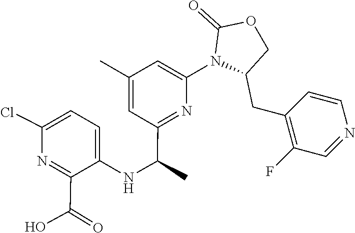 [SMILES: Cc1cc([C@@H](C)Nc2ccc(Cl)nc2C(=O)O)nc(N2C(=O)OC[C@@H]2Cc2ccncc2F)c1]

To a solution of methyl 6-chloro-3-(((R)-1-(6-((S)-4-((3-fluoropyridin-4-yl)methyl)-2-oxooxazolidin-3-yl)-4-methylpyridin-2-yl)ethyl)amino)picolinate (90.0 mg, 0.180 mmol) in THF (5.0 mL) was added LiOH (37.8 mg, 0.900 mmol) in water (1.0 mL). The reaction mixture heated to 55° C. for 2 h then diluted with 1N HCl solution (aq.) to pH ˜7 and concentrated. The crude residue was purified by prep-HPLC on a C18 column (55-100% MeCN/0.1% TFA (aq.)) to afford the TFA salt of the title compound (26.3 mg, 0.053 mmol, 29.9% yield). ¹H NMR (400 MHz, DMSO-d₆) δ 8.64 (d, J=8.0 Hz, 1H), 8.49 (d, J=1.2 Hz, 1H), 8.34 (d, J=4.0 Hz, 1H), 7.79 (s, 1H), 7.43-7.38 (m, 2H), 7.30 (d, J=8.0 Hz, 1H), 7.03 (s, 1H), 5.25-5.20 (m, 1H), 4.82 (p, J=6.8 Hz, 1H), 4.49 (d, J=8.0 Hz, 1H), 4.26 (dd, J=8.0, 4.0 Hz, 1H), 3.33-3.23 (m, 2H), 2.33 (s, 3H), 1.47 (d, J=4.0 Hz, 3H). LCMS calc. for C₂₃H₂₂ClFN₅O₄[M+H]⁺: m/z=486.1; Found: 486.1.

### Examples 22-47

Examples 22-47 listed in Tables 5 and 6 were synthesized according to procedures analogous to Example 9 (Method A), Example 20 (Method B), or Example 21 (Method C). All examples were prepared as the formate salt unless otherwise noted.

TABLE 5

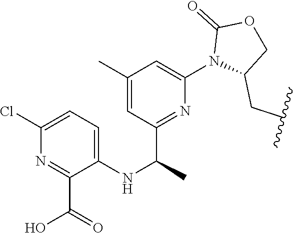 [SMILES: *C[C@H]1COC(=O)N1c1cc(C)cc([C@@H](C)Nc2ccc(Cl)nc2C(=O)O)n1]

Examples 22-47

| | | | LCMS |
|---|---|---|---|
| Example | Method | R | [M + H]⁺ |
| 22 | A | 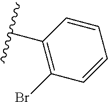 [SMILES: *c1ccccc1Br] | 545.2, 547.2 |
| 23 | A | 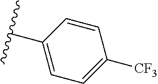 [SMILES: *c1ccc(C(F)(F)F)cc1] | 535.3 |
| 24 | A | 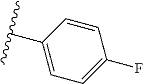 [SMILES: *c1ccc(F)cc1] | 485.1 |
| 25 | C | 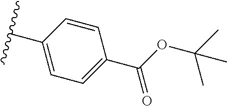 [SMILES: *c1ccc(C(=O)OC(C)(C)C)cc1] | 567.2 |
| 26 | B | 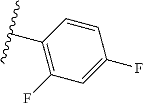 [SMILES: *c1ccc(F)cc1F] | 503.1 |
| 27 | C | 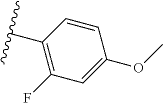 [SMILES: *c1ccc(OC)cc1F] | 515.1 |
| 28 | B | 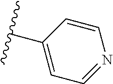 [SMILES: *c1ccncc1] | 468.1 |
| 29 | C | 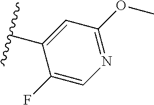 [SMILES: *c1cc(OC)ncc1F] | 516.1 |
| 30 | C | 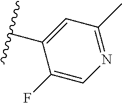 [SMILES: *c1cc(C)ncc1F] | 500.1 |
| 31 | A | 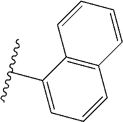 [SMILES: *c1cccc2ccccc12] | 517.2 |
| 32 | B | 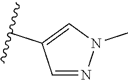 [SMILES: *c1cnn(C)c1] | 471.2 |
| 33 | B | 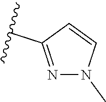 [SMILES: *c1ccn(C)n1] | 471.1 |
| 34 | B | 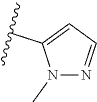 [SMILES: *c1ccnn1C] | 471.1 |
| 35 | B | 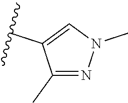 [SMILES: *c1cn(C)nc1C] | 485.2 |
| 36 | C | 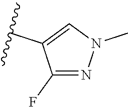 [SMILES: *c1cn(C)nc1F] | 489.1 |
| 37 | B | 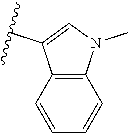 [SMILES: *c1cn(C)c2ccccc12] | 520.2 |
| 38 | C | 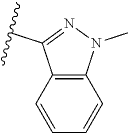 [SMILES: *c1nn(C)c2ccccc12] | 521.2 |
| 39 | C | 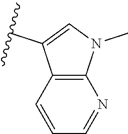 [SMILES: *c1cn(C)c2ncccc12] | 521.2 |
| 40 | C | 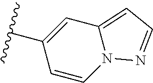 [SMILES: *c1ccn2nccc2c1] | 507.1 |
| 41 | C | 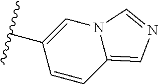 [SMILES: *c1ccc2cncn2c1] | 507.1 |
| 42 | C | 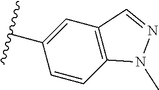 [SMILES: *c1ccc2c(cnn2C)c1] | 521.2 |
| 43 | C | 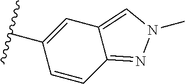 [SMILES: *c1ccc2nn(C)cc2c1] | 521.2 |
| 44 | C | 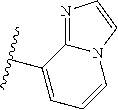 [SMILES: *c1cccn2ccnc12] | 507.1 |
| 45 | C | 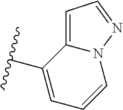 [SMILES: *c1cccn2nccc12] | 507.1 |
| 46 | C | 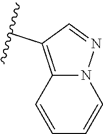 [SMILES: *c1cnn2ccccc12] | 507.1 |
| 47 | B | 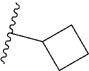 [SMILES: *C1CCC1] | 445.2 |

TABLE 6
Examples 22-47

| Ex- | | |
|---|---|---|
| am- | | |
| ple | Compound name | NMR |
| 22 | 3-(((R)-1-(6-((S)-4- | — |
| | (2-Bromobenzyl)- | |
| | 2-oxooxazolidin-3-yl)-4- | |
| | methylpyridin-2- | |
| | yl)ethyl)amino)-6- | |
| | chloropicolinic | |
| | acid, TFA | |
| 23 | 6-Chloro-3-(((R)-1- | ¹H NMR (300 MHz, DMSO-d₆) δ 13.03 |
| | (4-methyl-6-((S)- | (bs, 1H), 8.73 (d, J = 7.6 Hz, 1H), 7.85 |
| | 2-oxo-4-(4-(tri- | (s, 1H), 7.69 (d, J = 8.0 Hz, 2H), 7.49 |
| | fluoromethyl) | (d, J = 8.0 Hz, 2H), 7.42 (q, J = 9.0 Hz, |
| | benzyl)oxazolidin- | 2H), 7.10 (s, 1H), 5.22-5.09 (m, 1H), |
| | 3-yl)pyridin-2- | 4.99-4.85 (m, 1H), 4.43 (t, J = 8.5 Hz, |
| | yl)ethyl)amino)picolinic | 1H), 4.27 (dd, J = 8.8, 2.9 Hz, 1H), 3.39 |
| | acid, TFA | (s, 1H), 3.19 (dd, J = 13.3, 8.1 Hz, 1H), |
| | | 2.38 (s, 3H), 1.51 (d, J = 6.5 Hz, 3H). |
| 24 | 6-Chloro-3-(((R)- | ¹H NMR (400 MHz, DMSO-d₆) δ 12.98 |
| | 1-(6-((S)-4-(4- | (bs, 1H), 8.70 (d, J = 7.6 Hz, 1H), 7.83 |
| | fluorobenzyl)-2- | (s, 1H), 7.40 (dd, J = 20.6, 9.0 Hz, 2H), |
| | oxooxazolidin-3-yl)- | 7.26 (dd, J = 8.4, 5.6 Hz, 2H), 7.11 (dd, |
| | 4-methylpyridin-2- | J = 19.8, 11.0 Hz, 3H), |
| | yl)ethyl)amino)picolinic | 5.07 (s, 1H), 4.96- |
| | acid, TFA | 4.85 (m, 1H), 4.40 (t, J = 8.4 Hz, 1H), |
| | | 4.25 (dd, J = 8.8, 3.2 Hz, 1H), 3.23 (d, |
| | | J = 10.8 Hz, 1H), 3.06 (dd, J = 13.6, 8.0 |
| | | Hz, 1H), 2.35 (s, 3H), 1.49 (d, J = 6.4 |
| | | Hz, 3H). |
| 25 | 3-(((R)-1-(6-((S)- | ¹H NMR (400 MHz, DMSO-d₆) δ 12.99 |
| | 4-(4-(tert-Butoxy- | (bs, 1H), 8.71 (d, J = 7.6 Hz, 1H), 7.87- |
| | carbonyl)benzyl)- | 7.73 (m, 3H), 7.48-7.26 (m, 4H), 7.05 |
| | 2-oxooxazolidin-3-yl)- | (d, J = 1.2 Hz, 1H), 5.11 |
| | 4-methylpyridin-2- | (dd, J = 7.2, 3.6 Hz, 1H), 4.88 |
| | yl)ethyl)amino)-6- | (t, J = 6.8 Hz, 1H), 4.39 (t, |
| | chloropicolinic acid | J = 8.4 Hz, 1H), 4.23 (dd, J = 8.8, 2.8 |
| | | Hz, 1H), 3.26 (d, J = 3.2 Hz, 1H), 3.15 |
| | | (dd, J = 13.6, 7.8 Hz, 1H), 2.34 (s, 3H), |
| | | 1.52 (s, 12H). |
| 26 | 6-Chloro-3-(((R)-1- | ¹H NMR (400 MHz, DMSO-d₆) δ 12.96 |
| | (6-((S)-4-(2,4- | (bs, 1H), 8.65 (d, J = 7.6 Hz, 1H), 7.79 |
| | difluorobenzyl)-2- | (t, J = 1.2 Hz, 1H), 7.44-7.30 (m, 3H), |
| | oxooxazolidin-3- | 7.19 (dd, J = 9.6, 2.8 Hz, 1H), 7.07- |
| | yl)-4-methylpyridin-2- | 6.99 (m, 2H), 5.23-5.11 (m, 1H), 4.84 |
| | yl)ethyl)amino)picolinic | (p, J = 6.8 Hz, 1H), 4.44 (t, J = 8.4 Hz, |
| | acid | 1H), 4.23 (dd, J = 8.8, 2.8 Hz, 1H), 3.19 |
| | | (dd, J = 13.6, 5.6 Hz, 2H), 2.33 (s, 3H), |
| | | 1.48 (d, J = 6.4 Hz, 3H). |
| 27 | 6-Chloro-3-(((R)- | ¹H NMR (400 MHz, DMSO-d₆) δ 8.64 |
| | 1-(6-((S)-4-(2- | (d, J = 8.0 Hz, 1H), 7.79 (s, 1H), 7.41- |
| | fluoro-4- | 7.32 (m, 2H), 7.16 (t, J = 8.0 Hz, 1H), |
| | methoxybenzyl)-2- | 7.02 (s, 1H), 6.79-6.69 (m, 2H), 5.13- |
| | oxooxazolidin-3-yl)-4- | 5.09 (m, 1H), 4.86-4.83 (m, 1H), 4.44 |
| | methylpyridin-2- | 4.39 (m, 1H), 4.21-4.18 (m, 1H), 3.72 |
| | yl)ethyl)amino)picolinic | (s, 3H), 3.17-3.03 (m, 2H), 2.32 (s, 3H), |
| | acid | 1.47 (d, J = 8.0 Hz, 3H). |
| 28 | 6-Chloro-3-(((R)-1- | |
| | (4-methyl-6-((S)- | |
| | 2-oxo-4-(pyridin-4- | |
| | ylmethyl)oxazolidin- | |
| | 3-yl)pyridin-2- | |
| | yl)ethyl)amino)picolinic | |
| | acid, TFA | |
| 29 | 6-Chloro-3-(((R)- | ¹H NMR (400 MHz, DMSO-d₆) δ 12.97 |
| | 1-(6-((S)-4-((5- | (bs, 1H), 8.64 (d, J = 7.6 Hz, 1H), 8.06 |
| | fluoro-2- | (d, J = 1.6 Hz, 1H), 7.79 |
| | methoxypyridin-4- | (s, 1H), 7.42 (d, J = 8.8 Hz, 1H), |
| | yl)methyl)-2- | 7.33 (d, J = 8.8 Hz, 1H), |
| | oxooxazolidin-3-yl)-4- | 7.02 (s, 1H), 6.81 (d, J = 4.8 Hz, 1H), |
| | methylpyridin-2- | 5.21 (q, J = 6.0 Hz, 1H), 4.83 (p, J = 6.4 |
| | yl)ethyl)amino)picolinic | Hz, 1H), 4.49 (t, J = 8.4 Hz, 1H), 4.29 |
| | acid | (dd, J = 9.2, 2.8 Hz, 1H), 3.78 (d, J = 1.2 |
| | | Hz, 3H), 3.22 (t, J = 5.2 Hz, 2H), 2.33 |
| | | (s, 3H), 1.47 (d, J = 6.4 Hz, 3H). |
| 30 | 6-Chloro-3-(((R)- | ¹H NMR (400 MHz, DMSO-d₆) δ 12.97 |
| | 1-(6-((S)-4-((5- | (bs, 1H), 8.64 (d, J = 7.6 Hz, 1H), 8.06 |
| | fluoro-2-methylpyridin- | (d, J = 1.6 Hz, 1H), 7.79 |
| | 4-yl)methyl)- | (s, 1H), 7.42 (d, J = 8.8 Hz, 1H), |
| | 2-oxooxazolidin-3-yl)- | 7.33 (d, J = 8.8 Hz, 1H), |
| | 4-methylpyridin-2- | 7.02 (s, 1H), 6.81 (d, J = 4.8 Hz, 1H), |
| | yl)ethyl)amino)picolinic | 5.21 (q, J = 6.0 Hz, 1H), 4.83 (p, J = 6.4 |
| | acid | Hz, 1H), 4.49 (t, J = 8.4 Hz, 1H), 4.29 |
| | | (dd, J = 9.2, 2.8 Hz, 1H), 3.78 (d, J = |
| | | 1.2 Hz, 3H), 3.22 (t, J = 5.2 Hz, 2H), |
| | | 2.33 (s, 3H), 1.47 (d, J = 6.4 Hz, 3H). |
| 31 | 6-Chloro-3-(((R)-1- | ¹H NMR (400 MHz, DMSO-d₆) δ 8.74 |
| | (4-methyl-6-((S)- | (d, J = 6.8 Hz, 1H), 8.61 (d, J = 8.4 Hz, |
| | 4-(naphthalen- | 1H), 7.98-7.82 (m, 3H), 7.58-7.37 (m, |
| | 1-ylmethyl)-2- | 5H), 7.27 (d, J = 9.2 Hz, |
| | oxooxazolidin- | 1H), 7.04 (d, J = 1.2 Hz, 1H), |
| | 3-yl)pyridin-2- | 5.32 (dd, J = 10.0, 5.6 Hz, 1H), 4.88 |
| | yl)ethyl)amino)picolinic | (p, J = 6.4 Hz, 1H), 4.28 (d, J = |
| | acid | 5.6 Hz, 2H), 4.02 (dd, J = 13.2, 3.6 Hz, |
| | | 1H), 3.14 (dd, J = 13.6, 10.0 Hz, 1H), |
| | | 2.35 (s, 3H), 1.65 (d, J = 6.8 Hz, 3H). |
| 32 | 6-Chloro-3-(((R)-1- | ¹H NMR (300 MHz, DMSO-d₆) δ 13.01 |
| | (4-methyl-6-((S)- | (bs, 1H), 8.72 (d, J = 7.7 Hz, 1H), 7.84 |
| | 4-((1-methyl- | (s, 1H), 7.50 (s, 1H), 7.47-7.38 (m, |
| | 1H-pyrazol-4- | 2H), 7.21 (s, 1H), 7.07 (s, 1H), 5.01 |
| | yl)methyl)-2- | (dt, J = 11.5, 3.5 Hz, 1H), 4.98-4.85 |
| | oxooxazolidin-3- | (m, 1H), 4.45 (t, J = 8.5 Hz, 1H), 4.20 |
| | yl)pyridin-2- | (dd, J = 8.6, 3.4 Hz, 1H), 3.79 (s, 3H), |
| | yl)ethyl)amino)picolinic | 2.99 (qd, J = 14.4, 5.3 Hz, 2H), 2.37 (s, |
| | acid, TFA | 3H), 1.48 (d, J = 6.5 Hz, 3H). |
| 33 | 6-Chloro-3-(((R)-1- | ¹H NMR (400 MHz, DMSO-d₆) δ 8.64 |
| | (4-methyl-6-((S)- | (d, J = 7.6 Hz, 1H), 7.80 (t, J = 1.2 Hz, |
| | 4-((1-methyl- | 1H), 7.56 (d, J = 2.0 Hz, 1H), 7.38 (d, |
| | 1H-pyrazol-3- | J = 1.6 Hz, 2H), 7.04 (d, |
| | yl)methyl)-2- | J = 1.2 Hz, 1H), 5.93 (d, J = 2.4 |
| | oxooxazolidin-3- | Hz, 1H), 5.09 (dd, J = |
| | yl)pyridin-2- | 7.2, 3.6 Hz, 1H), 4.85 (p, J = 6.8 Hz, |
| | yl)ethyl)amino)picolinic | 1H), 4.46 (t, J = 8.4 Hz, 1H), 4.27 (dd, |
| | acid | J = 8.4, 3.6 Hz, 1H), 3.76 (s, 3H), 3.14- |
| | | 3.01 (m, 2H), 2.33 (s, 3H), 1.47 (d, J = |
| | | 6.4 Hz, 3H). |
| 34 | 6-Chloro-3-(((R)- | ¹H NMR (400 MHz, DMSO-d₆) δ 8.82 |
| | 1-(4-methyl-6-((S)- | (s, 1H), 7.81 (s, 1H), 7.37-7.28 (m, 2H), |
| | 4-((1-methyl- | 7.20 (d, J = 8.8 Hz, 1H), 7.00 (s, 1H), |
| | 1H-pyrazol-5- | 6.18 (d, J = 1.6 Hz, 1H), 5.13 (dd, J = |
| | yl)methyl)-2- | 8.0, 4.4 Hz, 1H), 4.74 (d, J = 7.6 Hz, |
| | oxooxazolidin-3- | 1H), 4.47 (t, J = 8.4 Hz, 1H), 4.24 (dd, |
| | yl)pyridin-2- | J = 8.8, 3.2 Hz, 1H), 3.75 (s, 3H), 3.36- |
| | yl)ethyl)amino)picolinic | 3.33 (m, 1H), 3.13 (dd, J = 14.8, 8.8 Hz, |
| | acid | 1H), 2.32 (s, 3H), 1.48 (d, J = 6.8 Hz, |
| | | 3H). |
| 35 | 6-Chloro-3-(((R)- | ¹H NMR (400 MHz, DMSO-d₆) δ 8.68 |
| | 1-(6-((S)-4-((1,3- | (d, J = 7.6 Hz, 1H), 7.82 (d, J = 1.2 Hz, |
| | dimethyl-1H- | 1H), 7.47 (s, 1H), 7.42 (d, J = 8.8 Hz, |
| | pyrazol-4-yl)methyl)-2- | 1H), 7.33 (d, J = 9.2 Hz, 1H), 7.03 (d, |
| | oxooxazolidin-3-yl)-4- | J = 1.2 Hz, 1H), 5.00 (dd, |
| | methylpyridin-2- | J = 8.0, 3.2 Hz, 1H), 4.85 (p, |
| | yl)ethyl)amino)picolinic | J = 6.4 Hz, 1H), 4.42 (t, J = |
| | acid | 8.4 Hz, 1H), 4.19 (dd, J = 8.8, 3.2 Hz, |
| | | 1H), 3.69 (s, 3H), 2.96 (dd, J = 14.4, 3.2 |
| | | Hz, 1H), 2.81 (dd, J = 14.0, 8.0 Hz, 1H), |
| | | 2.33 (s, 3H), 1.96 (s, 3H), 1.48 (d, J = |
| | | 6.4 Hz, 3H). |
| 36 | 6-Chloro-3-(((R)- | ¹H NMR (400 MHz, DMSO-d₆) δ 12.96 |
| | 1-(6-((S)-4-((3- | (bs, 1H), 8.68 (d, J = 7.6 Hz, 1H), 7.80 |
| | fluoro-1-methyl- | (t, J = 1.2 Hz, 1H), 7.49 (d, J = 2.4 Hz, |
| | 1H-pyrazol-4- | 1H), 7.45-7.32 (m, 2H), 7.03 (s, 1H), |
| | yl)methyl)-2- | 5.01 (dd, J = 7.2, 3.6 Hz, 1H), 4.86 (p, |
| | oxooxazolidin-3-yl)-4- | J = 6.8 Hz, 1H), 4.49 (t, |
| | methylpyridin-2- | J = 8.6 Hz, 1H), 4.20 (dd, |
| | yl)ethyl)amino)picolinic | J = 8.8, 3.2 Hz, 1H), 3.67 (s, |
| | acid | 3H), 2.92 (dd, J = 14.8, 5.2 Hz, 2H), |
| | | 2.33 (s, 3H), 1.46 (d, J = 6.4 Hz, 3H). |
| 37 | 6-Chloro-3-(((R)-1- | ¹H NMR (400 MHz, CDCl₃) |
| | (4-methyl-6-((S)- | δ 10.78 (bs, 1H), 8.76 (d, |
| | 4-((1-methyl-1H- | J = 6.4 Hz, 1H), 8.14 (s, |
| | indol-3-yl)methyl)- | 1H), 7.86 (d, J = 8.0 Hz, 1H), 7.45 (d, |
| | 2-oxooxazolidin- | J = 8.4 Hz, 1H), 7.40-7.33 (m, 1H), 7.31 |
| | 3-yl)pyridin-2- | (d, J = 8.8 Hz, 1H), 7.25-7.16 (m, 2H), |
| | yl)ethyl)amino)picolinic | 7.14 (s, 1H), 6.99 (s, 1H), 5.52-5.46 |
| | acid, TFA | (m, 1H), 4.82 (p, J = 6.8 Hz, 1H), 4.50- |
| | | 4.29 (m, 2H), 3.91 (s, 3H), 3.74 (dd, J = |
| | | 14.4, 3.2 Hz, 1H), 3.15 (dd, J = 14.4, |
| | | 9.6 Hz, 1H), 2.50 (s, 3H), 1.79 |
| | | (d, J = 6.8 Hz, 3H). |
| 38 | 6-Chloro-3-(((R)-1- | ¹H NMR (400 MHz, DMSO-d₆) δ 8.82 |
| | (4-methyl-6-((S)- | (s, 1H), 7.78 (s, 1H), 7.57 (d, J = 8.4 |
| | 4-((1-methyl- | Hz, 1H), 7.49 (d, J = 8.0 Hz, 1H), 7.36- |
| | 1H-indazol-3- | 7.29 (m, 3H), 7.05 (s, 1H), 6.98-6.94 |
| | yl)methyl)-2- | (m, 1H), 5.32-5.27 (m, 1H), 4.86-4.80 |
| | oxooxazolidin-3- | (m, 1H), 4.49 (t, J = 8.4 Hz, 1H), 4.39 |
| | yl)pyridin-2- | (dd, J = 8.8, 3.2 Hz, 1H), 3.99 (s, 3H), |
| | yl)ethyl)amino)picolinic | 3.55 (dd, J = 14.4, 3.2 Hz, 1H), 3.41 (d, |
| | acid | J = 6.4 Hz, 1H), 2.33 (s, 3H), 1.53 (d, |
| | | J = 6.5 Hz, 3H). |
| 39 | 6-Chloro-3-(((R)-1- | ¹H NMR (400 MHz, DMSO-d₆) δ 8.78 |
| | (4-methyl-6-((S)- | (d, J = 7.2 Hz, 1H), 8.22 (dd, J = 4.8, |
| | 4-((1-methyl- | 1.6 Hz, 1H), 7.86-7.79 (m, 2H), 7.45- |
| | 1H-pyrrolo[2,3- | 7.35 (m, 3H), 7.08 (s, 1H), 6.93 (dd, J = |
| | b]pyridin-3- | 7.6, 4.8 Hz, 1H), 5.19 (dd, J = 8.4, 3.2 |
| | yl)methyl)-2- | Hz, 1H), 4.91 (t, J = 6.8 Hz, 1H), 4.41 |
| | oxooxazolidin- | (t, J = 8.4 Hz, 1H), 4.25 (dd, J = 8.4, 3.2 |
| | 3-yl)pyridin-2- | Hz, 1H), 3.78 (s, 3H), 3.39 (s, 1H), 3.13 |
| | yl)ethyl)amino)picolinic | (dd, J = 14.0, 8.4 Hz, 1H), 2.35 (s, 3H), |
| | acid | 1.53 (d, J = 6.5 Hz, 3H). |
| 40 | 6-Chloro-3-(((R)- | ¹H NMR (400 MHz, DMSO-d₆) δ 13.00 |
| | 1-(4-methyl-6-((S)- | (bs, 1H), 8.73 (s, 1H), 8.59 (d, J = 7.2 |
| | 2-oxo-4-(pyrazolo[1,5- | Hz, 1H), 7.96 (d, J = 2.4 Hz, 1H), 7.84 |
| | α]pyridin-5- | (s, 1H), 7.57 (s, 1H), 7.42 |
| | ylmethyl)oxazolidin- | (d, J = 8.8 Hz, 1H), 7.35 |
| | 3-yl)pyridin-2- | (d, J = 8.8 Hz, 1H), 7.07 (s, |
| | yl)ethyl)amino)picolinic | 1H), 6.71 (d, J = 5.6 Hz, 1H), 6.51 (d, |
| | acid | J = 2.0 Hz, 1H), 5.16 (s, 1H), 4.91-4.87 |
| | | (m, 1H), 4.45 (t, J = 8.4 Hz, 1H), 4.32 |
| | | (dd, J = 8.8, 2.8 Hz, 1H), 3.28 (d, J = |
| | | 3.2 Hz, 1H), 3.11 (d, J = 5.2 Hz, 1H), |
| | | 2.36 (s, 3H), 1.49 (d, J = 6.4 Hz, 3H). |
| 41 | 6-Chloro-3-(((R)- | ¹H NMR (400 MHz, DMSO-d₆) δ 13.00 |
| | 1-(6-((S)-4- | (bs, 1H), 8.74 (d, J = 7.6 Hz, 1H), 8.30 |
| | (imidazo[1,5- | (s, 1H), 8.25 (s, 1H), 7.84 |
| | α]pyridin-6-ylmethyl)- | (d, J = 1.2 Hz, 1H), 7.47 (d, |
| | 2-oxooxazolidin- | J = 9.6 Hz, 1H), 7.42 (d, J = |
| | 3-yl)-4- | 8.8 Hz, 1H), 7.34 (d, J = 9.2 Hz, 1H), |
| | methylpyridin-2- | 7.31 (s, 1H), 7.06 (s, 1H), 6.63 (dd, J = |
| | yl)ethyl)amino)picolinic | 9.2, 1.2 Hz, 1H), 5.14-5.19 (m, 1H), |
| | acid | 4.88 (p, J = 6.4 Hz, 1H), 4.45 (t, J = 8.4 |
| | | Hz, 1H), 4.32 (dd, J = 8.8, 2.8 Hz, 1H), |
| | | 3.18 (dd, J = 13.6, 3.2 Hz, 1H), 2.94 |
| | | (dd, J = 13.6, 8.4 Hz, 1H), 2.35 |
| | | (s, 3H), 1.49 (d, J = 6.4 Hz, 3H). |
| 42 | 6-Chloro-3-(((R)-1- | ¹H NMR (400 MHz, DMSO-d₆) |
| | (4-methyl-6-((S)- | δ 8.76 (d, J = 8.0 Hz, |
| | 4-((1-methyl- | 1H), 7.96 (s, 1H), 7.85 (s, |
| | 1H-indazol-5- | 1H), 7.63 (s, 1H), 7.56 (d, J = 8.0 Hz, |
| | yl)methyl)-2- | 1H), 7.54-7.42 (m, 2H), 7.41-7.23 (m, |
| | oxooxazolidin-3- | 1H), 7.09 (s, 1H), 5.15-5.13 (m, 1H), |
| | yl)pyridin-2- | 4.95-4.92 (m, 1H), 4.40-4.29 (m, 2H), |
| | yl)ethyl)amino)picolinic | 4.05 (s, 3H), 3.39-3.35 (m, 1H), 3.15- |
| | acid | 3.10 (m, 1H), 2.37 (s, 3H), 1.52 (d, J = |
| | | 8.0 Hz, 3H). |
| 43 | 6-Chloro-3-(((R)-1- | ¹H NMR (400 MHz, DMSO-d₆) δ 8.76 |
| | (4-methyl-6-((S)- | (d, J =8.0 Hz, 1H), 8.24 (s, 1H), 7.84 (s, |
| | 4-((2-methyl- | 1H), 7.50 (d, J = 8.0 Hz, 2H), 7.42-7.35 |
| | 2H-indazol-5- | (m, 2H), 7.11-7.07 (m, 2H), 5.15-5.11 |
| | yl)methyl)-2- | (m, 2H), 4.39-4.26 (m, 2H), 4.13 (s, |
| | oxooxazolidin-3- | 3H), 3.30-3.25 (m, 1H), 3.06-3.00 (m, |
| | yl)pyridin-2- | 1H), 2.36 (s, 3H), 1.51 (d, J = 8.0 Hz, |
| | yl)ethyl)amino)picolinic | 3H). |
| | acid | |
| 44 | 6-Chloro-3-(((R)- | ¹H NMR (400 MHz, DMSO-d₆) δ 8.68 |
| | 1-(6-((S)-4- | (s, 1H), 8.48 (d, J = 6.4 Hz, |
| | (imidazo[1,2- | 1H), 8.00 (s, 1H), 7.81 |
| | α]pyridin-8-ylmethyl)- | (s, 1H), 7.64 (s, 1H), 7.41- |
| | 2-oxooxazolidin-3-yl)- | 7.35 (m, 2H), 7.04 (s, 1H), 6.96 (s, 1H), |
| | 4-methylpyridin-2- | 6.85 (t, J = 6.4 Hz, 1H), 5.38-5.32 (m, |
| | yl)ethyl)amino)picolinic | 1H), 4.82 (d, J = 6.4 Hz, 1H), 4.54- |
| | acid | 4.50 (m, 1H), 4.41 (t, J = 8.3 Hz, 1H), |
| | | 3.63 (dd, J = 13.6, 3.6 Hz, 1H), 3.42 (d, |
| | | J = 7.6 Hz, 1H), 2.34 (s, 3H), 1.49 (d, |
| | | J = 6.4 Hz, 3H). |
| 45 | 6-Chloro-3-(((R)- | ¹H NMR (400 MHz, DMSO-d₆) δ 8.61 |
| | 1-(4-methyl-6-((S)- | (dd, J = 16.0, 7.0 Hz, 2H), 7.94 (d, J = |
| | 2-oxo-4-(pyrazolo[1, | 2.2 Hz, 1H), 7.83 (s, 1H), 7.41 (d, J = |
| | 5-α]pyridin-4- | 8.8 Hz, 1H), 7.27 (d, J = 9.2 Hz, 1H), |
| | ylmethyl)oxazolidin- | 7.10 (d, J = 6.8 Hz, 1H), 7.05 (s, 1H), |
| | 3-yl)pyridin-2- | 6.85-6.78 (m, 2H), 5.33-5.28 (m, 1H), |
| | yl)ethyl)amino)picolinic | 4.82-4.78 (m, 1H), 4.40 (t, J = 8.4 Hz, |
| | acid, TFA | 1H), 4.22 (dd, J = 8.8, 2.8 Hz, 1H), 3.55 |
| | | (dd, J = 13.6, 3.6 Hz, 1H), 3.20 (dd, J = |
| | | 13.6, 8.8 Hz, 1H), 2.34 (s, 3H), 1.51 (d, |
| | | J = 6.6 Hz, 3H). |
| 46 | 6-Chloro-3-(((R)-1- | ¹H NMR (400 MHz, DMSO-d₆) δ 8.77 |
| | (4-methyl-6-((S)- | (d, J = 7.6 Hz, 1H), 8.63-8.57 (m, 1H), |
| | 2-oxo-4-(pyrazolo[1, | 7.86 (s, 1H), 7.80 (s, 1H), 7.47-7.36 |
| | 5-α]pyridin-3- | (m, 3H), 7.10-7.01 (m, 2H), |
| | ylmethyl)oxazolidin- | 6.82 (dd, J = 6.8, 1.2 Hz, |
| | 3-yl)pyridin-2- | 1H), 5.16 (dd, J = 7.2, 3.2 |
| | yl)ethyl)amino)picolinic | Hz, 1H), 4.91 (p, J = 6.4 Hz, 1H), 4.44 |
| | acid | (t, J = 8.4 Hz, 1H), 4.25 (dd, J = 8.8, 3.2 |
| | | Hz, 1H), 3.27 (dd, J = 14.4, 7.2 Hz, 2H), |
| | | 2.34 (s, 3H), 1.53 (d, J = 6.4 Hz, 3H). |
| 47 | 6-Chloro-3-(((R)- | ¹H NMR (400 MHz, DMSO-d₆) δ 7.71 |
| | 1-(6-((S)-4- | (s, 1H), 7.17 (s, 2H), 7.06 (s, 1H), 6.96 |
| | (cyclobutylmethyl)- | (s, 1H), 5.30 (t, J = 4.0 Hz, 1H), 4.83- |
| | 2-oxooxazolidin- | 4.80 (m, 1H), 4.46 (t, J = 8.0 Hz, 1H), |
| | 3-yl)-4-methylpyridin-2- | 4.18 (dd, J = 8.0, 4.0 Hz, 1H), 2.38- |
| | yl)ethyl)amino)picolinic | 2.33 (m, 1H), 2.28 (s, 3H), 2.00-1.93 |
| | acid, TFA | (m, 2H), 1.91-1.87 (m, 2H), 1.85-1.79 |
| | | (m, 2H), 1.76-1.70 (m, 2H), 1.44 (d, J = |
| | | 6.4 Hz, 3H). |

### Example 48. 6-Chloro-3-(((R)-1-(6-((S)-4-(3-cyanobenzyl)-2-oxooxazolidin-3-yl)-4-methylpyridin-2-yl)ethyl)amino)picolinic acid

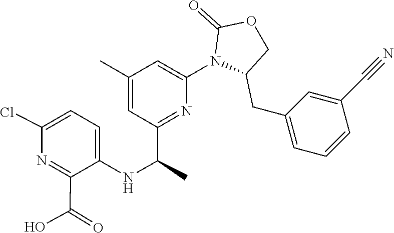 [SMILES: Cc1cc([C@@H](C)Nc2ccc(Cl)nc2C(=O)O)nc(N2C(=O)OC[C@@H]2Cc2cccc(C#N)c2)c1]

### Step 1. (S)-4-(3-Bromobenzyl)oxazolidin-2-one

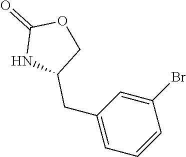 [SMILES: O=C1N[C@@H](Cc2cccc(Br)c2)CO1]

The title compound was synthesized by procedures analogous to those outlines in Example 9, Steps 1-2, using (S)-2-amino-3-(3-bromophenyl)propanoic acid instead of (S)-2-amino-3-(4-bromophenyl)propanoic acid. LC-MS calc. for C₁₀H₁₁BrNO₂ [M+H]⁺: m/z=256.0, 258.0; Found: 256.1, 258.1.

### Step 2. (S)-3-((2-Oxooxaolidin-4-yl)methyl)benzonitrile

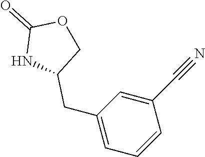 [SMILES: N#Cc1cccc(C[C@H]2COC(=O)N2)c1]

A mixture of (S)-4-(3-bromobenzyl)oxazolidin-2-one (65 mg, 0.25 mmol), zinc cyanide (35 mg, 0.38 mmol), zinc (3.3 mg, 51 μmol), 1,1′-bis(diphenylphosphino)ferrocene (14 mg, 25 μmol), and Pd₂(dba)₃ (12 mg, 13 μmol) in DMF (2.0 mL) was degassed with N₂. The reaction mixture was heated to 120° C. for 5 h. The mixture was cooled to rt, diluted with DCM (10 mL), filtered, and concentrated. The crude residue was purified by prep-HPLC on a C18 column (20-60% MeCN/0.1% TFA (aq.)) to afford the TFA salt of the title compound (18 mg, 89 μmol, 35% yield). LC-MS calc. for C₁₁H₁₁N₂O₂[M+H]⁺: m/z=203.1; Found: 203.2.

### Step 3. 6-Chloro-3-(((R)-1-(6-((S)-4-(3-cyanobenzyl)-2-oxooxaolidin-3-yl)-4-methylpyridin-2-yl)ethyl)amino)picolinic acid

The title compound was synthesized by procedures analogous to those outlines in Example 20, Steps 9-10. ¹H NMR (300 MHz, DMSO-d₆) δ 13.00 (bs, 1H), 8.75 (d, J=7.6 Hz, 1H), 7.84 (s, 1H), 7.79-7.71 (m, 2H), 7.55 (dd, J=9.9, 7.8 Hz, 2H), 7.43 (q, J=9.0 Hz, 2H), 7.08 (s, 1H), 5.16 (t, J=7.9 Hz, 1H), 5.00-4.88 (m, 1H), 4.44 (t, J=8.4 Hz, 1H), 4.34 (dd, J=8.8, 2.8 Hz, 1H), 3.29 (d, J=2.8 Hz, 1H), 3.20 (d, J=8.2 Hz, 1H), 2.37 (s, 3H), 1.51 (d, J=6.5 Hz, 3H). LC-MS calc. for C₂₅H₂₃ClN₅O₄ [M+H]⁺: m/z=492.1; Found: 492.3.

### Example 49. 6-Chloro-3-(((R)-1-(6-((S)-4-(4-cyano-2-fluorobenzyl)-2-oxooxazolidin-3-yl)-4-methylpyridin-2-yl)ethyl)amino)picolinic acid

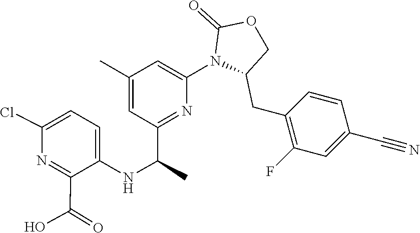 [SMILES: Cc1cc([C@@H](C)Nc2ccc(Cl)nc2C(=O)O)nc(N2C(=O)OC[C@@H]2Cc2ccc(C#N)cc2F)c1]

### Step 1. (S)-4-(4-Bromo-2-fluorobenzyl)oxazolidin-2-one

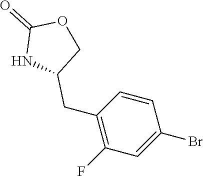 [SMILES: O=C1N[C@@H](Cc2ccc(Br)cc2F)CO1]

The title compound was synthesized by procedures analogous to those outlines in Example 20, Steps 1-3, using (S)-2-amino-3-(4-bromo-2-fluorophenyl)propanoic acid instead of (S)-2-amino-3-(2-fluoro-3-methylphenyl)propanoic acid. LC-MS calc. for C₁₀H₁₀BrFNO₂ [M+H]⁺: m/z=274.0, 276.0; Found: 274.1, 276.1.

### Step 2. (S)-3-Fluoro-4-((2-oxooxaolidin-4-yl)methyl)benzonitrile

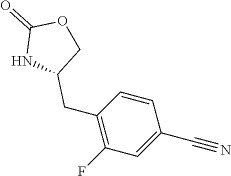 [SMILES: N#Cc1ccc(C[C@H]2COC(=O)N2)c(F)c1]

A mixture of (S)-4-(4-bromo-2-fluorobenzyl)oxazolidin-2-one (230 mg, 0.84 mmol), zinc cyanide (59 mg, 0.50 mmol), Pd(PPh₃)₄ (97 mg, 84 μmol) in DMF (3.0 mL) was degassed with N₂. The reaction mixture was heated to 120° C. for 5 h. The mixture was cooled to rt then diluted with EtOAc and water. The two phases were separated and the aqueous layer was extracted with EtOAc (20 mL×2). The combined organic layers were dried over sodium sulfate, filtered, and concentrated. The crude residue was purified by silica gel prep-TLC (33% EtOAc/petroleum ether) to afford the title compound (110 mg, 0.50 mmol, 60% yield). LC-MS calc. for C₁₁H₁₀FN₂O₂ [M+H]⁺: m/z=221.1; Found: 221.2.

### Step 3. 6-Chloro-3-(((R)-1-(6-((S)-4-(4-cyano-2-fluorobenyl)-2-oxooxazolidin-3-yl)-4-methylpyridin-2-yl)ethyl)amino)picolinic acid

The title compound was synthesized by procedures analogous to those outlines in Example 20, Steps 9-10. ¹H NMR (400 MHz, DMSO-d₆) δ 8.70 (d, J=7.3 Hz, 1H), 7.82 (dd, J=9.8, 1.6 Hz, 1H), 7.78 (s, 1H), 7.66 (dd, J=7.9, 1.6 Hz, 1H), 7.56 (t, J=7.6 Hz, 1H), 7.40 (d, J=8.9 Hz, 1H), 7.28 (d, J=z 9.0 Hz, 1H), 7.02 (s, 1H), 5.20 (dt, J=8.2, 4.0 Hz, 1H), 4.81 (p, J=6.8 Hz, 1H), 4.46 (t, J=8.5 Hz, 1H), 4.25 (dd, J=9.0, 2.6 Hz, 1H), 3.33-3.24 (m, 2H), 2.33 (s, 3H), 1.47 (d, J=6.5 Hz, 3H). LC-MS calc. for C₂₅H₂₂ClFN₅O₄[M+H]⁺: m/z=510.1; Found: 510.1.

### Example 50. 3-(((R)-1-(6-((S)-4-((1H-Indol-3-yl)methyl)-2-oxooxazolidin-3-yl)-4-methylpyridin-2-yl)ethyl)amino)-6-chloropicolinic acid

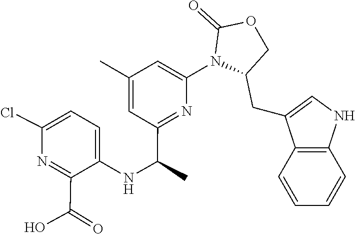 [SMILES: Cc1cc([C@@H](C)Nc2ccc(Cl)nc2C(=O)O)nc(N2C(=O)OC[C@@H]2Cc2c[nH]c3ccccc23)c1]

### Step 1. (S)-4-((1H-Indol-3-yl)methyl)oxazolidin-2-one

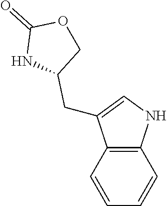 [SMILES: O=C1N[C@@H](Cc2c[nH]c3ccccc23)CO1]

The title compound was synthesized by procedures analogous to those outlines in Example 20, Steps 1-3, using L-tryptophan instead of (S)-2-amino-3-(2-fluoro-3-methylphenyl)propanoic acid. ¹H NMR (400 MHz, DMSO-d₆) δ 10.89 (s, 1H), 7.74 (s, 1H), 7.51 (d, J=8.0 Hz, 1H), 7.32 (d, J=8.0 Hz, 1H), 7.19 (d, J=4.0 Hz, 1H), 7.05 (t, J=8.0 Hz, 1H), 6.96 (t, J=8.0 Hz, 1H), 4.26 (t, J=8.0 Hz, 1H), 4.10-4.05 (m, 1H), 3.98-3.95 (m, 1H), 2.91 (dd, J=12.0, 4.0 Hz, 1H), 2.80 (dd, J=12.0, 4.0 Hz, 1H). LC-MS calc. for C₁₂H₁₃N₂O₂ [M+H]⁺: m/z=217.1; Found: 217.1.

### Step 2. tert-Butyl (S)-3-((2-oxooxazolidin-4-yl)methyl)-1H-indole-1-carboxylate

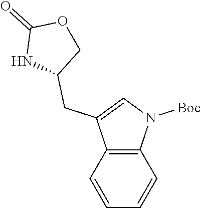 [SMILES: CC(C)(C)OC(=O)n1cc(C[C@H]2COC(=O)N2)c2ccccc21]

To a solution of (S)-4-((1H-indol-3-yl)methyl)oxazolidin-2-one (0.750 g, 3.47 mmol) in DCM (15 mL) was added TEA (1.05 g, 10.4 mmol) and Boc₂O (0.907 g, 4.16 mmol). The resulting mixture was stirred at 35° C. for 24 h. The mixture was diluted with water (30 ml) and extracted with DCM (20 ml×3). The combined organic layers were dried over sodium sulfate, filtered, and concentrated. The crude residue was purified by silica gel column chromatography (0-25% EtOAc/hexane) to afford the title compound (0.300 g, 0.948 mmol, 27.3% yield) as orange solid. ¹H NMR (400 MHz, DMSO-d₆) δ 8.01 (d, J=8.0 Hz, 1H), 7.76 (s, 1H), 7.63 (d, J=8.0 Hz, 1H), 7.56 (s, 1H), 7.31 (t, J=8.0 Hz, 1H), 7.23 (t, J=8.0 Hz, 1H), 4.33 (t, J=8.0 Hz, 1H), 4.15-4.09 (m, 1H), 4.00 (dd, J=8.0, 4.0 Hz, 1H), 2.86 (d, J=8.0 Hz, 2H), 1.61 (s, 9H).

### Step 3. tert-Butyl 3-(((S)-3-(6-((R)-1-((6-chloro-2-(methoxycarbonyl)pyridin-3-yl)amino)ethyl)-4-methylpyridin-2-yl)-2-oxooxazolidin-4-yl)methyl)-1H-indole-1-carboxylate

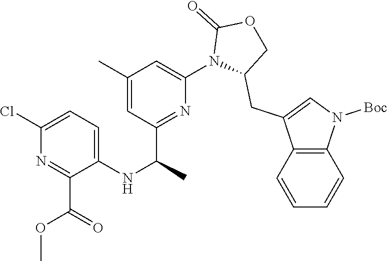 [SMILES: COC(=O)c1nc(Cl)ccc1N[C@H](C)c1cc(C)cc(N2C(=O)OC[C@@H]2Cc2cn(C(=O)OC(C)(C)C)c3ccccc23)n1]

The title compound was synthesized by procedures analogous to those outlines in Example 20, Step 9. ¹H NMR (400 MHz, DMSO-d₆) δ 8.53 (d, J=8.0 Hz, 1H), 8.00 (d, J=8.0 Hz, 1H), 7.81 (s, 1H), 7.61 (d, J=8.0 Hz, 1H), 7.55 (s, 1H), 7.41-7.34 (m, 2H), 7.28 (t, J=8.0 Hz, 1H), 7.10 (t, J=8.0 Hz, 1H), 7.05 (s, 1H), 5.26-5.20 (m, 1H), 4.91-4.85 (m, 1H), 4.45 (t, J=8.0 Hz, 1H), 4.27 (dd, J=8.0, 4.0 Hz, 1H), 3.74 (s, 3H), 3.38 (dd, J=16.0, 4.0 Hz, 1H), 3.12 (dd, J=16.0, 8.0 Hz, 1H), 2.32 (s, 3H), 1.58 (s, 9H), 1.52 (d, J=8.0 Hz, 3H).

### Step 4. Methyl 3-(((R)-1-(6-((S)-4-((1H-indol-3-yl)methyl)-2-oxooxazolidin-3-yl)-4-methylpyridin-2-yl)ethyl)amino)-6-chloropicolinate

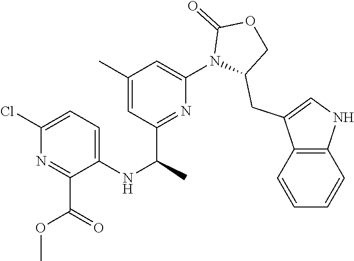 [SMILES: COC(=O)c1nc(Cl)ccc1N[C@H](C)c1cc(C)cc(N2C(=O)OC[C@@H]2Cc2c[nH]c3ccccc23)n1]

To a solution of tert-butyl 3-(((S)-3-(6-((R)-1-((6-chloro-2-(methoxycarbonyl)pyridin-3-yl)amino)ethyl)-4-methylpyridin-2-yl)-2-oxooxazolidin-4-yl)methyl)-1H-indole-1-carboxylate (15.0 mg, 0.024 mmol) in DCM (0.2 mL) was added formic acid (1.0 mL). The resulting mixture was stirred at 25° C. for 12 h then concentrated. The residue was diluted with sat. NaHCO₃ solution (aq.) (10 ml) and extracted with EtOAc (10 ml×2). The combined organic layers were dried over sodium sulfate, filtered, and concentrated to afford the title compound (13.0 mg, 0.025 mmol, 103% yield) which was used directly in the next step. LCMS calc. for C₂₇H₂₇ClN₅O₄ [M+H]⁺: m/z=520.2; Found: 520.2.

### Step 5. 3-(((R)-1-(6-((S)-4-((1H-Indol-3-yl)methyl)-2-oxooxaolidin-3-yl)-4-methylpyridin-2-yl)ethyl)amino)-6-chloropicolinic acid

The title compound was synthesized by procedures analogous to those outlines in Example 20, Step 10. ¹H NMR (400 MHz, DMSO-d₆) δ 10.92 (d, J=2.0 Hz, 1H), 8.83 (d, J=8.0 Hz, 1H), 7.81 (s, 1H), 7.54 (d, J=8.0 Hz, 1H), 7.35-7.29 (m, 3H), 7.20 (d, J=2.4 Hz, 1H), 7.04-7.00 (m, 2H), 6.84 (t, J=8.0 Hz, 1H), 5.22-5.17 (m, 1H), 4.88-4.82 (m, 1H), 4.36 (t, J=8.0 Hz, 1H), 4.23 (dd, J=8.0, 3.2 Hz, 1H), 3.39 (d, J=2.8 Hz, 1H), 3.06 (dd, J=16.0, 12.0 Hz, 1H), 2.32 (s, 3H), 1.53 (d, J=4.0 Hz, 3H). LCMS calc. for C₂₆H₂₅ClN₅O₄[M+H]⁺: m/z=506.2; Found: 506.3.

### Example 51. 6-Chloro-3-(((R)-1-(6-((S)-4-(4-(dimethylcarbamoyl)benzyl)-2-oxooxazolidin-3-yl)-4-methylpyridin-2-yl)ethyl)amino)picolinic acid

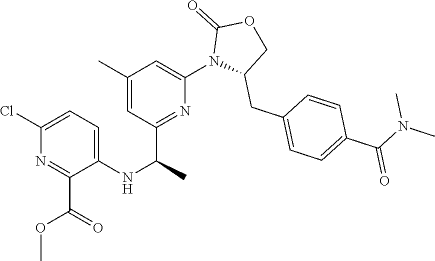 [SMILES: COC(=O)c1nc(Cl)ccc1N[C@H](C)c1cc(C)cc(N2C(=O)OC[C@@H]2Cc2ccc(C(=O)N(C)C)cc2)n1]

### Step 1. Methyl 3-(((R)-1-(6-((S)-4-(4-(tert-butoxycarbonyl)benyl)-2-oxooxaolidin-3-yl)-4-methylpyridin-2-yl)ethyl)amino)-6-chloropicolinate

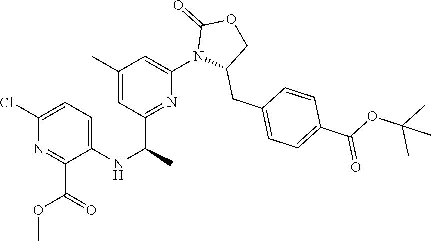 [SMILES: COC(=O)c1nc(Cl)ccc1N[C@H](C)c1cc(C)cc(N2C(=O)OC[C@@H]2Cc2ccc(C(=O)OC(C)(C)C)cc2)n1]

The title compound was synthesized by procedures analogous to those outlines in Example 21, Steps 1-4, using tert-butyl 4-iodobenzoate instead of 3-fluoro-4-iodopyridine. LCMS calc. for C₃₀H₃₃ClN₄NaO₆ [M+Na]⁺: m/z=603.2; Found: 603.3.

### Step 2. 4-(((S)-3-(6-((R)-1-((6-Chloro-2-(methoxycarbonyl)pyridin-3-yl)amino)ethyl)-4-methylpyridin-2-yl)-2-oxooxaolidin-4-yl)methyl)benoic acid

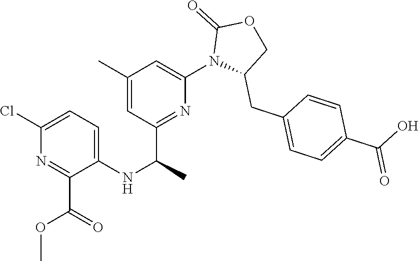 [SMILES: COC(=O)c1nc(Cl)ccc1N[C@H](C)c1cc(C)cc(N2C(=O)OC[C@@H]2Cc2ccc(C(=O)O)cc2)n1]

To a solution of methyl 3-(((R)-1-(6-((S)-4-(4-(tert-butoxycarbonyl)benzyl)-2-oxooxazolidin-3-yl)-4-methylpyridin-2-yl)ethyl)amino)-6-chloropicolinate (580 mg, 1.00 mmol) in DCM (10 mL) was added TFA (2.0 mL, 1.0 mmol) dropwise at 0° C. The resulting mixture was warmed to rt and stirred for 1 h. The reaction mixture was filtered and concentrated to afford the TFA salt of the title compound (500 mg, 0.952 mmol, 95.4% yield). LCMS calc. for C₂₆H₂₆ClN₄O₆ [M+H]⁺: m/z=525.2; Found: 525.1.

### Step 3. Methyl 6-chloro-3-(((R)-1-(6-((S)-4-(4-(dimethylcarbamoyl)benyl)-2-oxooxaolidin-3-yl)-4-methylpyridin-2-yl)ethyl)amino)picolinate

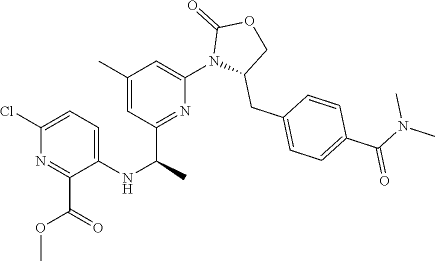 [SMILES: COC(=O)c1nc(Cl)ccc1N[C@H](C)c1cc(C)cc(N2C(=O)OC[C@@H]2Cc2ccc(C(=O)N(C)C)cc2)n1]

To a solution of 4-(((S)-3-(6-((R)-1-((6-chloro-2-(methoxycarbonyl)pyridin-3-yl)amino)ethyl)-4-methylpyridin-2-yl)-2-oxooxazolidin-4-yl)methyl)benzoic acid, TFA salt (100 mg, 0.191 mmol) in DMF (2.0 mL) was added dimethylamine hydrochloride (22.4 mg, 0.275 mmol), HATU (108 mg, 0.284 mmol) and DIEA (73.9 mg, 0.573 mmol). The resulting mixture was stirred at rt for 2 h before being diluted with water (10 ml) and extracted with EtOAc (10 mL×3). The combined organic layers were washed with brine, dried over Na₂SO₄, filtered, and concentrated to afford the title (70.0 mg, 0.127 mmol, 66.6% yield) which was used directly in the next step. LCMS calc. for C₂₈H₃₁ClN₅O₅[M+H]⁺: m/z=552.2; Found: 552.3.

### Step 4. 6-Chloro-3-(((R)-1-(6-((S)-4-(4-(dimethylcarbamoyl)benyl)-2-oxooxaolidin-3-yl)-4-methylpyridin-2-yl)ethyl)amino)picolinic acid

The title compound was synthesized by procedures analogous to those outlines in Example 20, Step 10. ¹H NMR (400 MHz, DMSO-d₆) δ 12.96 (bs, 1H), 8.70 (d, J=7.6 Hz, 1H), 7.81 (s, 1H), 7.39 (q, J=9.2 Hz, 2H), 7.33-7.25 (m, 4H), 7.05 (s, 1H), 5.10 (dd, J=8.4, 3.2 Hz, 1H), 4.89 (p, J=6.8 Hz, 1H), 4.39 (t, J=8.4 Hz, 1H), 4.24 (dd, J=8.8, 2.8 Hz, 1H), 3.28-3.25 (m, 1H), 3.05 (dd, J=13.2, 8.4 Hz, 1H), 2.97-2.83 (m, 6H), 2.34 (s, 3H), 1.48 (d, J=6.4 Hz, 3H).

LCMS calc. for C₂₇H₂₉ClN₅O₅ [M+H]⁺: m/z=538.2; Found: 538.0.

### Example 52. 6-Chloro-3-(((R)-1-(6-((S)-4-(4-(methoxy(methyl)carbamoyl)benzyl)-2-oxooxazolidin-3-yl)-4-methylpyridin-2-yl)ethyl)amino)picolinic acid

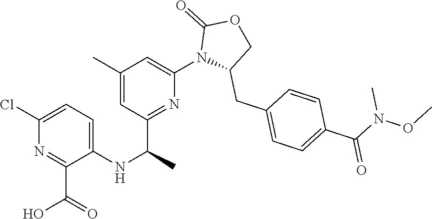 [SMILES: CON(C)C(=O)c1ccc(C[C@H]2COC(=O)N2c2cc(C)cc([C@@H](C)Nc3ccc(Cl)nc3C(=O)O)n2)cc1]

### Step 1. Methyl 6-chloro-3-(((R)-1-(6-((S)-4-(4-(methoxy(methyl)carbamoyl)benyl)-2-oxooxaolidin-3-yl)-4-methylpyridin-2-yl)ethyl)amino)picolinate

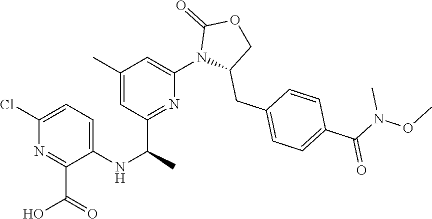 [SMILES: CON(C)C(=O)c1ccc(C[C@H]2COC(=O)N2c2cc(C)cc([C@@H](C)Nc3ccc(Cl)nc3C(=O)O)n2)cc1]

To a solution of 4-(((S)-3-(6-((R)-1-((6-chloro-2-(methoxycarbonyl)pyridin-3-yl)amino)ethyl)-4-methylpyridin-2-yl)-2-oxooxazolidin-4-yl)methyl)benzoic acid (100 mg, 0.190 mmol, Example 51, Step 2), N,O-dimethylhydroxylamine hydrochloride (23.3 mg, 0.239 mmol), and DIPEA (123 mg, 0.953 mmol) in DMF (5.0 mL) was added HATU (109 mg, 0.287 mmol). The reaction mixture was stirred at rt for 16 h before being poured into water (10 mL) and extracted with EtOAc (10 mL×2). The combined organic phase was dried over Na₂SO₄, filtered, and concentrated. The crude residue was purified by silica gel prep-TLC (100% EtOAc) to afford the title compound (60.0 mg, 0.106 mmol, 55.5% yield) as a colorless oil. LCMS calc. for C₂₈H₃₁ClN₅O₆ [M+H]⁺: m/z=568.2; Found: 568.3.

### Step 2. 6-Chloro-3-(((R)-1-(6-((S)-4-(4-(methoxy(methyl)carbamoyl)benyl)-2-oxooxaolidin-3-yl)-4-methylpyridin-2-yl)ethyl)amino)picolinic acid

The title compound was synthesized by procedures analogous to those outlines in Example 20, Step 10. ¹H NMR (400 MHz, DMSO-d₆) δ 8.72 (d, J=8.0 Hz, 1H), 7.84 (s, 1H), 7.52 (t, J=8.0 Hz, 2H), 7.53-7.40 (m, 2H), 7.39-7.31 (m, 2H), 7.08 (s, 1H), 5.15-5.11 (m, 1H), 4.93-4.89 (m, 1H), 4.41 (t, J=8.0 Hz, 1H), 4.27-4.24 (m, 1H), 3.52 (s, 3H), 3.34-3.30 (m, 1H), 3.24 (s, 3H), 3.11-3.06 (m, 1H), 2.36 (s, 3H), 1.50 (d, J=8.0 Hz, 3H). LCMS calc. for C₂₇H₂₉ClN₅O₆ [M+H]⁺: m/z=554.2; Found: 554.0.

### Example 53. 6-Chloro-3-(((R)-1-(4-methyl-6-((S)-2-oxo-4-phenethyloxazolidin-3-yl)pyridin-2-yl)ethyl)amino)picolinic acid

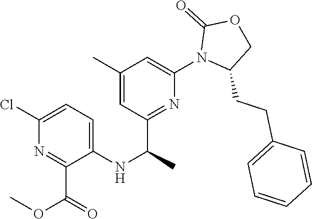 [SMILES: COC(=O)c1nc(Cl)ccc1N[C@H](C)c1cc(C)cc(N2C(=O)OC[C@@H]2CCc2ccccc2)n1]

### Step 1: Methyl 6-chloro-3-(((1R)-1-(4-methyl-6-(2-oxo-4-phenethyloxaolidin-3-yl)pyridin-2-yl)ethyl)amino)picolinate, isomers 1 and 2

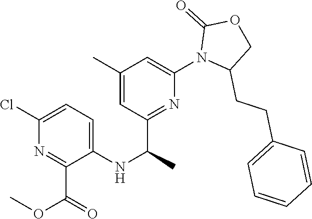 [SMILES: COC(=O)c1nc(Cl)ccc1N[C@H](C)c1cc(C)cc(N2C(=O)OCC2CCc2ccccc2)n1]

The title compounds were synthesized by procedures analogous to those outlines in Example 20, Step 9 using 4-phenethyloxazolidin-2-one instead of (S)-4-(2-fluoro-3-methylbenzyl)oxazolidin-2-one. The two isomers were separated by prep-HPLC on a C18 column (68-88% MeCN/0.1% TFA (aq.)) to afford the TFA salt of methyl 6-chloro-3-(((1R)-1-(4-methyl-6-(2-oxo-4-phenethyloxazolidin-3-yl)pyridin-2-yl)ethyl)amino)picolinate isomer 1 (8.8 mg, 0.018 mmol, 30% yield). LCMS calc. for C₂₆H₂₈ClN₄O₄[M+H]⁺ m/z=495.2; Found 495.3. And methyl 6-chloro-3-(((1R)-1-(4-methyl-6-(2-oxo-4-phenethyloxazolidin-3-yl)pyridin-2-yl)ethyl)amino)picolinate isomer 2 (11 mg, 0.021 mmol, 36% yield). LCMS calc. for C₂₆H₂₈ClN₄O₄ [M+H]⁺ m/z=495.2; Found: 495.3.

### Step 2: 6-Chloro-3-(((R)-1-(4-methyl-6-((S)-2-oxo-4-phenethyloxazolidin-3-yl)pyridin-2-yl)ethyl)amino)picolinic acid

The title compound was synthesized by procedures analogous to those outlines in Example 20, Step 10 from methyl 6-chloro-3-(((1R)-1-(4-methyl-6-(2-oxo-4-phenethyloxazolidin-3-yl)pyridin-2-yl)ethyl)amino)picolinate isomer 2. ¹H NMR (300 MHz, DMSO-d₆) δ 13.00 (bs, 1H), 8.65 (d, J=7.7 Hz, 1H), 7.77 (s, 1H), 7.34 (d, J=1.1 Hz, 2H), 7.28-7.13 (m, 5H), 7.02 (s, 1H), 4.98-4.88 (m, 1H), 4.87-4.76 (m, 1H), 4.56 (t, J=8.5 Hz, 1H), 4.39 (dd, J=8.7, 3.6 Hz, 1H), 2.80-2.57 (m, 2H), 2.33 (s, 3H), 2.28-2.14 (m, 1H), 2.07 (ddd, J=22.4, 10.4, 7.0 Hz, 1H), 1.41 (d, J=6.5 Hz, 3H). LCMS calc. for C₂₅H₂₆ClN₄O₄[M+H]⁺: m/z=481.2; Found: 481.2.

### Example 54. 3-(((R)-1-(6-((S)-4-Benzyl-4-methyl-2-oxooxazolidin-3-yl)-4-methylpyridin-2-yl)ethyl)amino)-6-chloropicolinic acid

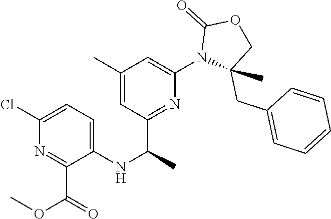 [SMILES: COC(=O)c1nc(Cl)ccc1N[C@H](C)c1cc(C)cc(N2C(=O)OC[C@@]2(C)Cc2ccccc2)n1]

The title compound was synthesized by procedures analogous to those outlined in Example 20 using (S)-2-amino-2-methyl-3-phenylpropanoic acid instead of (S)-2-amino-3-(2-fluoro-3-methylphenyl)propanoic acid. ¹H NMR (400 MHz, DMSO-d₆) δ 8.68 (d, J=7.6 Hz, 1H), 7.82 (d, J=1.2 Hz, 1H), 7.47 (s, 1H), 7.42 (d, J=8.8 Hz, 1H), 7.33 (d, J=9.2 Hz, 1H), 7.03 (d, J=1.2 Hz, 1H), 5.00 (dd, J=8.4, 3.2 Hz, 1H), 4.85 (t, J=6.8 Hz, 1H), 4.42 (t, J=8.4 Hz, 1H), 4.19 (dd, J=8.8, 3.2 Hz, 1H), 3.69 (s, 3H), 2.96 (dd, J=14.0, 3.2 Hz, 1H), 2.81 (dd, J=14.0, 8.0 Hz, 1H), 2.33 (s, 3H), 1.96 (s, 3H), 1.48 (d, J=6.4 Hz, 3H). LCMS calc. for C₂₅H₂₆ClN₄O₄[M+H]⁺: m/z=481.2; Found: 481.3.

### Example 55. 3-(((1R)-1-(6-(2-benzyl-5-oxopyrrolidin-1-yl)-4-methylpyridin-2-yl)ethyl)amino)-6-chloropicolinic acid

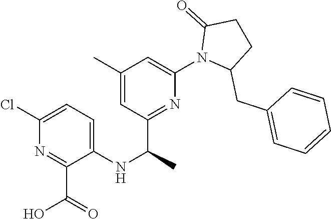 [SMILES: Cc1cc([C@@H](C)Nc2ccc(Cl)nc2C(=O)O)nc(N2C(=O)CCC2Cc2ccccc2)c1]

The title compound was synthesized by procedures analogous to those outlines in Example 20, Steps 4-10, using 5-benzylpyrrolidin-2-one instead of (S)-4-(2-fluoro-3-methylbenzyl)oxazolidin-2-one. ¹H NMR (300 MHz, DMSO-d₆) δ 12.97 (bs, 1H), 8.54 (d, J=7.1 Hz, 1H), 8.01 (s, 1H), 7.43 (d, J=8.9 Hz, 1H), 7.32-7.19 (m, 4H), 7.11 (dd, J=11.0, 4.9 Hz, 3H), 5.03 (t, J=5.9 Hz, 1H), 4.87 (t, J=6.8 Hz, 1H), 2.96 (dd, J=13.0, 3.0 Hz, 1H), 2.75 (dd, J=13.0, 8.1 Hz, 1H), 2.36 (s, 3H), 2.27 (dd, J=8.7, 4.0 Hz, 2H), 2.07 (dd, J=15.6, 5.8 Hz, 1H), 1.85 (dd, J=12.0, 5.5 Hz, 1H), 1.56 (d, J=6.6 Hz, 3H). LCMS calc. for C₂₅H₂₆ClN₄O₃[M+H]⁺: m/z=465.2; Found: 465.3.

### Example 56. 3-(((R)-1-(6-((S)-4-Benzyl-2-oxo-1,3-oxazinan-3-yl)-4-methylpyridin-2-yl)ethyl)amino)-6-chloropicolinic acid

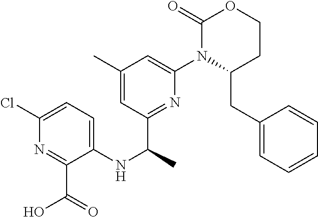 [SMILES: Cc1cc([C@@H](C)Nc2ccc(Cl)nc2C(=O)O)nc(N2C(=O)OCC[C@@H]2Cc2ccccc2)c1]

The title compound was synthesized by procedures analogous to those outlined in Example 9, Steps 1-2, using (S)-3-amino-4-phenylbutanoic acid instead of (S)-2-amino-3-(4-bromophenyl)propanoic acid and Example 20, Steps 4-10. ¹H NMR (300 MHz, DMSO-d₆) δ 8.63 (d, J=6.7 Hz, 1H), 7.36 (s, 3H), 7.23 (dd, J=12.2, 6.5 Hz, 5H), 7.11 (s, 1H), 5.06 (d, J=3.3 Hz, 1H), 4.95-4.83 (m, 1H), 4.57 (td, J=11.3, 2.9 Hz, 1H), 4.32 (dd, J=7.0, 3.9 Hz, 1H), 2.91 (dd, J=13.3, 4.4 Hz, 1H), 2.78 (dd, J=13.2, 9.9 Hz, 1H), 2.32 (s, 3H), 2.15-1.97 (m, 1H), 1.84 (dd, J=14.3, 2.8 Hz, 1H), 1.53 (d, J=6.6 Hz, 3H). LCMS calc. for C₂₅H₂₆ClN₄O₄[M+H]⁺: m/z=481.2; Found: 481.3.

### Example 57. 3-(((R)-1-(6-((S)-3-Benzyl-5-oxomorpholino)-4-methylpyridin-2-yl)ethyl)amino)-6-chloropicolinic acid

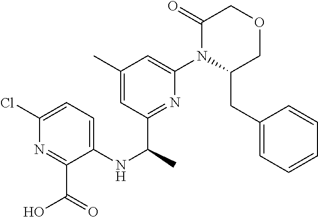 [SMILES: Cc1cc([C@@H](C)Nc2ccc(Cl)nc2C(=O)O)nc(N2C(=O)COC[C@@H]2Cc2ccccc2)c1]

The title compound was synthesized by procedures analogous to those outlined in Example 20, Steps 4-10 using (S)-5-benzylmorpholin-3-one instead of (S)-4-(2-fluoro-3-methylbenzyl)oxazolidin-2-one. ¹H NMR (300 MHz, DMSO-d₆) δ 8.65 (d, J=6.8 Hz, 1H), 7.65 (s, 1H), 7.38 (q, J=9.0 Hz, 2H), 7.23 (dd, J=10.4, 5.9 Hz, 5H), 7.13 (s, 1H), 5.05 (t, J=7.2 Hz, 1H), 4.99-4.84 (m, 1H), 4.40 (d, J=16.9 Hz, 1H), 4.24 (d, J=16.9 Hz, 1H), 3.86-3.81 (m, 2H), 2.95 (d, J=7.8 Hz, 2H), 2.32 (s, 3H), 1.53 (d, J=6.5 Hz, 3H). LCMS calc. for C₂₅H₂₆ClN₄O₄[M+H]⁺: m/z=481.2; Found: 481.2.

### Example 58. 6-Chloro-3-(((R)-1-(6-((S)-5-(4-fluorobenzyl)-3-methyl-2-oxoimidazolidin-1-yl)-4-methylpyridin-2-yl)ethyl)amino)picolinic acid

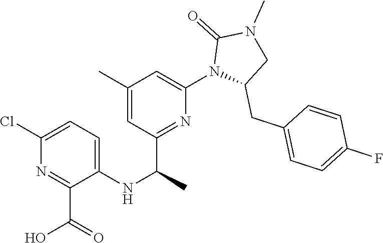 [SMILES: Cc1cc([C@@H](C)Nc2ccc(Cl)nc2C(=O)O)nc(N2C(=O)N(C)C[C@@H]2Cc2ccc(F)cc2)c1]

### Step 1. tert-Butyl (S)-(3-(4-fluorophenyl)-1-(methylamino)-1-oxopropan-2-yl)carbamate

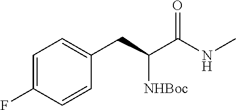 [SMILES: CNC(=O)[C@H](Cc1ccc(F)cc1)NC(=O)OC(C)(C)C]

To a solution of (S)-2-((tert-butoxycarbonyl)amino)-3-(4-fluorophenyl)propanoic acid (20 g, 75 mmol) in DCM (150 mL) was added CDI (13 g, 83 mmol) and the resulting mixture was stirred at rt for 2 h. Methylamine hydrochloride (5.3 g, 79 mmol) was added and the reaction mixture was stirred at rt for 2 h. The mixture was concentrated before being suspended in EtOH (60 ml) and stirred for 30 min. The solution was diluted with water (100 ml) and stirred for 30 min. The mixture was filtered and the solid was dried under vacuum at 50° C. to afford the title compound (16 g, 76% yield) as white solid. t_{R}=5.39 min (C18 column; 5-95% MeCN/0.05% TFA (aq.) for 1 min, 5-95% MeCN/0.05% TFA (aq.) over 4 min, 95% MeCN/0.05% TFA (aq.) for 5 min; 1 mL/min).

### Step 2. (S)-4-(4-Fluorobenyl)-1-methylimidaolidin-2-one

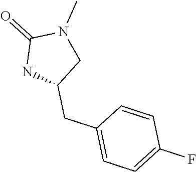

To a solution of tert-butyl (S)-(1-(4-fluorophenyl)-4-(methylamino)-4-oxobutan-2-yl)carbamate (1.0 g, 3.4 mmol) in 2:1 v/v toluene:THF (15 mL) was added a solution of Red-Al (5.7 mL, 16.9 mmol, 60 wt % in toluene) was added dropwise at 0° C. The reaction mixture was warmed to rt and stirred for 10 min. Then the reaction was heated to 40° C. for 16 h. The reaction mixture was cooled to 0° C. and diluted with 5N NaOH (aq.) (100 ml). The mixture was warmed to rt and stirred for 30 min. The mixture was diluted with toluene (30 ml) and washed with 5N NaOH (10 ml×2). The combined organic layers were dried over sodium sulfate, filtered, and concentrated. The crude residue was purified by silica gel column chromatography (0-50% EtOAc/heptanes) to afford the title compound (500 mg, 2.4 mmol, 71% yield) as off white solid. ¹H NMR (400 MHz, CDCl₃) δ 7.18-7.08 (m, 2H), 7.04-6.92 (m, 2H), 3.89-3.77 (m, 1H), 3.49 (t, J=8.6 Hz, 1H), 3.13 (dd, J=8.9, 6.1 Hz, 1H), 2.84-2.72 (m, 5H).

### Step 3. Methyl 6-chloro-3-(((R)-1-(6-((S)-5-(4-fluorobenyl)-3-methyl-2-oxoimidazolidin-1-yl)-4-methylpyridin-2-yl)ethyl)amino)picolinate

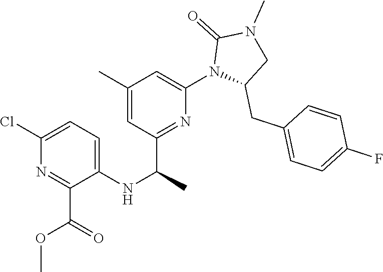 [SMILES: COC(=O)c1nc(Cl)ccc1N[C@H](C)c1cc(C)cc(N2C(=O)N(C)C[C@@H]2Cc2ccc(F)cc2)n1]

The title compound was synthesized by procedures analogous to those outlines in Example 20, Step 9. LCMS calc. for C₂₆H₂₈ClFN₅O₃[M+H]+ m/z=512.2; Found: 512.6.

### Step 4. 6-Chloro-3-(((R)-1-(6-((S)-5-(4-fluorobenyl)-3-methyl-2-oxoimidaolidin-1-yl)-4-methylpyridin-2-yl)ethyl)amino)picolinic acid

The title compound was synthesized by procedures analogous to those outlines in Example 20, Step 10. ¹H NMR (400 MHz, DMSO-d₆) δ 9.99 (d, J=7.3 Hz, 1H), 7.87 (s, 1H), 7.39 (dd, J=8.1, 5.7 Hz, 2H), 7.11 (dd, J=24.4, 15.6 Hz, 3H), 6.99 (s, 1H), 6.83 (s, 1H), 4.86 (t, J=8.8 Hz, 1H), 4.62-4.51 (m, 1H), 3.36 (t, J=9.1 Hz, 1H), 3.29 (s, 1H), 3.16 (dd, J=9.1, 2.8 Hz, 1H), 2.78 (d, J=9.3 Hz, 1H), 2.68 (s, 3H), 2.26 (s, 3H), 1.47 (d, J=6.5 Hz, 3H). LCMS calc. for C₂₅H₂₆ClFN₅O₃[M+H]+ m/z=498.2; Found: 498.2.

### Example 59. 6-Chloro-3-(((R)-1-(6-((S)-5-(2,4-difluorobenzyl)-3-methyl-2-oxoimidazolidin-1-yl)-4-methylpyridin-2-yl)ethyl)amino)picolinic acid

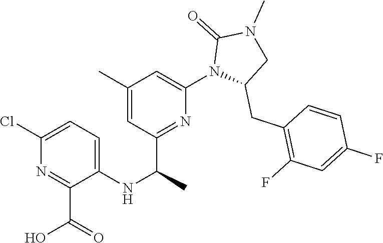 [SMILES: Cc1cc([C@@H](C)Nc2ccc(Cl)nc2C(=O)O)nc(N2C(=O)N(C)C[C@@H]2Cc2ccc(F)cc2F)c1]

### Step 1. Methyl (S)-2-((tert-butoxycarbonyl)amino)-3-(2,4-difluorophenyl)propanoate

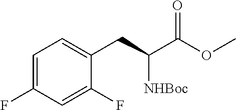 [SMILES: COC(=O)[C@H](Cc1ccc(F)cc1F)NC(=O)OC(C)(C)C]

The title compound was synthesized by procedures analogous to those outlines in Example 21, Step 1 using 1-bromo-2,4-difluorobenzene instead of 3-fluoro-4-iodopyridine. LCMS calc. for C₁₅H₂₀F₂NO₄ [M+H]+ m/z=316.1; Found: 315.6.

### Step 2. (S)-2-((tert-butoxycarbonyl)amino)-3-(2,4-difluorophenyl)propanoic acid

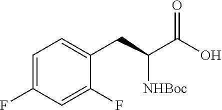 [SMILES: CC(C)(C)OC(=O)N[C@@H](Cc1ccc(F)cc1F)C(=O)O]

To a mixture of methyl (S)-2-((tert-butoxycarbonyl)amino)-3-(2,4-difluorophenyl)propanoate (8.4 g, 27 mmol) in THF (80 mL) and H₂O (30 mL) was added LiOH·H₂O (2.2 g, 53 mmol). The reaction mixture was stirred at rt for 1 hr. The mixture was acidified with 0.5N HCl (aq.) to pH=3-4, then extracted with EtOAc (100 mL×2). The combined organic layers were washed with brine, dried over Na₂SO₄, and concentrated to afford the title compound (7.8 g, 26 mmol, 97% yield). t_{R}=5.68 min (C18 column; 5-95% MeCN/0.05% TFA (aq.) for 1 min, 5-95% MeCN/0.05% TFA (aq.) over 4 min, 95% MeCN/0.05% TFA (aq.) for 5 min; 1 mL/min).

### Step 3. 6-Chloro-3-(((R)-1-(6-((S)-5-(2,4-difluorobenzyl)-3-methyl-2-oxoimidazolidin-1-yl)-4-methylpyridin-2-yl)ethyl)amino)picolinic acid

The title compound was synthesized by procedures analogous to those outlines in Example 58, Steps 1-2 and Example 20 Steps 4-10. ¹H NMR (400 MHz, DMSO-d₆) δ 12.97 (bs, 1H), 8.98 (s, 1H), 7.89 (s, 1H), 7.41 (dd, J=15.4, 8.4 Hz, 1H), 7.32 (d, J=8.8 Hz, 1H), 7.27-7.15 (m, 2H), 7.03 (td, J=8.5, 2.4 Hz, 1H), 6.84 (s, 1H), 4.95 (t, J=8.6 Hz, 1H), 4.77-4.66 (m, 1H), 3.43 (t, J=9.0 Hz, 1H), 3.24 (d, J=10.6 Hz, 1H), 3.15 (dd, J=9.3, 2.7 Hz, 1H), 2.93 (dd, J=13.5, 8.5 Hz, 1H), 2.68 (s, 3H), 2.27 (s, 3H), 1.47 (d, J=6.5 Hz, 3H). LCMS calc. for C₂₅H₂₅C₁F₂N₅O₃ [M+H]+ m/z=516.2; Found: 516.1.

### Example 60. 3-(((R)-1-(6-((S)-5-Benzyl-3-methyl-2-oxoimidazolidin-1-yl)-4-methylpyridin-2-yl)ethyl)amino)-6-chloropicolinic acid

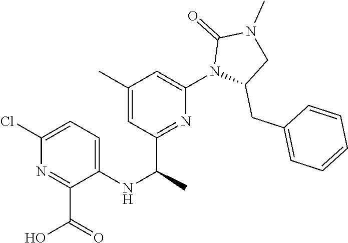 [SMILES: Cc1cc([C@@H](C)Nc2ccc(Cl)nc2C(=O)O)nc(N2C(=O)N(C)C[C@@H]2Cc2ccccc2)c1]

The title compound was synthesized by procedures analogous to those outlines in Example 58, Steps 1-4 using (tert-butoxycarbonyl)-L-phenylalanine instead of (S)-2-((tert-butoxycarbonyl)amino)-3-(4-fluorophenyl)propanoic acid. ¹H NMR (400 MHz, DMSO-d₆) δ 8.74 (s, 1H), 7.94 (s, 1H), 7.44-7.36 (m, 2H), 7.33-7.19 (m, 5H), 6.90 (s, 1H), 4.89 (dd, J=21.7, 12.5 Hz, 2H), 3.34 (dd, J=17.8, 9.5 Hz, 2H), 3.18 (dd, J=9.0, 2.5 Hz, 1H), 2.74 (dd, J=12.7, 9.4 Hz, 1H), 2.69 (s, 3H), 2.30 (s, 3H), 1.48 (d, J=6.4 Hz, 3H). LCMS calc. for C₂₅H₂₇ClN₅O₃[M+H]+ m/z=480.2; Found: 480.1.

### Example 61. 6-Chloro-3-(((R)-1-(3-fluoro-5-((S)-4-(2-fluorobenzyl)-2-oxooxazolidin-3-yl)phenyl)ethyl)amino)picolinic acid

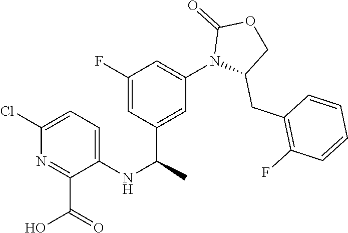 [SMILES: C[C@@H](Nc1ccc(Cl)nc1C(=O)O)c1cc(F)cc(N2C(=O)OC[C@@H]2Cc2ccccc2F)c1]

The title compound was synthesized by procedures analogous to those outlines in Example 20, Steps 2-3 using (S)-2-((tert-butoxycarbonyl)amino)-3-(2-fluorophenyl)propanoic acid instead of (tert-butoxycarbonyl)-L-phenylalanine and Example 9, Steps 3-7. ¹H NMR (400 MHz, DMSO-d₆) δ 13.06 (bs, 1H), 8.27 (d, J=4.0 Hz, 1H), 7.39-7.33 (m, 3H), 7.29-7.24 (m, 1H), 7.21-7.17 (m, 1H), 7.11 (d, J=8.0 Hz, 1H), 7.08-7.05 (m, 1H), 7.02-6.99 (m, 2H), 4.87-4.81 (m, 1H), 4.78-4.71 (m, 1H), 4.39 (t, J=8.0 Hz, 1H), 4.15 (dd, J=8.0, 4.0 Hz, 1H), 3.03 (dd, J=16.0, 4.0 Hz, 1H), 2.84 (dd, J=16.0, 8.0 Hz, 1H), 1.50 (d, J=4.0 Hz, 3H). LCMS calc. for C₂₄H₂₁ClF₂N₃O₄ [M+H]⁺: m/z=488.1; Found: 488.1.

### Example 62. 6-Chloro-3-(((R)-1-(3-fluoro-5-((S)-4-(3-methylbenzyl)-2-oxooxazolidin-3-yl)phenyl)ethyl)amino)picolinic acid

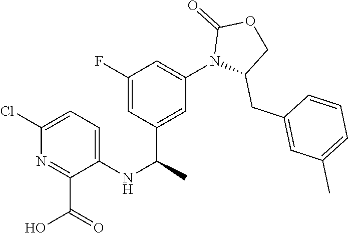 [SMILES: Cc1cccc(C[C@H]2COC(=O)N2c2cc(F)cc([C@@H](C)Nc3ccc(Cl)nc3C(=O)O)c2)c1]

The title compound was synthesized by procedures analogous to those outlines in Example 20, Steps 2-3 using (S)-2-((tert-butoxycarbonyl)amino)-3-(m-tolyl)propanoic acid instead of (tert-butoxycarbonyl)-L-phenylalanine and Example 9, Steps 3-7. ¹H NMR (400 MHz, DMSO-d₆) δ 13.05 (bs, 1H), 8.27 (d, J=8.0 Hz, 1H), 7.41-7.38 (m, 2H), 7.33 (d, J=8.0 Hz, 1H), 7.11-7.06 (m, 2H), 7.01 (t, J=8.0 Hz, 2H), 6.90 (s, 1H), 6.85 (d, J=8.0 Hz, 1H), 4.89-4.83 (m, 1H), 4.82-4.75 (m, 1H), 4.35 (t, J=8.0 Hz, 1H), 4.14 (dd, J=12.0, 4.0 Hz, 1H), 2.87 (dd, J=12.0, 4.0 Hz, 1H), 2.78 (dd, J=16.0, 8.0 Hz, 1H), 2.18 (s, 3H), 1.52 (d, J=8.0 Hz, 3H). LCMS calc. for C₂₅H₂₃ClFN₃NaO₄ [M+Na]⁺: m/z=506.1; Found: 506.2.

### Example A: PI3K Pathway Activation Assay

The inhibitory activity of the compounds of the disclosure was evaluated by measuring phosphorylation of AKT on Ser473 as a readout of the PI3K pathway using HTRF (CisBio catalog number: 64AKSPE). These studies were conducted in the T-47D (heterozygous PIK3CA H1047R) and SKBR3 (PIK3CA WT) cell lines. Cells were maintained in a 37° C. incubator at 5% CO₂ in the following media: T-47D: RPMI 1640, ATCC© Modification (Gibco, A10491-01) supplemented with 10% v/v FBS (Gibco, 26140-079), 1% penicillin streptomycin (Gibco, 15140-122), and 7.4 ug/mL insulin (MilliporeSigma, I9278); SKBR3: McCoy's 5a (Modified) Medium (Gibco, 16600-082) supplemented with 10% v/v FBS (Gibco, 26140-079). Cells were seeded in 384-well plates at a density of 4,000 cells/well. Compounds dissolved in DMSO were added using a digital dispense (D300E, Tecan) 10-point serial dilution. After two hours of treatment, cells were lysed for thirty minutes and then incubated with detection reagents per HTRF kit material and manufacturer's instructions. Fluorescence signal was measured with a multimode plate reader (Envision 2105, Perkin Elmer). Fluorescent signal was normalized to background and DMSO controls to obtain percent inhibition/activity for each compound. The results are summarized in Table 5.

TABLE 5
Biological Data

| Example | T47D pAKT IC₅₀ | SKBR3 PAKT IC₅₀ |
|---|---|---|
| 1 | +++ | −− |
| 2 | +++ | −− |
| 3 | +++ | −− |
| 4 | +++ | −− |
| 5 | +++ | −− |
| 6 | +++ | −− |
| 7 | +++ | −− |
| 8 | ++ | −− |
| 9 | ++ | −− |
| 10 | ++++ | −− |
| 11 | ++++ | + |
| 12 | ++ ++ | ++ |
| 13 | +++ | −− |
| 14 | +++ | −− |
| 15 | ++ | −− |
| 16 | +++ | −− |
| 17 | ++ | −− |
| 18 | ++++ | + |
| 19 | ++ | −− |
| 20 | ++++ | + |
| 21 | ++++ | ++ |
| 22 | +++ | −− |
| 23 | ++++ | + |
| 24 | ++++ | + |
| 25 | +++ | −− |
| 26 | ++++ | + |
| 27 | ++++ | + |
| 28 | +++ | −− |
| 29 | ++++ | + |
| 30 | +++ | −− |
| 31 | +++ | −− |
| 32 | +++ | −− |
| 33 | +++ | −− |
| 34 | +++ | −− |
| 35 | +++ | −− |
| 36 | +++ | −− |
| 37 | ++++ | −− |
| 38 | +++ | −− |
| 39 | ++++ | −− |
| 40 | ++++ | −− |
| 41 | +++ | −− |
| 42 | ++++ | −− |
| 43 | ++++ | −− |
| 44 | +++ | −− |
| 45 | ++++ | −− |
| 46 | ++++ | −− |
| 47 | ++++ | −− |
| 48 | ++++ | + |
| 49 | ++++ | + |
| 50 | +++ | −− |
| 51 | +++ | −− |
| 52 | +++ | −− |
| 53 | +++ | −− |
| 54 | +++ | −− |
| 55 | ++ | −− |
| 56 | +++ | −− |
| 57 | +++ | −− |
| 58 | ++++ | + |
| 59 | +++ | + |
| 60 | ++++ | ++ |
| 61 | ++++ | ++ |
| 62 | +++ | −− |

In Table 5, a “+” denotes an IC₅₀ value of >10000 nM; a “++” denotes an IC₅₀ value of 1000 nM < IC50 ≤10000 nM; a “+++” denotes an IC₅₀ value of 100 nM < IC50 <1000 nM; a “++++” denotes an IC₅₀ value of <100 nM.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A compound of Formula (I) 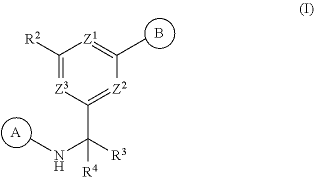
or a pharmaceutically acceptable salt, N-oxide, or stereoisomer thereof, wherein:
Ring A is aryl or a 5-7 membered heteroaryl ring comprising 1-4 heteroatoms selected from N, O, and S, wherein in the aryl or 5-7 membered heteroaryl ring is optionally substituted with one or more groups independently selected from D, halogen, C₁-C₈ alkoxy, C₁-C₈ haloalkoxy, C₁-C₈ alkyl, haloalkyl, —OH, —CN, —NO2, —C₂-C₆ alkenyl, —C₂-C₆ alkynyl, aryl, heteroaryl, cycloalkyl, cycloalkenyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)R^{c}R^{d}, —P(O)(OR^{b})(OR^{b}), —B(OR^{c})(OR^{d}), —SiR^{b}₃, —S(O)₂R^{b}, —C(O)NR^{b}OR^{b}, —S(O)₂OR^{b}, —OS(O)₂OR^{b}, and —OPO(OR^{b})(OR^{b}); wherein each C₁-C₈ alkyl or haloalkyl is optionally substituted by 1-6 groups selected from D, halogen, —OH, —CN, —OR^{a}, —SR^{a}, —NR^{a}R^{d}, or NR^{c}R^{d};
Ring B is 4-13 membered heterocycloalkyl, 4-10 membered heterocycloalkenyl, or 4-10 membered heteroaryl, wherein the 4-13 membered heterocycloalkyl, 4-10 membered heterocycloalkenyl, or 4-10 membered heteroaryl is optionally substituted with one or more groups independently selected from D, oxo, ═NR^{a}, ═N—OR^{a}, ═N—CN, ═S, halogen, C₁-C₈ alkoxide, C₁-C₁₀ alkyl, haloalkyl, —OH, —CN, —NO2, —C₂-C₁₀ alkenyl, —C₂-C₁₀ alkynyl, aryl, heteroaryl, cycloalkyl, cycloalkenyl, heterocycloalkyl, heterocycloalkenyl, C₀-C₄alk-aryl, C₀-C₄alk-heteroaryl, —OR^{a}, —SR^{a}, —NR^{c}R^{d}, —NR^{a}R^{c}, —C(O)R^{b}, —OC(O)R^{b}, —C(O)OR^{b}, —C(O)OR^{c}, —C(O)NR^{c}R^{d}, —C(═NR)NR^{b}R^{c}, —C(═NOR)NR^{b}R^{c}, —C(═NCN)NR^{b}R^{c}, —C(═NR^{b})NR^{c}R^{d}, —C(═NOR^{b})NR^{c}R^{d}, —C(═NCN)NR^{c}R^{d}, —P(OR^{c})₂, —P(O)R^{c}R^{b}, —P(O)R^{c}R^{d}, —P(O)OR^{c}OR^{b}, —S(O)R^{b}, —S(O)NR^{c}R^{d}, —S(O)₂NR^{c}R^{d}, —S(O)(═NR^{b})R^{b}, —SF₅, —P(O)R^{b}R^{c}, —P(O)(OR^{b})(OR^{b}), —B(OR^{c})(OR^{d}), —S(O)₂R^{b}, —C(O)NR^{b}OR^{b}, —SiR^{b}₃, —S(O)₂OR^{b}, —OS(O)₂OR^{b}, —OPO(OR^{b})(OR^{b}) and -L-W; wherein each C₁-C₈ alkoxide, C₁-C₁₀ alkyl, haloalkyl, —C₂-C₁₀ alkenyl, —C₂-C₁₀ alkynyl, aryl, heteroaryl, cycloalkyl, cycloalkenyl, heterocycloalkyl, heterocycloalkenyl, C₀-C₄alk-aryl, or C₀-C₄alk-heteroaryl is optionally substituted by 1-6 R^{f} groups;
L is absent or is C₁-C₈ alkylene, —O—, —N(R^{a})—, —S— or 3-10 membered cycloalkylene;
W is a 5-10 membered heteroaryl ring comprising 1-4 heteroatoms selected from N, O, and S; a 5-12 membered heterocyclic group comprising 1-4 heteroatoms selected from N, O, and S; C₁-C₈ alkyl; haloalkyl; —C₂-C₆ alkenyl; —C₂-C₆ alkynyl; aryl; cycloalkyl; cycloalkenyl; heterocyclo-alkenyl; NR^{c}R^{d}; OR^{b}; or SR^{b}; each of which is optionally substituted by 1-6 R^{f} groups;
Z¹, Z², and Z³ are each independently CR² or N;
each R² is independently H, D, halogen, C₁-C₈ alkoxide, C₁-C₈ alkyl, haloalkyl, —OH, —CN, —NO2, —C₂-C₆ alkenyl, —C₂-C₆ alkynyl, aryl, heteroaryl, cycloalkyl, cycloalkenyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)R^{c}R^{d}, —P(O)(OR^{b})(OR^{b}), —B(OR^{c})(OR^{d}), SiR^{b}₃, —S(O)₂R^{b}, —C(O)NR^{b}OR^{b}, —S(O)₂OR^{b}, —OS(O)₂OR^{b}, or —OPO(OR^{b})(OR^{b}); wherein said C₁-C₈ alkyl is optionally substituted by 1-6 groups selected from D, halogen, —OH, —CN, —OR^{a}, —SR^{a}, —NR^{a}R^{d}, or NR^{c}R^{d};
each R³ and R⁴ is independently H, D, C₁-C₈ alkyl, haloalkyl, or CN; wherein said C₁-C₈ alkyl is optionally substituted by 1-6 groups selected from D, halogen, —OH, —CN, —OR^{a}, —SR^{a}, —NR^{a}R^{d}, or —NR^{c}R^{d};
or R³ and R⁴, together with the atom to which they are both attached, are combined to form a C₃-C₇ cycloalkyl or C₄-C₈ heterocycloalkyl, wherein the C₃-C₇ cycloalkyl or C₄-C₈ heterocycloalkyl is optionally substituted by 1-6 groups selected from D, halogen, —OH, —CN, —OR^{a}, —SR^{a}, —NR^{a}R^{d}, or —NR^{c}R^{d};
each R^{a} is independently H, D, —C(O)R^{b}, —C(O)OR^{c}, —C(O)NR^{c}R^{d}, —C(═NR)NR^{b}R^{c}, —C(═NOR^{b})NR^{b}R^{c}, —C(═NCN)NR^{b}R^{c}, —C(═NR^{b})NR^{c}R^{d}, —C(═NOR^{b})NR^{c}R^{d}, —C(═NCN)NR^{c}R^{d}, —P(OR^{c})₂, —P(O)R^{c}R^{b}, —P(O)R^{c}R^{d}, —P(O)OR^{c}OR^{b}, —S(O)R^{b}, —S(O)NR^{c}R^{d}, —S(O)₂R^{b}, —S(O)₂NR^{c}R^{d}, —SiR^{b}₃, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl, wherein each —C₁-C₁₀alkyl, —C₂-C₁₀ alkenyl, —C₂-C₁₀ alkynyl, aryl, cycloalkyl, cycloalkenyl, heteroaryl, heterocycloalkyl, and heterocycloalkenyl of R^{a} is optionally substituted by 1-6 R^{f} groups;
each R^{b} is independently H, D, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl; wherein each —C₁-C₆ alkyl, —C₂-C₆ alkenyl, —C₂-C₆ alkynyl, aryl, cycloalkyl, cycloalkenyl, heteroaryl, heterocycloalkyl, and heterocycloalkenyl of R^{b} is optionally substituted by 1-6 R^{f} groups;
each R^{c} or R^{d} is independently H, D, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl; wherein each —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl of R^{c} or R^{d} is optionally substituted by 1-6 R^{f} groups;
or R^{c} and R^{d}, together with the atom to which they are both attached, form a monocyclic or multicyclic heterocycloalkyl, or a monocyclic or multicyclic heterocycloalkenyl group, wherein the monocyclic or multicyclic heterocycloalkyl, or a monocyclic or multicyclic heterocycloalkenyl group is optionally substituted by 1-6 R^{f} groups;
each R^{f} is independently D, oxo, halogen, C₁-C₈ alkoxide, C₁-C₈ alkyl, haloalkyl, —OH, —CN, —NO2, —C₂-C₆ alkenyl, —C₂-C₆ alkynyl, aryl, heteroaryl, cycloalkyl, cycloalkenyl, heterocycloalkyl, heterocycloalkenyl, —OR^{g0}, —SR^{g0}, —NR^{g2}R^{g3}, —NR^{g0}R^{g2}, —C(O)R^{g1}, —OC(O)R^{g1}, —C(O)OR^{g1}, —C(O)NR^{g2}R^{g3}, —S(O)R^{g1}, —S(O)₂NR^{g2}R^{g3}, —S(O)(═NR^{g1})R^{g1}, —SF₅, —P(O)R^{g1}R^{g1}, —P(O)R^{g2}R^{g3}, —P(O)(OR^{g1})(OR^{g1}), SiR^{g1}₃, —B(OR^{g2})(OR^{g3}), —S(O)₂R^{g1}, —C(O)NR^{g1}OR^{g1}, —S(O)₂OR^{g1}, —OS(O)₂OR^{g1}, or —OPO(OR^{g1})(OR^{g1}); wherein each C₁-C₈ alkoxide, C₁-C₈ alkyl, haloalkyl, —C₂-C₆ alkenyl, —C₂-C₆ alkynyl, aryl, heteroaryl, cycloalkyl, cycloalkenyl, heterocycloalkyl, or heterocycloalkenyl is optionally substituted by 1-6 R^{g} groups;
each R^{g0} is independently H, D, —C(O)R^{g1}, —C(O)OR^{g2}, —C(O)NR^{g2}R^{g3}, —C(═NR^{g1})N R^{g2}R^{g3}, —C(═NOR^{g1})NR^{g2}R^{g3}, —C(═NCN)NR^{g1}R^{g2}, —C(═NR^{g1})NR^{g2}R^{g3}, —C(═NOR^{g1})NR^{g2}R^{g3}, —C(═NCN)NR^{g2}R^{g3}, —P(OR^{g2})₂, —P(O)R^{g2}R^{g1}, —P(O)R^{g2}R^{g3}, —P(O)OR^{g2}OR^{g1}, —S(O)R^{g1}, —S(O)NR^{g2}R^{g3}, —S(O)₂R^{g1}, —S(O)₂NR^{g2}R^{g3}, —SiR^{g1}₃, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl, wherein each —C₁-C₁₀alkyl, —C₂-C₁₀ alkenyl, —C₂-C₁₀ alkynyl, aryl, cycloalkyl, cycloalkenyl, heteroaryl, heterocycloalkyl, and heterocycloalkenyl is optionally substituted by 1-6 R^{g} groups;
each R^{g1} is independently H, D, C₁-C₈ alkyl, haloalkyl, —C₂-C₆ alkenyl, —C₂-C₆ alkynyl, aryl, heteroaryl, cycloalkyl, cycloalkenyl, heterocycloalkyl, or heterocycloalkenyl, wherein each C₁-C₈ alkyl, haloalkyl, —C₂-C₆ alkenyl, —C₂-C₆ alkynyl, aryl, heteroaryl, cycloalkyl, cycloalkenyl, heterocycloalkyl, or heterocycloalkenyl is optionally substituted by 1-6 R^{g} groups;
each R^{g2} or R^{g3} is independently H, D, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl; wherein each —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, or heterocycloalkyl is optionally substituted by 1-6 R^{g} groups; or R^{g2} and R^{g3}, together with the atom to which they are both attached, form a monocyclic or multicyclic heterocycloalkyl, or a monocyclic or multicyclic heterocycloalkenyl group, wherein the monocyclic or multicyclic heterocycloalkyl, or monocyclic or multicyclic heterocycloalkenyl group is optionally substituted by 1-6 R^{g} groups;
each R^{g} is independently:
D; —CN; —NO2; oxo; halogen; —SF₅; —OR^{h}; —SR^{h}; —NR^{h}R^{h}; —SiR^{h}₃; —C(O)R^{h}; —OC(O)R^{h}; —C(O)OR^{h}; —OC(O)OR^{h}; —C(O)NR^{h}OR^{h}; —S(O)R^{h}; —S(O)₂R^{h}; —S(O)₂OR^{h}; —OS(O)₂OR^{h}; —S(O)(═NR^{h})R^{h}; —P(O)(OR^{h})(OR^{h}); —P(O)R^{h}R^{h}; —OPO(OR^{h})(OR^{h}); —C(O)NR^{h}R^{h}; —OC(O)NR^{h}; —S(O)₂NR^{h}R^{h}; —B(OR^{h})(OR^{h}); —OC(═NR^{h})NR^{h}R^{h}; —OC(═NOR^{h})NR^{h}R^{h}; —OC(═NCN)NR^{h}; —OP(OR^{h})₂; —OP(O)R^{h}R^{h}; —OP(O)OR^{h}OR^{h}; —OS(O)R^{h}; —OS(O)NR^{h}R^{h}; —OS(O)₂R^{h}; —OS(O)₂NR^{h}R^{h}; —OSiR^{h}₃; —S—C(O)R^{h}; —S—C(O)OR^{h}; —S—C(O)NR^{h}R^{h}; —S—C(═NR^{h})NR^{h}R^{h}; —S—C(═NOR^{h})NR^{h}R^{h}; —S—C(═NCN)NR^{h}R^{h}; —S—P(OR^{h})₂; —S—P(O)R^{h}R^{h}; —S—P(O)OR^{h}OR^{h}; —S(O)R^{h}; —S(O)NR^{h}R^{h}; —S(O)₂R^{h}; —S(O)₂NR^{h}R^{h}; —SSiR^{h}₃; —NR^{h}C(O)R^{h}; —NR^{h}C(O)OR^{h}; —NR^{h}C(O)NR^{h}R^{h}; —NR^{h}C(═NR^{h})NR^{h}R^{h}; —NR^{h}C(═NOR^{h})NR^{h}R^{h}; —NR^{h}C(═NCN)NR^{h}; —NR^{h}P(OR^{h})₂; —NR^{h}P(O)R^{h}R^{h}; —NR^{h}P(O)OR^{h}OR^{h}; —NR^{h}S(O)R^{h}; —NR^{h}S(O)NR^{h}R^{h}; —NR^{h}S(O)₂R^{h}; —NR^{h}S(O)₂NR^{h}R^{h}; —NR^{h}SiR^{h}₃; C₁-C₈ alkoxide; C₁-C₈ alkyl; haloalkyl; —C₂-C₆ alkenyl; —C₂-C₆ alkynyl; aryl; heteroaryl; cycloalkyl; cycloalkenyl; heterocycloalkyl; or heterocycloalkenyl;
wherein the C₁-C₈ alkoxide, C₁-C₈ alkyl, haloalkyl, —C₂-C₆ alkenyl, —C₂-C₆ alkynyl, aryl, heteroaryl, cycloalkyl, cycloalkenyl, heterocycloalkyl, or heterocycloalkenyl is optionally substituted by 1-6 groups selected from D, halogen, —CN, —NO2, oxo, —OR^{h}; —SR^{h}; or —NR^{h};
wherein each R^{h} is independently H, D, C₁-C₈alkyl, haloalkyl, —C₂-C₆ alkenyl, —C₂-C₆ alkynyl, aryl, heteroaryl, cycloalkyl, cycloalkenyl, heterocycloalkyl, or heterocycloalkenyl; or
wherein two R^{h} attached to the same atom may form a monocyclic or multicyclic heterocycloalkyl, or a monocyclic or multicyclic heterocycloalkenyl group.

2. The compound of claim 1, that is a compound of Formula (II) 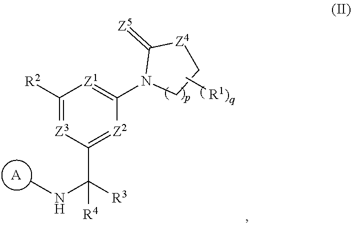
or a pharmaceutically acceptable salt, N-oxide, or stereoisomer thereof; wherein
Z⁴ is O, NR^{a}, C(R¹)₂, or S;
Z⁵ is O, NR^{a}, N—OR^{a}, N—CN, or S;
p is 1, 2 or 3; and
q is 0, 1, 2, 3, 4, 5, 6, 7, or 8;
each R¹ is independently H, D, oxo, ═NR^{a}, ═N—OR^{a}, ═N—CN, ═S, halogen, C₁-C₈ alkoxide, C₁-C₁₀ alkyl, haloalkyl, —OH, —CN, —NO2, —C₂-C₁₀ alkenyl, —C₂-C₁₀ alkynyl, aryl, heteroaryl, cycloalkyl, cycloalkenyl, heterocycloalkyl, heterocycloalkenyl, C₀-C₄alk-aryl, C₀-C₄alk-heteroaryl, —OR^{a}, —SR^{a}, —NR^{c}R^{d}, —NR^{a}R^{c}, —C(O)R^{b}, —OC(O)R^{b}, —C(O)OR^{b}, —C(O)OR^{c}, —C(O)NR^{c}R^{d}, —C(═NR)NR^{b}R^{c}, —C(═NOR^{b})NR^{b}R^{c}, —C(═NCN)NR^{b}R^{c}, —C(═NR^{b})NR^{c}R^{d}, —C(═NOR^{b})NR^{c}R^{d}, —C(═NCN)NR^{c}R^{d}, —P(OR^{c})₂, —P(O)R^{c}R^{b}, —P(O)R^{c}R^{d}, —P(O)OR^{c}OR^{b}, —S(O)R^{b}, —S(O)NR^{c}R^{d}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}), —S(O)₂R^{b}, —C(O)NR^{b}OR^{b}, —SiR^{b}₃, —S(O)₂OR^{b}, —OS(O)₂OR^{b}, —OPO(OR^{b})(OR^{b}) or -L-W; wherein each C₁-C₈ alkoxide, C₁-C₁₀ alkyl, haloalkyl, —C₂-C₁₀ alkenyl, —C₂-C₁₀ alkynyl, aryl, heteroaryl, cycloalkyl, cycloalkenyl, heterocycloalkyl, heterocycloalkenyl, C₀-C₄alk-aryl, or C₀-C₄alk-heteroaryl is optionally substituted by 1-6 R^{f} groups.

3-4. (canceled)

5. The compound of claim 1, wherein Ring A is: 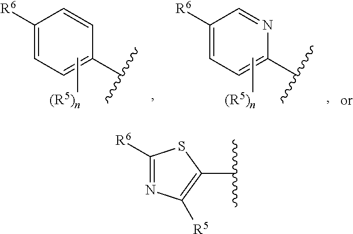
wherein
n is 1, 2, or 3;
each R⁵ is independently H, D, halogen, C₁-C₈ alkoxide, C₁-C₈ alkyl, haloalkyl, —OH, —CN, —NO2, —C₂-C₆ alkenyl, —C₂-C₆ alkynyl, aryl, heteroaryl, cycloalkyl, cycloalkenyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)R^{c}R^{d}, —P(O)(OR^{b})(OR^{b}), SiR^{b}₃, —B(OR^{c})(OR^{d}), —S(O)₂R^{b}, —C(O)NR^{b}OR^{b}, —S(O)₂OR^{b}, —OS(O)₂OR^{b}, or —OPO(OR^{b})(OR^{b}); wherein said C₁-C₈ alkyl or haloalkyl is optionally substituted by 1-6 groups selected from D, halogen, —OH, —CN, —OR^{a}, —SR^{a}, —NR^{a}R^{d}, or NR^{c}R^{d}; and
R⁶ is —F, —Cl, —Br, —I, C₁-C₄-alkyl, C₁-C₄-alkoxy, C₁-C₄-haloalkoxy, or —CN.

6-8. (canceled)

9. The compound of claim 5, that is a compound of Formula (III) 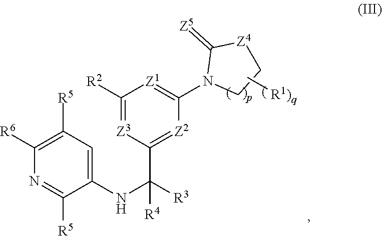
or a pharmaceutically acceptable salt, N-oxide, or stereoisomer thereof.

10. The compound of claim 9, that is a compound of Formula (IV) or (V) 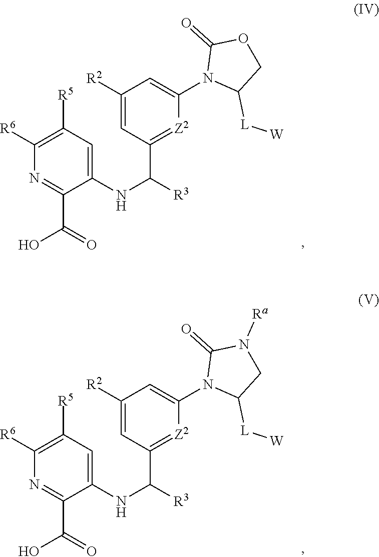
or a pharmaceutically acceptable salt, N-oxide, or stereoisomer thereof.

11. The compound of claim 10, wherein R^{a} in Formula (V) is H, C₁-C₆ alkyl, or 3-6 membered cycloalkyl.

12. (canceled)

13. The compound of claim 5, wherein at least one R⁵ is —CO₂H, —CONH₂, —COOCH₃, —C(O)H, or —CN.

14. (canceled)

15. The compound of claim 1, wherein at least one R² is H, C₁-C₈ alkyl, CD₃, CF₃, halogen, CN or CHF₂.

16-21. (canceled)

22. The compound of claim 1, wherein R³ is H or CD₃ or C₁-C₈ alkyl.

23. (canceled)

24. The compound of claim 1, wherein R⁴ is H or C₁-C₈ alkyl.

25. (canceled)

26. The compound of claim 1, wherein each Z¹, Z² and Z³ is CR².

27. The compound of claim 1, wherein Z¹ and Z³ are CR².

28. The compound of claim 1, wherein at least one of Z¹, Z² and Z³ is N.

29-30. (canceled)

31. The compound of claim 5, wherein R⁶ is —F, —Cl, —CH₃, —OCH₃ or —OCF₂H.

32-37. (canceled)

38. The compound of claim 2, wherein Z⁴ and Z⁵ are O.

39. The compound of claim 2, wherein Z⁴ is —NR^{a}.

40. (canceled)

41. The compound of claim 2, wherein R^{a} is —CH₃.

42. (canceled)

43. The compound of claim 1, wherein Ring B is substituted with at least one -L-W.

44. The compound of claim 2, wherein at least one R¹ is -L-W.

45. The compound of claim 1, wherein W is aryl optionally substituted by 1-6 R^{f} groups.

46. (canceled)

47. The compound of claim 1, wherein W is phenyl, 4-fluorophenyl or 2,4-difluorophenyl.

48. (canceled)

49. The compound of claim 1, wherein W is a 5-12 membered heterocyclic group comprising 1-4 heteroatoms selected from N, O, and S, wherein the 5-12 membered heterocyclic group is optionally substituted by 1-6 R^{f} groups.

50. The compound of claim 1, wherein W is a 5-10 membered heteroaryl ring comprising 1-4 heteroatoms selected from N, O, and S, wherein the 5-10 membered heteroaryl ring is optionally substituted by 1-6 R^{f} groups.

51. The compound of claim 1, wherein L is absent or is C₁-C₈ alkylene.

52. (canceled)

52. The compound of claim 1, that is
3-((1-(3-((S)-4-Benzyl-2-oxooxazolidin-3-yl)-5-methylphenyl)ethyl)amino)-6-chloro-picolinic acid;
3-(1-(3-((S)-4-Benzyl-2-oxooxazolidin-3-yl)-5-methylphenyl)ethyl)amino)-6-chloro-picolinic acid;
3-((1-(3-((S)-4-Benzyl-2-oxooxazolidin-3-yl)-5-methylphenyl)ethyl)amino)-6-fluoro-picolinic acid;
3-((1-(3-((S)-4-benzyl-2-oxooxazolidin-3-yl)-5-fluorophenyl)ethyl)amino)-6-chloro-picolinic acid;
3-((1-(3-((S)-4-Benzyl-2-oxooxazolidin-3-yl)-5-fluorophenyl)ethyl)amino)-6-fluoro-picolinic acid;
3-((1-(3-((S)-4-Benzyl-2-oxooxazolidin-3-yl)-5-chlorophenyl)ethyl)amino)-6-chloro-picolinic acid;
3-((1-(3-((S)-4-Benzyl-2-oxooxazolidin-3-yl)-5-chlorophenyl)ethyl)amino)-6-fluoro-picolinic acid;
3-((1-(3-((S)-4-Benzyl-2-oxooxazolidin-3-yl)-5-(trifluoromethyl)phenyl)ethyl)amino)-6-fluoropicolinic acid;
3-(((R)-1-(3-((S)-4-(4-Bromobenzyl)-2-oxooxazolidin-3-yl)-5-methylphenyl)ethyl) amino)-6-chloropicolinic acid;
3-(((R)-1-(3-((S)-4-Benzyl-2-oxooxazolidin-3-yl)-5-fluorophenyl)ethyl)amino)-6-chloro-picolinic acid;
3-[1-[6-[(4S)-4-Benzyl-2-oxo-1,3-oxazolidin-3-yl]-4-methyl-2-pyridinyl]ethylamino]-6-fluoropyridine-2-carboxylic acid;
3-(((R)-1-(6-((S)-4-Benzyl-2-oxooxazolidin-3-yl)-4-methylpyridin-2-yl)ethyl)amino)-6-chloropicolinic acid;
3-((1-(6-((S)-4-Benzyl-2-oxooxazolidin-3-yl)-4-methylpyridin-2-yl)ethyl)amino)picolinic acid;
3-((1-(6-((S)-4-Benzyl-2-oxooxazolidin-3-yl)-4-methylpyridin-2-yl)ethyl)amino)-6-methylpicolinic acid;
3-((1-(6-((S)-4-Benzyl-2-oxooxazolidin-3-yl)-4-methylpyridin-2-yl)ethyl)amino)-6-(trifluoromethyl)picolinic acid;
3-((1-(6-((S)-4-Benzyl-2-oxooxazolidin-3-yl)-4-methylpyridin-2-yl)ethyl)amino)-6-methoxypicolinic acid;
2-((1-(6-((S)-4-Benzyl-2-oxooxazolidin-3-yl)-4-methylpyridin-2-yl)ethyl)amino)benzoic acid;
3-((1-(6-((S)-4-Benzyl-2-oxooxazolidin-3-yl)-4-methylpyridin-2-yl)ethyl)amino)-6-bromopicolinic acid;
3-((1-(6-((S)-4-Benzyl-2-oxooxazolidin-3-yl)-4-methylpyridin-2-yl)ethyl)amino)-6-cyanopicolinic acid;
or a pharmaceutically acceptable salt, N-oxide, or stereoisomer thereof.

53. The compound of claim 1, that is
6-Chloro-3-(((R)-1-(6-((S)-4-(2-fluoro-3-methylbenzyl)-2-oxooxazolidin-3-yl)-4-methyl-pyridin-2-yl)ethyl)amino)picolinic acid;
6-Chloro-3-(((R)-1-(6-((S)-4-((3-fluoropyridin-4-yl)methyl)-2-oxooxazolidin-3-yl)-4-methylpyridin-2-yl)ethyl)amino)picolinic acid;
3-(((R)-1-(6-((S)-4-(2-Bromobenzyl)-2-oxooxazolidin-3-yl)-4-methylpyridin-2-yl)ethyl) amino)-6-chloropicolinic acid;
6-Chloro-3-(((R)-1-(4-methyl-6-((S)-2-oxo-4-(4-(trifluoromethyl)benzyl)oxazolidin-3-yl)pyridin-2-yl)ethyl)amino)picolinic acid;
6-Chloro-3-(((R)-1-(6-((S)-4-(4-fluorobenzyl)-2-oxooxazolidin-3-yl)-4-methylpyridin-2-yl)ethyl)amino)picolinic acid;
3-(((R)-1-(6-((S)-4-(4-(tert-Butoxycarbonyl)benzyl)-2-oxooxazolidin-3-yl)-4-methylpyridin-2-yl)ethyl)amino)-6-chloropicolinic acid;
6-Chloro-3-(((R)-1-(6-((S)-4-(2,4-difluorobenzyl)-2-oxooxazolidin-3-yl)-4-methylpyridin-2-yl)ethyl)amino)picolinic acid;
6-Chloro-3-(((R)-1-(6-((S)-4-(2-fluoro-4-methoxybenzyl)-2-oxooxazolidin-3-yl)-4-methyl-pyridin-2-yl)ethyl)amino)picolinic acid;
6-Chloro-3-(((R)-1-(4-methyl-6-((S)-2-oxo-4-(pyridin-4-ylmethyl)oxazolidin-3-yl)pyridin-2-yl)ethyl)amino)picolinic acid;
6-Chloro-3-(((R)-1-(6-((S)-4-((5-fluoro-2-methoxypyridin-4-yl)methyl)-2-oxooxazolidin-3-yl)-4-methylpyridin-2-yl)ethyl)amino)picolinic acid;
6-Chloro-3-(((R)-1-(6-((S)-4-((5-fluoro-2-methylpyridin-4-yl)methyl)-2-oxooxazolidin-3-yl)-4-methylpyridin-2-yl)ethyl)amino)picolinic acid;
6-Chloro-3-(((R)-1-(4-methyl-6-((S)-4-(naphthalen-1-ylmethyl)-2-oxooxazolidin-3-yl)pyridin-2-yl)ethyl)amino)picolinic acid;
6-Chloro-3-(((R)-1-(4-methyl-6-((S)-4-((1-methyl-1H-pyrazol-4-yl)methyl)-2-oxooxazolidin-3-yl)pyridin-2-yl)ethyl)amino)picolinic acid;
6-Chloro-3-(((R)-1-(4-methyl-6-((S)-4-((1-methyl-1H-pyrazol-3-yl)methyl)-2-oxooxazolidin-3-yl)pyridin-2-yl)ethyl)amino)picolinic acid;
6-Chloro-3-(((R)-1-(4-methyl-6-((S)-4-((1-methyl-1H-pyrazol-5-yl)methyl)-2-oxooxazolidin-3-yl)pyridin-2-yl)ethyl)amino)picolinic acid;
6-Chloro-3-(((R)-1-(6-((S)-4-((1,3-dimethyl-1H-pyrazol-4-yl)methyl)-2-oxooxazolidin-3-yl)-4-methylpyridin-2-yl)ethyl)amino)picolinic acid;
6-Chloro-3-(((R)-1-(6-((S)-4-((3-fluoro-1-methyl-1H-pyrazol-4-yl)methyl)-2-oxooxazolidin-3-yl)-4-methylpyridin-2-yl)ethyl)amino)picolinic acid;
6-Chloro-3-(((R)-1-(4-methyl-6-((S)-4-((1-methyl-1H-indol-3-yl)methyl)-2-oxooxazolidin-3-yl)pyridin-2-yl)ethyl)amino)picolinic acid;
6-Chloro-3-(((R)-1-(4-methyl-6-((S)-4-((1-methyl-1H-indazol-3-yl)methyl)-2-oxooxazolidin-3-yl)pyridin-2-yl)ethyl)amino)picolinic acid;
6-Chloro-3-(((R)-1-(4-methyl-6-((S)-4-((1-methyl-1H-pyrrolo[2,3-b]pyridin-3-yl)methyl)-2-oxooxazolidin-3-yl)pyridin-2-yl)ethyl)amino)picolinic acid;
6-Chloro-3-(((R)-1-(4-methyl-6-((S)-2-oxo-4-(pyrazolo[1,5-a]pyridin-5-ylmethyl) oxazolidin-3-yl)pyridin-2-yl)ethyl)amino)picolinic acid;
6-Chloro-3-(((R)-1-(6-((S)-4-(imidazo[1,5-a]pyridin-6-ylmethyl)-2-oxooxazolidin-3-yl)-4-methylpyridin-2-yl)ethyl)amino)picolinic acid;
6-Chloro-3-(((R)-1-(4-methyl-6-((S)-4-((1-methyl-1H-indazol-5-yl)methyl)-2-oxooxazolidin-3-yl)pyridin-2-yl)ethyl)amino)picolinic acid;
6-Chloro-3-(((R)-1-(4-methyl-6-((S)-4-((2-methyl-2H-indazol-5-yl)methyl)-2-oxooxazolidin-3-yl)pyridin-2-yl)ethyl)amino)picolinic acid;
6-Chloro-3-(((R)-1-(6-((S)-4-(imidazo[1,2-a]pyridin-8-ylmethyl)-2-oxooxazolidin-3-yl)-4-methylpyridin-2-yl)ethyl)amino)picolinic acid;
6-Chloro-3-(((R)-1-(4-methyl-6-((S)-2-oxo-4-(pyrazolo[1,5-a]pyridin-4-ylmethyl) oxazolidin-3-yl)pyridin-2-yl)ethyl)amino)picolinic acid;
6-Chloro-3-(((R)-1-(4-methyl-6-((S)-2-oxo-4-(pyrazolo[1,5-a]pyridin-3-ylmethyl) oxazolidin-3-yl)pyridin-2-yl)ethyl)amino)picolinic acid;
6-Chloro-3-(((R)-1-(6-((S)-4-(cyclobutylmethyl)-2-oxooxazolidin-3-yl)-4-methylpyridin-2-yl)ethyl)amino)picolinic acid;
6-Chloro-3-(((R)-1-(6-((S)-4-(3-cyanobenzyl)-2-oxooxazolidin-3-yl)-4-methylpyridin-2-yl)ethyl)amino)picolinic acid;
6-Chloro-3-(((R)-1-(6-((S)-4-(4-cyano-2-fluorobenzyl)-2-oxooxazolidin-3-yl)-4-methyl-pyridin-2-yl)ethyl)amino)picolinic acid;
3-(((R)-1-(6-((S)-4-((1H-Indol-3-yl)methyl)-2-oxooxazolidin-3-yl)-4-methylpyridin-2-yl)ethyl)amino)-6-chloropicolinic acid;
6-Chloro-3-(((R)-1-(6-((S)-4-(4-(dimethylcarbamoyl)benzyl)-2-oxooxazolidin-3-yl)-4-methylpyridin-2-yl)ethyl)amino)picolinic acid;
6-Chloro-3-(((R)-1-(6-((S)-4-(4-(methoxy(methyl)carbamoyl)benzyl)-2-oxooxazolidin-3-yl)-4-methylpyridin-2-yl)ethyl)amino)picolinic acid;
6-Chloro-3-(((R)-1-(4-methyl-6-((S)-2-oxo-4-phenethyloxazolidin-3-yl)pyridin-2-yl)ethyl) amino)picolinic acid;
3-(((R)-1-(6-((S)-4-Benzyl-4-methyl-2-oxooxazolidin-3-yl)-4-methylpyridin-2-yl)ethyl) amino)-6-chloropicolinic acid;
3-(((1R)-1-(6-(2-benzyl-5-oxopyrrolidin-1-yl)-4-methylpyridin-2-yl)ethyl)amino)-6-chloropicolinic acid;
3-(((R)-1-(6-((S)-4-Benzyl-2-oxo-1,3-oxazinan-3-yl)-4-methylpyridin-2-yl)ethyl)amino)-6-chloropicolinic acid;
3-(((R)-1-(6-((S)-3-Benzyl-5-oxomorpholino)-4-methylpyridin-2-yl)ethyl)amino)-6-chloropicolinic acid;
6-Chloro-3-(((R)-1-(6-((S)-5-(4-fluorobenzyl)-3-methyl-2-oxoimidazolidin-1-yl)-4-methylpyridin-2-yl)ethyl)amino)picolinic acid;
6-Chloro-3-(((R)-1-(6-((S)-5-(2,4-difluorobenzyl)-3-methyl-2-oxoimidazolidin-1-yl)-4-methylpyridin-2-yl)ethyl)amino)picolinic acid;
3-(((R)-1-(6-((S)-5-Benzyl-3-methyl-2-oxoimidazolidin-1-yl)-4-methylpyridin-2-yl)ethyl) amino)-6-chloropicolinic acid;
6-Chloro-3-(((R)-1-(3-fluoro-5-((S)-4-(2-fluorobenzyl)-2-oxooxazolidin-3-yl)phenyl)ethyl) amino)picolinic acid;
6-Chloro-3-(((R)-1-(3-fluoro-5-((S)-4-(3-methylbenzyl)-2-oxooxazolidin-3-yl)phenyl)ethyl) amino)picolinic acid;
or a pharmaceutically acceptable salt, N-oxide, or stereoisomer thereof.

54. The compound of claim 1, in the form of a pharmaceutically acceptable salt.

55. A pharmaceutical composition comprising a compound according to claim 1, or a pharmaceutically acceptable salt thereof, and a pharmaceutically acceptable excipient.

56. A method of treating a disease or disorder associated with modulation of phosphoinositide 3-kinase (PI3K), comprising administering to a patient in need thereof a therapeutically effective amount of a compound of claim 1 or a pharmaceutical composition comprising the compound.

57. The method of claim 56, wherein the PI3K is PI3Kα.

58. The method of claim 56, wherein the PI3K associated with the disease or disorder has a H1047R mutation.

59. The method of claim 56, wherein the disease or disorder is a cancer.

60. The method of claim 59, wherein the cancer is endometrial cancer, gastric cancer, leukemia, lymphoma, sarcoma, colorectal cancer, lung cancer, ovarian cancer, skin cancer, head and neck cancer, breast cancer, brain cancer, cervical cancer, bladder cancer, esophageal cancer, pancreatic cancer, bone cancer, hepatobiliary cancer, medulloblastoma, kidney cancer or prostate cancer.

61. The method of claim 56, wherein the disease or disorder is CLOVES syndrome (congenital lipomatous overgrowth, vascular malformations, epidermal naevi, scoliosis/skeletal and spinal syndrome), or PIK3CA-related overgrowth syndrome (PROS).

62. A method of inhibiting phosphoinositide 3-kinase (PI3K),
comprising administering to a patient in need thereof a therapeutically effective amount of a compound of claim 1 or a pharmaceutical composition comprising the compound.

63. A method of treating cancer or a disorder, the method comprising administering to a patient in need thereof a therapeutically effective amount of a compound of claim 1 or a pharmaceutical composition comprising the compound.

64. The method of claim 63, wherein the cancer is endometrial cancer, gastric cancer, leukemia, lymphoma, sarcoma, colorectal cancer, lung cancer, ovarian cancer, skin cancer, head and neck cancer, breast cancer, brain cancer, or prostate cancer.

65. (canceled)

66. The method of claim 63, wherein the disorder is CLOVES syndrome (congenital lipomatous overgrowth, vascular malformations, epidermal naevi, scoliosis/skeletal and spinal syndrome) or PIK3CA-related overgrowth syndrome (PROS).

67-68. (canceled)
